(19) United States
(12) Patent Application Publication
(10) Pub. No.: US 2013/0023534 A1
(43) Pub. Date: Jan. 24, 2013

(54) PYRAZOLYL-PYRIMIDINES AS KINASE INHIBITORS

(71) Applicants: Linda N. Casillas, Collegeville, PA (US); Subhas J. Chakravorty, Collegeville, PA (US); Patrick Eidam, Collegeville, PA (US); Pamela A. Haile, King of Prussia, PA (US); Terry Vincent Hughes, Collegeville, PA (US); Ami Lakdawala Shah, King of Prussia, PA (US); Lara Kathryn Leister, Collegeville, PA (US); Nathan Andrew Miller, Collegeville, PA (US); Attiq Rahman, Collegeville, PA (US); Clark A. Sehon, King of Prussia, PA (US); Gren Z. Wang, Collegeville, PA (US); Daohua Zhang, Collegeville, PA (US)

(72) Inventors: Linda N. Casillas, Collegeville, PA (US); Subhas J. Chakravorty, Collegeville, PA (US); Patrick Eidam, Collegeville, PA (US); Pamela A. Haile, King of Prussia, PA (US); Terry Vincent Hughes, Collegeville, PA (US); Ami Lakdawala Shah, King of Prussia, PA (US); Lara Kathryn Leister, Collegeville, PA (US); Nathan Andrew Miller, Collegeville, PA (US); Attiq Rahman, Collegeville, PA (US); Clark A. Sehon, King of Prussia, PA (US); Gren Z. Wang, Collegeville, PA (US); Daohua Zhang, Collegeville, PA (US)

(21) Appl. No.: 13/637,430

(22) PCT Filed: Mar. 26, 2011
(86) PCT No.: PCT/US11/30104

§ 371 Date: Sep. 26, 2012

Related U.S. Application Data
(60) Provisional application No. 61/317,918, filed on Mar. 26, 2010.

(51) Int. Cl. A61K 31/5377; C07D 413/14; C07D 401/14; C07D 409/14; C07D 417/14; C07D 401/12; C07D 413/12; A61K 31/506; C07D 405/14

(52) U.S. Cl. 514/236.5; 514/275; 544/122; 544/324

(57) ABSTRACT

Disclosed are compounds having the formula (I): wherein Z, n, R¹, R^{1A}, R³, R⁴, and R⁵ are as defined herein, and methods of making and using the same.

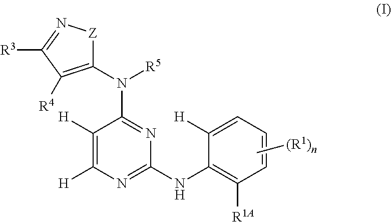

### BACKGROUND OF THE INVENTION

1. Field of the Invention

The present invention relates to pyrazolyl-pyrimidinyl diamines that inhibit RIP2 kinase and methods of making and using the same. Specifically, the present invention relates to substituted pyrazoles as RIP2 kinase inhibitors.

2. Background of the Invention

Receptor interacting protein-2 (RIP2) kinase, which is also referred to as CARD3, RICK, CARDIAK, or RIPK2, is a TKL family serine/threonine protein kinase involved in innate immune signaling. RIP2 kinase is composed of an N-terminal kinase domain and a C-terminal caspase-recruitment domain (CARD) linked via an intermediate (IM) region ((1998) J. Biol. Chem. 273, 12296-12300; (1998) Current Biology 8, 885-889; and (1998) J. Biol. Chem. 273, 16968-16975). The CARD domain of RIP2 kinase mediates interaction with other CARD-containing proteins, such as NOD1 and NOD2 ((2000) J. Biol. Chem. 275, 27823-27831 and (2001) EMBO reports 2, 736-742). NOD1 and NOD2 are cytoplasmic receptors which play a key role in innate immune surveillance. They recognize both gram positive and gram negative bacterial pathogens and are activated by specific peptidoglycan motifs, diaminopimelic acid (i.e., DAP) and muramyl dipeptide (MDP), respectively ((2007) J Immunol 178, 2380-2386).

Following activation, RIP2 kinase associates with NOD1 or NOD2 and appears to function principally as a molecular scaffold to bring together other kinases (TAK1, IKKα/β/γ) involved in NF-κB and mitogen-activated protein kinase activation ((2006) Nature Reviews Immunology 6, 9-20). RIP2 kinase undergoes a K63-linked polyubiquitination on lysine-209 which facilitates TAK1 recruitment ((2008) EMBO Journal 27, 373-383). This post-translational modification is required for signaling as mutation of this residue prevents NOD1/2 mediated NF-kB activation. RIP2 kinase also undergoes autophosphorylation on serine-176, and possibly other residues ((2006) Cellular Signalling 18, 2223-2229). Studies using kinase dead mutants (K47A) and non-selective small molecule inhibitors have demonstrated that RIP2 kinase activity is important for regulating the stability of RIP2 kinase expression and signaling ((2007) Biochem J 404, 179-190 and (2009) J. Biol. Chem. 284, 19183-19188).

Dysregulation of RIP2-dependent signaling has been linked to autoinflammatory diseases. Gain-of-function mutations in the NACHT-domain of NOD2 cause Blau Syndrome/Early-onset Sarcoidosis, a pediatric granulomateous disease characterized by uveitis, dermatitis, and arthritis ((2001) Nature Genetics 29, 19-20; (2005) Journal of Rheumatology 32, 373-375; (2005) Current Rheumatology Reports 7, 427-433; (2005) Blood 105, 1195-1197; (2005) European Journal of Human Genetics 13, 742-747; (2006) American Journal of Ophthalmology 142, 1089-1092; (2006) Arthritis & Rheumatism 54, 3337-3344; (2009) Arthritis & Rheumatism 60, 1797-1803; and (2010) Rheumatology 49, 194-196). Mutations in the LRR-domain of NOD2 have been strongly linked to susceptibility to Crohn's Disease ((2002) Am. J. Hum. Genet. 70, 845-857; (2004) European Journal of Human Genetics 12, 206-212; (2008) Mucosal Immunology (2008) 1 (Suppl 1), S5-S9. 1, S5-S9; (2008) Inflammatory Bowel Diseases 14, 295-302; (2008) Experimental Dermatology 17, 1057-1058; (2008) British Medical Bulletin 87, 17-30; (2009) Inflammatory Bowel Diseases 15, 1145-1154 and (2009) Microbes and Infection 11, 912-918). Mutations in NOD1 have been associated with asthma ((2005) Hum. Mol. Genet. 14, 935-941) and early-onset and extra-intestinal inflammatory bowel disease ((2005) Hum. Mol. Genet. 14, 1245-1250). Genetic and functional studies have also suggested a role for RIP2-dependent signaling in a variety of other granulomateous disorders, such as sarcoidosis ((2009) Journal of Clinical Immunology 29, 78-89 and (2006) Sarcoidosis Vasculitis and Diffuse Lung Diseases 23, 23-29) and Wegner's Granulomatosis ((2009) Diagnostic Pathology 4, 23).

A potent, selective, small molecule inhibitor of RIP2 kinase activity would block RIP2-dependent pro-inflammatory signaling and thereby provide a therapeutic benefit in autoinflammatory diseases characterized in increased and/or dysregulated RIP2 kinase activity.

### SUMMARY OF THE INVENTION

The invention is directed to novel pyrazolyl-pyrimidinyl diamines. Specifically, the invention is directed to a compound according to Formula (I):

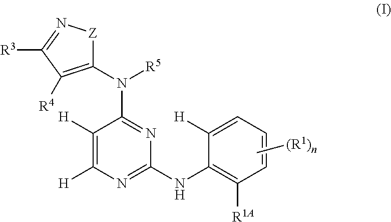

wherein:

R^{1A} is H, methyl or methoxy;

n is 1, 2 or 3;

each R¹ is independently selected from halogen, hydroxy, (C₁-C₆)alkyl, cyano, cyano(C₁-C₆)alkyl-, halo(C₁-C₆)alkyl, (C₁-C₄ alkyl)(C₁-C₄ alkyl)amino-halo(C₂-C₆)alkyl, —OR^{x}, —SR^{x}, —SO₂R^{x}, —NR^{z}SO₂R^{x}, —COOR^{x}, —CONR^{y}R^{z}, —SO₂NR^{y}R^{z}, —SO₂— heterocycloalkyl, heterocycloalkyl, oxazolyl or benzoxazolyl,

and wherein any of said heterocycloalkyl (that is, the heterocycloalkyl group and the heterocycloalkyl moiety of the —SO₂heterocycloalkyl and —NH-heterocycloalkyl groups) is a 4-7 membered non-aromatic ring containing one heteroatom selected from N, O and S, or containing one nitrogen atom and one additional heteroatom selected from N, O and S; which heterocycloalkyl is optionally substituted by 1-5 substituents independently selected from hydroxy, halogen, (C₁-C₆)alkyl, halo(C₁-C₄)alkyl, —CO(C₁-C₆)alkyl, —SO₂(C₁-C₆)alkyl, amino(C₁-C₄)alkyl-, (C₁-C₄ alkyl)amino(C₁-C₄)alkyl-, (C₁-C₄ alkyl)(C₁-C₄ alkyl)amino(C₁-C₄)alkyl- and oxo,

R^{x} is selected from (C₁-C₆)alkyl, halo(C₁-C₆)alkyl, (C₃-C₇)cycloalkyl, hydroxy(C₂-C₆)alkyl-, (C₁-C₆)alkoxyCO(C₁-C₆)alkyl-, amino(C₂-C₆)alkyl-, ((C₁-C₄)alkyl)amino(C₂-C₆)alkyl-, and ((C₁-C₄)alkyl)((C₁-C₄)alkyl)amino(C₂-C₆)alkyl-,

R^{y} is selected from H, (C₁-C₆)alkyl, (C₃-C₇)cycloalkyl, hydroxy(C₂-C₆)alkyl-, amino(C₂-C₆)alkyl-, ((C₁-C₄)alkyl)amino(C₂-C₆)alkyl-, and ((C₁-C₄)alkyl)((C₁-C₄)alkyl)amino(C₂-C₆)alkyl-, and

R^{z} is H or (C₁-C₆)alkyl;

or one of R^{1A}, taken together with an adjacent R¹ group and the carbon atoms connecting the R^{1A} and R¹ groups form a 5-6 membered, aromatic or non-aromatic heterocyclic ring containing 1 or 2 heteroatom ring moieties independently selected from —NH—, —O—, —S— and —SO₂—, or two adjacent R¹ groups taken together with the carbon atoms connecting the two groups form a 5-6 membered, aromatic or non-aromatic heterocyclic ring containing 1 or 2 heteroatom ring moieties independently selected from —NH—, —O—, —S— and —SO₂—,

Z is O or NR²;

R² is H, (C₁-C₄)alkyl, (C₃-C₆)cycloalkyl, hydroxy(C₂-C₄)alkyl-, (C₁-C₆)alkoxy(C₂-C₄)alkyl-, amino(C₂-C₄)alkyl-, (C₁-C₆)alkylamino(C₂-C₄)alkyl-, ((C₁-C₆)alkyl)((C₁-C₆)alkyl)amino(C₂-C₄)alkyl-, —CO₂H, —CO₂(C₁-C₆)alkyl, —CONH₂, —CONH(C₁-C₆)alkyl, —CON((C₁-C₆)alkyl)((C₁-C₆)alkyl), —(C₁-C₄)alkylCO₂H, —(C₁-C₄)alkylCO₂(C₁-C₆)alkyl, —(C₁-C₄)alkylCONH₂, —(C₁-C₄)alkylCONH(C₁-C₆)alkyl, —(C₁-C₄)alkylCON((C₁-C₆)alkyl)((C₁-C₆)alkyl), phenyl, 5-6 membered heterocycloalkyl, 5-6 membered heterocycloalkyl-(C₁-C₄)alkyl-, or 5-6 membered heteroaryl, where said (C₃-C₆)cycloalkyl, phenyl, 5-6 membered heterocycloalkyl, 5-6 membered heterocycloalkyl-(C₁-C₄)alkyl-, or 5-6 membered heteroaryl is optionally substituted by 1-3 substituents independently selected from halogen, hydroxy, (C₁-C₆)alkyl, (C₁-C₄)haloalkyl, (C₁-C₆)alkoxy, (C₁-C₄)haloalkoxy, —CO₂H, —CO₂(C₁-C₄)alkyl and —CO₂(C₁-C₄)alkylphenyl;

R³ is selected from H, methyl, trifluoromethyl and phenyl;

R⁴ is selected from H and methyl; or

R³ and R⁴ taken together with the atoms through which they are attached form, a 5-6 membered non-aromatic carbocyclic ring; and

R⁵ is H or (C₁-C₄)alkyl; or

R⁴ and R⁵ taken together with the atoms through which they are attached form a 5-6 membered, unsubstituted non-aromatic heterocyclic ring;

wherein at least one of R², R³, and R⁴ is not H; or preferably, at least two of R², R³, and R⁴ are not H;

provided that the compound is not:
N²-(3-{[2-(diethylamino)ethyl]oxy}phenyl)-N⁴-(1,3-dimethyl-1H-pyrazol-5-yl)-2,4-pyrimidinediamine;
N²-[3-{[2-(diethylamino)ethyl]oxy}-4-(methyloxy)phenyl]-N⁴-(1-ethyl-1H-pyrazol-5-yl)-2,4-pyrimidinediamine;
N²-[3-{[2-(diethylamino)ethyl]oxy}-4-(methyloxy)phenyl]-N⁴-(3-methyl-1-phenyl-1H-pyrazol-5-yl)-2,4-pyrimidinediamine;
N²-[2-methyl-4-(methyloxy)phenyl]-N⁴-(3-phenyl-5-isoxazolyl)-2,4-pyrimidinediamine; or
N⁴-(3-phenyl-5-isoxazolyl)-N²-{4-[(trifluoromethyl)oxy]phenyl}-2,4-pyrimidinediamine;

or a salt, particularly a pharmaceutically acceptable salt, thereof.

The present invention is also directed to a method of inhibiting RIP2 kinase which comprises contacting the kinase with a compound or salt, thereof, according to Formula (I-A).

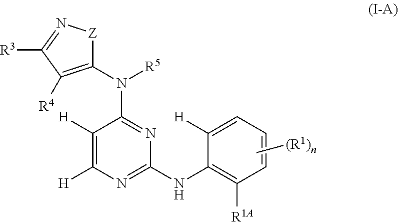

wherein:
R^{1A} is H, methyl or methoxy;

n is 1, 2 or 3;

each R¹ is independently selected from halogen, hydroxy, (C₁-C₆)alkyl, cyano, cyano(C₁-C₆)alkyl-, halo(C₁-C₆)alkyl, (C₁-C₄ alkyl)(C₁-C₄ alkyl)amino-halo(C₂-C₆)alkyl, —OR^{x}, —SR^{x}, —SO₂R^{x}, —NR^{z}SO₂R^{x}, —COOR^{x}, —CONR^{y}R^{z}, —SO₂NR^{y}R^{z}, —SO₂— heterocycloalkyl, heterocycloalkyl, oxazolyl or benzoxazolyl,

and wherein any of said heterocycloalkyl (that is, the heterocycloalkyl group and the heterocycloalkyl moiety of the —SO₂heterocycloalkyl and —NH-heterocycloalkyl groups) is a 4-7 membered non-aromatic ring containing one heteroatom selected from N, O and S, or containing one nitrogen atom and one additional heteroatom selected from N, O and S; which heterocycloalkyl is optionally substituted by 1-5 substituents independently selected from hydroxy, halogen, (C₁-C₆)alkyl, halo(C₁-C₄)alkyl, —CO(C₁-C₆)alkyl, —SO₂(C₁-C₆)alkyl, amino(C₁-C₄)alkyl-, (C₁-C₄ alkyl)amino(C₁-C₄)alkyl-, (C₁-C₄ alkyl)(C₁-C₄ alkyl)amino(C₁-C₄)alkyl- and oxo,

R^{x} is selected from (C₁-C₆)alkyl, halo(C₁-C₆)alkyl, (C₃-C₇)cycloalkyl, hydroxy(C₂-C₆)alkyl-, (C₁-C₆)alkoxyCO(C₁-C₆)alkyl-, amino(C₂-C₆)alkyl-, ((C₁-C₄)alkyl)amino(C₂-C₆)alkyl-, and ((C₁-C₄)alkyl)((C₁-C₄)alkyl)amino(C₂-C₆)alkyl-,

R^{y} is selected from H, (C₁-C₆)alkyl, (C₃-C₇)cycloalkyl, hydroxy(C₂-C₆)alkyl-, amino(C₂-C₆)alkyl-, ((C₁-C₄)alkyl)amino(C₂-C₆)alkyl-, and ((C₁-C₄)alkyl)((C₁-C₄)alkyl)amino(C₂-C₆)alkyl-, and

R^{z} is H or (C₁-C₆)alkyl;

or one of R^{1A}, taken together with an adjacent R¹ group and the carbon atoms connecting the R^{1A} and R¹ groups form a 5-6 membered, aromatic or non-aromatic heterocyclic ring containing 1 or 2 heteroatom ring moieties independently selected from —NH—, —O—, —S— and —SO₂—, or two adjacent R¹ groups taken together with the carbon atoms connecting the two groups form a 5-6 membered, aromatic or non-aromatic heterocyclic ring containing 1 or 2 heteroatom ring moieties independently selected from —NH—, —O—, —S— and —SO₂—,

Z is O or NR²;

R² is H, (C₁-C₄)alkyl, (C₃-C₆)cycloalkyl, hydroxy(C₂-C₄)alkyl-, (C₁-C₆)alkoxy(C₂-C₄)alkyl-, amino(C₂-C₄)alkyl-, (C₁-C₆)alkylamino(C₂-C₄)alkyl-, ((C₁-C₆)alkyl)((C₁-C₆)alkyl)amino(C₂-C₄)alkyl-, —CO₂H, —CO₂(C₁-C₆)alkyl, —CONH₂, —CONH(C₁-C₆)alkyl, —CON((C₁-C₆)alkyl)((C₁-C₆)alkyl), —(C₁-C₄)alkylCO₂H, —(C₁-C₄)alkylCO₂(C₁-C₆)alkyl, —(C₁-C₄)alkylCONH₂, —(C₁-C₄)alkylCONH(C₁-C₆)alkyl, —(C₁-C₄)alkylCON((C₁-C₆)alkyl)((C₁-C₆)alkyl), phenyl, 5-6 membered heterocycloalkyl, 5-6 membered heterocycloalkyl-(C₁-C₄)alkyl-, or 5-6 membered heteroaryl, where said (C₃-C₆)cycloalkyl, phenyl, 5-6 membered heterocycloalkyl, 5-6 membered heterocycloalkyl-(C₁-C₄)alkyl-, or 5-6 membered heteroaryl is optionally substituted by 1-3 substituents independently selected from halogen, hydroxy, (C₁-C₆)alkyl, (C₁-C₄)haloalkyl, (C₁-C₆)alkoxy, (C₁-C₄)haloalkoxy, —CO₂H, —CO₂(C₁-C₄)alkyl and —CO₂(C₁-C₄)alkylphenyl;

R³ is selected from H, methyl, trifluoromethyl and phenyl;

R⁴ is selected from H and methyl; or

R³ and R⁴ taken together with the atoms through which they are attached form, a 5-6 membered non-aromatic carbocyclic ring; and

R⁵ is H or (C₁-C₄)alkyl; or

R⁴ and R⁵ taken together with the atoms through which they are attached form a 5-6 membered, unsubstituted non-aromatic heterocyclic ring;

wherein at least one of R², R³, and R⁴ is not H; or preferably, at least two of R², R³, and R⁴ are not H;

or a salt, particularly a pharmaceutically acceptable salt, thereof.

The compounds of the invention (that is the compounds of Formula (I) and (I-A), and salts thereof), are inhibitors of RIP2 kinase and can be useful for the treatment of RIP2-mediated diseases and disorders, particularly uveitis, dermatitis, arthritis Crohn's disease, asthma, early-onset and extra-intestinal inflammatory bowel disease, and granulomateous disorders, such as adult sarcoidosis, Blau syndrome, early-onset sarcoidosis, and Wegner's Granulomatosis. Accordingly, the invention is further directed to pharmaceutical compositions comprising a compound of the invention.

The invention is still further directed to methods of inhibiting RIP2 kinase and treatment of conditions associated therewith using a compound of the invention or a pharmaceutical composition comprising a compound of the invention.

### DETAILED DESCRIPTION OF THE INVENTION

The alternative definitions for the various groups and substituent groups of Formula I provided throughout the specification are intended to particularly describe each compound species disclosed herein, individually, as well as groups of one or more compound species. The scope of this invention includes any combination of these group and substituent group definitions. The compounds of the invention are only those which are contemplated to be “chemically stable” as will be appreciated by those skilled in the art.

In one embodiment of the compounds of this invention, R^{1A} is H. In a further embodiment, R^{1A} is methyl. In yet another embodiment, R^{1A} is methoxy.

In a further embodiment, each R¹ is independently selected from halogen, hydroxy, (C₁-C₄)alkyl, cyano, cyano(C₁-C₄)alkyl, halo(C₁-C₄)alkyl, (C₁-C₄ alkyl)(C₁-C₄ alkyl)amino-halo(C₂-C₄)alkyl, —OR^{x}, —SR^{x}, —SO₂R^{x}, —NR^{z}SO₂R^{x}, —COOR^{x}, —CONR^{y}R^{z}, —SO₂NR^{y}R^{z}, —SO₂-heterocycloalkyl, heterocycloalkyl, oxazolyl or benzoxazolyl,

wherein any of said heterocycloalkyl is a 5-6 membered non-aromatic ring containing one heteroatom selected from N, O and S, or containing one nitrogen atom and one additional heteroatom selected from N, O and S; which heterocycloalkyl is optionally substituted by 1-3 substituents independently selected from hydroxy, halogen, (C₁-C₄)alkyl, halo(C₁-C₄)alkyl, —CO(C₁-C₄)alkyl, amino(C₁-C₄)alkyl-, (C₁-C₄ alkyl)amino(C₁-C₄)alkyl-, (C₁-C₄ alkyl)(C₁-C₄ alkyl)amino(C₁-C₄)alkyl- and oxo,

R^{x} is selected from (C₁-C₄)alkyl, halo(C₁-C₄)alkyl, (C₃-C₆)cycloalkyl, hydroxy(C₂-C₄)alkyl-, (C₁-C₄)alkoxyCO(C₁-C₄)alkyl-, amino(C₂-C₄)alkyl-, ((C₁-C₄)alkyl)amino(C₂-C₄)alkyl-, and ((C₁-C₄)alkyl)((C₁-C₄)alkyl)amino(C₂-C₄)alkyl-,

R^{y} is selected from H, (C₁-C₄)alkyl, (C₃-C₆)cycloalkyl, hydroxy(C₂-C₄)alkyl-, amino(C₂-C₄)alkyl-, ((C₁-C₄)alkyl)amino(C₂-C₄)alkyl-, and ((C₁-C₄)alkyl)((C₁-C₄)alkyl)amino(C₂-C₄)alkyl-, and

R^{z} is H or (C₁-C₄)alkyl;

or one of R^{1A} taken together with an adjacent R¹ group and the carbon atoms connecting the R^{1A} and R¹ groups, or two adjacent R¹ groups taken together with the carbon atoms connecting the two R¹ groups, form a 5 membered, aromatic or non-aromatic heterocyclic ring containing an —O—, —S—, —SO₂— or —SO₂NH— ring moiety.

In a still further embodiment, each R¹ is independently selected from halogen, hydroxy, (C₁-C₄)alkyl, cyano, cyano(C₁-C₄)alkyl, trifluoromethyl, (C₁-C₄ alkyl)(C₁-C₄ alkyl)amino-halo(C₂-C₄)alkyl, —OR^{x}, —SR^{x}, —SO₂R^{x}, —NHSO₂R^{x}, —COOR^{x}, —CONR^{y}R^{z}, —SO₂NR^{y}R^{z}, —SO₂-heterocycloalkyl, heterocycloalkyl, oxazo-2-yl or benzoxazol-2-yl,

wherein any of said heterocycloalkyl is a 5-6 membered non-aromatic ring containing one heteroatom selected from N, O and S, or containing one nitrogen atom and one additional heteroatom selected from N, O and S; which heterocycloalkyl is optionally substituted by 1-3 substituents independently selected from hydroxy and (C₁-C₄)alkyl,

R^{x} is selected from (C₁-C₄)alkyl, halo(C₁-C₂)alkyl, (C₅-C₆)cycloalkyl, hydroxy(C₂-C₄)alkyl-, (C₁-C₄)alkoxyCO(C₁-C₄)alkyl-, and ((C₁-C₄)alkyl)((C₁-C₄)alkyl)amino(C₂-C₄)alkyl-,

R^{y} is selected from H, (C₁-C₄)alkyl, (C₅-C₆)cycloalkyl, hydroxy(C₂-C₄)alkyl-, and ((C₁-C₄)alkyl)((C₁-C₄)alkyl)amino(C₂-C₄)alkyl-, and

R^{z} is H or (C₁-C₄)alkyl;

or one of R^{1A} taken together with an adjacent R¹ group and the carbon atoms connecting the R^{1A} and R¹ groups, or two adjacent R¹ groups taken together with the carbon atoms connecting the two R¹ groups, form a 5 membered, aromatic or non-aromatic heterocyclic ring containing an —O—, —S—, —SO₂— or —SO₂NH— ring moiety.

In another embodiment, one R¹ is —SO₂R^{x}, —SO₂NR^{y}R^{z}, —SO₂-heterocycloalkyl or heterocycloalkyl, wherein

R^{x} is (C₁-C₄)alkyl, trifluoromethyl, hydroxy(C₂-C₄)alkyl-, cyclopentyl, cyclohexyl;

R^{y} is H, (C₁-C₂)alkyl, hydroxy(C₂-C₃)alkyl-, (C₁-C₂ alkyl)(C₁-C₂ alkyl)amino(C₂-C₃ alkyl)-, cyclopentyl, or piperidinyl, where the piperidinyl is optionally substituted by 1 or 2 substituents independently selected from hydroxy and (C₁-C₂)alkyl;

R^{z} is H or (C₁-C₂ alkyl),

any of said heterocycloalkyl is an optionally substituted 5-6 membered non-aromatic heterocyclic ring, wherein the 5 or 6-membered non-aromatic heterocyclic ring contains one heteroatom selected from N and O, or contains one nitrogen atom and one additional heteroatom selected from N and O, and is optionally substituted by 1-3 independently selected (C₁-C₂)alkyl substituents,

and, when n is 2 or 3, each other R¹ is independently selected from halogen, (C₁-C₂)alkyl, halo(C₁-C₂)alkyl, hydroxy, (C₁-C₂)alkoxy, halo(C₁-C₂)alkoxy, and —SO₂(C₁-C₄)alkyl.

In another embodiment of this invention, each R¹ is independently selected from halogen, (C₁-C₄)alkoxy, —SO₂(C₁-C₄)alkyl, —SO₂NR^{y}R^{z}, and an optionally substituted 6-membered non-aromatic heterocyclic ring,

wherein R^{y} is H, (C₁-C₂ alkyl), or (C₁-C₂ alkyl)(C₁-C₂ alkyl)amino(C₂-C₃ alkyl)-, and R^{z} is H or (C₁-C₂ alkyl), or R^{y} and R^{z}, taken together are —CH₂CH₂CH₂CH₂—,

and wherein the 6-membered non-aromatic heterocyclic ring contains one heteroatom selected from N, O and S, or contains one nitrogen atom and one additional heteroatom selected from N, O and S, and is optionally substituted by 1-4 substituents independently selected from (C₁-C₄)alkyl and when the 6-membered non-aromatic heterocyclic rings contains a nitrogen atom, the nitrogen atom is optionally substituted by (C₁-C₄)alkyl, —CO(C₁-C₆)alkyl, amino(C₂-C₄ alkyl)-, (C₁-C₄ alkyl)amino(C₂-C₄ alkyl)-, or (C₁-C₄ alkyl)(C₁-C₄ alkyl)amino(C₂-C₄ alkyl)-.

In yet another embodiment of this invention, each R¹ is independently selected from chloro, fluoro, methoxy, —SO₂(CH₃), —SO₂pyrrolidin-1-yl, —SO₂NH₂, —SO₂N(CH₃)₂, —SO₂N(CH₃)(CH₂CH₂N(CH₃)₂), and 4-methyl-piperazin-1-yl.

In another embodiment, n is 2 or 3 and each R¹ is independently selected from (C₁-C₄)alkoxy.

In yet another embodiment, n is 1, 2 or 3, one R¹ is —SO₂R^{x}, wherein R^{x} is (C₁-C₄)alkyl, trifluoromethyl, hydroxy(C₂-C₄)alkyl-, cyclopentyl, cyclohexyl, and

each other R¹ is independently selected from halogen, (C₁-C₂)alkyl, halo(C₁-C₂)alkyl, hydroxy, (C₁-C₂)alkoxy, halo(C₁-C₂)alkoxy, —SO₂(C₁-C₄)alkyl, —CO₂(C₁-C₄)alkyl and an optionally substituted 5 or 6-membered non-aromatic heterocyclic ring, wherein the 5-6 membered non-aromatic heterocyclic ring contains one heteroatom selected from N and O, or contains one nitrogen atom and one additional heteroatom selected from N and O, and is optionally substituted by 1-3 independently selected (C₁-C₂)alkyl substituents.

In yet another embodiment, n is 1, 2 or 3, one R¹ is —SO₂NR^{y}R^{z}, wherein R^{y} is H, (C₁-C₂)alkyl, hydroxy(C₂-C₃)alkyl-, (C₁-C₂ alkyl)(C₁-C₂ alkyl)amino(C₂-C₃ alkyl)-, cyclopentyl, or piperidinyl, where the piperidinyl is optionally substituted by 1 or 2 substituents independently selected from hydroxy and (C₁-C₂)alkyl, R^{z} is H or (C₁-C₂ alkyl),

and each other R¹ is independently selected from halogen, (C₁-C₂)alkyl, halo(C₁-C₂)alkyl, and (C₁-C₂)alkoxy.

In yet another embodiment, n is 1, 2 or 3, one R¹ is —SO₂-heterocycloalkyl, wherein said heterocycloalkyl is an optionally substituted 5-6 membered non-aromatic heterocyclic ring, wherein the 5 or 6-membered non-aromatic heterocyclic ring contains one heteroatom selected from N and O, or contains one nitrogen atom and one additional heteroatom selected from N and O, and is optionally substituted by 1-3 independently selected (C₁-C₂)alkyl substituents,

and each other R¹ is independently selected from halogen, (C₁-C₂)alkyl, halo(C₁-C₂)alkyl, and (C₁-C₂)alkoxy.

In a further embodiment, n is 1 or 2 and one R¹ is heterocycloalkyl, wherein said heterocycloalkyl is an optionally substituted 5-6 membered non-aromatic heterocyclic ring, wherein the 5 or 6-membered non-aromatic heterocyclic ring contains one heteroatom selected from N and O, or contains one nitrogen atom and one additional heteroatom selected from N and O, and is optionally substituted by 1-3 independently selected (C₁-C₂)alkyl substituents.

and each other R¹ is independently selected from halogen and (C₁-C₂)alkyl.

In yet another embodiment of this invention, each R¹ is independently selected from hydroxy, cyano, chloro, fluoro, —OCH₃, —OCH₂CH₃, —OCHF₂, —CH₃, —CF₃, —CH(CF₃)N(CH₃)₂, —CN, —C(CN)(CH₃)₂, —CONH₂, —CO₂CH₂CH₃, —S—CH(CH₃)₂, —S—C(CH₃)₂CH₂OH, —S—C(CH₃)₂CO₂CH₂CH₃, —SO₂CH₃, —SO₂CF₃, —SO₂CH₂CH₃, —SO₂CH(CH₃)₂, —SO₂C(CH₃)₃, —SO₂CH₂CH₂OH, —SO₂C(CH₃)₂CH₂OH, —SO₂CH(CH₃)CH₂OH, —SO₂-cyclopentyl, —SO₂-cyclohexyl, —SO₂NH₂, —SO₂N(CH₃)₂, —NHSO₂CH₃, —SO₂N(CH₂CH₃)₂, —SO₂NH-cyclopentyl, —SO₂NHCH₂CH₂OH, —SO₂N(CH₃)(CH₂CH₂N(CH₃)₂), —SO₂-pyrrolidin-1-yl, —SO₂-morpholin-4-yl, —SO₂-(3R)-3-methyl-morpholin-4-yl, —SO₂-(3S)-3-methyl-morpholin-4-yl, —SO₂-tetrahydropyran-4-yl, —SO₂-(2-methyl-tetrahydrofuran-3-yl), —SO₂-(4-hydroxy-piperidin-1-yl), -pyrrolidin-1-yl, 4-methyl-piperazin-1-yl, oxazol-2-yl and benzoxazol-2-yl. In another embodiment of this invention, R¹ is —SO₂CH₃, and R^{1A} taken together with an adjacent R¹ group form a —OCH₂CH₂— moiety. In a further embodiment, R^{1A} taken together with an adjacent R¹ group form a —CH═CH₂S— moiety or R^{1A} is H and two adjacent R¹ groups form a —SO₂NHCH₂— or —CH₂SO₂CH₂— moiety;

two adjacent R¹ groups form a —SO₂NHCH₂— or —CH₂SO₂CH₂ moiety.

In one embodiment of this invention, Z is O.

In a further embodiment of this invention, Z is NR²; where R² is H, (C₁-C₄)alkyl (specifically, methyl), hydroxy(C₂-C₄)alkyl- (specifically, hydroxyethyl-), or an optionally substituted phenyl or pyridyl, where the optionally substituted phenyl or pyridyl is optionally substituted with 1-2 substituents independently selected from halogen (specifically chloro and/or fluoro), (C₁-C₄)alkyl (specifically, methyl), (C₁-C₄)alkoxy (specifically, methoxy), and carboxy.

In another embodiment of this invention, Z is NR²; where R² is H, (C₁-C₄)alkyl, hydroxy(C₂-C₄)alkyl-, (C₁-C₆)alkoxy(C₂-C₄)alkyl-, amino(C₂-C₄)alkyl-, (C₁-C₆)alkylamino(C₂-C₄)alkyl-, ((C₁-C₆)alkyl)((C₁-C₆)alkyl)amino(C₂-C₄)alkyl, 5-6 membered cycloalkyl, phenyl, 5-6 membered heterocycloalkyl, 5-6 membered heterocycloalkylmethyl-, or 5-6 membered heteroaryl, where said 5-6 membered cycloalkyl, phenyl, 5-6 membered heterocycloalkyl, 5-6 membered heterocycloalkylmethyl-, or 5-6 membered heteroaryl is optionally substituted by 1-3 substituents independently selected from halogen, hydroxy, (C₁-C₄)alkyl, (C₁-C₄)haloalkyl, (C₁-C₄)alkoxy, (C₁-C₄)haloalkoxy, —CO₂H, —CO₂(C₁-C₄)alkyl and —CO₂(C₁-C₄)alkylphenyl.

In a still further embodiment of this invention, Z is NR²; where R² is H, (C₁-C₄)alkyl (specifically, methyl), hydroxy(C₂-C₄)alkyl- (specifically, hydroxyethyl-), (C₁-C₄)alkoxy(C₂-C₄)alkyl- (specifically, methoxyethyl-), or an optionally substituted cyclohexyl, phenyl, tetrahydropyranyl, tetrahydropyranylmethyl-, piperidinyl, or pyridyl, where the optionally substituted cyclohexyl, phenyl, tetrahydropyranyl, piperidinyl, or pyridyl is optionally substituted by 1-2 substituents independently selected from halogen (specifically chloro and/or fluoro), hydroxy, (C₁-C₄)alkyl (specifically, methyl), (C₁-C₄)alkoxy (specifically, methoxy), and carboxy. In another embodiment, when Z is NR²; and R² is optionally substituted piperidinyl, the piperidinyl is optionally substituted by —CO₂(C₁-C₂)alkylphenyl (specifically, benzyloxycarbonyl).

In a further embodiment of this invention, Z is NR²; where R² is H, methyl, 2-hydroxyethyl-, phenyl, 3-chloro-phenyl, 4-chloro-phenyl, 3-chloro-4-methyl-phenyl, 3-carboxy-phenyl, 2-methyl-phenyl, 3-methyl-phenyl, 4-methyl-phenyl, 3,4-dimethyl-phenyl, 3-carboxy-phenyl, 3-methoxy-phenyl, 4-methoxy-phenyl, pyrid-2-yl, pyrid-3-yl, pyrid-4-yl, 5-chloro-pyrid-2-yl, 6-methyl-pyrid-2-yl, 6-methyl-pyrid-3-yl, 5-methyl-pyrid-2-yl.

In a still further embodiment of this invention, Z is NR²; where R² is H, methyl, 2-hydroxyethyl-, 2-methoxyethyl-, cyclohexyl, 2-hydroxy-cyclohexyl (specifically, (1S,2S)-cyclohexanol), 1-benzyloxycarbonyl-piperidin-4-yl, phenyl, 3-chloro-phenyl, 4-chloro-phenyl, 3-chloro-4-methyl-phenyl, 3-carboxy-phenyl, 2-methyl-phenyl, 3-methyl-phenyl, 4-methyl-phenyl, 3,4-dimethyl-phenyl, 3-methoxy-phenyl, 4-methoxy-phenyl, pyrid-2-yl, pyrid-3-yl, pyrid-4-yl, 5-chloro-pyrid-2-yl, 6-methyl-pyrid-2-yl, 6-methyl-pyrid-3-yl, 6-methoxy-pyrid-3-yl, 5-methyl-pyrid-2-yl, 5-methoxy-pyrid-2-yl, tetrahydropyran-4-yl or tetrahydropyran-4-yl-methyl-.

In one embodiment, R³ is phenyl. In another embodiment, R³ is trifluoromethyl. In other embodiments, R³ is H or methyl. In specific embodiments, R³ is methyl.

In another embodiment, R⁴ is H or methyl. In specific embodiments, R⁴ is methyl.

In another embodiment, R³ and R⁴ taken together with the atoms through which they are attached form a 5 or 6 membered unsubstituted non-aromatic carbocyclic ring; specifically R³ and R⁴ taken together are —CH₂CH₂CH₂—.

In another embodiment, R⁵ is H or methyl. In a further embodiment, R⁴ and R⁵ taken together are —CH₂CH₂—.

The invention is further directed to a compound according to Formula (I-B),

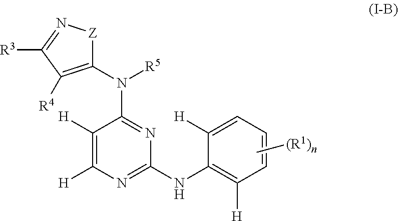

wherein:

n is 1, 2 or 3;

R¹ is halogen, (C₁-C₆)haloalkoxy, —OR^{x}—SO₂R^{x}, —SO₂NR^{x}R^{z} or heterocycloalkyl,

wherein said heterocycloalkyl is a 5-6 membered non-aromatic ring containing one heteroatom selected from N, O and S, or containing one nitrogen atom and optionally containing 1 additional heteroatom selected from N, O and S; which is optionally substituted by 1-5 substituents independently selected from (C₁-C₆)alkyl, (C₁-C₄)haloalkyl, —CO(C₁-C₆)alkyl, aminoC₁-C₄ alkyl-, (C₁-C₄ alkyl)aminoC₁-C₄ alkyl-, (C₁-C₄ alkyl)(C₁-C₄ alkyl)aminoC₁-C₄ alkyl-, and oxo; and

wherein R^{x} and R^{y} are selected from H, (C₁-C₆ alkyl), (C₃-C₇)cycloalkyl, amino(C₂-C₆ alkyl)-, (C₁-C₄ alkyl)amino(C₂-C₆ alkyl)-, and (C₁-C₄ alkyl)(C₁-C₄ alkyl)amino(C₂-C₆ alkyl)-, and

R^{z} is H or (C₁-C₆)alkyl, or

R^{y} and R^{z} taken together with the nitrogen atom to which they are attached form a 4-7 membered non-aromatic heterocyclic ring optionally containing 1 additional heteroatom selected from N, O and S; which is optionally substituted by 1-5 substituents independently selected from (C₁-C₆)alkyl, (C₁-C₄)haloalkyl, —CO(C₁-C₆)alkyl, amino(C₁-C₄ alkyl)-, (C₁-C₄ alkyl)amino(C₁-C₄ alkyl)-, (C₁-C₄ alkyl)(C₁-C₄ alkyl)amino(C₁-C₄ alkyl)-, and oxo;

Z is NR²;

R² is H, (C₁-C₄)alkyl, (C₃-C₆)cycloalkyl, hydroxy(C₂-C₄)alkyl-, (C₁-C₆)alkoxy(C₂-C₄)alkyl-, amino(C₂-C₄)alkyl-, (C₁-C₆)alkylamino(C₂-C₄)alkyl-, ((C₁-C₆)alkyl)((C₁-C₆)alkyl)amino(C₂-C₄)alkyl, —CO₂H, —CO₂(C₁-C₆)alkyl, —CONH₂, —CONH(C₁-C₆)alkyl, —CON((C₁-C₆)alkyl)((C₁-C₆)alkyl), —(C₁-C₄)alkylCO₂H, —(C₁-C₄)alkylCO₂(C₁-C₆)alkyl, —(C₁-C₄)alkylCONH₂, —(C₁-C₄)alkylCONH(C₁-C₆)alkyl, —(C₁-C₄)alkylCON((C₁-C₆)alkyl)((C₁-C₆)alkyl), phenyl or 5-6 membered heteroaryl, where said phenyl or 5-6 membered heteroaryl is optionally substituted by 1-3 substituents independently selected from halogen, (C₁-C₆)alkyl, (C₁-C₄)haloalkyl, (C₁-C₆)alkoxy, (C₁-C₄)haloalkoxy, —CO₂H and —CO₂(C₁-C₄)alkyl;

R³ is selected from H, methyl, trifluoromethyl and phenyl;

R⁴ is selected from H and methyl; or

R³ and R⁴ taken together with the atoms through which they are attached form, a 5-6 membered non-aromatic carbocyclic ring; and

R⁵ is H or (C₁-C₄)alkyl;

wherein at least one of R², R³, and R⁴ is not H; or preferably, at least two of R², R³, and R⁴ are not H;

provided that the compound is not:

N²-(3-{[2-(diethylamino)ethyl]oxy}phenyl)-N⁴-(1,3-dimethyl-1H-pyrazol-5-yl)-2,4-pyrimidinediamine;

N²-[3-{[2-(diethylamino)ethyl]oxy}-4-(methyloxy)phenyl]-N⁴-(1-ethyl-1H-pyrazol-5-yl)-2,4-pyrimidinediamine; or

N²-[3-{[2-(diethylamino)ethyl]oxy}-4-(methyloxy)phenyl]-N⁴-(3-methyl-1-phenyl-1H-pyrazol-5-yl)-2,4-pyrimidinediamine;

or a salt, particularly a pharmaceutically acceptable salt, thereof.

The invention is further directed to a compound according to Formula (I-B), as defined above, where Z is O and the compound is not:

N²-[2-methyl-4-(methyloxy)phenyl]-N⁴-(3-phenyl-5-isoxazolyl)-2,4-pyrimidinediamine; or

N⁴-(3-phenyl-5-isoxazolyl)-N²-{4-[(trifluoromethyl)oxy]phenyl}-2,4-pyrimidinediamine;

or a salt, particularly a pharmaceutically acceptable salt, thereof.

The invention is further directed to a compound according to Formula (I) or Formula (I-A), wherein R^{1A} is H or a compound according to Formula (I-B) wherein:

n is 1, 2 or 3 and each R¹ is independently selected from halogen, (C₁-C₄)alkoxy, —SO₂(C₁-C₄)alkyl, —SO₂NR^{y}R^{z}, and an optionally substituted 6-membered non-aromatic heterocyclic ring (optionally substituted as defined above),

wherein R^{y}H, (C₁-C₂ alkyl), or (C₁-C₂ alkyl)(C₁-C₂ alkyl)amino(C₂-C₃ alkyl)-, and R^{z} is H or (C₁-C₂ alkyl), or R^{y} and R^{z}, taken together are —CH₂CH₂CH₂CH₂—;

Z is NR²; where R² is H, (C₁-C₄)alkyl, hydroxy(C₂-C₄)alkyl-, (C₁-C₆)alkoxy(C₂-C₄)alkyl-, amino(C₂-C₄)alkyl-, (C₁-C₆)alkylamino(C₂-C₄)alkyl-, ((C₁-C₆)alkyl)((C₁-C₆)alkyl)amino(C₂-C₄)alkyl, phenyl or 5-6 membered heteroaryl, where said phenyl or 5-6 membered heteroaryl is optionally substituted by 1-3 substituents independently selected from halogen, (C₁-C₄)alkyl, (C₁-C₄)haloalkyl, (C₁-C₄)alkoxy, (C₁-C₄)haloalkoxy, —CO₂H and —CO₂(C₁-C₄)alkyl;

R³ is trifluoromethyl or R³ is phenyl or R³ is H or methyl;

R⁴ is H or methyl; or

R³ and R⁴ taken together with the atoms through which they are attached form a 5 or 6 membered unsubstituted non-aromatic carbocyclic ring; and

R⁵ is H or methyl;

provided that at least one of R², R³, and R⁴ is not H; or preferably, at least two of R², R³, and R⁴ are not H;

or a salt, particularly a pharmaceutically acceptable salt, thereof.

The invention is further directed to a compound according to Formula (I) or Formula (I-A), wherein R^{1A} is H or a compound according to Formula (I-B) wherein:

n is 1, 2 or 3;

each R¹ is independently selected from chloro, fluoro, methoxy, —SO₂(CH₃), —SO₂pyrrolidin-1-yl, —SO₂NH₂, —SO₂N(CH₃)₂, —SO₂N(CH₃)(CH₂CH₂N(CH₃)₂), and 4-methyl-piperazin-1-yl;

Z is NR², where R² is H, methyl, —CH₂CH₂OH, phenyl, 3-chloro-phenyl, 4-chloro-phenyl, 3-chloro-4-methyl-phenyl, 2-methyl-phenyl, 3-methyl-phenyl, 4-methyl-phenyl, 3,4-dimethyl-phenyl, 3-carboxy-phenyl, 3-methoxy-phenyl, 4-methoxy-phenyl, pyrid-2-yl, pyrid-3-yl, pyrid-4-yl, 5-chloro-pyrid-2-yl, 6-methyl-pyrid-2-yl, 6-methyl-pyrid-3-yl, 5-methyl-pyrid-2-yl;

R³ is trifluoromethyl or phenyl or R³ is H or methyl;

R⁴ is H or methyl;

or R³ and R⁴, taken together are —CH₂CH₂CH₂—; and

R⁵ is H or methyl;

provided that at least one of R², R³, and R⁴ is not H; or preferably, at least two of R², R³, and R⁴ are not H;

or a salt, particularly a pharmaceutically acceptable salt, thereof.

The invention is further directed to a compound according to Formula (I) or Formula (I-A), wherein:

R^{1A} is H, methyl or methoxy;

each R¹ is independently selected from halogen, hydroxy, (C₁-C₄)alkyl, cyano, cyano(C₁-C₄)alkyl, trifluoromethyl, (C₁-C₄ alkyl)(C₁-C₄ alkyl)amino-halo(C₂-C₄)alkyl, —OR^{x}, —SR^{x}, —SO₂R^{x}, —NHSO₂R^{x}, —COOR^{x}, —CONR^{y}R^{z}, —SO₂NR^{y}R^{z}, —SO₂-heterocycloalkyl, heterocycloalkyl, oxazo-2-yl or benzoxazol-2-yl,

wherein any of said heterocycloalkyl is a 5-6 membered non-aromatic ring containing one heteroatom selected from N, O and S, or containing one nitrogen atom and one additional heteroatom selected from N, O and S; which heterocycloalkyl is optionally substituted by 1-3 substituents independently selected from hydroxy and (C₁-C₄)alkyl,

R^{x} is selected from (C₁-C₄)alkyl, halo(C₁-C₂)alkyl, (C₅-C₆)cycloalkyl, hydroxy(C₂-C₄)alkyl-, (C₁-C₄)alkoxyCO(C₁-C₄)alkyl-, and ((C₁-C₄)alkyl)((C₁-C₄)alkyl)amino(C₂-C₄)alkyl-,

R^{y} is selected from H, (C₁-C₄)alkyl, (C₅-C₆)cycloalkyl, hydroxy(C₂-C₄)alkyl-, and ((C₁-C₄)alkyl)((C₁-C₄)alkyl)amino(C₂-C₄)alkyl-, and

R^{z} is H or (C₁-C₄)alkyl;

or one of R^{1A} taken together with an adjacent R¹ group and the carbon atoms connecting the R^{1A} and R¹ groups, or two adjacent R¹ groups taken together with the carbon atoms connecting the two R¹ groups, form a 5 membered, aromatic or non-aromatic heterocyclic ring containing an —O—, —S—, —SO₂— or —SO₂NH— ring moiety;

Z is O or NR²; where R² is H, (C₁-C₄)alkyl, hydroxy(C₂-C₄)alkyl-, (C₁-C₄)alkoxy(C₂-C₄)alkyl-, or an optionally substituted cyclohexyl, phenyl, tetrahydropyranyl, tetrahydropyranylmethyl-, piperidinyl, or pyridyl, where the optionally substituted cyclohexyl, phenyl, tetrahydropyranyl, piperidinyl, or pyridyl is optionally substituted by 1-2 substituents independently selected from halogen, hydroxy, (C₁-C₄)alkyl, (C₁-C₄)alkoxy, and carboxy;

or Z is NR²; where R² is an optionally substituted piperidinyl, said piperidinyl is optionally substituted by —CO₂(C₁-C₂)alkylphenyl;

R³ is H, methyl, trifluoromethyl or phenyl;

R⁴ is H or methyl;

or R³ and R⁴ taken together are —CH₂CH₂CH₂—;

R⁵ is H or methyl;

or R⁴ and R⁵ taken together are —CH₂CH₂—;

provided that at least one of R², R³, and R⁴ is not H; or preferably, at least two of R², R³, and R⁴ are not H;

or a salt, particularly a pharmaceutically acceptable salt, thereof.

The invention is further directed to a compound according to Formula (I) or Formula (I-A), wherein:

R^{1A} is H, methyl or methoxy, n is 1, 2 or 3, and each R¹ is independently selected from hydroxy, cyano, chloro, fluoro, —OCH₃, —OCH₂CH₃, —OCHF₂, —CH₃, —CF₃, —CH(CF₃)N(CH₃)₂, —CN, —C(CN)(CH₃)₂, —CONH₂, —CO₂CH₂CH₃, —S—CH(CH₃)₂, —S—C(CH₃)₂CH₂OH, —S—C(CH₃)₂CO₂CH₂CH₃, —SO₂CH₃, —SO₂CF₃, —SO₂CH₂CH₃, —SO₂CH(CH₃)₂, —SO₂C(CH₃)₃, —SO₂CH₂CH₂OH, —SO₂C(CH₃)₂CH₂OH, —SO₂CH(CH₃)CH₂OH, —SO₂-cyclopentyl, —SO₂-cyclohexyl, —SO₂NH₂, —SO₂N(CH₃)₂, —NHSO₂CH₃, —SO₂N(CH₂CH₃)₂, —SO₂NH-cyclopentyl, —SO₂NHCH₂CH₂OH, —SO₂N(CH₃)(CH₂CH₂N(CH₃)₂), —SO₂-pyrrolidin-1-yl, —SO₂-morpholin-4-yl, —SO₂-(3R)-3-methyl-morpholin-4-yl, —SO₂-(3S)-3-methyl-morpholin-4-yl, —SO₂-tetrahydropyran-4-yl, —SO₂-(2-methyl-tetrahydrofuran-3-yl), —SO₂-(4-hydroxy-piperidin-1-yl), -pyrrolidin-1-yl, 4-methyl-piperazin-1-yl, oxazol-2-yl and benzoxazol-2-yl,

or n is 2, R¹ is —SO₂CH₃, and R^{1A} taken together with an adjacent R¹ group form a —OCH₂CH₂— moiety;

or n is 1 and R^{1A} taken together with an adjacent R¹ group form a —CH═CH₂S— moiety;

or R^{1A} is H, n is 2, and two adjacent R¹ groups form a —SO₂NHCH₂— or —CH₂SO₂CH₂— moiety;

Z is O or Z is NR²; where R² is H, methyl, 2-hydroxyethyl-, 2-methoxyethyl-, cyclohexyl, 2-hydroxy-cyclohexyl, 1-benzyloxycarbonyl-piperidin-4-yl, phenyl, 3-chloro-phenyl, 4-chloro-phenyl, 3-chloro-4-methyl-phenyl, 3-carboxy-phenyl, 2-methyl-phenyl, 3-methyl-phenyl, 4-methyl-phenyl, 3,4-dimethyl-phenyl, 3-methoxy-phenyl, 4-methoxy-phenyl, pyrid-2-yl, pyrid-3-yl, pyrid-4-yl, 5-chloro-pyrid-2-yl, 6-methyl-pyrid-2-yl, 6-methyl-pyrid-3-yl, 6-methoxy-pyrid-3-yl, 5-methyl-pyrid-2-yl, 5-methoxy-pyrid-2-yl, tetrahydropyran-4-yl or tetrahydropyran-4-yl-methyl-;

R³ is H, methyl, trifluoromethyl or phenyl;

R⁴ is H or methyl;

or R³ and R⁴ taken together are —CH₂CH₂CH₂—;

R⁵ is H or methyl;

or R⁴ and R⁵ taken together are —CH₂CH₂—.

The present invention is further directed to a method of inhibiting RIP2 kinase which method comprises contacting the kinase with a compound according to Formula (I), (I-A) or (I-B), or a salt, particularly a pharmaceutically acceptable salt, thereof. The compounds of the invention (that is a compounds of Formula (I), (I-A) or (I-B) and salts thereof), are inhibitors of RIP2 kinase and may be useful for the treatment of RIP2 kinase-mediated diseases and disorders. Accordingly, the invention is further directed to a method of treating a RIP2 kinase-mediated disease or condition in a patient (particularly, a human) which comprises administering to the patient a therapeutically effective amount of a compound according to Formula (I), (I-A) or (I-B) or a pharmaceutically acceptable salt thereof. The present invention is also directed to pharmaceutical compositions comprising a compound of the invention. The invention is still further directed to the use of a compound of the invention or a pharmaceutical composition comprising a compound of the invention to inhibit RIP2 kinase and/or treat a RIP2 kinase-mediated disease or disorder.

As used herein, the term “alkyl” represents a saturated, straight or branched hydrocarbon moiety, which may be unsubstituted or substituted by one, or more of the substituents defined herein. Exemplary alkyls include, but are not limited to methyl (Me), ethyl (Et), propyl, isopropyl, butyl, isobutyl, t-butyl and pentyl. The term “C₁-C₄” refers to an alkyl containing from 1 to 4 carbon atoms.

When the term “alkyl” is used in combination with other substituent groups, such as “haloalkyl” or “hydroxyalkyl” or “arylalkyl”, the term “alkyl” is intended to encompass a divalent straight or branched-chain hydrocarbon radical. For example, “arylalkyl” is intended to mean the radical—alkylaryl, wherein the alkyl moiety thereof is a divalent straight or branched-chain carbon radical and the aryl moiety thereof is as defined herein, and is represented by the bonding arrangement present in a benzyl group (—CH₂-phenyl).

As used herein, the term “alkenyl” refers to a straight or branched hydrocarbon moiety containing at least 1 and up to 3 carbon-carbon double bonds. Examples include ethenyl and propenyl.

As used herein, the term “alkynyl” refers to a straight or branched hydrocarbon moiety containing at least 1 and up to 3 carbon-carbon triple bonds. Examples include ethynyl and propynyl.

As used herein, the term “cycloalkyl” refers to a non-aromatic, saturated, cyclic hydrocarbon ring. The term “(C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

“Alkoxy” refers to a group containing an alkyl radical attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include, but are not limited to, methoxy, ethoxy, n-propoxy, isopropoxy, n-butoxy, s-butoxy, and t-butoxy.

“Alkylthio-” refers to a group containing an alkyl radical attached through a sulfur linking atom. The term “(C₁-C₄)alkylthio-” refers to a straight- or branched-chain hydrocarbon radical having at least 1 and up to 4 carbon atoms attached through a sulfur linking atom. Exemplary “(C₁-C₄)alkylthio-” groups useful in the present invention include, but are not limited to, methylthio-, ethylthio-, n-propylthio-, isopropylthio-, n-butylthio-, s-butylthio-, and t-butylthio-.

“Cycloalkyloxy” and “cycloalkylthio” refers to a group containing a saturated carbocyclic ring atoms attached through an oxygen or sulfur linking atom, respectively. Examples of “cycloalkyloxy” moieties include, but are not limited to, cyclopropoxy, cyclobutoxy, cyclopentyloxy, cyclohexyloxy, and the like.

“Aryl” represents a group or moiety comprising an aromatic, monovalent monocyclic or bicyclic hydrocarbon radical containing from 6 to 10 carbon ring atoms, which may be unsubstituted or substituted by one or more of the substituents defined herein, and to which may be fused one or more cycloalkyl rings, which may be unsubstituted or substituted by one or more substituents defined herein.

Generally, in the compounds of this invention, aryl is phenyl.

Heterocyclic groups may be heteroaryl or heterocycloalkyl groups.

“Heterocycloalkyl” represents a group or moiety comprising a non-aromatic, monovalent monocyclic or bicyclic radical, which is saturated or partially unsaturated, containing 3 to 10 ring atoms, which includes 1 to 4 heteroatoms selected from nitrogen, oxygen and sulfur, and which may be unsubstituted or substituted by one or more of the substituents defined herein. Illustrative examples of heterocycloalkyls include, but are not limited to, azetidinyl, pyrrolidyl (or pyrrolidinyl), piperidinyl, piperazinyl, morpholinyl, tetrahydro-2H-1,4-thiazinyl, tetrahydrofuryl (or tetrahydrofuranyl), dihydrofuryl, oxazolinyl, thiazolinyl, pyrazolinyl, tetrahydropyranyl, dihydropyranyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, oxabicylo[2.2.1]heptyl and 1,5,9-triazacyclododecyl.

Generally, in the compounds of this invention, heterocycloalkyl groups are 5-membered and/or 6-membered heterocycloalkyl groups, such as pyrrolidyl (or pyrrolidinyl), tetrahydrofuryl (or tetrahydrofuranyl), tetrahydrothienyl, dihydrofuryl, oxazolinyl, thiazolinyl or pyrazolinyl, piperidyl (or piperidinyl), piperazinyl, morpholinyl, tetrahydropyranyl, dihydropyranyl, 1,3-dioxanyl, tetrahydro-2H-1,4-thiazinyl, 1,4-dioxanyl, 1,3-oxathianyl, and 1,3-dithianyl.

“Heteroaryl” represents a group or moiety comprising an aromatic monovalent monocyclic or bicyclic radical, containing 5 to 10 ring atoms, including 1 to 4 heteroatoms selected from nitrogen, oxygen and sulfur, which may be unsubstituted or substituted by one or more of the substituents defined herein. This term also encompasses bicyclic heterocyclic-aryl compounds containing an aryl ring moiety fused to a heterocycloalkyl ring moiety, containing 5 to 10 ring atoms, including 1 to 4 heteroatoms selected from nitrogen, oxygen and sulfur, which may be unsubstituted or substituted by one or more of the substituents defined herein. Illustrative examples of heteroaryls include, but are not limited to, thienyl, pyrrolyl, imidazolyl, pyrazolyl, furyl (or furanyl), isothiazolyl, furazanyl, isoxazolyl, oxazolyl, oxadiazolyl, thiazolyl, pyridyl (or pyridinyl), pyrazinyl, pyrimidinyl, pyridazinyl, triazinyl, tetrazinyl, triazolyl, tetrazolyl, benzo[b]thienyl, isobenzofuryl, 2,3-dihydrobenzofuryl, chromenyl, chromanyl, indolizinyl, isoindolyl, indolyl, indazolyl, purinyl, isoquinolyl, quinolyl, phthalazinyl, naphthridinyl, quinzolinyl, benzothiazolyl, benzimidazolyl, tetrahydroquinolinyl, cinnolinyl, pteridinyl, isothiazolyl.

Generally, the heteroaryl groups present in the compounds of this invention are 5-membered and/or 6-membered monocyclic heteroaryl groups. Selected 5-membered heteroaryl groups contain one nitrogen, oxygen or sulfur ring heteroatom, and optionally contain 1, 2 or 3 additional nitrogen ring atoms. Selected 6-membered heteroaryl groups contain 1, 2, 3 or 4 nitrogen ring heteroatoms. Selected 5- or 6-membered heteroaryl groups include thienyl, pyrrolyl, imidazolyl, pyrazolyl, furyl, isothiazolyl, furazanyl, isoxazolyl, oxazolyl, oxadiazolyl, thiazolyl, triazolyl, and tetrazolyl or pyridyl, pyrazinyl, pyrimidinyl, pyridazinyl, and triazinyl.

It is to be understood that the terms heterocycle, heterocyclic, heteroaryl, heterocycloalkyl, are intended to encompass stable heterocyclic groups where a ring nitrogen heteroatom is optionally oxidized (e.g., heterocyclic groups containing an N-oxide, such as pyridine-N-oxide) or where a ring sulfur heteroatom is optionally oxidized (e.g., heterocyclic groups containing sulfones or sulfoxide moieties, such as tetrahydrothienyl-1-oxide (a tetramethylene sulfoxide) or tetrahydrothienyl-1,1-dioxide (a tetramethylene sulfone)).

“Oxo” represents a double-bonded oxygen moiety; for example, if attached directly to a carbon atom forms a carbonyl moiety (C═O). The terms “halogen” and “halo” represent chloro, fluoro, bromo or iodo substituents. “Hydroxy” or “hydroxyl” is intended to mean the radical —OH.

As used herein, the term “compound(s) of the invention” means a compound of Formula (I), (I-A) or (I-B) (as defined above) in any form, i.e., any salt or non-salt form (e.g., as a free acid or base form, or as a pharmaceutically acceptable salt thereof) and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” means unsubstituted groups or rings (e.g., cycloalkyl, heterocycle, and heteroaryl rings) and groups or rings substituted with one or more specified substituents.

Specific compounds of this invention include N²-[3,4-bis(methyloxy)phenyl]-N⁴-[3-methyl-1-(4-methylphenyl)-1H-pyrazol-5-yl]-2,4-pyrimidinediamine, N²-[3,4-bis(methyloxy)phenyl]-N⁴-[1-(3,4-dimethylphenyl)-3-methyl-1H-pyrazol-5-yl]-2,4-pyrimidinediamine, N²-[3,4-bis(methyloxy)phenyl]-N⁴-[3-methyl-1-(3-methylphenyl)-1H-pyrazol-5-yl]-2,4-pyrimidinediamine, N⁴-methyl-N⁴-(1,3,4-trimethyl-1H-pyrazol-5-yl)-N²-[3,4,5-tris(methyloxy)phenyl]-2,4-pyrimidinediamine, N⁴-[3,4-dimethyl-1-(2-pyridinyl)-1H-pyrazol-5-yl]-N²-[4-fluoro-3-(methyloxy)phenyl]-2,4-pyrimidinediamine, and the compounds of Examples 1-178, in free base form, or in the form of a salt, particularly a pharmaceutically acceptable salt, thereof.

Compound names were generated using the software naming program ACD/Name Pro V6.02 available from Advanced Chemistry Development, Inc., 110 Yonge Street, 14^{th} Floor, Toronto, Ontario, Canada, M5C 1T4 (http://www.acdlabs.com/). It will be appreciated by those skilled in the art that many of the compounds of this invention, as well as compounds used in the preparation of the compounds of Formula (I), (I-A) or (I-B) may exist in tautomeric forms. The program used to name the compounds of this invention will only name one of such tautomeric forms at a time. It is to be understood that any reference to a named compound or a structurally depicted compound is intended to encompass all tautomers of such compounds and any mixtures of tautomers thereof.

The compounds according to Formula (I), (I-A) or (I-B) may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in a compound of this invention, or in any chemical structure illustrated herein, is not specified the structure is intended to encompass all individual stereoisomers and all mixtures thereof. Thus, compounds according to Formula (I), (I-A) or (I-B) containing one or more chiral center may be used as racemic mixtures, enantiomerically enriched mixtures, or as enantiomerically pure individual stereoisomers.

Individual stereoisomers of a compound according to according to Formula (I), (I-A) or (I-B) which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation. When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

Because of their potential use in medicine, the salts of the compounds of according to Formula (I), (I-A) or (I-B) are preferably pharmaceutically acceptable salts. Suitable pharmaceutically acceptable salts include those described by Berge, Bighley and Monkhouse J. Pharm. Sci (1977) 66, pp 1-19. Salts encompassed within the term “pharmaceutically acceptable salts” refer to non-toxic salts of the compounds of this invention.

When a compound of the invention is a base (contain a basic moiety), a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, and the like, or with a pyranosidyl acid, such as glucuronic acid or galacturonic acid, or with an alpha-hydroxy acid, such as citric acid or tartaric acid, or with an amino acid, such as aspartic acid or glutamic acid, or with an aromatic acid, such as benzoic acid or cinnamic acid, or with a sulfonic acid, such as p-toluenesulfonic acid, methanesulfonic acid, ethanesulfonic acid or the like.

Suitable addition salts are formed from acids which form non-toxic salts and examples include acetate, p-aminobenzoate, ascorbate, aspartate, benzenesulfonate, benzoate, bicarbonate, bismethylenesalicylate, bisulfate, bitartrate, borate, calcium edetate, camsylate, carbonate, clavulanate, citrate, cyclohexylsulfamate, edetate, edisylate, estolate, esylate, ethanedisulfonate, ethanesulfonate, formate, fumarate, gluceptate, gluconate, glutamate, glycollate, glycollylarsanilate, hexylresorcinate, hydrabamine, hydrobromide, hydrochloride, dihydrochloride, hydrofumarate, hydrogen phosphate, hydroiodide, hydromaleate, hydrosuccinate, hydroxynaphthoate, isethionate, itaconate, lactate, lactobionate, laurate, malate, maleate, mandelate, mesylate, methylsulfate, monopotassium maleate, mucate, napsylate, nitrate, N-methylglucamine, oxalate, oxaloacetate, pamoate (embonate), palmate, palmitate, pantothenate, phosphate/diphosphate, pyruvate, polygalacturonate, propionate, saccharate, salicylate, stearate, subacetate, succinate, sulfate, tannate, tartrate, teoclate, tosylate, triethiodide, trifluoroacetate and valerate.

Other exemplary acid addition salts include pyrosulfate, sulfite, bisulfite, decanoate, caprylate, acrylate, isobutyrate, caproate, heptanoate, propiolate, oxalate, malonate, suberate, sebacate, butyne-1,4-dioate, hexyne-1,6-dioate, chlorobenzoate, methylbenzoate, dinitrobenzoate, hydroxybenzoate, methoxybenzoate, phthalate, phenylacetate, phenylpropionate, phenylbutrate, lactate, γ-hydroxybutyrate, mandelate, and sulfonates, such as xylenesulfonate, propanesulfonate, naphthalene-1-sulfonate and naphthalene-2-sulfonate.

If an inventive basic compound is isolated as a salt, the corresponding free base form of that compound may be prepared by any suitable method known to the art, including treatment of the salt with an inorganic or organic base, suitably an inorganic or organic base having a higher pKₐ than the free base form of the compound.

When a compound of the invention is an acid (contains an acidic moiety), a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines, such as N-methyl-D-glucamine, diethylamine, isopropylamine, trimethylamine, ethylene diamine, dicyclohexylamine, ethanolamine, piperidine, morpholine, and piperazine, as well as inorganic salts derived from sodium, calcium, potassium, magnesium, manganese, iron, copper, zinc, aluminum, and lithium.

Certain of the compounds of this invention may form salts with one or more equivalents of an acid (if the compound contains a basic moiety) or a base (if the compound contains an acidic moiety). The present invention includes within its scope all possible stoichiometric and non-stoichiometric salt forms.

Compounds of the invention having both a basic and acidic moiety may be in the form of zwitterions, acid-addition salt of the basic moiety or base salts of the acidic moiety.

This invention also provides for the conversion of one pharmaceutically acceptable salt of a compound of this invention, e.g., a hydrochloride salt, into another pharmaceutically acceptable salt of a compound of this invention, e.g., a sodium salt.

For solvates of the compounds of the invention, or salts thereof that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The subject invention also includes isotopically-labeled compounds which are identical to those recited in according to Formula (I), (I-A) or (I-B) but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography).

Because the compounds of according to Formula (I), (I-A) or (I-B) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

### General Synthetic Methods

The compounds of according to Formula (I), (I-A) or (I-B) may be obtained by using synthetic procedures illustrated in the Schemes below or by drawing on the knowledge of a skilled organic chemist. The synthesis provided in these Schemes are applicable for producing compounds of the invention having a variety of different R¹ and R² groups employing appropriate precursors, which are suitably protected if needed, to achieve compatibility with the reactions outlined herein. Subsequent deprotection, where needed, affords compounds of the nature generally disclosed. While the Schemes are shown with compounds only of according to Formula (I), (I-A) or (I-B), they are illustrative of processes that may be used to make the compounds of the invention.

Intermediates (compounds used in the preparation of the compounds of the invention) may also be present as salts. Thus, in reference to intermediates, the phrase “compound(s) of formula (number)” means a compound having that structural formula or a pharmaceutically acceptable salt thereof.

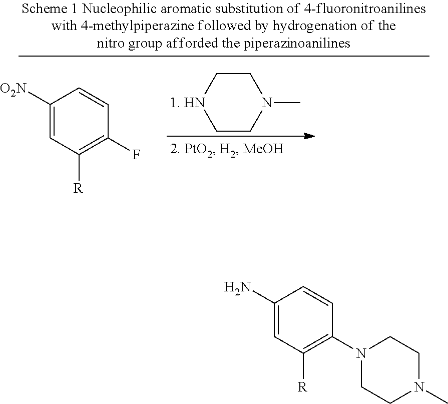

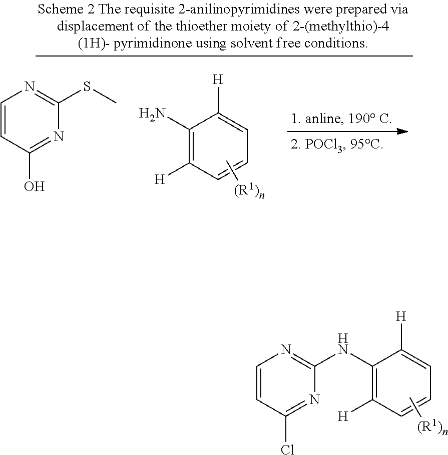

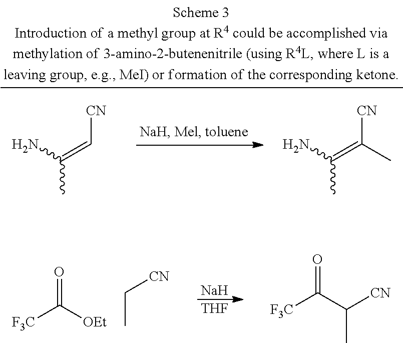

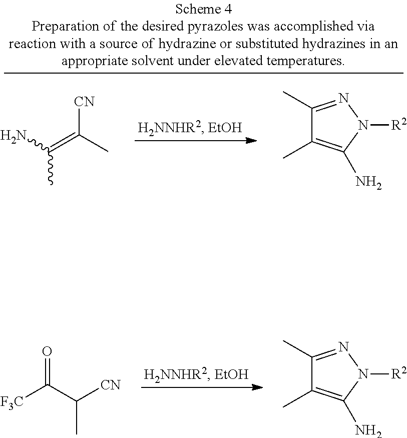

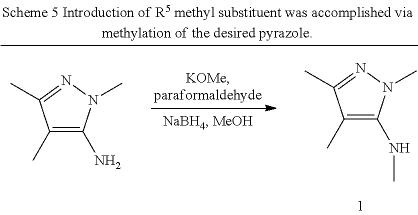

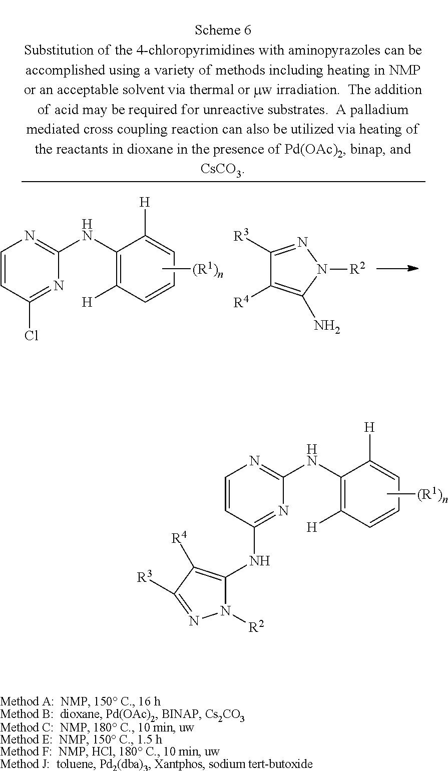

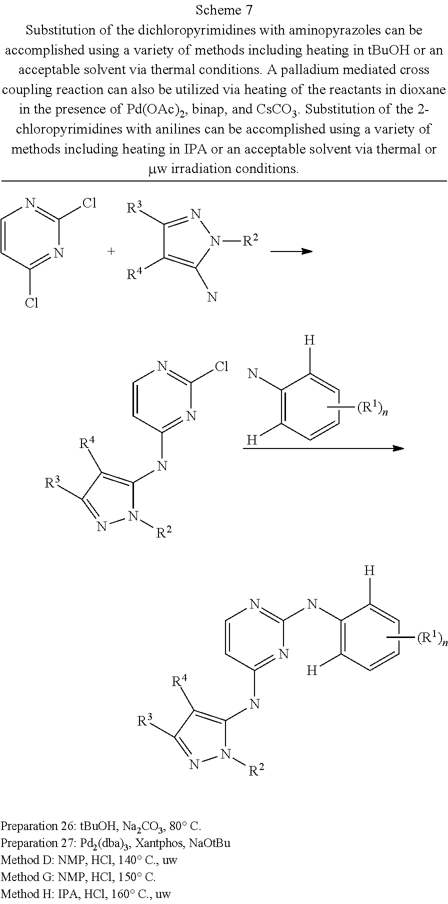

The present invention is also directed to a method of inhibiting RIP2 kinase which comprises contacting the kinase with a compound according to Formula (I), (I-A) or (I-B), or a salt, particularly a pharmaceutically acceptable salt, thereof. This invention is also directed to a method of treatment of a RIP2-mediated disease or disorder comprising administering a therapeutically effective amount of a compound of according to Formula (I), (I-A) or (I-B), or a salt thereof, particularly a pharmaceutically acceptable salt thereof, to a patient, specifically a human, in need thereof. As used herein, “patient” refers to a human or other mammal.

The compounds of this invention may be particularly useful for treatment of RIP2-mediated diseases or disorders, particularly, uveitis, interleukin-1 converting enzyme (ICE, also known as Caspase-1) associated fever syndrome, dermatitis, type 2 diabetes mellitus, acute lung injury, arthritis (specifically rheumatoid arthritis), inflammatory bowel disorders (such as ulcerative colitis and Crohn's disease), prevention of ischemia reperfusion injury in solid organ transplant, liver diseases (non-alcohol steatohepatitis, alcohol steatohepatitis, autoimmune hepatitis), allergic diseases (such as asthma), autoimmune diseases (such as systemic lupus erythematosus and Multiple Sclerosis), transplant reactions (such as graft versus host disease) and granulomateous disorders, such as adult sarcoidosis, Blau syndrome, early-onset sarcoidosis, cutaneous sarcoidosis, Wegner's granulomatosis, and interstitial pulmonary disease. The compounds of this invention may be particularly useful in the treatment of uveitis, ICE fever, Blau Syndrome/early-onset sarcoidosis, ulcerative colitis, Crohn's disease, Wegener's granulamatosis and sarcoidosis.

Treatment of RIP2-mediated disease conditions, or more broadly, treatment of immune mediated disease, such as, but not limited to, allergic diseases, autoimmune diseases, prevention of transplant rejection and the like, may be achieved using a compound of this invention of as a monotherapy, or in dual or multiple combination therapy, particularly for the treatment of refractory cases, such as in combination with other anti-inflammatory and/or anti-TNF agents, which may be administered in therapeutically effective amounts as is known in the art. For example, the compounds of this invention may be administered in combination with corticosteroids and/or anti-TNF agents to treat Blau syndrome/early-onset sarcoidosis; or in combination with anti-TNF biologics or other anti-inflammatory biologics to treat Crohn's Disease; or in combination with low-dose corticosteroids and/or methotrexate to treat Wegener's granulamatosis or sarcoidosis or interstitial pulmonary disease; or in combination with a biologic (e.g. anti-TNF, anti-IL-6, etc.) to treat rheumatoid arthritis; or in combination with anti-IL6 and or methotrexate to treat ICE fever.

Examples of suitable anti-inflammatory agents include corticosteroids, particularly low-dose corticosteroids (such as Deltasone® (prednisone)) and anti-inflammatory biologics (such as Acterma® (anti-IL6R mAb) and Rituximab® (anti-CD20 mAb)). Examples of suitable anti-TNF agents include anti-TNF biologics (such as Enbrel® (etanecerpt)), Humira® (adalimumab), Remicade® (infliximab) and Simponi® (golimumab)).

This invention also provides a compound of according to Formula (I), (I-A) or (I-B), or a salt thereof, particularly a pharmaceutically acceptable salt thereof, for use in the treatment or prophylaxis of RIP2-mediated diseases or disorders, for example those diseases and disorders mentioned hereinabove.

The invention also provides the use of a compound of according to Formula (I), (I-A) or (I-B), or a salt thereof, particularly a pharmaceutically acceptable salt thereof, in the manufacture of a medicament for the treatment or prophylaxis of RIP2-mediated diseases or disorders, for example those diseases and disorders mentioned hereinabove.

A therapeutically “effective amount” is intended to mean that amount of a compound that, when administered to a patient in need of such treatment, is sufficient to effect treatment, as defined herein. Thus, e.g., a therapeutically effective amount of a compound of according to Formula (I), (I-A) or (I-B), or a pharmaceutically acceptable salt thereof, is a quantity of an inventive agent that, when administered to a human in need thereof, is sufficient to modulate or inhibit the activity of RIP2 kinase such that a disease condition which is mediated by that activity is reduced, alleviated or prevented. The amount of a given compound that will correspond to such an amount will vary depending upon factors such as the particular compound (e.g., the potency (pIC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmaceutical characteristics), disease or condition and its severity and the specific composition and method being used, but can nevertheless be determined by one of skill in the art.

“Treating” or “treatment” is intended to mean at least the mitigation of a disease condition in a patient. The methods of treatment for mitigation of a disease condition include the use of the compounds in this invention in any conventionally acceptable manner, for example for prevention, retardation, prophylaxis, therapy or cure of a mediated disease. Specific diseases and conditions that may be particularly susceptible to treatment using a compound of this invention are described herein.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

For use in therapy, the compounds of the invention will be normally, but not necessarily, formulated into a pharmaceutical composition prior to administration to a patient. Accordingly, the invention is also directed to pharmaceutical compositions comprising a compound of the invention and a pharmaceutically-acceptable excipient.

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound of this invention (i.e., a compound of according to Formula (I), (I-A) or (I-B) or a salt, particularly a pharmaceutically acceptable salt, thereof). When prepared in unit dosage form, the pharmaceutical compositions may contain from 1 mg to 1000 mg of a compound of this invention.

The pharmaceutical compositions of the invention typically contain one compound of the invention. However, in certain embodiments, the pharmaceutical compositions of the invention contain more than one compound of the invention. In addition, the pharmaceutical compositions of the invention may optionally further comprise one or more additional pharmaceutically active compounds.

As used herein, “pharmaceutically-acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically-acceptable are avoided. In addition, each excipient must of course be of sufficiently high purity to render it pharmaceutically-acceptable.

The compounds of the invention and the pharmaceutically-acceptable excipient or excipients will typically be formulated into a dosage form adapted for administration to the patient by the desired route of administration. Conventional dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company) The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

Names for the intermediate and final compounds described herein were generated using a commercially available software naming program. It will be appreciated by those skilled in the art that in certain instances such programs will name a structurally depicted compound (e.g., intermediates of Preparation 23) as a single tautomer of that compound. It is to be understood that any reference to a named compound or a structurally depicted compound is intended to encompass all tautomers of such compounds and any mixtures of tautomers thereof.

In the following experimental descriptions, the following abbreviations may be used:

| Abbreviation | Meaning |
|---|---|
| aq | aqueous |
| brine | saturated aqueous NaCl |
| CH₂Cl₂, DCM | methylene chloride |
| CH₃CN or MeCN | acetonitrile |
| CH₃SNa | sodium methyl mercaptide |
| d | day |
| DIEA | diisopropylethylamine |
| DMF | N,N-dimethylformamide |
| DMSO | Dimethylsulfoxide |
| equiv | equivalents |
| Et | ethyl |
| Et₃N | triethylamine |
| Et₂O | diethyl ether |
| EtOAc | ethyl acetate |
| h, hr | hour |
| HATU | 2-(1H-7-azabenzotriazol-1-yl)--1,1,3,3-tetramethyl- |
| | uronium hexafluorophosphate |
| HCl | hydrochloric acid |
| i-Pr₂NEt | N′,N′-diisopropylethylamine |
| KOMe | potassium methoxide |
| LCMS | liquid chromatography-mass spectroscopy |
| Me | methyl |
| MeI | methyl iodide |
| MeOH or CH₃OH | methanol |
| MgSO₄ | magnesium sulfate |
| min | minute |
| MS | mass spectrum |
| μw | microwave |
| NMP | N-methyl-2-pyrrolidine |
| NaH | sodium hydride |
| NaHCO₃ | sodium bicarbonate |
| Na₂SO₄ | sodium sulfate |
| NH₄Cl | ammonium chloride |
| Pd/C | palladium on carbon |
| Ph | phenyl |
| rt | room temperature |
| satd | saturated |
| SPE | solid phase extraction |
| TFA | trifluoroacetic acid |
| THF | tetrahydrofuran |
| t_{R} | retention time |
| ptfe | polytetrafluoroethylene |
| binap | 2,2′-bis(diphenylphosphino)-1,1′-binaphthalene |

### Preparation 1

### [3-Chloro-4-(4-methyl-1-piperazinyl)phenyl]amine

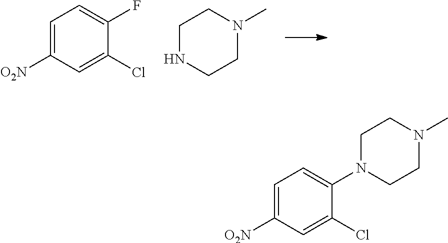

1-(2-Chloro-4-nitrophenyl)-4-methylpiperazine: 4-methylpiperizine (17.2 g, 172 mmol) was added to a flask containing 2-chloro-1-fluoro-4-nitrobenzene (10.05 g, 57.2 mmol). The reaction exothermed and was stirred for one hour before being treated with water. The resulting precipitate was collected by filtration, washed with water, and air dried to give the title compound as a brown solid (14.1 g, 96%). ¹H NMR (400 MHz, DMSO-d₆) δ ppm 2.24 (s, 3H) 2.43-2.55 (m, 4H) 2.43-2.55 (m, 4H) 3.11-3.24 (m, 4H) 7.28 (d, J=9.03 Hz, 1H) 8.14 (dd, J=9.03, 2.76 Hz, 1H) 8.21 (d, J=2.51 Hz, 1H); MS (m/z) 255 (M+H⁺).

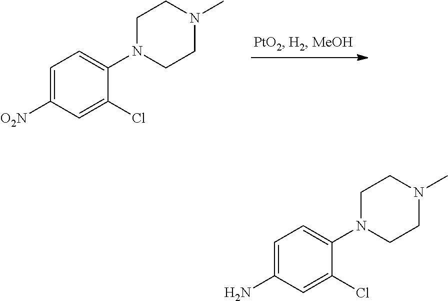 [SMILES: CN1CCN(c2ccc([N+](=O)[O-])cc2Cl)CC1 ; CN1CCN(c2ccc(N)cc2Cl)CC1]

[3-Chloro-4-(4-methyl-1-piperazinyl)phenyl]amine: 1-(2-chloro-4-nitrophenyl)-4-methylpiperazine (13.5 g, 52.8 mmol) was dissolved in Methanol (200 mL), and treated with platinum(IV) oxide (0.120 g, 0.528 mmol). The reaction was evacuated and back filled with H₂ twice, then stirred for 48 hours under an H₂ atmosphere. The crude mixture was filtered through a pad of celite, washed with MeOH, and concentrated to give the title compound as an orange solid (12 g, 100%). ¹H NMR (400 MHz, METHANOL-d₄) δ ppm 2.35 (s, 3H) 2.62 (br. s., 4H) 2.95 (br. s., 4H) 6.63 (dd, J=8.53, 2.76 Hz, 1H) 6.75-6.81 (m, 1H) 6.90-6.97 (m, 1H); MS (m/z) 226 (M+H⁺).

### Preparation 2

### 3,5-dinitrophenyl methyl sulfone

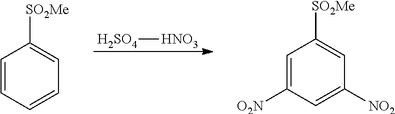

To an ice-bath cooled solution of nitric acid (90%) (20 mL) and fuming sulfuric acid (40 mL) was slowly added (methylsulfonyl)benzene (10.0 g, 64.0 mmol) in 4 portions. The ice-bath was removed then the reaction mixture was slowly heated to 140 deg C. in an oil bath for 16 h. The reaction mixture was cooled to rt then slowly poured over solid ice while swirling. The solid was collected by filtration then washed with water (300 ml), ethanol (80 mL) and ethylether (100 mL). The solid was suspended and stirred in DMSO (40 mL) for 5-10 min then filtered. The solid was washed successively with water, ethanol, then ethyl ether to give the title compound as a white solid (4.04 g, 25.6%). ¹H NMR (DMSO-d₆) δ 9.10 (t, 1H), 9.02 (d, J=2.0 Hz, 2H), 3.51 (s, 3H); MS (m/z) 247.1 (M+H⁺).

### Preparation 3

### 1-(methyloxy)-3-(methylsulfonyl)-5-nitrobenzene

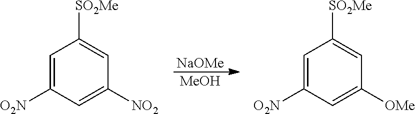 [SMILES: "MeOH" ; CS(=O)(=O)c1cc([N+](=O)[O-])cc([N+](=O)[O-])c1 ; COc1cc([N+](=O)[O-])cc(S(C)(=O)=O)c1 ; "NaOMe"]

To a suspension of 3,5-dinitrophenyl methyl sulfone (1.5 g, 6.09 mmol) in methanol (15.0 mL) was added sodium methoxide (1.672 g, 7.74 mmol) as a solution in methanol (25% w/w). The RM was heated to 70° C. for 2 h then the reaction mixture was poured onto ice. The solid was collected by filtration and washed with water, ethanol, and ethylether to yield the title compound as a light brown solid (1.16 g, 82%). ¹H NMR (DMSO-d₆) δ 8.21-8.24 (m, 1H), 8.05-8.07 (m, 1H), 7.89-7.91 (m, 1H), 4.00 (s, 3H), 3.38 (s, 3H).

### Preparation 4

### Ethyl 2-[(3-aminophenyl)thio]-2-methylpropanoate

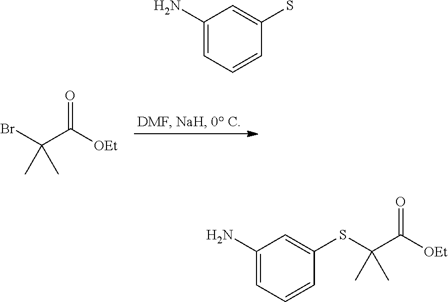

A solution of 3-aminothiophenol (97 mg, 0.775 mmol) in N,N-Dimethylformamide (DMF) (1823 μl) at 0° C. was treated with sodium hydride (30.9 mg, 1.286 mmol) and stirred for 20 min. Ethyl 2-bromo-2-methylpropanoate (114 μl, 0.775 mmol) was added and the reaction was warmed to rt over 16 hours. The reaction was diluted with DCM washing with water and brine and the organic solution was dried over Na₂SO₄, filtered, and concentrated onto silica. The crude material was purified via flash chromatography eluting with 0-50% EtOAc/hexanes on a 40 g column. ¹H NMR (DMSO-d₆) δ 6.99 (t, J=7.8 Hz, 1H), 6.62-6.68 (m, 1H), 6.59 (dd, J=8.1, 2.0 Hz, 1H), 6.53 (d, J=8.1 Hz, 1H), 5.22 (s, 2H), 4.03 (q, J=7.1 Hz, 2H), 1.40 (s, 6H), 1.13 (t, J=7.1 Hz, 3H); MS (m/z) 239 (M⁺).

The following intermediates, used for the preparation of named example compounds, were synthesized using methods analogous to the ones described above.

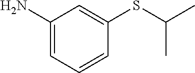

### Preparation 5

### 2-[(3-aminophenyl)thio]-2-methyl-1-propanol

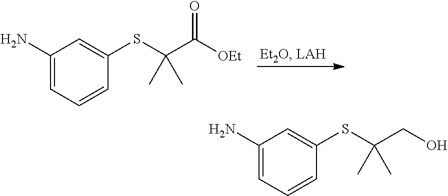 [SMILES: CCOC(=O)C(C)(C)Sc1cccc(N)c1 ; CC(C)(CO)Sc1cccc(N)c1]

A solution of ethyl 2-[(3-aminophenyl)thio]-2-methylpropanoate (85 mg, 0.355 mmol) in Diethyl ether (1421 μl) was treated with LAH (355 μl, 0.355 mmol) and stirred for 1 hour. There was no more starting material based on LCMS analysis and two new product peaks, one with the desired mass and the other unidentified. The reaction was diluted with EtOAc and satd aq NH4Cl and stirred vigorously overnight. The biphasic mixture was filtered through celite to remove the aluminum salts and the aqueous layer was extracted with DCM. The combined extracts were dried over Na2SO4, filtered, and concentrated to give the desired product. ¹H NMR (DMSO-d₆) δ 6.98 (t, J=7.7 Hz, 1H), 6.72 (t, J=2.0 Hz, 1H), 6.62 (s, 1H), 6.54-6.59 (m, 1H), 5.17 (s, 2H), 4.82 (t, J=5.9 Hz, 1H), 3.28 (d, J=5.8 Hz, 2H), 1.13 (s, 6H); MS (m/z) 197 (M⁺).

The following intermediates, used for the preparation of named example compounds, were synthesized using methods analogous to the ones described above.

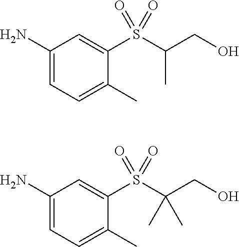 [SMILES: Cc1ccc(N)cc1S(=O)(=O)C(C)CO ; Cc1ccc(N)cc1S(=O)(=O)C(C)(C)CO]

### Preparation 6

### 2-[(3-aminophenyl)sulfonyl]-2-methyl-1-propanol

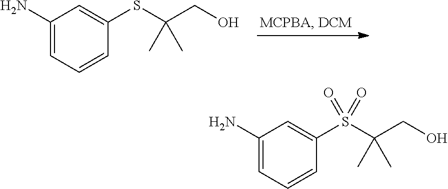

A solution of 2-[(3-aminophenyl)thio]-2-methyl-1-propanol (33.0 mg, 0.167 mmol) in Dichloromethane (DCM) (836 μl) was treated with MCPBA (57.7 mg, 0.335 mmol) and the reaction was stirred for 1 hour at rt. The mixture was diluted with DCM to dissolve the precipitate then the solution was concentrated onto silica and the crude product purified via flash chromatography using a 12 g column eluting with 0-10% MeOH/DCM. The fractions containing product were concentrated and the resulting residue was dissolved in MeOH and loaded onto a 0.5 g SCX cartridge. The cartridge was flushed with four volumes of MeOH and then the product was extracted with 2N NH3 in MeOH using three volumes. The extracts were concentrated to give the desired product as a white solid (23 mg, 60%). ¹H NMR (DMSO-d₆) δ 7.25 (t, J=7.8 Hz, 1H), 6.97-7.01 (m, 1H), 6.83-6.90 (m, 2H), 5.65 (s, 2H), 5.02 (t, J=6.1 Hz, 1H), 3.47 (d, J=6.1 Hz, 2H), 1.17 (s, 6H); MS (m/z) 229 (M⁺).

### Preparation 7

### 2,2,2-trifluoro-1-(3-nitrophenyl)ethanamine

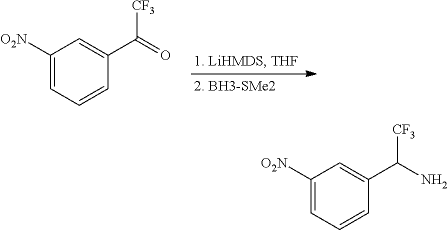 [SMILES: O=C(c1cccc([N+](=O)[O-])c1)C(F)(F)F ; NC(c1cccc([N+](=O)[O-])c1)C(F)(F)F]

2,2,2-Trifluoro-1-(3-nitrophenyl)ethanone (5.00 g, 22.82 mmol) was dissolved in toluene (30 mL) at room temperature. A solution of 1M LiHMDS in THF (25.6 mL, 25.6 mmol) was added into the reaction solution slowly over 10 min period of time. The mixture was stirred at room temperature for 15 min, then BH₃.DMS (4.40 mL, 46.3 mmol) was added. The reaction mixture was stirred at room temperature for 2 h. The reaction mixture was then quenched with Ice-water mixture. The quenched reaction mixture was partitioned between water and dichloromethane. The organic layer was washed by brine, dried over MgSO₄, filtered, and concentrated to about 10 ml of the toluene solution. A solution of 3 ml 4N HCl in dioxane was added dropwise. The resulting white precipitate was collected by filtration, and then was dried under high vacuum for 16 h to give the desired product as a white solid (5.3 g, 91% yield). ¹H NMR (400 MHz, DMSO-d₆) δ 8.40 (dd, J=8.2, 1.4 Hz, 1H), 8.17 (d, J=7.8 Hz, 1H), 7.86 (t, J=8.1 Hz, 1H), 7.05 (m, 1H), 5.87 (m, 1H), 3.89 (s, 2H); MS (m/z) 221 (M+H⁺).

### Preparation 8

### 2,2,2-trifluoro-N,N-dimethyl-1-(3-nitrophenyl)ethanamine

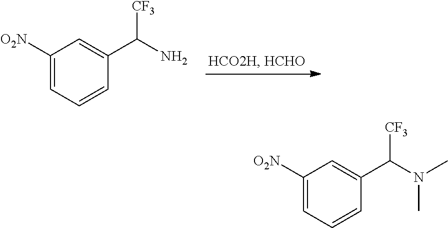 [SMILES: NC(c1cccc([N+](=O)[O-])c1)C(F)(F)F ; CN(C)C(c1cccc([N+](=O)[O-])c1)C(F)(F)F]

2,2,2-Trifluoro-1-(3-nitrophenyl)ethanamine (1 g, 4.54 mmol) was dissolved in formic acid (3484 μl, 91 mmol) at room temperature. Paraformaldehyde (546 mg, 18.17 mmol) was added to the reaction mixture, and then the mixture was stirred at 100° C. for 3 h.

The reaction mixture was added into 150 ml of sat. Na₂CO₃(aq) slowly, and then was extracted by dichloromethane. The organic layer was washed by brine, dried over MgSO₄ and concentrated to a brown oil. The crude oil was purified by Isco Combiflash (5%-20% EtOAc/Hexane; 40 g column). Collected fractions were combined and concentrated to give the desired product as a colorless oil (600 mg, 53% yield). MS (m/z) 221 (M+H⁺).

### Preparation 9

### 4-[(3-bromo-5-methylphenyl)sulfonyl]tetrahydro-2H-pyran

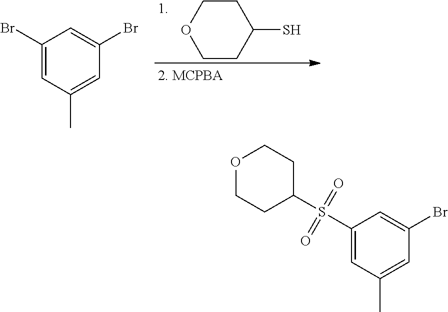

To a mixture of sodium hydride (0.528 g, 13.20 mmol) in N,N-Dimethylformamide (DMF) (30 mL) was added tetrahydro-2H-pyran-4-thiol (1.703 g, 14.40 mmol) and the mixture was stirred at room temperature for 30 minutes. Then 1,3-dibromo-5-methylbenzene (1.50 g, 6.00 mmol) was added and reaction was then heated at 80° C. for 16 hours. The reaction was cooled to room temperature and 1 mL 6M NaOH (aq) and 30 mL water were added and mixture was stirred for 10 minutes. Hexanes were added, layers were separated and hexanes were washed again with brine. Organics were concentrated to give 2.43 g light yellow non-viscous oil, 86% pure, MS (m/z) 287/289. Br pattern, (M+H⁺). 4-[(3-bromo-5-methylphenyl)thio]tetrahydro-2H-pyran (2.43 g, 86% pure) was dissolved in Dichloromethane (DCM) (50.0 mL) and cooled in an ice/water bath. Next MCPBA (3.36 g, 15.00 mmol) was added in portions and reaction was stirred at room temperature for 1 hour. Reaction was diluted with satd. NaHCO₃, layers were separated and organics were washed with 2M NaOH and brine. Organics were concentrated and dried to give the title compound as a light yellow solid in 75% purity (2.08 g, 67%) MS (m/z) 319/321, Br pattern, (M+H⁺). Product was used as-is in the next reaction.

The following intermediate, used for the preparation of named example compounds, was synthesized using methods analogous to the ones described above.

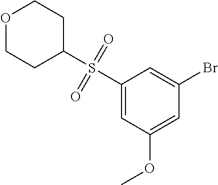 [SMILES: COc1cc(Br)cc(S(=O)(=O)C2CCOCC2)c1]

### Preparation 10

### 1-bromo-3-[(1,1-dimethylethyl)sulfonyl]-5-methylbenzene

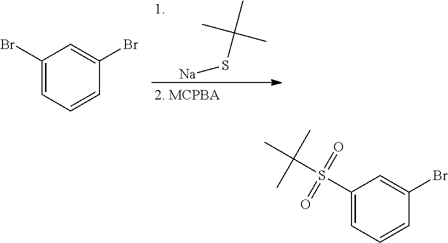

1,3-Dibromobenzene (1.023 mL, 8.48 mmol) was dissolved in N-Methyl-2-pyrrolidone (NMP) (20 mL) and t-butylthiol sodium salt (3.17 g, 25.4 mmol) was slowly added at room temperature. Reaction was slightly exothermic and turned medium pink/red color. Reaction was heated at 80° C. for 5 days. Reaction was cooled to room temperature and 0.5 mL 6M NaOH (aq) and 20 mL water were added and mixture was stirred for 10 minutes. Hexanes were added, layers were separated and hexanes were washed again with brine. Organics were concentrated to give 3.0 g light yellow liquid, a mixture of ˜2:1 desired product:bis-alkylated product. MS (m/z), did not ionize. Crude product was used as-is in next reaction. 1-bromo-3-[(1,1-dimethylethyl)thio]benzene (3.00 g, 12.24 mmol) was dissolved in Dichloromethane (DCM) (50 mL) and cooled in an ice/water bath, MCPBA (6.86 g, 30.6 mmol) was added in portions and reaction was stirred at room temperature for 120 minutes. Reaction was diluted with satd. NaHCO₃, layers were separated and organics were washed with 2M NaOH and brine. Organics were concentrated and purified by column chromatography to give the title compound as a white solid (1.90 g, 90% yield for oxidation, based on 62% purity of starting material). ¹H NMR (400 MHz, CHLOROFORM-d) δ 8.05 (t, J=1.77 Hz, 1H), 7.82 (m, 2H), 7.46 (t, J=7.83 Hz, 1H), 1.38 (s, 9H); MS (m/z) 277/279.

The following intermediates, used for the preparation of named example compounds, were synthesized using methods analogous to the ones described above.

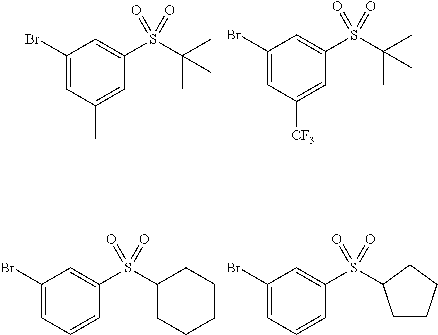

### Preparation 11

### 3-bromo-N,N,5-trimethylbenzenesulfonamide

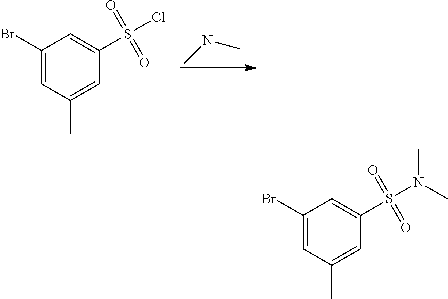

3-Bromo-5-methylbenzenesulfonyl chloride (500 mg, 1.855 mmol) was dissolved in Dichloromethane (DCM) (15 mL) and cooled in an ice/water bath. Then dimethylamine (2.78 mL, 5.56 mmol) was added and reaction was stirred at room temperature for 20 minutes. Reaction was diluted with satd. NaHCO₃, layers were separated and organics were dried over sodium sulfate. Organics were concentrated and dried to give the title compound as a light orange solid (513 mg, 96%). ¹H NMR (400 MHz, CHLOROFORM-d) δ 7.74 (s, 1H), 7.58 (s, 1H), 7.53 (s, 1H), 2.75 (s, 6H), 2.45 (s, 3H); MS (m/z) 278/280 (M+H⁺).

The following intermediates, used for the preparation of named example compounds, were synthesized using methods analogous to the ones described above.

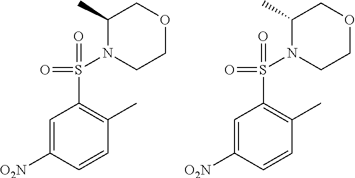 [SMILES: Cc1ccc([N+](=O)[O-])cc1S(=O)(=O)N1CCOC[C@@H]1C ; Cc1ccc([N+](=O)[O-])cc1S(=O)(=O)N1CCOC[C@H]1C]

### Preparation 12

### 1-bromo-3-methyl-5-(methylsulfonyl)benzene

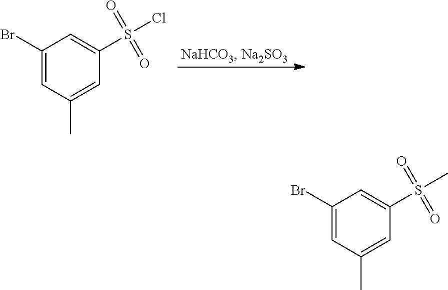

To a solution of sodium sulfite (1.870 g, 14.84 mmol) and sodium bicarbonate (1.309 g, 15.58 mmol) in Water (10 mL) was added 3-bromo-5-methylbenzenesulfonyl chloride (2.00 g, 7.42 mmol) and Ethanol (5.00 mL). Mixture was heated at 50° C. for 45 minutes and concentrated to dryness. Crude was suspended in N,N-Dimethylformamide (DMF) (15 mL), iodomethane (2.315 mL, 37.1 mmol) was added and mixture was stirred at room temperature for 15 minutes. Reaction was diluted with diethyl ether and satd. NaHCO₃ and layers were separated. Organics were washed with water, dried over sodium sulfate, filtered, concentrated and dried to give title compound as a light yellow solid (1.64 g, 84%). ¹H NMR (400 MHz, CHLOROFORM-d) δ 7.91 (s, 1H), 7.71 (s, 1H), 7.63 (s, 1H), 3.08 (s, 3H), 2.46 (s, 3H); MS (m/z) 249/251 (M+H⁺).

The following intermediates, used for the preparation of named example compounds, were synthesized using methods analogous to the ones described above.

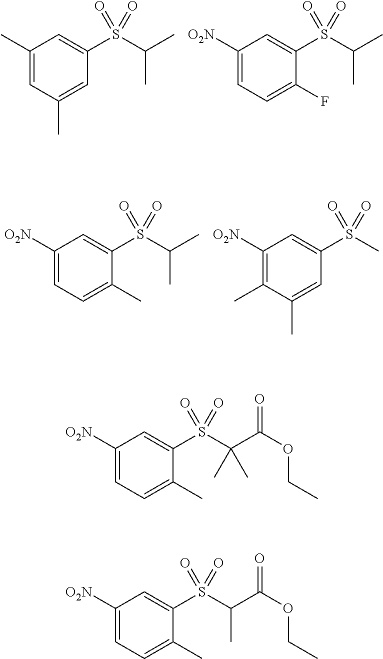

### Preparation 13

### 3-[(diphenylmethylidene)amino]-N,N,5-trimethylbenzenesulfonamide

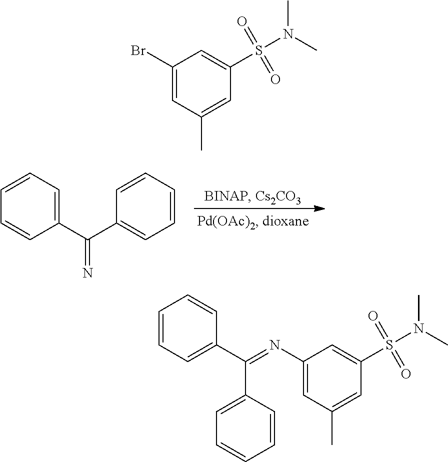 [SMILES: Cc1cc(Br)cc(S(=O)(=O)N(C)C)c1 ; N=C(c1ccccc1)c1ccccc1 ; Cc1cc(N=C(c2ccccc2)c2ccccc2)cc(S(=O)(=O)N(C)C)c1]

3-Bromo-N,N,5-trimethylbenzenesulfonamide (510 mg, 1.833 mmol), benzophenone imine (0.369 mL, 2.200 mmol), cesium carbonate (836 mg, 2.57 mmol) and BINAP (114 mg, 0.183 mmol) and palladium(II) acetate (41.2 mg, 0.183 mmol) were mixed in 1,4-Dioxane (8 mL) and nitrogen was bubbled through for 5 minutes. The reaction was microwaved at 130° C. for 30 minutes, diluted with water and DCM and layers were separated. Organics were concentrated and purified by chromatography (25 g silica column; 3-20% E/H, 25 min.) to give the title compound as a yellow foam (532 mg, 73%). ¹H NMR (400 MHz, CHLOROFORM-d) δ 7.71-7.77 (m, 2H), 7.49-7.55 (m, 1H), 7.41-7.47 (m, 3H), 7.29-7.32 (m, 2H), 7.11-7.18 (m, 3H), 6.97 (s, 1H), 6.78 (s, 1H), 2.43 (s, 6H), 2.36 (s, 3H); MS (m/z) 379.2 (M+H⁺).

### 3-amino-N,N,5-trimethylbenzenesulfonamide

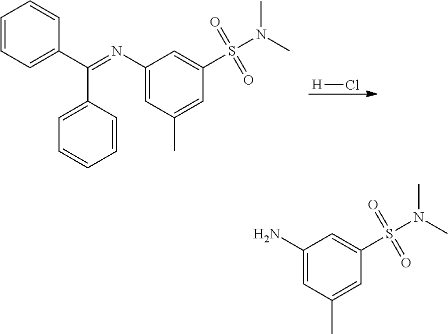

3-[(Diphenylmethylidene)amino]-N,N,5-trimethylbenzenesulfonamide (529 mg, 1.258 mmol) was dissolved in Tetrahydrofuran (THF) (7 mL) and HCl (0.839 mL, 5.03 mmol, 6M aqueous) was added. Reaction was stirred at room temperature for 60 minutes and then concentrated. Crude was partitioned between ethyl acetate and satd. NaHCO₃ and layers were separated. Organics were concentrated and triturated in diethyl ether, filtered and dried to give the title compound as a pale yellow solid (220 mg, 81%). ¹H NMR (400 MHz, CHLOROFORM-d) δ 6.96 (s, 1H), 6.88 (s, 1H), 6.70 (s, 1H), 3.87 (br. s., 2H), 2.72 (s, 6H), 2.34 (s, 3H); MS (m/z) 215.0 (M+H⁺).

The following intermediates, used for the preparation of named example compounds, were synthesized using methods analogous to the ones described above.

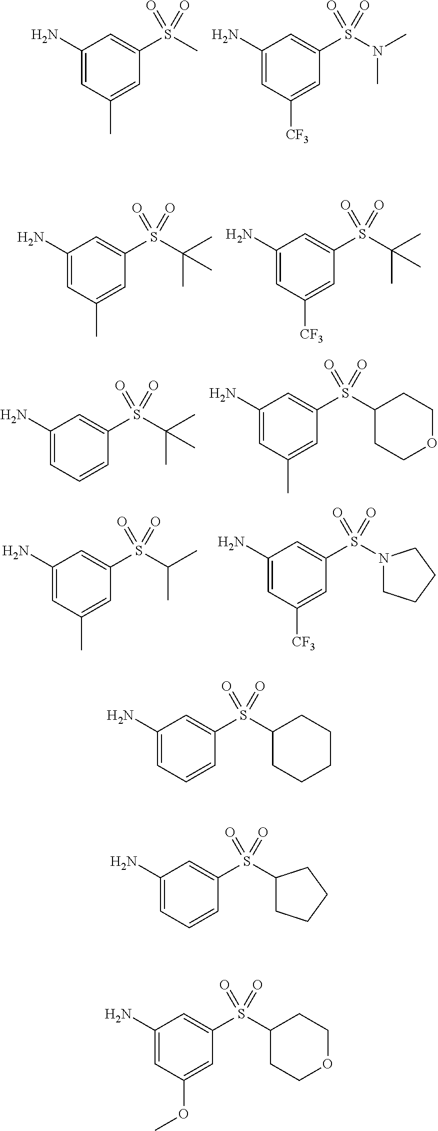

### Preparation 14

### 1,2-dimethyl-3-(methylsulfonyl)-5-nitrobenzene 2,3-dimethyl-5-nitrophenyl methyl sulfone

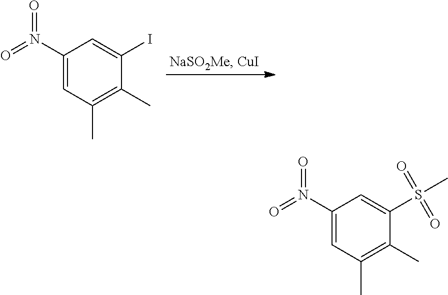

A mixture of 1-iodo-2,3-dimethyl-5-nitrobenzene (1.20 g, 4.03 mmol), sodium methanesulfinate (0.581 g, 4.83 mmol) and copper(I) iodide (1.151 g, 6.04 mmol) in N,N-Dimethylformamide (DMF) (6 mL) was heated at 110° C. for 2 hours. Reaction turned orange and a precipitate formed when it got up to temperature, brown after 2 h. More sodium methanesulfinate (0.581 g, 4.83 mmol) was added and mixture was heated another 1 hour. Then more copper(I) iodide (1.151 g, 6.04 mmol) was added and mixture was heated another 4 hours. Reaction was diluted with water and ethyl acetate and filtered to remove insolubles. Filtrate layers were separated and organics were concentrated and purified by column chromatography using an ethyl acetate/hexanes gradient to give the title compound as a light yellow solid (262 mg, 28%). ¹H NMR (400 MHz, CHLOROFORM-d) δ 8.83 (m, 1H), 8.31 (m, 1H), 3.18 (s, 3H), 2.77 (s, 3H), 2.52 (s, 3H); MS (m/z) 230.1 (M+H⁺).

The following intermediates, used for the preparation of named example compounds, were synthesized using methods analogous to the ones described above.

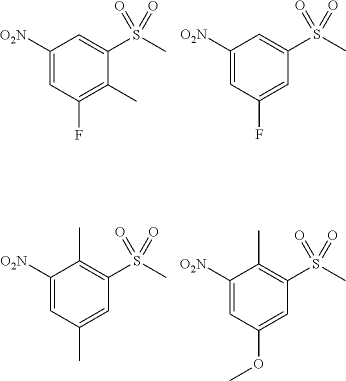

### Preparation 15

### 1-[2-methyl-3-(methylsulfonyl)-5-nitrophenyl]pyrrolidine

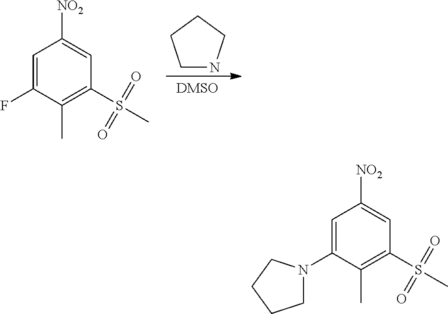

A mixture of 1-fluoro-2-methyl-3-(methylsulfonyl)-5-nitrobenzene (92.0 mg, 0.394 mmol), and pyrrolidine (140 mg, 1.972 mmol) were heated at 100.0° C. for 30 min. The reaction mixture was cooled to rt then quenched with cold water. The suspended solid was collected by filtration then purified by FCC using a Biotage unit [EtOAc-Hex: 10-35%] to yield the title compound (57.0 mg; 51.0%). MS (m/z) 285.2 (M+H⁺).

The following intermediates, used for the preparation of named example compounds, were synthesized using methods analogous to the ones described above.

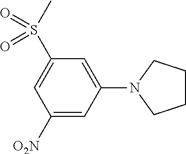 [SMILES: CS(=O)(=O)c1cc(N2CCCC2)cc([N+](=O)[O-])c1]

### Preparation 16

### 3-(methylsulfonyl)-5-nitrophenol

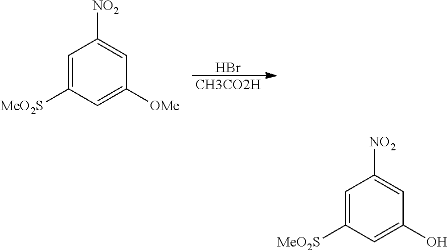

A solution of 1-(methyloxy)-3-(methylsulfonyl)-5-nitrobenzene (435.0 mg, 1.881 mmol) in 6.0 mL of a solution of HBr in Acetic acid (40% w/w) was heated at 90.0° C. in an oil bath. The reaction mixture was poured onto solid ice then the aqueous suspension was extracted with EtOAc. The organic layer was washed with sat.aq NaHCO₃, dried over sodium sulfate, filtered then concentrated. The residue was purified by FCC using a Biotage unit [EtOAc-Hex:10 to 40%] to yield the title compound (191.0 mg, 46.7%). MS (m/z): 218.0 (M+H⁺).

### Preparation 17

### 1-[(difluoromethyl)oxy]-3-(methylsulfonyl)-5-nitrobenzene

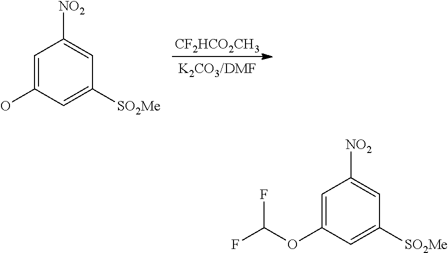

To a solution of 3-(methylsulfonyl)-5-nitrophenol (66.0 mg, 0.304 mmol) in DMF (2.5 mL) was added potassium carbonate (147 mg, 1.064 mmol). The mixture was stirred for 20 min at rt then methyl chloro(difluoro)acetate (0.081 mL, 0.760 mmol) was added and the reaction mixture was heated to 90° C.

After 1 h the reaction mixture was cooled to rt then diluted with EtOAc and washed with water. The organic layer was dried over sodium sulfate, filtered then concentrated. The residue was purified by FCC using a Biotage unit[EtOAc-Hex: 10 to 50%]. ¹H NMR (DMSO-d₆) δ 8.50-8.55 (m, 1H), 8.36-8.40 (m, 1H), 8.17-8.20 (m, 1H), 7.38-7.78 (m, 1H), 3.43 (s, 3H); MS (m/z): 268.3 (M+H⁺).

### Preparation 18

### [3-[(difluoromethyl)oxy]-5-(methylsulfonyl)phenyl]amine

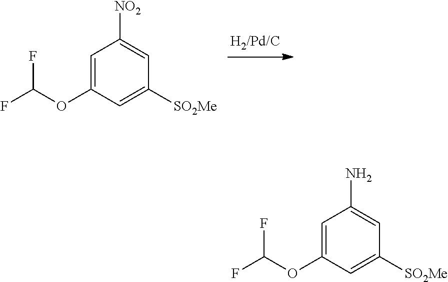 [SMILES: CS(=O)(=O)c1cc(OC(F)F)cc([N+](=O)[O-])c1 ; CS(=O)(=O)c1cc(N)cc(OC(F)F)c1]

To a solution of 1-[(difluoromethyl)oxy]-3-(methylsulfonyl)-5-nitrobenzene (110.0 mg, 0.412 mmol) in 4 mL ethyl acetate-ethanol (3:1) was added Pd/C (10% w/w) (43.8 mg, 0.041 mmol). The mixture was stirred under hydrogen gas which was supplied from a balloon. After 4 h the reaction mixture was filtered then concentrated in-vacuo to yield the title compound. MS (m/z): 238.1 (M+H⁺).

### Preparation 19

### ethyl 3-(methylthio)-5-nitrobenzoate

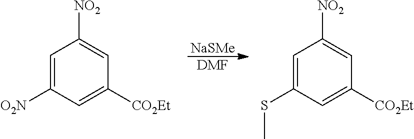

To a suspension of sodium thiomethoxide (0.482 g, 6.87 mmol) in DMF at 0° C. was added ethyl 3,5-dinitrobenzoate (1.50 g, 6.25 mmol). The reaction mixture was stirred overnight allowing it to reach rt. The reaction mixture was quenched with water then extracted with EtOAc and dried over sodium sulfate. The organic layer was concentrated in-vacuo then the residue was purified by FCC on a Biotage unit [EtOAc-Hex: 10-15%] to yield the title compound (300.0 mg, 14.9%). MS (m/z) 242.2 (M+H⁺).

### Preparation 20

### ethyl 3-(methylsulfonyl)-5-nitrobenzoate

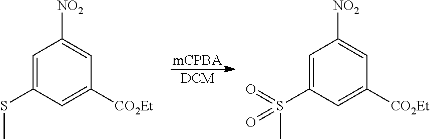

To a cooled mixture of ethyl 3-(methylthio)-5-nitrobenzoate (125.0 mg, 0.518 mmol) and sodium bicarbonate (174 mg, 2.072 mmol) in dichloromethane (5.0 mL) was added mCPBA (244.0 mg, 1.089 mmol). The reaction mixture was removed from the ice-bath then stirred overnight at rt. After 20 h then reaction mixture was quenched by pouring it over aq. sodium carbonate solution (13% w/w). The mixture was extracted with CH₂Cl₂ then the organic layer was dried over sodium sulfate, filtered and concentrated in-vacuo. The residue was purified by FCC using a Biotage unit [EtOAc-Hex:10-35%] to give the title compound (94.0 mg, 66.4%). ¹H NMR (DMSO-d₆) δ 8.88-8.92 (m, 1H), 8.85-8.86 (m, 1H), 8.74-8.78 (m, 1H), 4.45 (q, J=7.2 Hz, 2H), 3.45 (s, 3H), 1.39 (t, J=7.1 Hz, 3H); MS (m/z): 274.1 (M+H⁺).

### Preparation 21

### [3,4-dimethyl-5-(methylsulfonyl)phenyl]amine

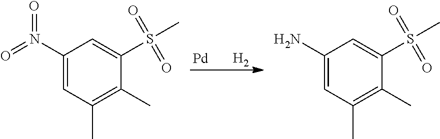

1,2-Dimethyl-3-(methylsulfonyl)-5-nitrobenzene (255 mg, 1.112 mmol) was suspended in Ethanol (7 mL) and palladium on carbon (118 mg, 0.111 mmol) was added. Mixture was purged with nitrogen and then put under vacuum. Then vacuum was released with hydrogen (balloon) and reaction was stirred at room temperature overnight. Mixture was filtered through Celite, rinsing with methanol and filtrate was concentrated and dried to give the title compound as a dark green sticky oil (213 mg, 86%) in 90% purity. ¹H NMR (400 MHz, DMSO-d₆) δ 7.06 (d, J=2.27 Hz, 1H), 6.70 (m, 1H), 5.33 (s, 2H), 3.11 (s, 3H), 2.36 (s, 3H), 2.19 (s, 3H); MS (m/z) 200.1 (M+H⁺).

The following intermediates, used for the preparation of named example compounds, were synthesized using methods analogous to the ones described above.

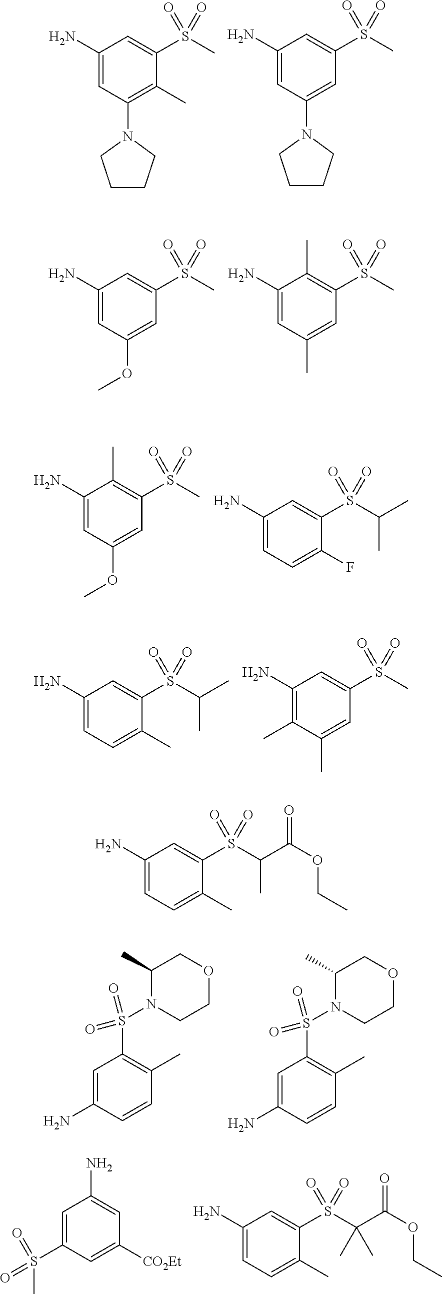

### Preparation 22

### N-[3,4-Bis(methyloxy)phenyl]-4-chloro-2-pyrimidinamine

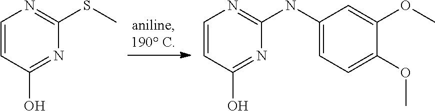

2-{[3,4-Bis(methyloxy)phenyl]amino}-4(1H)-pyrimidinone: 2-(methylthio)-4(1H)-pyrimidinone (8.2 g, 57.7 mmol) and [[3,4-bis(methyloxy)phenyl]amine 3,4-bis(methyloxy)aniline (9.28 g, 60.6 mmol) were heated at 180° C. for 2 hours. The resulting residue was cooled to rt and triturated with EtOH (4 mL). The resulting solid was filtered, washed with EtOH, and air dried air to give the title compound (12.4 g, 87%). ¹H NMR (400 MHz, DMSO-d₆) δ ppm 3.73 (s, 3H) 3.74 (s, 3H) 5.75 (d, J=6.02 Hz, 1H) 6.90 (d, J=8.78 Hz, 1H) 7.06 (br. s., 1H) 7.23 (br. s., 1H) 7.63-7.79 (m, 1H) 8.50-8.80 (m, 1H); MS (m/z) 248 (M+H⁺).

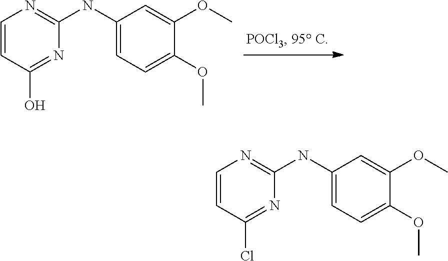 [SMILES: COc1ccc(Nc2nccc(O)n2)cc1OC ; COc1ccc(Nc2nccc(Cl)n2)cc1OC]

N-[3,4-Bis(methyloxy)phenyl]-4-chloro-2-pyrimidinamine: A flask was charged with 2-{[3,4-bis(methyloxy)phenyl]amino}-4(1H)-pyrimidinone (31 g, 125 mmol) followed by POCl₃ (180 mL, 1930 mmol). The reaction was heated to 95° C. for 4 hours then was cooled to rt and diluted with water. The solution was quenched with aq NaOH and the resulting precipitate collected via filtration (17 g). The filtrate was partially concentrated and more solid crashed out. The solid was isolated via filtration (4 g) and the filtrate was extracted with EtOAc. The combined extracts were concentrated onto silica and the crude material was purified via flash chromatography eluting with 20-60% THF/hexanes to give additional product (1.6 g). The three portions were combined to give the title compound. ¹H NMR (400 MHz, CHLOROFORM-d) δ ppm 3.89 (s, 3H) 3.92 (s, 3H) 6.73 (d, J=5.27 Hz, 1H) 6.87 (d, J=8.53 Hz, 1H) 7.01 (dd, J=8.53, 2.51 Hz, 1H) 7.21-7.28 (m, 1H) 7.32 (d, J=2.51 Hz, 1H) 8.27 (d, J=5.27 Hz, 1H); MS (m/z) 265 (M+H⁺).

The following intermediates used for the preparation of titled example compounds were synthesized using methods analogous to the ones described above.

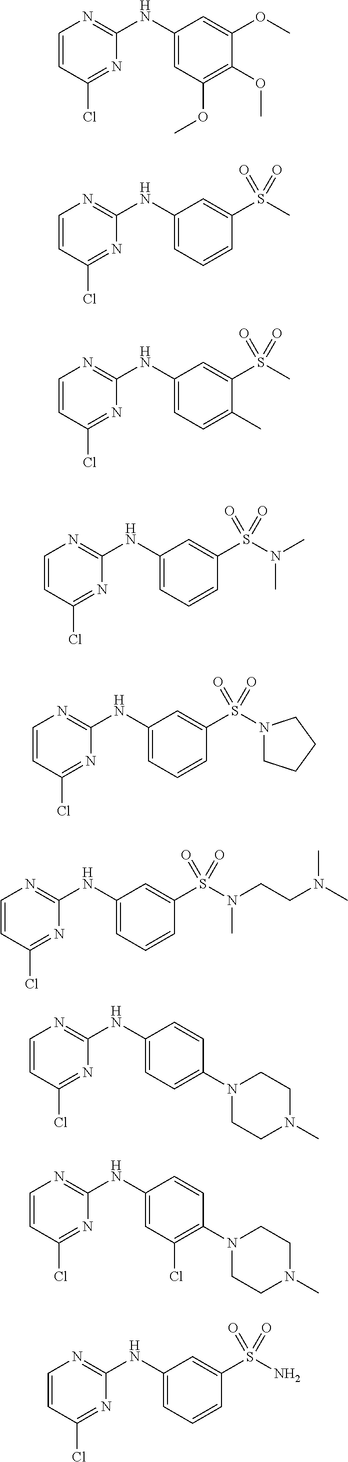

### Preparation 23

### 3,4-Dimethyl-1H-pyrazol-5-amine

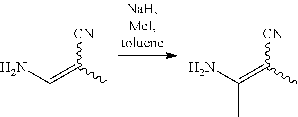

(2Z)-3-Amino-2-methyl-2-butenenitrile: To a suspension of NaH (11.69 g, 292 mmol) in toluene (100 mL) at 30° C. was added a solution of (2Z)-3-amino-2-butenenitrile (20 g, 244 mmol) in toluene (400 mL) and the reaction mixture was stirred for 10 min. then MeI (15.23 ml, 244 mmol) was added and the reaction was cooled to 40° C. with cold water. The reaction was then allowed to cool to 30° C. and stirred overnight. An orange solid formed and was collected via filtration washing with toluene. The solid was suspended in water (400 mL) and stirred for 1 hour. The solid was then filtered washing with water and air dried for 15 min, then placed under vacuum overnight (6.7 g, 29%). The mother liquor was concentrated under vacuum and the resulting residue dissolved in EtOAc to give a biphasic solution with mineral oil. The layers were separated and the EtOAc was removed under vacuum; the resulting solid was recrystallized from benzene to give the title compound (2.8 g, 12%). ¹H NMR (400 MHz, DMSO-d₆) δ ppm 1.57 (s, 3H) 1.92 (s, 3H) 6.12 (br. s., 2H); MS (m/z) 97 (M+H⁺).

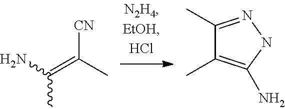

3,4-Dimethyl-1H-pyrazol-5-amine: To a solution of (2Z)-3-amino-2-methyl-2-butenenitrile (1.000 g, 10.40 mmol) in Ethanol (10.4 ml) was added hydrazine (0.602 ml, 10.40 mmol). The resulting mixture was heated to 75° C. for 16 hours open to atmosphere. The reaction was concentrated onto silica gel and purified via flash chromatography eluting with 0-10% MeOH in DCM over 37 min to give the titled compound as a yellow oil (710 mg, 61%). ¹H NMR (400 MHz, DMSO-d₆) δ ppm 1.72 (s, 3H) 1.99 (s, 3H) 3.99-4.50 (m, 2H) 10.72-11.07 (m, 1H); MS (m/z) 112 (M+H⁺).

The following intermediates used for the preparation of titled example compounds were synthesized using methods analogous to the ones described above.

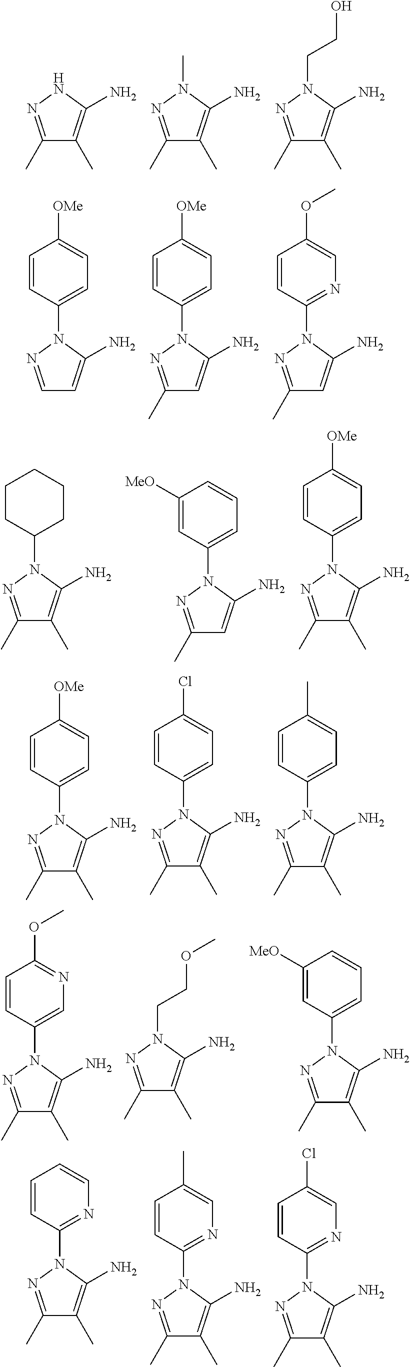

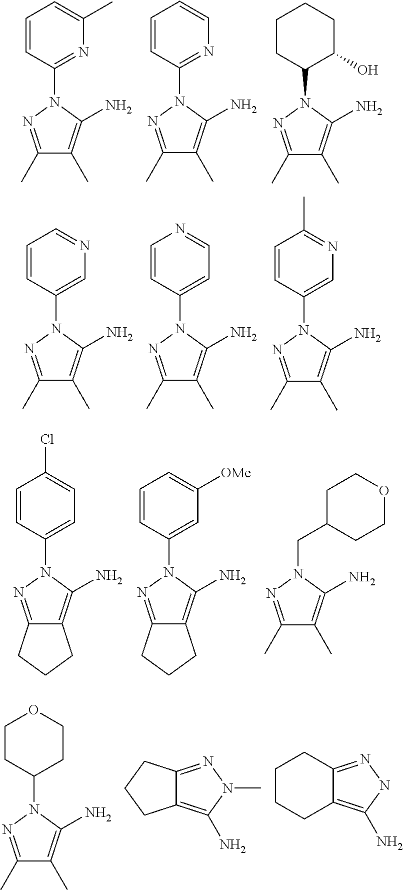

### Preparation 24

### N,1,3,4-Tetramethyl-1H-pyrazol-5-amine 1

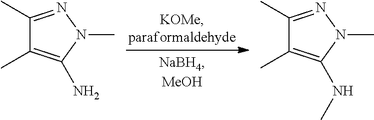

1,3,4-Trimethyl-1H-pyrazol-5-amine (100 mg, 0.799 mmol) was dissolved in Methanol (4 mL) and paraformaldehyde (72.0 mg, 2.397 mmol) and potassium methoxide (1.50 mL, 5.08 mmol) were added. The mixture was refluxed for 1 hour, then removed from heat. NaBH₄ (76 mg, 1.997 mmol) was added and the mixture was refluxed overnight. By LCMS, the mixture is 80:20 product:SM. The reaction was cooled to room temperature, a few drops satd. NaHCO₃ was added and mixture was concentrated to remove methanol. The crude material was diluted with brine and DCM, and organics were separated and concentrated. Crude product was purified by flash chromatography (10 g silica column; 0.5-5% MeOH/DCM, 20 min.) to give the title compound as a yellow solid (57 mg, 50%). ¹H NMR (400 MHz, DMSO-d₆) δ 4.50 (d, J=5.31 Hz, 1H), 3.45 (s, 3H), 2.67 (d, J=5.56 Hz, 3H), 1.93 (s, 3H), 1.85 (s, 3H); MS (m/z) 140.0 (M+H⁺).

### Preparation 25

### 1-[4-(Methyloxy)phenyl]-3-(trifluoromethyl)-1H-pyrazol-5-amine

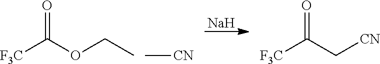

4,4,4-Trifluoro-3-oxobutanenitrile: To a suspension of NaH (0.845 g, 21.12 mmol) in Tetrahydrofuran (THF) (20 mL) was added ethyl trifluoroacetate (2 g, 14.08 mmol), followed by acetonitrile (6.93 g, 169 mmol), the resulting mixture was heated at 80° C. for 20 hours, then cooled to room temperature and concentrated. The residue was diluted with water and extracted with EtOAc (2×50 mL) the combined extracts were washed with 1N HCl, then brine, dried over MgSO4, and filtered. The solvent was removed to give desired product as an oil (3 g).

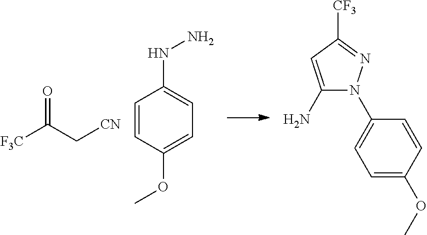

1-[4-(Methyloxy)phenyl]-3-(trifluoromethyl)-1H-pyrazol-5-amine: To a solution of 4,4,4-trifluoro-3-oxobutanenitrile (227 mg, 1.65 mmol) was added [4-(methyloxy)phenyl]hydrazine (736 mg, 4.22 mmol) followed by conc. HCl (5 mL). The mixture was heated at 90° C. for 3 hours, cooled to room temperature and concentrated. The resulting residue was redissolved in EtOAc and water and the organic layer was isolated and purified by flash chromatography to give title compound as a white solid (145 mg, 34%). MS (m/z) 258.1 (M+H)+

### Preparation 26

### 4-Methyl-1-[4-(methyloxy)phenyl]-3-(trifluoromethyl)-1H-pyrazol-5-amine

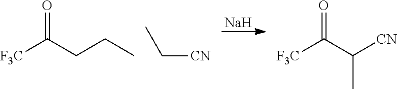

4,4,4-Trifluoro-2-methyl-3-oxobutanenitrile: To a suspension of NaH (0.845 g, 21.12 mmol) in tetrahydrofuran (THF) (20 mL) was added ethyl trifluoroacetate (2 g, 14.08 mmol) followed by propanenitrile (9.30 g, 169 mmol) and the resulting mixture was heated at 80° C. for 20 hours. The reaction was cooled to room temperature, concentrated, and the resulting residue was diluted with water and EtOAc. The aqueous layer was extracted with EtOAc (2×50 mL) and the combined extracts were washed with 1N HCl, then brine, dried over MgSO₄, and filtered. The solvent was removed via rotovap to give the product as an oil (3 g).

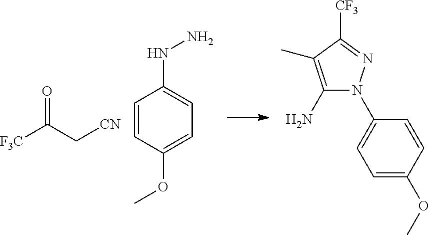

4-Methyl-1-[4-(methyloxy)phenyl]-3-(trifluoromethyl)-1H-pyrazol-5-amine: 4,4,4-Trifluoro-2-methyl-3-oxobutanenitrile (216 mg, 1.43 mmol) was dissolved in EtOH (50 mL). To this solution was added [4-(methyloxy)phenyl]hydrazine (639 mg, 3.66 mmol) followed by conc. HCl (5 mL). The mixture was heated at 90° C. for 3 hours, then cooled to room temperature and concentrated. The resulting residue was suspended between water and EtOAc. The aqueous layer was extracted with EtOAc and the combined extracts were dried over MgSO4, filtered, and concentrated. The crude material was purified by flash chromatography to give titled compound as a white solid (174 mg, 45%). MS (m/z) 272.1 (M+H)+

### Preparation 27

### 2-chloro-N-(3,4-dimethyl-1H-pyrazol-5-yl)-4-pyrimidinamine

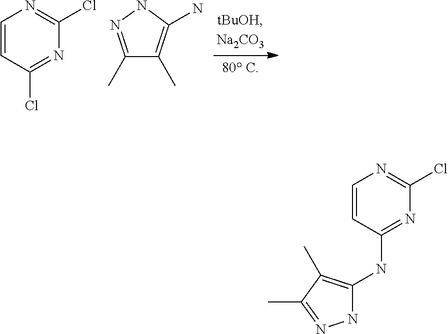

A mixture of 2,4-dichloropyrimidine (7.51 g, 50.4 mmol) and 3,4-dimethyl-1H-pyrazol-5-amine (5.60 g, 50.4 mmol) in 1-Butanol (252 ml) was treated with sodium carbonate (16.02 g, 151 mmol) and heated to 80° C. for 16 hours. The reaction was diluted with DCM and washed with water. The aqueous layer was extracted with DCM and the combined organic layers were dried over Na₂SO₄, filtered, and concentrated onto silica. The crude product was purified via flash chromatography using a 120 g column eluting with 0-100% EtOAc/hexanes. ¹H NMR (DMSO-d₆) δ 12.17 (s, 1H), 9.71 (s, 1H), 8.12 (d, J=6.1 Hz, 1H), 6.99 (br. s., 1H), 2.14 (s, 3H), 1.81 (s, 3H); MS (m/z) 223 (M⁺).

### Preparation 28

### 2-chloro-N-(3,4-dimethyl-5-isoxazolyl)-4-pyrimidinamine

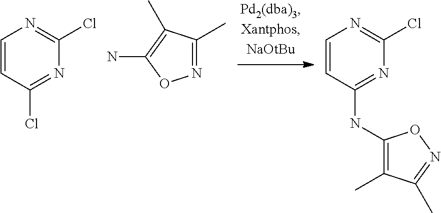

2,4-Dichloropyrimidine (4.78 g, 32.1 mmol) and 3,4-dimethyl-5-isoxazolamine (3.00 g, 26.8 mmol) were combined in Toluene (100 mL) and Pd₂(dba)₃ (1.225 g, 1.338 mmol), Xantphos (1.548 g, 2.68 mmol) and sodium tert-butoxide (5.29 g, 55.0 mmol) were added. Nitrogen was bubbled through the mixture for several minutes and it was then heated at 100° C. for 2 hours. The reaction was cooled to room temperature, filtered through Celite, rinsing liberally with acetone, then methanol. Filtrate was concentrated and partitioned between ethyl acetate and water and a small amount of an insoluble solid was filtered. Layers were separated, organics were concentrated to a solid which was triturated in DCM and filtered to give the title compound as a light orange solid (1.35 g, 21%). Filtrate was purified by column chromatography using an ethyl acetate/hexanes gradient to give the title compound as a light orange solid (721 mg, 12%). ¹H NMR (400 MHz, DMSO-d₆) δ 10.67 (s, 1H), 8.31 (d, J=5.81 Hz, 1H), 6.82 (d, J=5.81 Hz, 1H), 2.18 (s, 3H), 1.83 (s, 3H); MS (m/z) 225.2/227.2 (M+H⁺).

### Preparation 29

### 2-chloro-N-(3,4-dimethyl-5-isoxazolyl)-N-methyl-4-pyrimidinamine

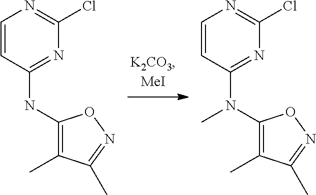 [SMILES: Cc1noc(Nc2ccnc(Cl)n2)c1C ; Cc1noc(N(C)c2ccnc(Cl)n2)c1C]

To a solution of 2-chloro-N-(3,4-dimethyl-5-isoxazolyl)-4-pyrimidinamine (1.35 g, 5.71 mmol) in N,N-Dimethylformamide (DMF) (15 mL) under nitrogen was added potassium carbonate (1.578 g, 11.42 mmol) and iodomethane (0.428 mL, 6.85 mmol). Reaction was stirred at room temperature for 60 minutes. Reaction was diluted with water and diethyl ether and layers were separated. Organics were washed with brine, dried over sodium sulfate and concentrated to give the title compound as an orange solid (1.30 g, 91%). ¹H NMR (400 MHz, DMSO-d₆) δ 8.29 (d, J=6.06 Hz, 1H), 6.63 (d, J=5.81 Hz, 1H), 3.36 (s, 3H), 2.23 (s, 3H), 1.78 (s, 3H); MS (m/z) 239.2/241.2 (M+H⁺).

### Preparation 30

### 1,1-dimethylethyl 3-[(2-chloro-4-pyrimidinyl)(methyl)amino]-4,5-dimethyl-1H-pyrazole-1-carboxylate

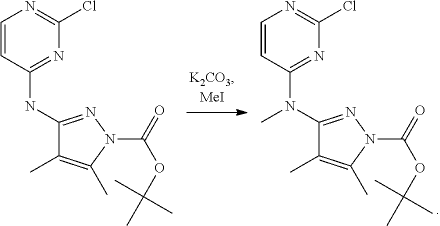 [SMILES: Cc1c(Nc2ccnc(Cl)n2)nn(C(=O)OC(C)(C)C)c1C ; Cc1c(N(C)c2ccnc(Cl)n2)nn(C(=O)OC(C)(C)C)c1C ; "."]

To a solution of 1,1-dimethylethyl 3-[(2-chloro-4-pyrimidinyl)amino]-4,5-dimethyl-1H-pyrazole-1-carboxylate (1.12 g, 2.214 mmol, 64% pure) in N,N-Dimethylformamide (DMF) (8 mL) under nitrogen was added potassium carbonate (0.612 g, 4.43 mmol) and iodomethane (0.180 mL, 2.88 mmol). Reaction was stirred at room temperature for 60 minutes. Reaction was diluted with satd. NaCl and diethyl ether and layers were separated. Organics were concentrated and purified by column chromatography using an ethyl acetate/hexanes gradient to give the title compound as a light yellow solid (416 mg, 55%). ¹H NMR (400 MHz, DMSO-d₆) δ 8.15 (d, J=6.06 Hz, 1H), 6.50 (br. s., 1H), 3.31 (s, 3H), 2.43 (s, 3H), 1.76 (s, 3H), 1.58 (s, 9H); MS (m/z) 338.3 (M+H⁺).

### Preparation 31

### 2-chloro-N-(4,5-dimethyl-1H-pyrazol-3-yl)-N-methyl-4-pyrimidinamine

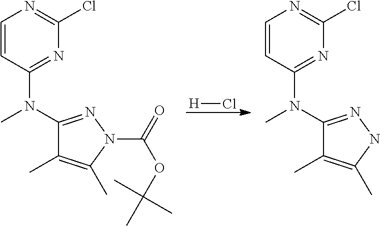

1,1-Dimethylethyl 3-[(2-chloro-4-pyrimidinyl)(methyl)amino]-4,5-dimethyl-1H-pyrazole-1-carboxylate (410 mg, 1.214 mmol) was dissolved in Dichloromethane (DCM) (15 mL) and hydrogen chloride (3.03 mL, 6.07 mmol, 2M in diethyl ether) was added. Reaction was stirred at room temperature for 2 days, basified with satd. NaHCO₃ and layers were separated. Organics were dried over sodium sulfate, concentrated and dried to give the title compounds as a sticky yellow oil which started to solidify upon standing (306 mg, 99%). ¹H NMR (400 MHz, DMSO-d₆) δ 12.50 (br. s., 1H), 8.02 (br. s., 1H), 6.18 (br. s., 1H), 3.29 (s, 3H), 2.19 (s, 3H), 1.73 (s, 3H); MS (m/z) 238.2 (M+H⁺).

### Example 1

### N⁴-(3,4-Dimethyl-1H-pyrazol-5-yl)-N²-[3,4,5-tris(methyloxy)phenyl]-2,4-pyrimidinediamine

### Coupling Method A

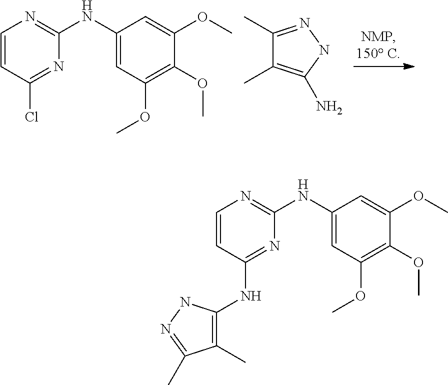

A solution of 4-chloro-N-[3,4,5-tris(methyloxy)phenyl]-2-pyrimidinamine (639 mg, 2.159 mmol) and 3,4-dimethyl-1H-pyrazol-5-amine (240 mg, 2.159 mmol) in N-Methyl-2-pyrrolidone (NMP) (5400 μl) was heated to 150° C. overnight. The crude reaction was filtered through a 0.2 μm ptfe frit and purified via mass directed prep HPLC using a Sunfire 5 μm, 30×75 mm, C18 column eluting with 20-60% MeCN/water (with 0.1% TFA) over a 16 min gradient. The pure fractions were combined, neutralized with NaHCO₃, and extracted with DCM. The combined extracts were dried over MgSO₄, filtered, and concentrated to give the titled compound (443 mg, 55%). ¹H NMR (400 MHz, DMSO-d₆) δ ppm 1.77 (br. s., 3H) 2.13 (br. s., 3H) 3.59 (s, 3H) 3.68 (br. s., 6H) 6.10-6.21 (m, 1H) 7.13 (br. s., 2H) 7.90-7.99 (m, 1H) 8.67-8.84 (m, 1H) 8.84-8.96 (m, 1H) 12.03-12.11 (m, 1H); MS (m/z) 371 (M+H⁺).

### Example 2

### N²-[3,4-Bis(methyloxy)phenyl]-N⁴-(3,4-dimethyl-1-phenyl-1H-pyrazol-5-yl)-2,4-pyrimidinediamine

### Coupling Method B

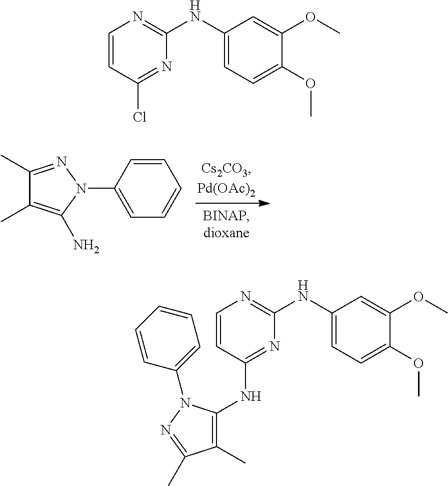

A vial was charged with N-[3,4-bis(methyloxy)phenyl]-4-chloro-2-pyrimidinamine (50.0 mg, 0.188 mmol) and 3,4-dimethyl-1-phenyl-1H-pyrazol-5-amine (0.188 mmol). Cesium carbonate (184 mg, 0.565 mmol) was added followed by binap (11.72 mg, 0.019 mmol), 1,4-Dioxane (941 μl) and finally palladium(II) acetate (4.22 mg, 0.019 mmol). The reaction was heated to 90° C. and stirred for 16 hours then diluted with MeOH and purified via prep HPLC using a Sunfire 5 μm, 30×75 mm, C18 column eluting with 20-60% MeCN/water (with 0-1% TFA). The fractions containing product were concentrated to afford the titled compound as the TFA salt (21.3 mg, 21%). ¹H NMR (400 MHz, DMSO-d₆) δ ppm 1.84 (br. s., 3H) 2.21 (s, 3H) 3.49-3.67 (m, 3H) 3.74 (s, 3H) 6.29-6.44 (m, 1H) 6.79-7.03 (m, 2H) 7.03-7.19 (m, 1H) 7.32 (br. s., 1H) 7.43 (d, J=4.28 Hz, 4H) 7.80-8.00 (m, 1H) 10.00-10.45 (m, 2H); MS (m/z) 417 (M+H⁺).

### Example 3

### 3-[(4-{[3,4-Dimethyl-1-(2-pyridinyl)-1H-pyrazol-5-yl]amino}-2-pyrimidinyl)amino]-N,N-dimethylbenzenesulfonamide

### Coupling Method C

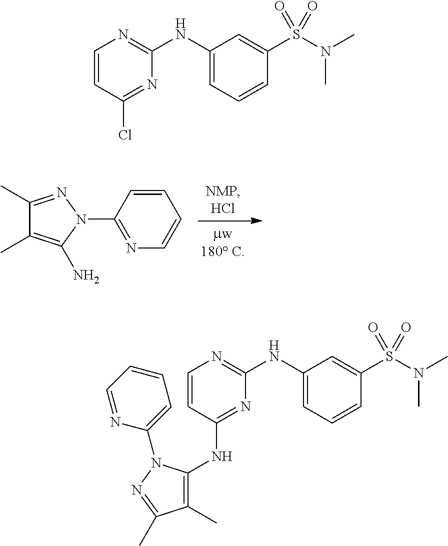 [SMILES: CN(C)S(=O)(=O)c1cccc(Nc2nccc(Cl)n2)c1 ; Cc1nn(-c2ccccn2)c(N)c1C ; Cc1nn(-c2ccccn2)c(Nc2ccnc(Nc3cccc(S(=O)(=O)N(C)C)c3)n2)c1C]

A microwave vial was charged with 3-[(4-chloro-2-pyrimidinyl)amino]-N,N-dimethylbenzenesulfonamide (100 mg, 0.32 mmol), 3,4-dimethyl-1-(2-pyridinyl)-1H-pyrazol-5-amine (60.2 mg, 0.32 mmol), and N-Methyl-2-pyrrolidone (2 ml). 2 drops of 4N HCl in dioxane was added to the reaction mixture. The reaction vial was put in an Emrys Optimizer (150 W, absorption normal, 180° C., 90 min). The crude mixture was loaded onto a Strata SCX column (55 um, 70 A, 5 g/20 ml Giga Tubes). The column was first flushed with 20 ml of MeOH, followed by 20 ml of 1N NH3 in MeOH. The collected 1N NH3 in MeOH fraction was concentrated and the crude residue was purified via prep HPLC using a Sunfire (5 μm, 30×150 mm, C18 column) eluting with 20-60% MeCN/water (with 0.1% TFA). The fractions containing the product were combined and concentrated to afford the titled compound as the TFA salt (41.8 mg, 23%). ¹H NMR (500 MHz, DMSO-d₆) δ ppm 1.85 (s, 3H), 2.24 (s, 3H), 2.59 (s, 6H), 6.30 (br. s., 1H), 7.25 (dd, J=6.8, 5.1 Hz, 1H), 7.32 (d, J=7.1 Hz, 1H), 7.44 (t, J=7.9 Hz, 1H), 7.68 (d, J=8.3 Hz, 1H), 7.83-7.97 (m, 3H), 8.02 (d, J=6.3 Hz, 1H), 8.35 (d, J=4.9 Hz, 1H), 9.90 (br. s., 1H); MS (m/z) 465 (M+H⁺).

### Example 4

### 3-({4-[(3,4-Dimethyl-1H-pyrazol-5-yl)amino]-2-pyrimidinyl}amino)-N,N-dimethyl-benzenesulfonamide

### Coupling Method D

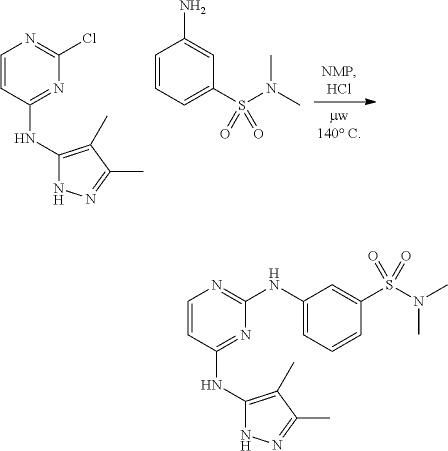 [SMILES: Cc1n[nH]c(Nc2ccnc(Cl)n2)c1C ; CN(C)S(=O)(=O)c1cccc(N)c1 ; Cc1n[nH]c(Nc2ccnc(Nc3cccc(S(=O)(=O)N(C)C)c3)n2)c1C]

A microwave vial was charged with 2-chloro-N-(3,4-dimethyl-1H-pyrazol-5-yl)-4-pyrimidinamine (50 mg, 0.224 mmol), 3-amino-N,N-dimethylbenzene-sulfonamide (44.8 mg, 0.224 mmol), and isopropanol (2 ml). 2 drops of 4N HCl in dioxane was added to the reaction mixture. The reaction vial was put in an Emrys Optimizer (150 W, absorption normal, 140° C., 10 min). The crude mixture was loaded onto a Strata SCX column (55 um, 70 A, 5 g/20 ml Giga Tubes). The column was first flushed with 20 ml of MeOH, followed by 20 ml of 1N NH3 in MeOH. The collected 1N NH3 in MeOH fraction was concentrated and the crude residue was purified via prep HPLC using a Sunfire (5 μm, 30×150 mm, C18 column) eluting with 10-40% MeCN/water (with 0.1% TFA). The fractions containing the product were combined and concentrated to afford the titled compound as the TFA salt (67 mg, 60%). ¹H NMR (500 MHz, DMSO-d₆) δ ppm 1.80 (s, 3H), 2.17 (s, 3H), 2.61 (s, 6H), 6.42 (d, J=6.6 Hz, 1H), 7.38 (d, J=7.3 Hz, 1H), 7.53 (t, J=7.8 Hz, 1H), 7.85 (br. s., 1H), 8.01 (d, J=6.6 Hz, 1H), 8.15 (br. s., 1H), 9.94 (br. s., 1H), 10.17 (br. s., 1H); MS (m/z) 388 (M+H⁺).

### Example 5

### N4-(3,4-Dimethyl-1H-pyrazol-5-yl)-N4-methyl-N2-[3,4,5-tris(methyloxy)phenyl]-2,4-pyrimidinediamine

### Coupling Method E

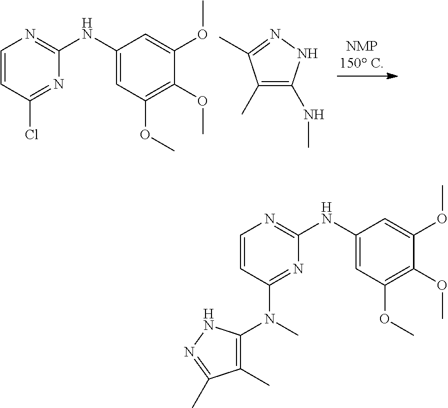

4-Chloro-N-[3,4,5-tris(methyloxy)phenyl]-2-pyrimidinamine (65 mg, 0.220 mmol) and N,3,4-trimethyl-1H-pyrazol-5-amine (36 mg, 0.259 mmol) were dissolved in N-Methyl-2-pyrrolidone (NMP) (2 mL) and the reaction was heated at 150° C. for 1.5 hours. The reaction was concentrated and purified via mass directed, prep HPLC using a Sunfire, 30×150 mm, C18 column eluting with 5-100% acetonitrile/water (with 0.1% TFA) over a 10.5 minute gradient. Fractions containing product were concentrated to give the title compound (15.5 mg, 18%). ¹H NMR (500 MHz, DMSO-d₆) δ 12.35 (br. s., 1H), 9.06 (br. s., 1H), 7.85 (d, J=5.86 Hz, 1H), 7.21 (s, 2H), 5.64 (br. s., 1H), 3.74 (s, 6H), 3.61 (s, 3H), 3.36 (s, 3H), 2.17 (s, 3H), 1.73 (s, 3H); MS (m/z) 385 (M+H⁺).

### Example 6

### N⁴-(3,4-Dimethyl-1H-pyrazol-5-yl)-N²-[4-fluoro-3-(methylsulfonyl)phenyl]-2,4-pyrimidinediamine

### Coupling Method F

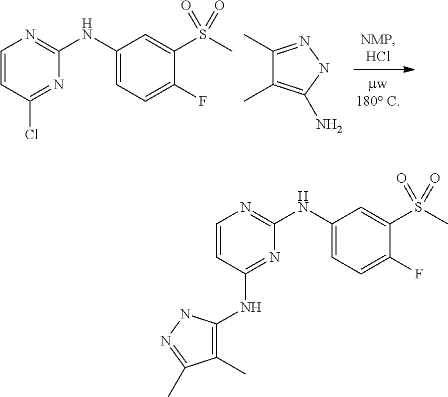

A microwave vial was charged with 4-chloro-N-[4-fluoro-3-(methylsulfonyl)phenyl]-2-pyrimidinamine (50 mg, 0.166 mmol), 3,4-dimethyl-1H-pyrazol-5-amine (18.4 mg, 0.166 mmol), and N-Methyl-2-pyrrolidone (2 ml). The reaction vial was put in an Emrys Optimizer (150 W, absorption normal, 180° C., 20 min). The crude mixture was loaded onto a Strata SCX column (55 um, 70 A, 5 g/20 ml Giga Tubes). The column was first flushed with 20 ml of MeOH, followed by 20 ml of 1N NH₃ in MeOH. The collected 1N NH₃ in MeOH fraction was concentrated and the crude residue was purified via prep HPLC using a Sunfire (5 μm, 30×150 mm, C18 column) eluting with 10-40% MeCN/water (with 0.1% TFA). The fractions containing the product were combined and concentrated to afford the title compound as the TFA salt (81 mg, 42%). ¹H NMR (400 MHz, DMSO-d₆) δ ppm 1.81 (s, 3H) 2.17 (s, 3H) 3.33 (s, 3H) 6.43 (d, J=6.53 Hz, 1H) 7.37-7.54 (m, 1H) 7.88-8.07 (m, 2H) 8.16-8.31 (m, 1H) 9.84-10.42 (m, 2H); MS (m/z) 377 (M+H)⁺.

### Example 7

### N4-(3,4-dimethyl-5-isoxazolyl)-N2-[3-(methylsulfonyl)phenyl]-2,4-pyrimidinediamine

### Coupling Method G

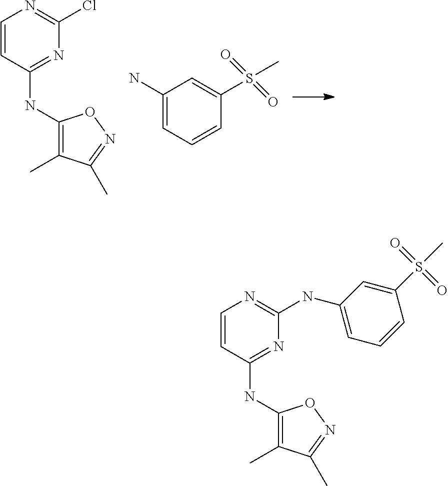 [SMILES: Cc1noc(Nc2ccnc(Cl)n2)c1C ; CS(=O)(=O)c1cccc(N)c1 ; Cc1noc(Nc2ccnc(Nc3cccc(S(C)(=O)=O)c3)n2)c1C]

2-Chloro-N-(3,4-dimethyl-5-isoxazolyl)-4-pyrimidinamine (60 mg, 0.267 mmol) was dissolved in N-Methyl-2-pyrrolidone (NMP) (2 mL) and [3-(methylsulfonyl)phenyl]amine (66.6 mg, 0.321 mmol) and 2 drops 4M HCl in dioxane were added. Reaction was heated at 150° C. for 3 hours. Reaction was cooled to room temperature, partitioned between ethyl acetate and satd. NaHCO₃ and layers were separated. Organics were concentrated and purified by column chromatography using a methanol/DCM gradient to give the title compound as a tan solid (68 mg, 70%). ¹H NMR (400 MHz, DMSO-d₆) δ 9.87 (s, 1H), 9.66 (s, 1H), 8.25 (s, 1H), 8.18 (d, J=5.56 Hz, 1H), 8.12 (dd, J=1.39, 7.96 Hz, 1H), 7.39-7.51 (m, 2H), 6.28 (d, J=5.56 Hz, 1H), 3.16 (s, 3H), 2.20 (s, 3H), 1.82 (s, 3H); MS (m/z) 360.1 (M+H⁺).

### Example 8

### N4-(3,4-dimethyl-1H-pyrazol-5-yl)-N2-{3-methyl-5-[(1-methylethyl)sulfonyl]phenyl}-2,4-pyrimidinediamine

### Coupling Method H

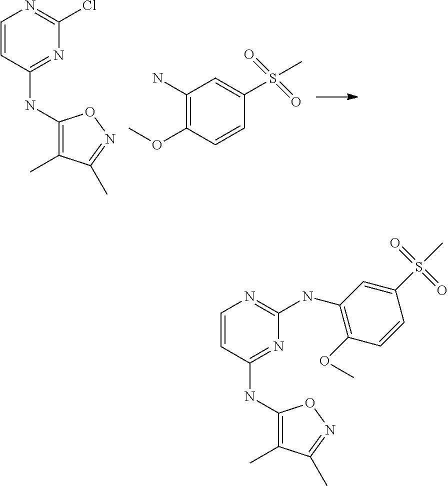

2-Chloro-N-(3,4-dimethyl-1H-pyrazol-5-yl)-4-pyrimidinamine (50 mg, 0.224 mmol) and {3-methyl-5-[(1-methylethyl)sulfonyl]phenyl}amine (50 mg, 0.234 mmol) were taken up in Isopropanol (3 mL) and HCl (drop) was added. The mixture was heated in a microwave to 160° C. for 20 minutes. The solid was collected by filtration and washed with iPrOH. The solid was dried under vacuum to give N4-(3,4-dimethyl-1H-pyrazol-5-yl)-N2-{3-methyl-5-[(1-methylethyl)sulfonyl]phenyl}-2,4-pyrimidinediamine (14 mg, 0.027 mmol, 12.17% yield) as a white solid. ¹H NMR (400 MHz, DMSO-d₆) δ 12.51 (s, 1H), 10.47 (br. s., 1H), 9.71 (br. s., 1H), 8.29 (br. s., 1H), 7.90-8.10 (m, 1H), 7.78 (br. s., 1H), 7.27-7.46 (m, 1H), 6.33-6.53 (m, 1H), 3.95 (br. s., 7H), 3.13 (br. s., 5H), 2.16 (s, 4H), 1.78 (br. s., 4H); MS (m/z) 389 (M+H⁺).

### Example 9

### N4-{3,4-dimethyl-1-[6-(methyloxy)-3-pyridinyl]-1H-pyrazol-5-yl}-N2-[3-(methylsulfonyl)phenyl]-2,4-pyrimidinediamine

### Coupling Method J

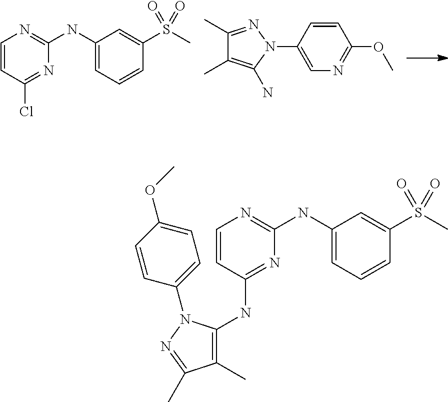

3,4-Dimethyl-1-[6-(methyloxy)-3-pyridinyl]-1H-pyrazol-5-amine (50 mg, 0.229 mmol), 4-chloro-N-[3-(methylsulfonyl)phenyl]-2-pyrimidinamine (65.0 mg, 0.229 mmol), Pd2(dba)3 (4.20 mg, 4.58 μmol), Xantphos (5.30 mg, 9.16 μmol), and sodium tert-butoxide (66.0 mg, 0.687 mmol) were mixed in Toluene (5 ml). The reaction mixture was heated to 150° C. for 20 minutes in a microwave. The reaction mixture was partitioned between water and DCM. The organic layer was washed by brine, dried over MgSO4 and concentrated to a brown residue. The residue was dissolved in 2 ml of DMSO purified by HPLC using a Sunfire (5 μm, 30×150 mm, C18 column) eluting with 10-40% MeCN/water (with 0.1% TFA). The fractions containing the product were combined and concentrated to afford the title compound as the TFA salt (133 mg, 0.034 mmol, 15% yield). ¹H NMR (DMSO-d₆) δ 9.85 (br. s., 1H), 9.51 (br. s., 1H), 8.20 (br. s., 1H), 8.08-8.17 (m, 1H), 8.03 (d, J=6.1 Hz, 1H), 7.85-7.99 (m, 1H), 7.76 (br. s., 1H), 7.48 (br. s., 2H), 6.87 (d, J=8.8 Hz, 1H), 3.83 (s, 3H), 3.14 (br. s., 3H), 2.22 (s, 3H), 1.85 (s, 3H); MS (m/z) 466 (M+H⁺).

### Example 10

### N4-(4,5-dimethyl-1H-pyrazol-3-yl)-N2-[3-(methylsulfonyl)-5-(1-pyrrolidinyl)phenyl]-2,4-pyrimidinediamine

### Coupling Method K

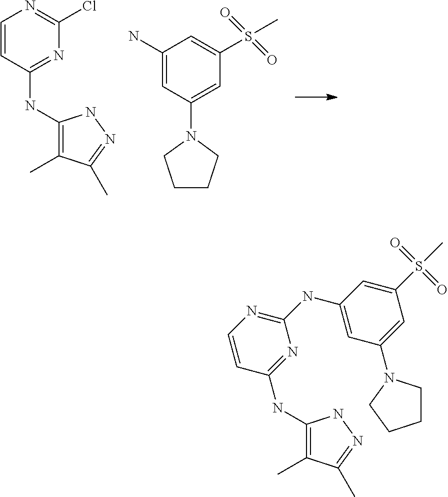 [SMILES: Cc1n[nH]c(Nc2ccnc(Cl)n2)c1C ; CS(=O)(=O)c1cc(N)cc(N2CCCC2)c1 ; Cc1n[nH]c(Nc2ccnc(Nc3cc(N4CCCC4)cc(S(C)(=O)=O)c3)n2)c1C]

A mixture of 2-chloro-N-(4,5-dimethyl-1H-pyrazol-3-yl)-4-pyrimidinamine (48.0 mg, 0.215 mmol), [3-(methylsulfonyl)-5-(1-pyrrolidinyl)phenyl]amine (56.0 mg, 0.233 mmol) and HCl (20.0 μl, 0:080 mmol, 4M in 1,4 dioxane) in Isopropanol (2.0 mL) were heated in oil bath at 100° C. The desired product which precipitated from the reaction mixture was collected by filtration then dissolved into 50 mL MeOH-DCM (10% v/v). The solution was basified by washing it with 2M aq sodium carbonate solution then the organic layer was dried over sodium sulfate, filtered and concentrated in-vacuo to yield the title compound (75.0 mg, 80%). ¹H NMR (CHLOROFORM-d) δ 9.36 (br. s., 1H), 8.55 (br. s., 1H), 8.11 (d, J=5.8 Hz, 1H), 7.71 (br. s., 1H), 7.40 (s, 1H), 6.64 (s, 1H), 6.13 (d, J=5.8 Hz, 1H), 3.32-3.42 (m, 4H), 3.12 (s, 3H), 2.27 (s, 3H), 1.99-2.11 (m, 4H), 1.91 (s, 3H). MS (m/z) 428.2 (M+H⁺).

### Example 11

### 3-(methylsulfonyl)-5-({4-[(1,3,4-trimethyl-1H-pyrazol-5-yl)amino]-2-pyrimidinyl}amino)benzoic acid

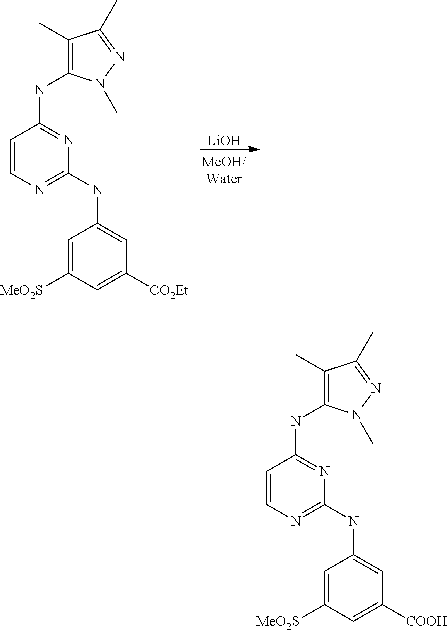 [SMILES: CCOC(=O)c1cc(Nc2nccc(Nc3c(C)c(C)nn3C)n2)cc(S(C)(=O)=O)c1 ; "LiOH" ; Cc1nn(C)c(Nc2ccnc(Nc3cc(C(=O)O)cc(S(C)(=O)=O)c3)n2)c1C]

A mixture of ethyl 3-(methylsulfonyl)-5-({4-[(1,3,4-trimethyl-1H-pyrazol-5-yl)amino]-2-pyrim-idinyl}amino) benzoate (13.0 mg, 0.029 mmol) and LiOH (4.90 mg, 0.205 mmol) in methanol-water (0.80 mL, 3:1) were stirred at rt for 20 h. The solvent was thoroughly evaporated to get a residue which was diluted with ACN and evaporated again. The residue was diluted with water then acidified to pH˜6 using dilute cold HCl. The mixture was extracted 3 times with a solution of CHCl₃-EtOH (10:1). The organic layer was dried over sodium sulfate, filtered then concentrated in-vacuo to yield the title compound (11.0 mg, 90.0%) as a white solid. ¹H NMR (DMSO-d₆) δ 13.39 (br. s., 1H), 9.72 (s, 1H), 8.97 (br. s., 1H), 8.62 (br. s., 1H), 8.56 (s, 1H), 8.11 (d, J=5.8 Hz, 1H), 7.89 (s, 1H), 3.53 (s, 3H), 3.20 (s, 3H), 2.08 (s, 3H), 1.79 (s, 3H); MS (m/z): 417.2 (M+H⁺).

The following compounds, isolated as trifluoroacetate (TFA) salts where indicated, were prepared using procedures analogous to those described above (Method A, Method B, Method C, Method D, Method E, Method F).

| | | | | MS | |
|---|---|---|---|---|---|
| | | | | (M + | Meth- |
| Ex | Name | Structure | 1H NMR | H)⁺ | od |
| 12 | N²-[3,4- bis(methyloxy) phenyl]-N⁴-[1-(5- chloro-2- pyridinyl)-3,4- dimethyl-1H- pyrazol-5-yl]-2,4- pyrimidinediamine trifluoroacetate | 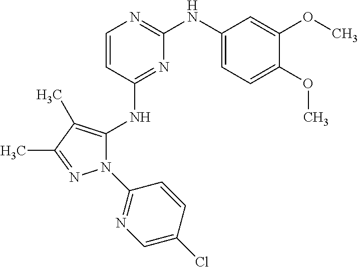 | ¹H NMR (DMSO-d₆) δ 10.58 (br. s., 1H), 10.15 (br. s., 1H), 8.38 (d, J = 2.3 Hz, 1H), 8.01 (d, J = 8.8 Hz, 1H), 7.85-7.93 (m, 1H), 7.60-7.70 (m, 1H), 6.95-7.04 (m, 1 H), 6.86-6.93 (m, 1H), 6.76- 6.86 (m, 1H), 6.26-6.46 (m, 1H), 3.75 (s, 3H), 3.61 (br. s., 3H), 2.22 (s, 3H), 1.84 (s, 3H) | 452 | A |
| 13 | N²-[3,4- bis(methyloxy) phenyl]-N⁴-[3,4- dimethyl-1-(6- methyl-3- pyridinyl)-1H- pyrazol-5-yl]-2,4- pyrimidinediamine trifluoroacetate | 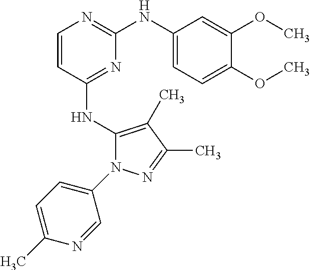 | ¹H NMR (DMSO-d₆) δ 9.92-10.44 (m, 1H), 8.50- 8.60 (m, 1H), 7.86-8.01 (m, 1H), 7.71-7.81 (m, 1H), 7.32-7.40 (m, 1H), 7.01-7.18 (m, 1H), 6.82- 6.97 (m, 2H), 6.30-6.47 (m, 1H), 3.75 (s, 3H), 3.54- 3.71 (m, 2H), 2.22 (s, 3H), 1.85 (s, 3H) | 432 | B |
| 14 | N²-[3,4- bis(methyloxy) phenyl]-N⁴-[3,4- dimethyl-1-(4- pyridinyl)-1H- pyrazol-5-yl]-2,4- pyrimidinediamine trifluoroacetate | 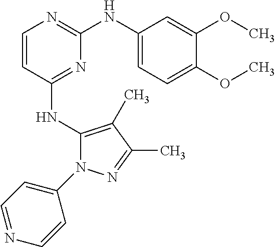 [SMILES: COc1ccc(Nc2nccc(Nc3c(C)c(C)nn3-c3ccncc3)n2)cc1OC] | ¹H NMR (DMSO-d₆) δ 10.52 (br. s., 1H), 10.09 (br. s., 1H), 8.67 (d, J = 5.0 Hz, 3H), 7.99 (br. s., 1H), 7.70-7.80 (m, 2H), 6.90-7.03 (m, 1H), 6.74- 6.90 (m, 2H), 6.41-6.56 (m, 1H), 3.74 (s, 3H), 3.50- 3.66 (m, 3H), 2.26 (s, 3H), 1.87 (s, 3H) | 418 | B |
| 15 | N²-[3,4- bis(methyloxy) phenyl]-N⁴-[3,4- dimethyl-1-(3- pyridinyl)-1H- pyrazol-5-yl]-2,4- pyrimidinediamine trifluoroacetate | 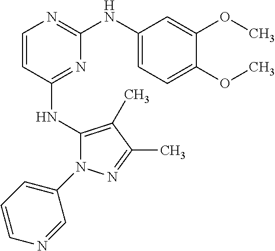 [SMILES: COc1ccc(Nc2nccc(Nc3c(C)c(C)nn3-c3cccnc3)n2)cc1OC] | ¹H NMR (DMSO-d₆) δ 10.52 (br. s., 1H), 10.25 (br. s., 1H), 8.67 (br. s., 1H), 8.47-8.57 (m, 1H), 7.75-7.99 (m, 2H), 7.50 (br. s., 1H), 7.01 (br. s., 1H), 6.90 ( br. s., 1H), 6.80 (br. s., 1H), 6.41 (br. s., 1H), 3.75 (s, 3H), 3.51- 3.69 (m, 3H), 2.23 (s, 3H), 1.86 (br. s., 3H) | 418 | B |
| 16 | N²-[3,4- bis(methyloxy) phenyl]-N⁴-[3,4- dimethyl-1-(6- methyl-2- pyridinyl)-1H- pyrazol-5-yl]-2,4- pyrimidinediamine trifluoroacetate | 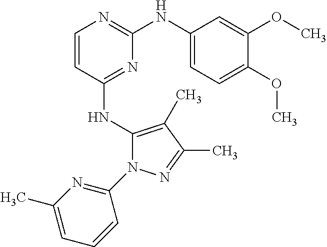 | ¹H NMR (DMSO-d₆) δ 10.60 (br. s., 1H), 10.27 (br. s., 1H), 7.91 (br. s., 1H), 7.77 (t, J = 7.8 Hz, 1H), 7.48 (d, J = 8.1 Hz, 1H), 7.12 (d, J = 7.6 Hz, 1H), 6.94-7.04 (m, 1H), 6.85-6.94 (m, 1H), 6.80 (br. s., 1H), 6.30-6.55 (m, 1H), 3.75 (s, 3H), 3.61 (br. s., 3H), 2.33 (s, 3H), 2.22 (s, 3H), 1.86 (s, 3H) | 432 | A |
| 17 | N²-[3,4- bis(methyloxy) phenyl]-N⁴-[3,4- dimethyl-1-(5- methyl-2- pyridinyl)-1H- pyrazol-5-yl]-2,4- pyrimidinediamine trifluoroacetate | 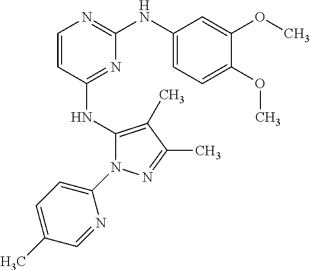 | ¹H NMR (DMSO-d₆) δ 10.62 (br. s., 1H), 10.22 (br. s., 1H), 8.19 (s, 1H), 7.87 (br. s., 1H), 7.73 (d, J = 8.1 Hz, 1H), 7.56 (d, J = 8.3 Hz, 1H), 6.99 (br. s., 1H), 6.86-6.93 (m, 1H), 6.77-6.86 (m, 1H), 6.25- 6.48 (m, 1H), 3.75 (s, 3H), 3.62 (br. s., 3H), 2.29 (s, 3H), 2.21 (s, 3H), 1.82 (s, 3H) | 432 | A |
| 18 | N⁴-{3,4-dimethyl- 1-[4-(methyloxy) phenyl]-1H- pyrazol-5-yl}-N²- [3- (methylsulfonyl) phenyl]-2,4- pyrimidinediamine trifluoroacetate | 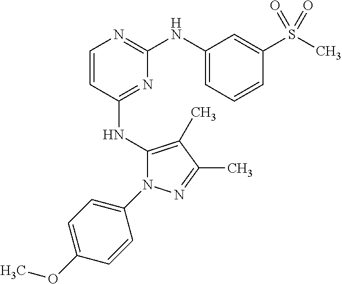 | ¹H NMR (DMSO-d₆) δ 9.98-10.12 (m, 1H), 9.41- 9.80 (m, 1H), 8.03 (d, J = 6.0 Hz, 2H), 7.80-7.99 (m, 1H), 7.51 (br. s., 2H), 7.33 (d, J = 8.3 Hz, 2H), 6.96 (d, J = 8.8 Hz, 2H), 6.23-6.48 (m, 1H), 3.74 (s, 3H), 3.10-3.19 (m, 3H), 2.22 (s, 3H), 1.84 (s, 3H) | 465 | A |
| 19 | N²-[3- (methylsulfonyl) phenyl]-N⁴- (1,3,4-trimethyl- 1H-pyrazol-5-yl)- 2,4- pyrimidinediamine trifluoroacetate | 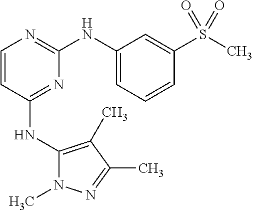 [SMILES: Cc1nn(C)c(Nc2ccnc(Nc3cccc(S(C)(=O)=O)c3)n2)c1C] | ¹H NMR (DMSO-d₆) □: 10.27 (br. s., 1H), 9.84 (br. s., 1H), 8.02-8.13 (m, 3H), 7.86-7.99 (m, 1H), 7.54-7.60 (m, 1H), 7.43- 7.54 (m, 1H), 6.13-6.49 (m, 1H), 3.53 (s, 3H), 3.17 (s, 3H), 2.10 (s, 3H), 1.78 (s, 3H) | 373 | A |
| 20 | N²-[3,4- bis(methyloxy) phenyl]-N⁴-[4- methyl-1-[4- (methyloxy) phenyl]-3- (trifluoromethyl)- 1H-pyrazol-5-yl]- 2,4- pyrimidinediamine | 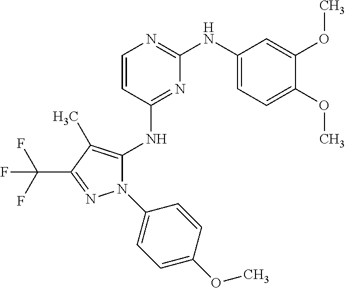 [SMILES: COc1ccc(-n2nc(C(F)(F)F)c(C)c2Nc2ccnc(Nc3ccc(OC)c(OC)c3)n2)cc1] | ¹H NMR (CHLOROFORM- d) δ 7.99 (d, J = 5.5 Hz, 1H), 7.34-7.62 (m, 1H), 7.29 (d, J = 9.1 Hz, 2H), 7.04 (s, 1H), 6.97 (dd, J = 8.6, 2.5 Hz, 1H), 6.86 (d, J = 8.8 Hz, 2H), 6.76 (d, J = 8.6 Hz, 1H), 5.79 (d, J = 5.8 Hz, 1H), 3.83 (s, 3H), 3.74-3.80 (m, 6H), 2.05 (s, 3H) | 501 | B |
| 21 | N²-[3,4- bis(methyloxy) phenyl]-N⁴-[1-[4- (methyloxy) phenyl]-3- (trifluoromethyl)- 1H-pyrazol-5-yl]- 2,4- pyrimidinediamine | 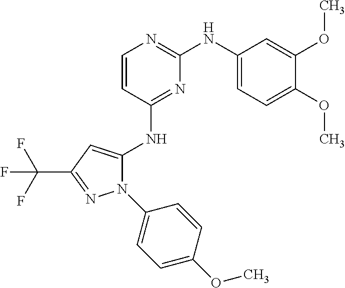 [SMILES: COc1ccc(-n2nc(C(F)(F)F)cc2Nc2ccnc(Nc3ccc(OC)c(OC)c3)n2)cc1] | ¹H NMR (CHLOROFORM- d) δ 8.03 (d, J = 4.8 Hz, 1H), 7.56-7.73 (m, 1H), 7.38 (d, J = 8.8 Hz, 2H), 7.01-7.10 (m, 2H), 6.98 (d, J = 8.8 Hz, 2H), 6.84 (d, J = 8.6 Hz, 1H), 6.78 (s, 1H), 6.06 (d, J = 5.8 Hz, 1H), 3.88 (s, 3H), 3.85 (s, 3H), 3.83 (s, 3H) | 487 | B |
| 22 | N²-[3-chloro-4-(4- methyl-1- piperazinyl)phenyl]- N⁴-[1-(4- chlorophenyl)-3- methyl-1H- pyrazol-5-yl]-2,4- pyrimidinediamine trifluoroacetate | 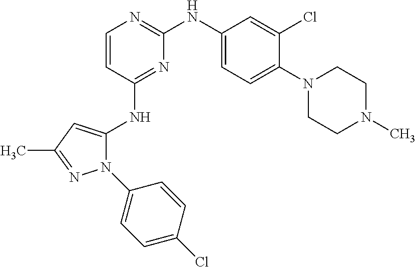 | ¹H NMR (DMSO-d₆) δ 9.70 (br. s., 1H), 8.02 (d, J = 6.0 Hz, 1H), 7.87 (s, 0H), 7.50 (s, 4H), 7.39- 7.46 (m, 1H), 7.10 (d, J = 8.8 Hz, 1H), 6.36 (s, 1H), 6.22 (br. s., 1H), 3.37 (br. s., 4H), 3.19 (d, J = 19.9 Hz, 2H), 2.89 (br. s., 5H), 2.26 (s, 3H) | 509 | B |
| 23 | N²-[3-chloro-4-(4- methyl-1- piperazinyl)phenyl]- N⁴-[3-methyl-1- (4-methylphenyl)- 1H-pyrazol-5-yl]- 2,4- pyrimidinediamine trifluoroacetate | 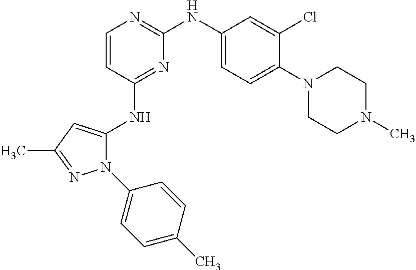 | ¹H NMR (DMSO-d₆) δ 9.64-10.18 (m, 3H), 8.02 (d, J = 6.3 Hz, 1H), 7.85 (d, J = 2.0 Hz, 1H), 7.42 (d, J = 8.1 Hz, 1H), 7.34 (d, J = 8.3 Hz, 2H), 7.24 (d, J = 8.3 Hz, 2H), 7.13 (d, J = 8.8 Hz, 1H), 6.34 (s, 1H), 6.18-6.29 (m, 1H), 3.54 (d, J = 11.8 Hz, 2H), 3.36 (d, J = 12.3 Hz, 2H), 3.21 (br. s., 2H), 2.92- 3.03 (m, 2H), 2.89 (s, 3H), 2.31 (s, 3H), 2.26 (s, 3H) | 489 | B |
| 24 | N²-[3,4- bis(methyloxy) phenyl]-N⁴-{3- methyl-1-[4- (methyloxy) phenyl]-1H- pyrazol-5-yl}-2,4- pyrimidinediamine | 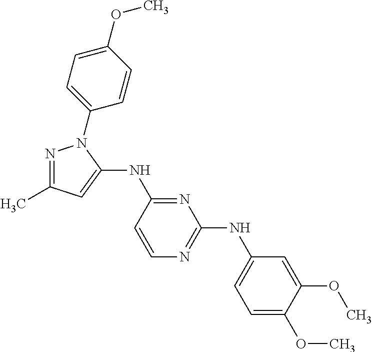 | ¹H NMR (DMSO-d₆) δ 7.89 (br. s., 1H), 7.30- 7.38 (m, 2H), 7.12-7.22 (m, 1H), 6.99 (d, J = 8.8 Hz, 4H), 6.86-6.94 (m, 1H), 6.31 (br. s., 1H), 6.20 (none, 1H), 3.77 (s, 3H), 3.74 (s, 3H), 3.67 (br. s., 3H), 2.22 (s, 3H) | 433 | B |
| 25 | N²-[3,4- bis(methyloxy) phenyl]-N⁴-{2-[3- (methyloxy) phenyl]-2,4,5,6- tetrahydro- cyclopenta[c] pyrazol-3-yl}-2,4- pyrimidinediamine trifluoroacetate | 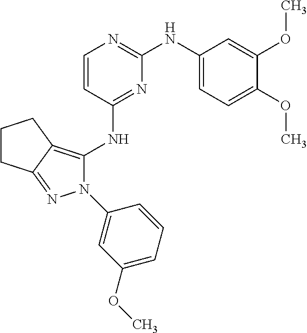 [SMILES: COc1cccc(-n2nc3c(c2Nc2ccnc(Nc4ccc(OC)c(OC)c4)n2)CCC3)c1] | ¹H NMR (DMSO-d₆) δ 7.91 (br. s., 1H), 7.36 (t, J = 8.3 Hz, 1H), 7.13 (br. s., 1H), 7.00-7.07 (m, 2H), 6.92 (br. s., 3H), 6.29 (br. s., 1H), 3.75 (s, 6H), 3.60 (br. s., 3H), 2.64-2.75 (m. 3H), 2.39-2.47 (m, 1H), 2.21-2.35 (m, 2H) | 459 | A |
| 26 | N²-[3,4- bis(methyloxy) phenyl]-N⁴-[2-(4- chlorophenyl)- 2,4,5,6- tetrahydro- cyclopenta[c] pyrazol-3-yl]-2,4- pyrimidinediamine trifluoroacetate | 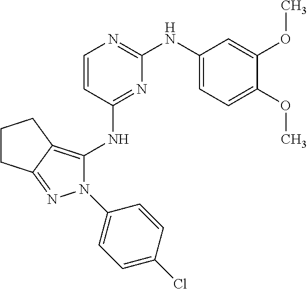 [SMILES: COc1ccc(Nc2nccc(Nc3c4c(nn3-c3ccc(Cl)cc3)CCC4)n2)cc1OC] | ¹H NMR (DMSO-d₆) δ 7.91 (br. s., 1H), 7.51 (d, J = 11.3 Hz, 4H), 7.14 (br. s., 1H), 6.92 (br. s., 2H), 6.16-6.34 (m, 1H), 3.75 (s, 3H), 3.59 (br. s., 3H), 2.68 (t, J = 7.1 Hz, 3H), 2.39-2.47 (m, 1H), 2.27 (br. s., 2H) | 463 | A |
| 27 | N²-[3,4- bis(methyloxy) phenyl]-N⁴-(4,5- dimethyl-1H- pyrazol-3-yl)-2,4- pyrimidinediamine | 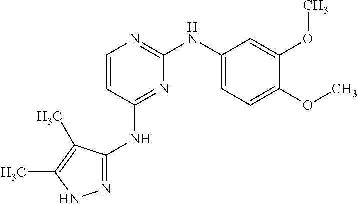 | ¹H NMR (DMSO-d₆) δ 12.05 (br. s., 1H), 8.82 (br. s., 1H), 8.67 (br. s., 1H), 7.96 (br. s., 1H), 7.43 (br. s., 1H), 7.18-7.29 (m, 1H), 6.81 (br. s., 1H), 6.15 (br. s., 1H), 3.70 (s, 6H), 2.14 (br. s., 3H), 1.79 (br. s., 3H) | 341 | A |
| 28 | N²-[3,4- bis(methyloxy) phenyl]-N⁴-{3,4- dimethyl-1-[3- (methyloxy) phenyl]-1H- pyrazol-5-yl}-2,4- pyrimidinediamine trifluoroacetate | 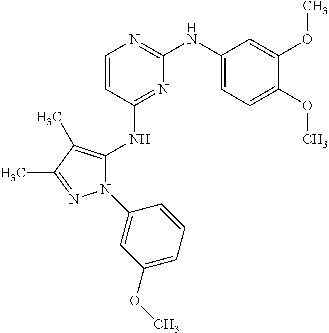 | ¹H NMR (DMSO-d₆) δ 10.03-10.73 (m, 2H), 7.76-8.01 (m, 1H), 7.33 (t, J = 8.1 Hz, 1H), 7.03 (br. s., 3H), 6.76-6.93 (m, 3H), 6.40 (br. s., 1H), 3.75 (s, 3H), 3.72 (br. s., 3H), 3.57 (none, 3H), 2.21 (s, 3H), 1.84 (br. s., 3H) | 447 | A |
| 29 | N²-[3,4- bis(methyloxy) phenyl]-N⁴-[1-(4- chlorophenyl)- 3,4-dimethyl-1H- pyrazol-5-yl]-2,4- pyrimidinediamine trifluoroacetate | 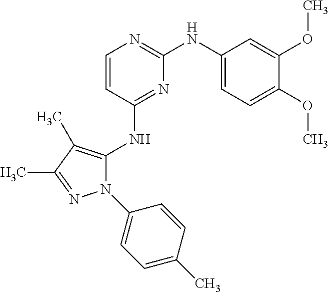 | ¹H NMR (DMSO-d₆) δ 9.69-10.51 (m, 2H), 7.90 (br. s., 1H), 7.49 (t, J = 6.8 Hz, 4H), 7.09 (br. s., 1H), 6.88 (br. s., 2H), 6.38 (br. s., 1H), 3.74 (s, 6H), 2.21 (s, 3H), 1.84 (s, 3H) | 433 | A |
| 30 | N²-[3,4- bis(methyloxy) phenyl]-N⁴-[1-(4- chlorophenyl)-3- methyl-1H- pyrazol-5-yl]-2,4- pyrimidinediamine | 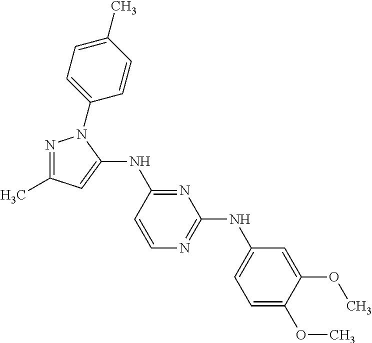 | ¹H NMR (DMSO-d₆) δ 7.92 (br. s., 1H), 7.49 (d, J = 5.4 Hz, 4H), 7.18 (br. s., 1H), 6.97-7.04 (m, 1H), 6.88 (br. s., 1H), 6.36 (br. s., 1H), 6.14-6.24 (m, 1H), 3.74 (s, 3H), 3.66 (s, 3H), 2.23 (s, 3H) | 437 | B |
| 31 | 3-({4-[(1,3,4- trimethyl-1H- pyrazol-5- yl)amino]-2- pyrimidinyl}amino) benzene sulfonamide trifluoroacetate | 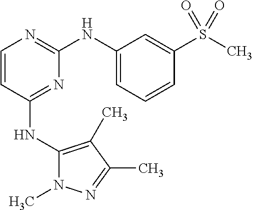 [SMILES: Cc1nn(C)c(Nc2ccnc(Nc3cccc(S(C)(=O)=O)c3)n2)c1C] | ¹H NMR (DMSO-d₆) δ 10.39 (br. s., 1H), 10.02 (br. s., 1H), 8.07 (d, J = 6.5 Hz, 1H), 7.67-7.95 (m, 2H), 7.48-7.53 (m, 1H), 7.39 (br. s., 3H), 3.53 (s, 3H), 2.11 (s, 3H), 1.78 (s, 3H) | 374 | A |
| 32 | 3-{[4-({3,4- dimethyl-1-[4- (methyloxy) phenyl]-1H- pyrazol-5- yl}amino)-2- pyrimidinyl]amino} benzene sulfonamide trifluoroacetate | 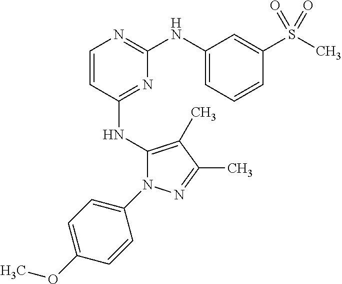 | ¹H NMR (DMSO-d₆) δ 9.58-10.37 (m, 1H), 8.01 (d, J = 6.3 Hz, 1H), 7.71- 7.92 (m, 1H), 7.48 (d, J = 7.1 Hz, 1H), 7.27-7.40 (m, 4H), 6.96 (d, J = 8.6 Hz, 2H), 3.71-3.77 (m, 3H), 2.19-2.25 (m, 3H), 1.84 (s, 3H) | 466 | A |
| 33 | 3-[(4-{[3,4- dimethyl-1-(4- methylphenyl)- 1H-pyrazol-5- yl]amino}-2- pyrimidinyl)amino] benzene sulfonamide trifluoroacetate | 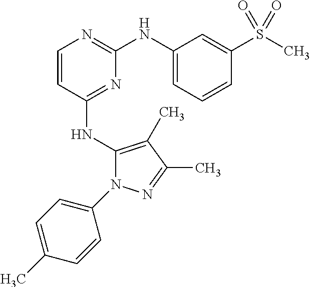 | ¹H NMR (DMSO-d₆) δ 9.80-10.27 (m, 1H), 8.01 (d, J = 6.3 Hz, 1H), 7.79- 7.91 (m, 1H), 7.42-7.49 (m, 1H), 7.28-7.37 (m, 3H), 7.21 (d, J = 8.1 Hz, 2H), 2.29 (s, 3H), 2.23 (s, 3H), 1.85 (s, 3H) | 450 | A |
| 34 | 3-[(4-{[1-(4- chlorophenyl)- 3,4-dimethyl-1H- pyrazol-5- yl]amino}-2- pyrimidinyl)amino] benzene sulfonamide trifluoroacetate | 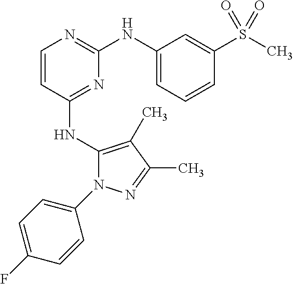 [SMILES: Cc1nn(-c2ccc(F)cc2)c(Nc2ccnc(Nc3cccc(S(C)(=O)=O)c3)n2)c1C] | ¹H NMR (DMSO-d₆) δ 9.56-10.10 (m, 1H), 8.03 (d, J = 6.0 Hz, 1H), 7.54- 7.99 (m, 2H), 7.27-7.52 (m, 6H), 2.23 (s, 3H), 1.85 (s, 3H) | 470 | A |
| 35 | N²-[3,4- bis(methyloxy) phenyl]-N⁴-[3,4- dimethyl-1-(2- pyridinyl)-1H- pyrazol-5-yl]-2,4- pyrimidinediamine trifluoroacetate | 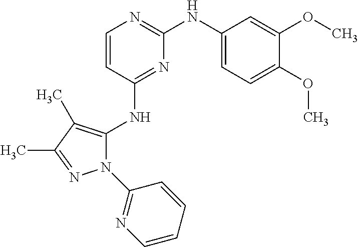 | ¹H NMR (DMSO-d₆) δ 10.47 (br. s., 1H), 10.03 (br. s., 1H), 8.35 (dd, J = 4.8, 1.0 Hz, 1H), 7.80- 8.03 (m, 3H), 7.28 (dd, J = 6.6, 5.1 Hz, 1H), 7.03 (br. s., 1H), 6.87 (br. s., 2H), 6.34 (br. s., 1H), 3.75 (br. s., 3H), 3.62 (br. s., 3H), 2.23 (s, 3H), 1.83 (s, 3H) | 418 | A |
| 36 | N²-[3,4- bis(methyloxy) phenyl]-N⁴- (1,3,4-trimethyl- 1H-pyrazol-5-yl)- 2,4- pyridmidinediamine trifluoroacetate | 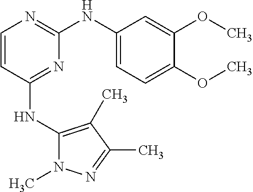 [SMILES: COc1ccc(Nc2nccc(Nc3c(C)c(C)nn3C)n2)cc1OC] | ¹H NMR (METHANOL-d₄) δ 7.81 (br. s., 1H), 6.81- 7.13 (m, 4H), 6.50 (br. s., 1H), 3.87 (s, 3H), 3.75 (br. s., 2H), 3.62 (br. s., 3H), 2.17 (s, 3H), 1.88 (none, 3H) | 355 | A |
| 37 | N⁴-[3-methyl-1- (4-methylphenyl)- 1H-pyrazol-5-yl]- N²-[4-(4-methyl- 1- piperazinyl)phenyl]- 2,4- pyrimidinediamine | 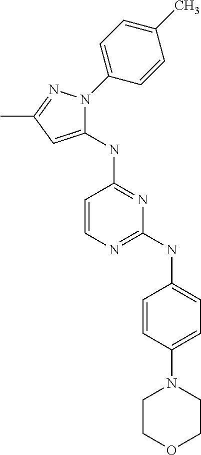 [SMILES: Cc1ccc(-n2nc(C)cc2Nc2ccnc(Nc3ccc(N4CCOCC4)cc3)n2)cc1] | ¹H NMR (400 MHz, DMSO-d₆) δ 10.07- 10.46 (m, 4H), 9.62-9.91 (m, 3H), 7.83-8.03 (m, 1H), 7.34 (d, J = 8.34 Hz, 4H), 7.28 (s, 2H), 6.99 (br. s., 2H), 6.24-6.40 (m, 2H), 3.73-3.88 (m, 2H), 3.44-3.61 (m, 2H), 3.11- 3.27 (m, 2H), 2.88 (d, J = 1.52 Hz, 6H), 2.33 (s, 4H), 2.25 (s, 3H) | 455 | B |
| 38 | N⁴-{3-methyl-1- [4-(methyloxy) phenyl]-1H- pyrazol-5-yl}-N²- [4-(4-methyl-1- piperazinyl)phenyl]- 2,4- pyrimidinediamine | 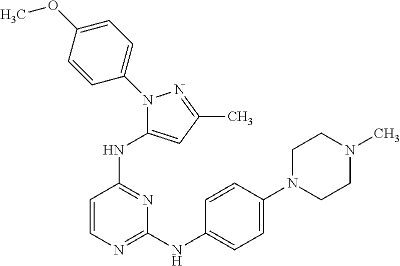 | ¹H NMR (DMSO-d₆, 400 MHz): δ 10.07- 10.49 (m, 5 H), 9.62-9.90 (m, 3 H), 7.85-8.04 (m, 1 H), 7.35 (d, J = 9.1 Hz, 4 H), 6.93-7.10 (m, 4 H), 6.33 (br. s., 2 H), 3.78 (s, 6 H), 3.53 (br. s., 2 H), 3.19 (br. s., 2 H), 2.88 (br. s., 6 H), 2.24 ppm (s, 3 H) | 471 | B |
| 39 | N⁴-[1-(3,4- dimethylphenyl)- 3-methyl-1H- pyrazol-5-yl]-N²- [4-(4-methyl-1- piperazinyl)phenyl]- 2,4- pyrimidinediamine | 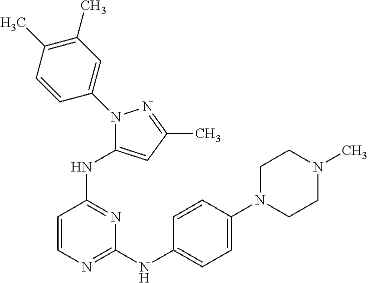 | ¹H NMR (DMSO-d₆, 400 MHz): δ 9.97-10.36 (m, 3 H), 9.61-9.84 (m, 2 H), 7.86-8.02 (m, 1 H), 7.33-7.42 (m, 2 H), 7.10- 7.29 (m, 3 H), 6.98 (d, J = 8.8 Hz, 2 H), 6.33 (br. s., 2 H), 3.73-3.86 (m, 2 H), 3.49-3.60 (m, 2 H), 3.10-3.27 (m, 2 H), 2.88 (d, J = 2.5 Hz, 6 H), 2.18- 2.29 ppm (m, 9 H) | 469 | B |
| 40 | N⁴-[3-methyl-1- (2-methylphenyl)- 1H-pyrazol-5-yl]- N²-[4-(4-methyl- 1- piperazinyl)phenyl]- 2,4- pyrimidinediamine | 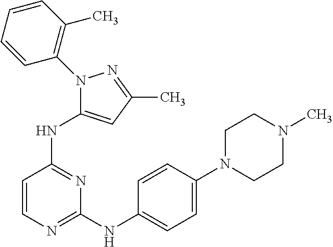 [SMILES: Cc1cc(Nc2ccnc(Nc3ccc(N4CCN(C)CC4)cc3)n2)n(-c2ccccc2C)n1] | ¹H NMR (DMSO-d₆, 400 MHz): δ 9.95-10.22 (m, 4 H), 9.64-9.84 (m, 3 H), 7.84-7.98 (m, 1 H), 7.37 (s, 5 H), 7.25-7.33 (m, 1 H), 7.16-7.24 (m, 1 H), 7.01 (d, J = 8.8 Hz, 2 H), 6.24-6.43 (m, 2 H), 3.69-3.93 (m, 2 H), 3.50- 3.60 (m, 2 H), 3.12-3.27 (m, 2 H), 2.82-3.01 (m, 5 H), 2.23 (s, 3 H), 2.01 ppm (s, 3 H) | 455 | B |
| 41 | 2-{3,4-dimethyl- 5-[(2-{[3,4,5- tris(methyloxy) phenyl]amino}-4- pyrimidinyl) amino]-1H- pyrazol-1- yl}ethanol | 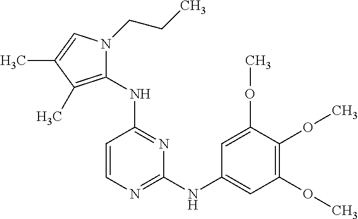 | ¹H NMR (DMSO-d₆) δ 9.61 (br. s., 1H), 7.97 (d, J = 5.5 Hz, 1H), 6.80-7.09 (m, 3 H), 3.85-3.91 (m, 2H), 3.76-3.82 (m, 1H), 3.58-3.72 (m, 11H), 2.09 (s, 3H), 1.75 (s, 3H) | 415 | A |
| 42 | N⁴-{3-methyl-1- [4-(methyloxy) phenyl]-1H- pyrazol-5-yl}-N²- [3,4,5- tris(methyloxy) phenyl]-2,4- pyrimidinediamine | 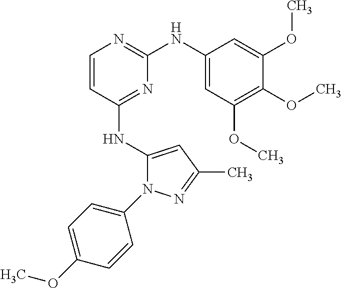 | ¹H NMR (400 MHz, DMSO-d₆) δ 7.90-8.00 (m, 2H), 7.30-7.40 (m, 2H), 6.97-7.04 (m, 2H), 6.86-6.96 (m, 2H), 6.31- 6.37 (m, 1H), 6.13-6.27 (m, 1H), 3.77 (s, 3H), 3.67 (s, 6H), 3.63 (s, 3H), 2.22 (s, 3H) | 463 | A |
| 43 | N⁴-{1,3,4- trimethyl-1H- pyrazol-5-yl}-N²- [3,4,5- tris(methyloxy) phenyl]-2,4- pyrimidinediamine | 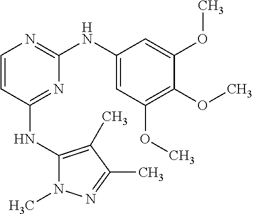 [SMILES: COc1cc(Nc2nccc(Nc3c(C)c(C)nn3C)n2)cc(OC)c1OC] | ¹H NMR (DMSO-d₆, 500 MHz): δ 9.78-10.50 (m, 2 H), 7.79-8.13 (m, 1 H), 6.64-7.02 (m, 2 H), 3.63 (s, 6 H), 3.53 (br. s., 5 H), 2.07 (s, 4 H), 1.77 ppm (s, 3 H) | 385 | A |
| 44 | N⁴-{3,4-dimethyl- 1-[4-(methyloxy)- phenyl]-1H- pyrazol-5-yl}-N²- [3,4,5- tris(methyloxy) phenyl]-2,4- pyrimidinediamine trifluoroacetate | 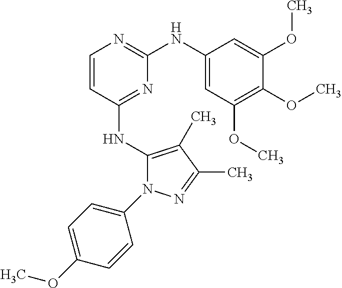 | ¹H NMR (DMSO-d₆) δ 8.95 (br. s., 1H), 8.85 (br. s., 1H), 7.93-7.99 (m, 1H), 7.34-7.39 (m, 2H), 7.04-7.12 (m, 2H), 6.93- 6.98 (m, 2H), 3.72-3.75 (m, 3H), 3.55-3.63 (m, 9H), 2.15-2.19 (m, 3H), 1.78-1.84 (m, 3H) | 477 | A |
| 45 | N⁴-[3,4-dimethyl- 1-(2-pyridinyl)- 1H-pyrazol-5-yl]- N²-[3,4,5- tris(methyloxy) phenyl]-2,4- pyrimidinediamine | 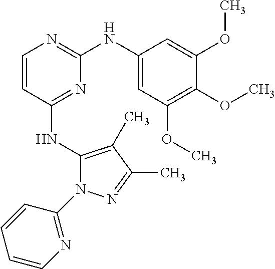 [SMILES: COc1cc(Nc2nccc(Nc3c(C)c(C)nn3-c3ccccn3)n2)cc(OC)c1OC] | ¹H NMR (DMSO-d₆, 500 MHz): δ 10.23- 10.49 (m, 1 H), 9.75- 10.10 (m, 1 H), 8.33 (d, J = 3.9 Hz, 1 H), 7.80-8.07 (m, 2 H), 7.64 (s, 1 H), 7.15-7.40 (m, 1 H), 6.75 (br. s., 2 H), 6.23-6.46 (m, 1 H), 3.64 (d, J = 3.4 Hz, 9 H), 2.21 (s, 3 H), 1.83 ppm (s, 3 H) | 448 | A |
| 46 | N²-[3,4- bis(methyloxy) phenyl]-N⁴-{3- methyl-1-[3- (methyloxy) phenyl]-1H- pyrazol-5-yl}-2,4- pyrimidinediamine | 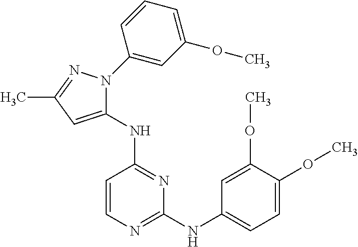 | ¹H NMR (DMSO-d₆, 400 MHz): δ 10.26- 10.46 (m, 1 H), 10.06- 10.19 (m, 1 H), 7.90 (d, J = 4.8 Hz, 1 H), 7.33-7.41 (m, 1 H), 7.12 (br. s., 1H), 7.00-7.06 (m, 2 H), 6.94 (br. s., 3 H), 6.38 (s, 1 H), 6.25-6.34 (m, 1 H), 3.75 (d, 9 H), 2.24 ppm (s, 3 H) | 433 | A |
| 47 | 3-{[4-({3-methyl- 1-[4-(methyloxy) phenyl]-1H- pyrazol-5- yl}amino)-2- pyrimidinyl]amino} benzene sulfonamide | 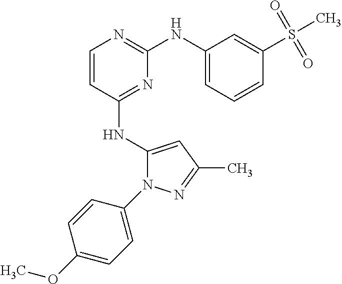 | ¹H NMR (400 MHz, DMSO-d₆) δ 9.98 (br. s., 1H), 9.74 (br. s., 1H), 8.02 (d, J = 6.27 Hz, 2H), 7.90 (d, J = 7.53 Hz, 1H), 7.32- 7.49 (m, 6H), 6.94-7.04 (m, 2H), 6.32-6.39 (m, 1H), 6.17-6.32 (m, 1H), 3.77 (s, 3H), 2.25 (s, 3 H) | 452 | A |
| 48 | N²-[3-chloro-4-(4- methyl-1- piperazinyl)phenyl]- N⁴-{3-methyl-1- [4-(methyloxy) phenyl]-1H- pyrazol-5-yl}-2,4- pyrimidinediamine | 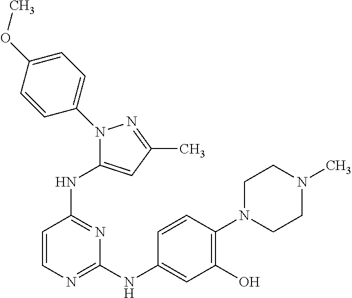 [SMILES: COc1ccc(-n2nc(C)cc2Nc2ccnc(Nc3ccc(N4CCN(C)CC4)c(O)c3)n2)cc1] | ¹H NMR (DMSO-d₆, 400 MHz): δ 9.64 (br. s., 2 H), 7.97-8.03 (m, 1 H), 7.86-7.93 (m, 1 H), 7.42- 7.50 (m, 1 H), 7.33-7.41 (m, 2 H), 7.12 (d, J = 8.8 Hz, 1 H), 6.96-7.03 (m, 2 H), 6.32 (s, 1 H), 6.21 (d, J = 6.0 Hz, 1 H), 3.77 (s, 3 H), 3.52 (br. s., 2 H), 3.34 (br. s., 2 H), 3.22 (br. s., 2 H), 2.86-3.01 (m, 5 H), 2.25 ppm (s, 3 H) | 455 | B |
| 49 | N²-[3,4- bis(methyloxy) phenyl]-N⁴-{1-[4- (methyloxy) phenyl]-1H- pyrazol-5-yl}-2,4- pyrimidinediamine | 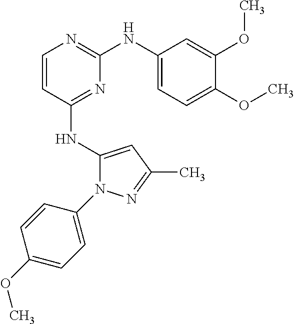 [SMILES: COc1ccc(-n2nc(C)cc2Nc2ccnc(Nc3ccc(OC)c(OC)c3)n2)cc1] | ¹H NMR (DMSO-d₆, 400 MHz): δ 10.35- 10.52 (m, 1 H), 10.18- 10.33 (m, 1 H), 7.93 (d, J = 13.1 Hz, 1 H), 7.71 (s, 1 H), 7.38 (d, J = 8.8 Hz, 2 H), 7.00-7.15 (m, 3 H), 6.93 (s, 2 H), 6.56 (br. s., 1 H), 6.30 (br. s., 1 H), 3.79 (s, 4 H), 3.76 (s, 3 H), 3.66 ppm (br. s., 3 H) | 419 | A |
| 50 | N²-[3,4- bis(methyloxy) phenyl]-N⁴-[3- methyl-1-(2- methylphenyl)- 1H-pyrazol-5-yl]- 2,4- pyrimidinediamine trifluoroacetate | 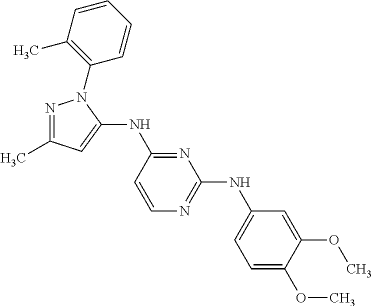 | ¹H NMR (DMSO-d₆) δ 9.57 (br. s., 1H), 7.86 (br. s., 1H), 7.35-7.38 (m, 3H), 7.25-7.32 (m, 1H), 7.17 (br. s., 1H), 6.98- 7.05 (m, 1H), 6.88-6.96 (m, 1H), 6.36 (br. s., 1H), 6.20 (s, 1H), 3.75 (s, 3H), 3.69 (s, 3H), 2.21 (s, 3H), 2.17 (s, 3H) | 417 | A |
| 51 | N²-[3,4- bis(methyloxy) phenyl]-N⁴-[1-(3- chlorophenyl)-3- methyl-1H- pyrazol-5-yl]-2,4- pyrimidinediamine trifluoroacetate | 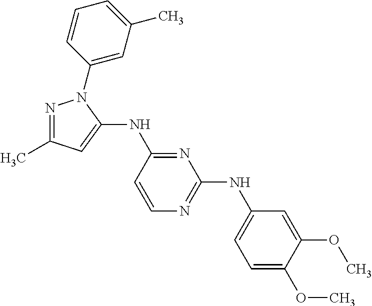 | ¹H NMR (DMSO-d₆) δ 7.87-7.95 (m, 1H), 7.51- 7.55 (m, 1H), 7.39-7.50 (m, 3H), 7.13 (br. s., 1H), 6.94-7.00 (m, 1H), 6.90 (br. s., 1H), 6.37 (d, J = 1.0 Hz, 1H), 6.24 (br. s., 1H), 3.75 (s, 3H), 3.66 (s, 3H), 2.24 (s, 3H) | 437 | A |
| 52 | N²-[3,4- bis(methyloxy) phenyl]-N⁴-[1-(3- chloro-4- methylphenyl)-3- methyl-1H- pyrazol-5-yl]-2,4- pyrimidinediamine trifluoroacetate | 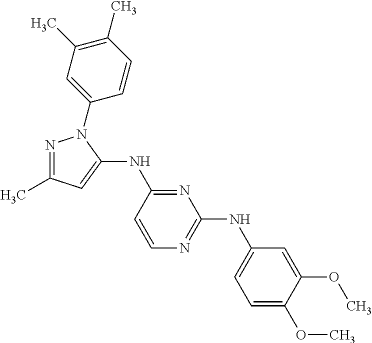 | ¹H NMR (DMSO-d₆) δ 7.91 (br. s., 1H), 7.50- 7.54 (m, 1H), 7.42 (d, J = 10.3 Hz, 1H), 7.32-7.36 (m, 1H), 7.13 (br. s., 1H), 6.93-7.00 (m, 1H), 6.91 (br. s., 1H), 6.36 (br. s., 1H), 6.18-6.29 (m, 1H), 3.75 (s, 3H), 3.67 (br. s., 3H), 2.34 (s, 3H), 2.23 (s, 3H) | 451 | A |
| 53 | N²-[3,4- bis(methyloxy) phenyl]-N⁴-[1-(4- chlorophenyl)-3- methyl-1H- pyrazol-5-yl]-2,4- pyrimidinediamine trifluoroacetate | 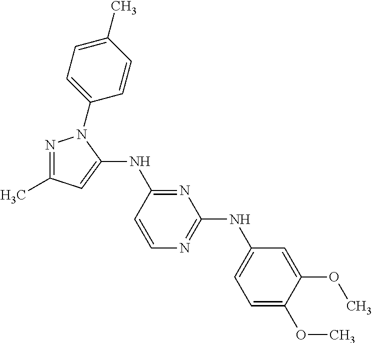 | ¹H NMR (DMSO-d₆) δ 7.92 (br. s., 1H), 7.49 (d, J = 5.4 Hz, 4H), 7.13-7.24 (m, 1H), 6.96-7.06 (m, 1H), 6.81-6.91 (m, 1H), 6.32-6.40 (m, 1H), 6.12- 6.24 (m, 1H), 3.74 (s, 3H), 3.66 (s, 3H), 2.23 (s, 3H) | 437 | A |
| 54 | 3-{5-[(2-{[3,4- bis(methyloxy) phenyl]amino}-4- pyrimidinyl)amino]- 3-methyl-1H- pyrazol-1- yl}benzoic acid trifluoroacetate | 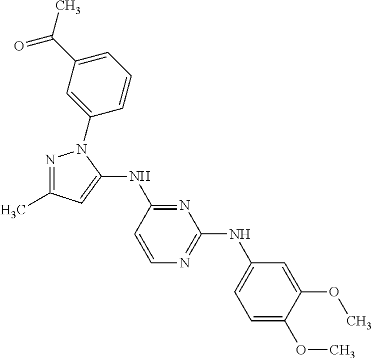 [SMILES: COc1ccc(Nc2nccc(Nc3cc(C)nn3-c3cccc(C(C)=O)c3)n2)cc1OC] | ¹H NMR (DMSO-d₆) δ 8.01-8.05 (m, 1H), 7.85- 7.92 (m, 2H), 7.68-7.74 (m, 1H), 7.54-7.61 (m, 1H), 7.09-7.16 (m, 1H), 6.92-7.00 (m, 1H), 6.85- 6.91 (m, 1H), 6.36-6.41 (m, 1H), 6.19-6.27 (m, 1H), 3.74 (s, 3H), 3.66 (s, 3H), 2.25 (s, 3H) | 447 | A |
| 55 | N²-[3,4- bis(methyloxy) phenyl]-N⁴-(1- methyl-3-phenyl- 1H-pyrazol-5-yl)- 2,4- pyrimidinediamine trifluoroacetate | 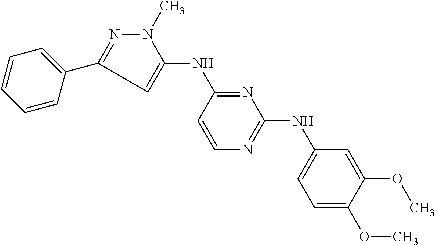 [SMILES: COc1ccc(Nc2nccc(Nc3cc(-c4ccccc4)nn3C)n2)cc1OC] | ¹H NMR (DMSO-d₆) δ 7.94-8.02 (m, 1H), 7.71 (br. s., 2H), 7.40 (t, J = 7.6 Hz, 2H), 7.27-7.33 (m, 1H), 7.13 (br. s., 1H), 7.02- 7.08 (m, 1H), 6.89 (br. s., 1H), 6.74-6.84 (m, 1H), 6.38-6.48 (m, 1H), 3.77 (s, 3H), 3.70 (s, 3H), 3.65 (br. s., 3H) | 403 | A |
| 56 | N²-[3,4- bis(methyloxy) phenyl]-N⁴-(1,3- dimethyl-1H- pyrazol-5-yl)-2,4- pyrimidinediamine trifluoroacetate | 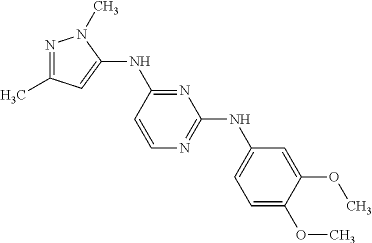 | ¹H NMR (DMSO-d₆) δ 7.85-8.00 (m, 1H), 7.10 (br. s., 1H), 6.95 (br. s., 2H), 6.31-6.47 (m, 1H), 6.12 (br. s., 1H), 3.76 (s, 3H), 3.68 (s, 3H), 3.61 (s, 3H), 2.10 (s, 3H) | 341 | A |
| 57 | N²-[3,4- bis(methyloxy) phenyl]-N⁴-(3- methyl-1-phenyl- 1H-pyrazol-5-yl)- 2,4- pyrimidinediamine trifluoroacetate | 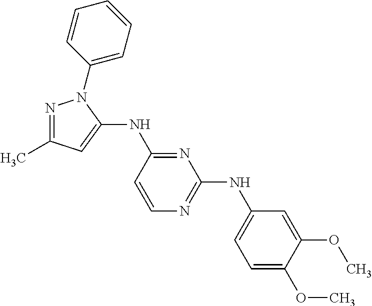 | ¹H NMR (DMSO-d₆) δ 7.85-7.92 (m, 1H), 7.45 (d, J = 4.4 Hz, 4H), 7.35 (d, J = 4.2 Hz, 1H), 7.14 (br. s., 1H), 6.94-7.01 (m, 1H), 6.92 (br. s., 1H), 6.33- 6.39 (m, 1H), 6.17-6.28 (m, 1H), 3.75 (s, 3H), 3.67 (s, 3H), 2.24 (s, 3H) | 403 | A |
| 58 | N⁴-(3,4-dimethyl- 1H-pyrazol-5-yl)- N²-[3-(1- pyrrolidinyl sulfonyl)phenyl]- 2,4- pyrimidinediamine trifluoroacetate | 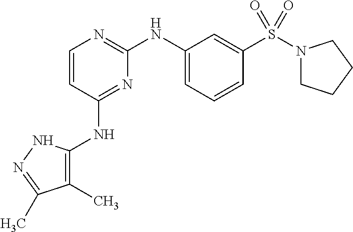 [SMILES: Cc1n[nH]c(Nc2ccnc(Nc3cccc(S(=O)(=O)N4CCCC4)c3)n2)c1C] | ¹H NMR (DMSO-d₆) δ 9.70-10.34 (m, 2H), 8.16 (br. s., 1H), 8.00 (d, J = 6.3 Hz, 1H), 7.81-7.97 (m, 1H), 7.36-7.60 (m, 2H), 6.42 (d, J = 6.6 Hz, 1H), 3.15 (t, J = 6.3 Hz, 4H), 2.17 (s, 3H), 1.81 (s, 3H), 1.66 (t, 4H) | 414 | C |
| 59 | N²-[3-(1- pyrrolidinyl sulfonyl)phenyl]- N⁴-(1,3,4- trimethyl-1H- pyrazol-5-yl)-2,4- pyrimidinediamine trifluoroacetate | 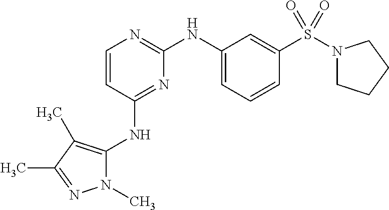 [SMILES: Cc1nn(C)c(Nc2ccnc(Nc3cccc(S(=O)(=O)N4CCCC4)c3)n2)c1C] | ¹H NMR (DMSO-d₆) δ 9.91 (br. s., 1H), 9.52 (br. s., 1H), 7.74-8.23 (m, 4H), 7.55 (t, J = 8.1 Hz, 1H), 7.38 (br. s., 2H), 3.52 (s, 3H), 3.07-3.21 (m, 4H), 2.10 (s, 3H), 1.77 (s, 3H), 1.58-1.71 (m, 4H) | 428 | C |
| 60 | N⁴-[3,4-dimethyl- 1-(2-pyridinyl)- 1H-pyrazol-5-yl]- N²-[3-(1- pyrrolidinyl sulfonyl)phenyl]- 2,4- pyrimidinediamine trifluoroacetate | 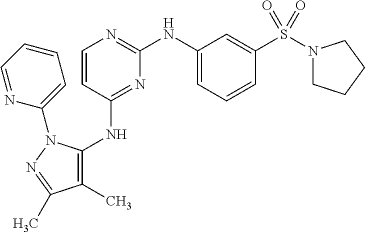 [SMILES: Cc1nn(-c2ccccn2)c(Nc2ccnc(Nc3cccc(S(=O)(=O)N4CCCC4)c3)n2)c1C] | ¹H NMR (DMSO-d₆) δ 9.54 (br. s., 1H), 9.42 (br. s., 1H), 8.35 (d, J = 3.7 Hz, 1H), 7.97-8.11 (m, 3H), 7.84-7.93 (m, 1H), 7.69 (d, J = 8.3 Hz, 1H), 7.39 (t, J = 7.9 Hz, 1H), 7.19-7.34 (m, 2H), 6.20 (br. s., 1H), 3.13 (br. s., 4H), 2.24 (s, 3H), 1.86 (s, 3H), 1.63 (br. s., 4H) | 491 | C |
| 61 | N⁴-[3,4-dimethyl- 1-(2-pyridinyl)- 1H-pyrazol-5-yl]- N²-[4-fluoro-3- (methyloxy) phenyl]-2,4- pyrimidinediamine | 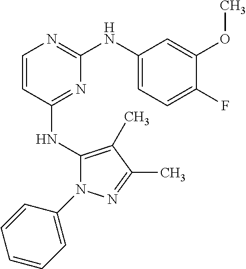 [SMILES: COc1cc(Nc2nccc(Nc3c(C)c(C)nn3-c3ccccc3)n2)ccc1F] | ¹H NMR (DMSO-d₆, 500 MHz): δ 10.16- 10.35 (m, 1 H), 9.83- 10.04 (m, 1 H), 8.31-8.36 (m, 1 H), 7.93-7.99 (m, 1 H), 7.85-7.92 (m, 1 H), 7.61-7.69 (m, 1 H), 7.22- 7.33 (m, 2 H), 7.08 (br. s., 1 H), 6.87-7.01 (m, 1 H), 6.26-6.38 (m, 1 H), 3.67 (s, 3 H), 2.22 (s, 3 H), 1.82 ppm (s, 3 H) | 406 | A |
| 62 | N4-(4,5-dimethyl- 1H-pyrazol-3-yl)- N2-[4-fluoro-3- (methylsulfonyl) phenyl]-2,4- pyrimidinediamine trifluoroacetate | 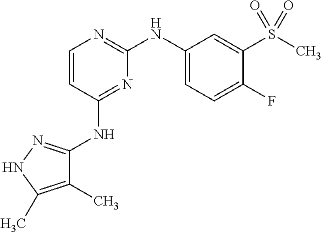 [SMILES: Cc1[nH]nc(Nc2ccnc(Nc3ccc(F)c(S(C)(=O)=O)c3)n2)c1C] | ¹H NMR (DMSO-d₆) δ 10.26 (br. s., 1H), 10.06 (br. s., 1H), 8.22 (br. s., 1H), 8.00 (d, J = 6.8 Hz, 2H), 7.46 (br. s., 1H), 6.43 (d, J = 6.5 Hz, 1H), 3.33 (s, 3H), 2.17 (s, 3H), 1.81 (s, 3H) | 377 | C |
| 63 | N-[2- (dimethylamino) ethyl]-3-({4-[(3,4- dimethyl-1H- pyrazol-5- yl)amino]-2- pyrimidinyl}amino)- N- methylbenzene sulfonamide trifluoroacetate | 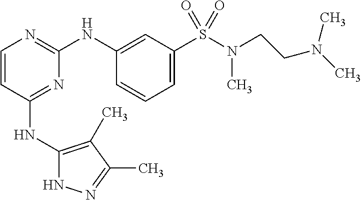 [SMILES: Cc1n[nH]c(Nc2ccnc(Nc3cccc(S(=O)(=O)N(C)CCN(C)C)c3)n2)c1C] | ¹H NMR (DMSO-d₆) δ 9.91 (br. s., 1H), 9.41 (br. s., 1H), 8.20 (br. s., 1H), 8.03 (d, J = 6.3 Hz, 2H), 7.52 (t, J = 8.0 Hz, 1H), 7.38 (br. s., 1H), 6.36 (d, J = 6.1 Hz, 1H), 3.22-3.37 (m, 4H), 2.85 (d, J = 2.3 Hz, 3H), 2.71 (s, 6H), 2.16 (s, 3H), 1.82 (s, 3H) | 445 | C |
| 64 | N,N-dimethyl-3- ({4-[(1,3,4- trimethyl-1H- pyrazol-5- yl)amino]-2- pyrimidinyl}amino) benzene sulfonamide trifluoroacetate | 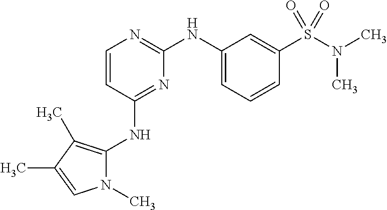 | ¹H NMR (DMSO-d₆) δ 9.99 (br. s., 1H), 9.75 (br. s., 1H), 8.05 (d, J = 6.3 Hz, 2H), 7.90 (br. s., 1H), 7.45 (br. s., 1H), 7.33 (d, J = 7.6 Hz, 1H), 6.19 (br. s., 1H), 3.25 (s, 3H), 2.60 (s, 6H), 2.10 (s, 3H), 1.77 (s, 3H) | 402 | C |
| 65 | 3-[(4-{[1-(2- hydroxyethyl)- 3,4-dimethyl-1H- pyrazol-5- yl]amino}-2- pyrimidinyl)amino]- N,N- dimethylbenzene sulfonamide | 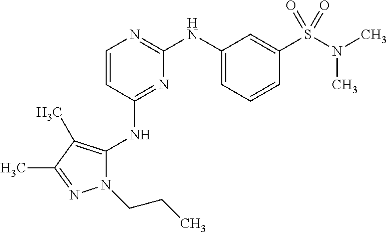 | ¹H NMR (DMSO-d₆) δ 9.47 (br. s., 1H), 8.70- 8.92 (m, 1H), 7.97-8.20 (m, 3H), 7.40 (br. s., 1H), 7.21 (br. s., 1H), 4.77 (br. s., 1H), 3.87 (t, J = 6.2 Hz, 2H), 3.61 (d, J = 5.1 Hz, 2H), 2.61 (s, 6H), 2.11 (s, 3H), 1.76 (s, 3H) | 432 | C |
| 66 | 3-{[4-({3,4- dimethyl-1-[4- (methyloxy) phenyl]-1H- pyrazol-5- yl}amino)-2- pyrimidinyl]amino}- N,N-dimethyl benzene sulfonamide trifluoroacetate | 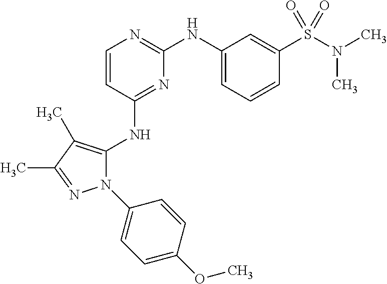 | ¹H NMR (DMSO-d₆) δ 9.79 (br. s., 1H), 9.37 (br. s., 1H), 7.86-8.16 (m, 3H), 7.23-7.53 (m, 5H), 6.95 (d, J = 8.8 Hz, 2H), 3.73 9s, 3H), 3.11-3.23 (m, 3H), 2.56-2.66 (m, 3H), 2.21 (s, 3H), 1.83 (s, 3H) | 494 | C |
| 67 | N4-{3,4-dimethyl- 1-[4-(methyloxy) phenyl}-1H- pyrazol-5-yl}-N2- [3-(1-pyrrolidinyl sulfonyl)phenyl]- 2,4- pyrimidinediamine trifluoroacetate | 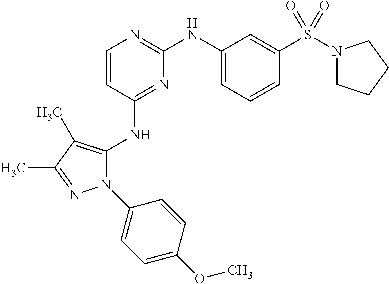 [SMILES: COc1ccc(-n2nc(C)c(C)c2Nc2ccnc(Nc3cccc(S(=O)(=O)N4CCCC4)c3)n2)cc1] | ¹H NMR (DMSO-d₆) δ 9.61 (br. s., 1H), 9.22 (s, 1H), 8.01 (br. s., 3H), 7.21- 7.44 (m, 5H), 6.95 (d, J = 8.8 Hz, 2H), 3.73 (s, 3H), 3.07-3.19 (m, 4H), 2.21 (s, 4H), 1.83 (s, 3H), 1.63 (br. s., 3H) | 520 | C |
| 68 | 2-{3,4-dimethyl- 5-[(2-{[3-(1- pyrrolidinyl sulfonyl)phenyl] amino}-4- pyrimidinyl)amino]- 1H-pyrazol-1- yl}ethanol trifluoroacetate | 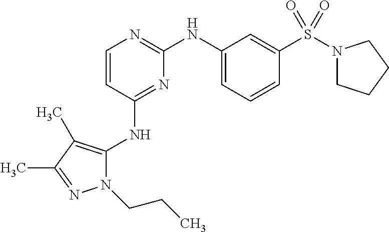 [SMILES: CCCn1nc(C)c(C)c1Nc1ccnc(Nc2cccc(S(=O)(=O)N3CCCC3)c2)n1] | ¹H NMR (DMSO-d₆) δ 9.34 (br. s., 1H), 8.37 (s 1H), 7.67-8.12 (m, 3H), 7.48 (br. s., 1H), 7.25 (br. s., 1H), 4.65 (br. s., 1H), 3.85 (t, J = 6.2 Hz, 2H), 3.62 (d, J = 5.1 Hz, 2H), 3.05 (s, 4H), 2.02 (s, 4H), 2.10 (s, 3H), 1.74 (s, 3H) | 458 | C |
| 69 | 2-{3,4-dimethyl-5- [(2-{[3- (methylsulfonyl) phenyl]amino}-4- pyrimidinyl)amino]- 1H-pyrazol-1- yl}ethanol | 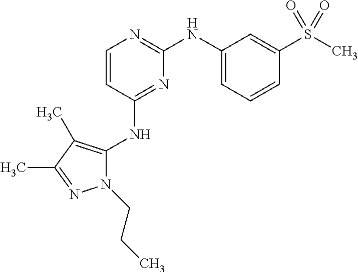 | ¹H NMR (METHANOL-d₄) δ 8.25 (br. s., 1H), 8.05 (d, J = 5.8 Hz, 1H), 7.89 (br. s., 1H), 7.40-7.53 (m, 2H), 4.04 (t, J = 6.1 Hz, 2H), 3.80 (d, J = 5.8 Hz, 2H), 3.10 (s, 3H), 2.23 (s, 3H), 2.05 (s, 2H), 1.87 (s, 3H) | 403 | A |
| 70 | N2-[4-fluoro-3- (methylsulfonyl) phenyl]-N4- (1,3,4-trimethyl- 1H-pyrazol-5-yl)- 2,4- pyrimidinediamine trifluoroacetate | 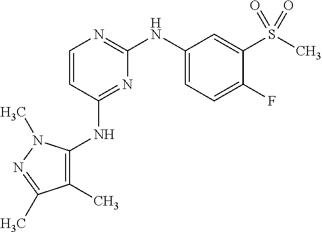 [SMILES: Cc1nn(C)c(Nc2ccnc(Nc3ccc(F)c(S(C)(=O)=O)c3)n2)c1C] | ¹H NMR (DMSO-d₆) δ 9.83 (br. s., 1H), 9.37 (br. s., 1H), 8.04 (d, J = 6.1 Hz, 3H), 7.33 (br. s., 1H), 3.51 (s, 3H), 3.30 (s, 3H), 2.09 (s, 3H), 1.77 (s, 3H) | 391 | C |
| 71 | N2-{3-[1- (dimethylamino)- 2,2,2- trifluoroethyl] phenyl}-N4- (1,3,4-trimethyl- 1H-pyrazol-5-yl)- 2,4- pyrimidinediamine trifluoroacetate | 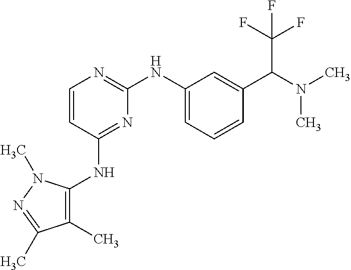 [SMILES: Cc1nn(C)c(Nc2ccnc(Nc3cccc(C(N(C)C)C(F)(F)F)c3)n2)c1C] | ¹H NMR (DMSO-d₆) δ 10.16 (br. s., 2H), 8.03 (d, J = 6.6 Hz, 1H), 7.38- 7.68 (m, 3H), 7.31 (br. s., 1H), 7.10 (br. s., 1H), 4.24 (br. s., 1H), 3.45 (s, 3H), 2.23 (br. s., 6H), 2.10 (s, 3H), 1.78 (s, 3H) | 420 | C |
| 72 | 2-[3-({4-[(4,5- dimethyl-1H- pyrazol-3- yl)amino]-2- pyrimidinyl}amino) phenyl]-2- methylpropane- nitrile | 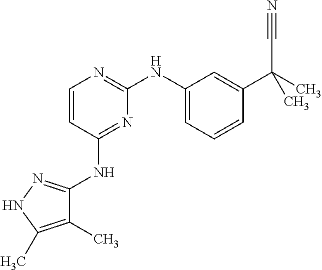 [SMILES: Cc1[nH]nc(Nc2ccnc(Nc3cccc(C(C)(C)C#N)c3)n2)c1C] | ¹H NMR (DMSO-d₆) δ 9.08 (br. s., 1H), 8.64 (br. s., 1H), 7.96 (br. s., 1H), 7.82 (br. s., 2H), 7.22 (br. s., 1H), 7.00 (br. s., 1H), 6.24 (br. s., 1H), 2.14 (br. s., 3H), 1.80 (br. s., 3H), 1.35 (s, 6H) | 348 | C |
| 73 | 2-{3-[(4-{[3,4- dimethyl-1-(2- pyridinyl)-1H- pyrazol-5- yl]amino}-2- pyrimidinyl)amino] phenyl}-2- methylpropane- nitrile | 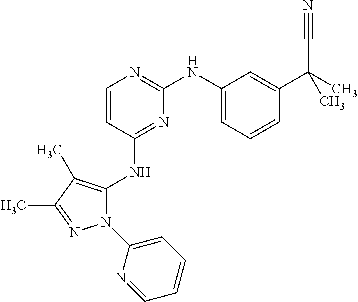 [SMILES: Cc1nn(-c2ccccn2)c(Nc2ccnc(Nc3cccc(C(C)(C)C#N)c3)n2)c1C] | ¹H NMR (DMSO-d₆) δ 9.22-9.32 (m, 1H), 9.19 (br. s., 1H), 8.35 (d, J = 3.9 Hz, 1H), 7.99 (d, J = 5.6 Hz, 1H), 7.81-7.92 (m, 1H), 7.74 (br. s., 1H), 7.62-7.70 (m, 2H), 7.15- 7.28 (m, 2H), 7.01 (d, J = 7.6 Hz, 1H), 6.13 (d, J = 5.6 Hz, 1H), 2.23 (s, 3H), 1.85 (s, 3H), 1.62 (s, 7H) | 425 | C |
| 74 | 2-{3-[(4-{[1-(2- hydroxyethyl)- 3,4-dimethyl-1H- pyrazol-5- yl]amino}-2- pyrimidinyl)amino] phenyl}-2- methylpropane- nitrile | 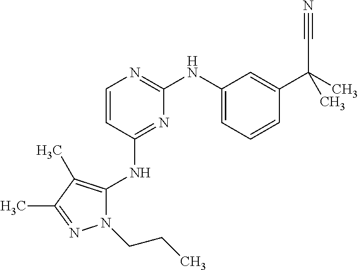 [SMILES: CCCn1nc(C)c(C)c1Nc1ccnc(Nc2cccc(C(C)(C)C#N)c2)n1] | ¹H NMR (DMSO-d₆) δ 9.20 (br. s., 1H), 8.72 (br. s., 1H), 8.01 (d, J = 5.6 Hz, 1H), 7.78 (br. s., 2H), 7.20 (br. s., 1H), 7.00 (d, J = 7.3 Hz, 1H), 4.76 (br. s., 1H), 3.86 (t, J = 6.6 Hz, 2H), 3.60 (q, J = 6.2 Hz, 2H), 2.10 (s, 3H), 1.76 (s, 3H), 1.62 (s, 6H) | 391 | C |
| 75 | 3-({4-[(3,4- dimethyl-1H- pyrazol-5- yl)amino]-2- pyrimidinyl} amino)-N,N- dimethyl-5- (trifluoromethyl) benzene sulfonamide | 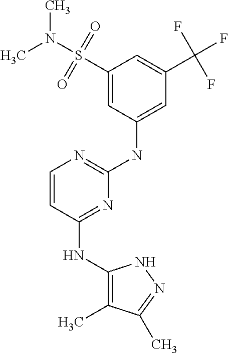 [SMILES: Cc1n[nH]c(Nc2ccnc(Nc3cc(C(F)(F)F)cc(S(=O)(=O)N(C)C)c3)n2)c1C] | ¹H NMR (400 MHz, DMSO-d₆) δ 10.23 (br. s., 1H), 9.65 (br. s., 1H), 8.53 (br. s., 1H), 8.22 (br. s., 1H), 8.07 (d, J = 6.06 Hz, 1H), 7.48 (s, 1H), 6.39 (d, J = 6.06 Hz, 1H), 2.66 (s, 6H), 2.15 (s, 3H), 1.79 (s, 3H) | 456 | G |
| 76 | 3-({4-[(3,4- dimethyl-1H- pyrazol-5- yl)amino]-2- pyrimidinyl}amino)- N,N,5- trimethylbenzene sulfonamide | 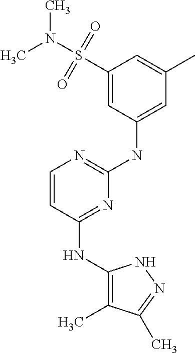 | ¹H NMR (400 MHz, DMSO-d₆) δ 12.34 (br. s., 1H), 10.17 (br. m., 2H), 7.86-8.06 (m, 2H), 7.60 (br. s., 1H), 7.21 (s, 1H), 6.39 (d, J = 6.57 Hz, 1H), 2.60 (s, 6H), 2.34 (s, 3H), 2.16 (s, 3H), 1.80 (s, 3H) | 402 | G |
| 77 | 2-methyl-2-[3-({4- [(1,3,4-trimethyl- 1H-pyrazol-5- yl)amino]-2- pyrimidinyl}amino) phenyl]propane- nitrile | 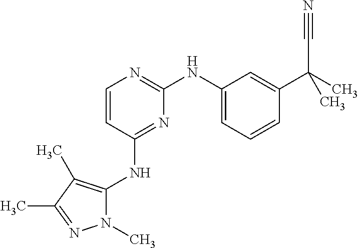 [SMILES: Cc1nn(C)c(Nc2ccnc(Nc3cccc(C(C)(C)C#N)c3)n2)c1C] | ¹H NMR (DMSO-d₆) δ 9.22 (s, 1H), 8.87 (br. s., 1H), 8.03 (d, J = 5.8 Hz, 1H), 7.77 (br. s., 2H), 7.20 (br. s., 1H), 7.02 (s, 1H), 3.51 (s, 3H), 2.09 (s, 3H), 1.78 (s, 3H), 1.62 (s, 6H) | 362 | C |
| 78 | 5-({4-[(3,4- dimethyl-1H- pyrazol-5- yl)amino]-2- pyrimidinyl}amino)- N-(2- hydroxyethyl)-2- methylbenzene sulfonamide | 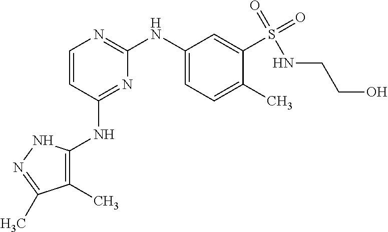 [SMILES: Cc1ccc(Nc2nccc(Nc3[nH]nc(C)c3C)n2)cc1S(=O)(=O)NCCO] | ¹H NMR (400 MHz, METHANOL-d₄) δ ppm 1.92 (br. s., 3 H) 2.24 (s, 3 H) 2.59 (s, 3 H) 3.01-3.09 (m, 2 H) 3.56 (t, J = 6.06 Hz, 2 H) 6.22 (br. s., 1H) 7.21-7.30 (m, 1 H) 7.78- 7.86 (m, 1 H) 7.98 (br. s., 1 H) 8.23 (br. s., 1 H) | 417 (M+) | F |
| 79 | N-cyclopentyl-3- ({4-[(3,4-dimethyl- 1H-pyrazol-5- yl)amino]-2- pyrimidinyl}amino) benzene sulfonamide | 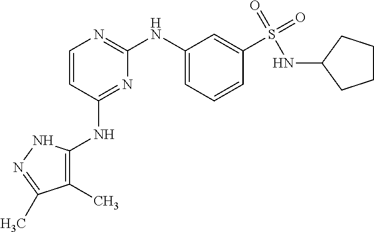 [SMILES: Cc1n[nH]c(Nc2ccnc(Nc3cccc(S(=O)(=O)NC4CCCC4)c3)n2)c1C] | ¹H NMR (400 MHz, METHANOL-d₄) δ ppm 1.46 (none, 4 H) 1.58- 1.82 (m, 4 H) 1.93 (br. s., 3 H) 2.25 (br. s., 3 H) 3.48- 3.62 (m, 1 H) 6.15-6.35 (m, 1 H) 7.35-7.51 (m, 2 H) 7.79-7.94 (m, 1 H) 7.94-8.09 (m, 1 H) 8.22- 8.39 (m, 1 H) | 427 (M+) | F |
| 80 | 5-({4-[(3,4- dimethyl-1H- pyrazol-5- yl)amino]-2- pyrimidinyl}amino)- 2-(ethyloxy)- N,N- dimethylbenzene sulfonamide | 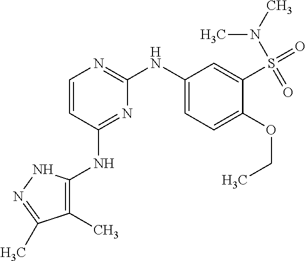 [SMILES: CCOc1ccc(Nc2nccc(Nc3[nH]nc(C)c3C)n2)cc1S(=O)(=O)N(C)C] | ¹H NMR (400 MHz, METHANOL-d₄) δ ppm 1.47 (t, J = 7.07 Hz, 3 H) 1.87-1.97 (m, 3 H) 2.05 (s, 2 H) 2.24 (br. s., 3 H) 2.85 (s, 6 H) 4.19 (m, J = 7.07, 7.07, 7.07 Hz, 2 H) 6.15-6.27 (m, 1 H) 7.05-7.16 (m, 1 H) 7.88- 7.98 (m, 2 H) 8.00-8.08 (m, 1 H) | 431 (M+) | F |
| 81 | 3-({4-[(3,4- dimethyl-1H- pyrazol-5- yl)amino]-2- pyrimidinyl}amino) benzamide | 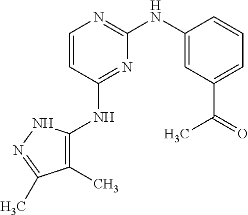 [SMILES: CC(=O)c1cccc(Nc2nccc(Nc3[nH]nc(C)c3C)n2)c1] | ¹H NMR (400 MHz, DMSO-d₆) δ ppm 1.83 (br. s., 3 H) 2.14 (br. s., 3 H) 6.16-6.38 (m, 1 H) 7.29 (br. s., 3 H) 7.72- 7.90 (m, 1 H) 7.92-8.16 (m, 3 H) 8.69-8.89 (m, 1 H) 9.07-9.27 (m, 1 H) 11.97-12.22 (m, 1 H) | 324 | F |
| 82 | N²-[3,4- bis(methyloxy) phenyl]-N⁴-(2- methyl-2,4,5,6- tetrahydro- cyclopenta[c]- pyrazol-3-yl)-2,4- pyrimidinediamine trifluoroacetate | 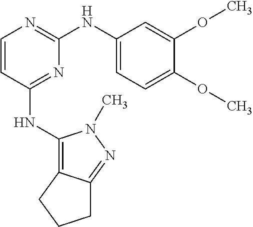 [SMILES: COc1ccc(Nc2nccc(Nc3c4c(nn3C)CCC4)n2)cc1OC] | ¹H NMR (DMSO-d₆) δ 9.70 (br. s., 1H), 8.55 (d, J = 5.4 Hz, 1H), 8.37 (s, 2H), 6.88-6.98 (m, 2 H), 6.82 (d, J = 5.4 Hz, 1H), 3.73 (s, 3H), 3.62 (br. s., 3H), 2.91-3.07 (m, 2H), 2.54-2.64 (m, 2H), 2.47 (d, J = 7.1 Hz, 2H) | 367 | C |
| 83 | N²-[3- (methylsulfonyl) phenyl]-N⁴-(2- methyl-2,4,5,6- tetrahydro- cyclopenta[c]- pyrazol-3-yl)-2,4- pyrimidinediamine trifluoroacetate | 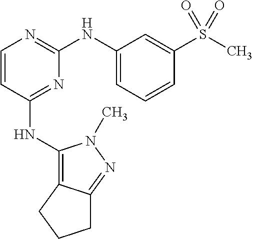 [SMILES: Cn1nc2c(c1Nc1ccnc(Nc3cccc(S(C)(=O)=O)c3)n1)CCC2] | ¹H NMR (DMSO- d₆) δ 10.32 (s, 1H), 8.67 (d, J = 5.4 Hz, 1H), 8.38-8.49 (m, 3H), 7.55-7.66 (m, 2H), 6.97 (d, J = 5.6 Hz, 1H), 3.75 (s, 3H), 3.20 (s, 3H), 3.08 (t, J = 7.2 Hz, 2H), 2.60 (dt, J = 13.7, 7.1 Hz, 2H), 2.38-2.48 (m, 2H) | 385 | C |
| 84 | N²-[3- (methylsulfonyl) phenyl]-N⁴- (1,4,5,6- tetrahydro- cyclopenta[c]- pyrazol-3-yl)-2,4- pyrimidinediamine trifluoroacetate | 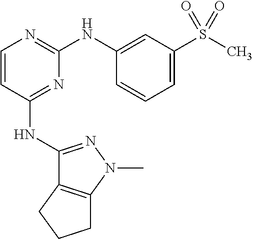 [SMILES: Cn1nc(Nc2ccnc(Nc3cccc(S(C)(=O)=O)c3)n2)c2c1CCC2] | ¹H NMR (METHANOL-d₄) δ 8.20 (br. s., 1H), 7.98 (d, J = 7.1 Hz, 1H), 7.89 (d, J = 7.8 Hz, 1H), 7.77 (d, J = 7.8 Hz, 1H), 7.61- 7.67 (m, 1H), 6.59 (br. s., 1H), 3.11 (s, 3H), 2.74- 2.82 (m, 2H), 2.68 (s, 2H), 2.45 (br. s., 2H) | 371 | C |
| 85 | N-[3-({4-[(3,4- dimethyl-1H- pyrazol-5- yl)amino]-2- pyrimidinyl}amino) phenyl]methane sulfonamide | 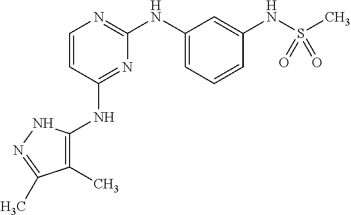 [SMILES: Cc1n[nH]c(Nc2ccnc(Nc3cccc(NS(C)(=O)=O)c3)n2)c1C] | ¹H NMR (400 MHz, DMSO-d₆) δ ppm 1.83 (br. s., 3 H) 2.16 (s, 3 H) 3.00 (s, 3 H) 6.39 (d, J = 6.57 Hz, 1 H) 6.86- 6.93 (m, 1 H) 7.22 (t, J = 7.83 Hz, 1 H) 7.35- 7.47 (m, 1 H) 7.54 (d, J = 7.58 Hz, 1 H) 7.97 (d, J = 6.32 Hz, 1 H) 9.69- 9.84 (m, 1 H) 9.84-10.12 (m, 1 H) 12.18-12.47 (m, 1 H) | 373 (M+) | F |
| 86 | 3-({4-[(3,4- dimethyl-1H- pyrazol-5- yl)amino]-2- pyrimidinyl}amino) benzonitrile | 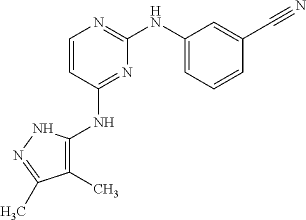 [SMILES: Cc1n[nH]c(Nc2ccnc(Nc3cccc(C#N)c3)n2)c1C] | ¹H NMR (400 MHz, DMSO-d₆) δ ppm 1.80 (br. s., 3 H) 2.18 (br. s., 3 H) 6.13-6.44 (m, 1 H) 7.19-7.52 (m, 2 H) 7.80- 8.13 (m, 2 H) 8.22-8.43 (m, 1 H) 8.85-9.10 (m, 1 H) 9.35-9.59 (m, 1 H) 11.94-12.21 (m, 1 H) | 305 (M+) | F |
| 87 | N4-(3,4-dimethyl- 1H-pyrazol-5-yl)- N2-[4- (methylsulfonyl phenyl ]-2,4- pyrimidinediamine hydrochloride | 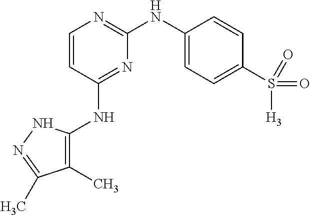 | ¹H NMR (400 MHz, DMSO-d₆) δ ppm 1.86 (br. s., 3 H) 2.21 (s, 3 H) 3.18 (s, 3 H) 6.51-6.61 (m, 1 H) 7.83 (br. s., 2 H) 7.90-8.02 (m, 2 H) 8.05- 8.18 (m, 1 H) 10.30- 10.51 (m, 1 H) 10.73- 10.90 (m, 1 H) | 358 (M⁺) | G |
| 88 | 2-{[3-({4-[(3,4- dimethyl-1H- pyrazol-5- yl)amino]-2- pyrimidinyl}amino) phenyl]sulfonyl} ethanol hydrochloride | 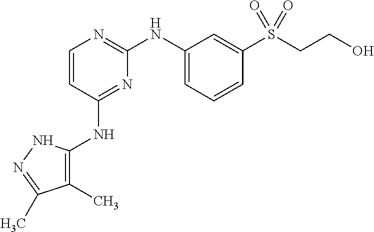 [SMILES: Cc1n[nH]c(Nc2ccnc(Nc3cccc(S(=O)(=O)CCO)c3)n2)c1C] | ¹H NMR (400 MHz, DMSO-d₆) δ ppm 1.83 (br. s., 3 H) 2.20 (s, 3 H) 3.38-3.56 (m, 2 H) 3.65- 3.79 (m, 2 H) 6.46-6.61 (m, 1 H) 7.51-7.69 (m, 2 H) 7.91-8.03 (m, 1 H) 8.03-8.21 (m, 2 H) 10.51- 10.70 (m, 1 H) 10.78- 10.97 (m, 1 H) | 388 (M⁺) | G |
| 89 | N4-(3,4-dimethyl- 1H-pyrazol-5-yl)- N2-{3-[(1- methylethyl)- sulfonyl]phenyl}- 2,4-pyrimidine diamine hydrochloride | 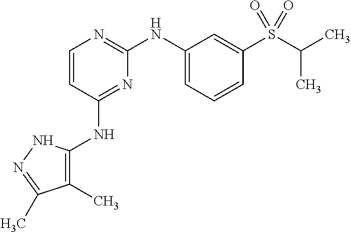 [SMILES: Cc1n[nH]c(Nc2ccnc(Nc3cccc(S(=O)(=O)C(C)C)c3)n2)c1C] | ¹H NMR (400 MHz, DMSO-d₆) δ ppm 1.16 (d, J = 6.82 Hz, 6 H) 1.82 (br. s., 3 H) 2.19 (s, 3 H) 3.77 (spt, J = 6.06 Hz, 1 H) 6.50 (d, J = 6.82 Hz, 1 H) 7.51- 7.66 (m, 2 H) 7.89-8.01 (m, 1 H) 8.04-8.11 (m, 1 H) 8.13-8.24 (m, 1 H) 10.30-10.51 (m, 1 H) 10.62-10.78 (m, 1 H) | 386 (M⁺) | G |
| 90 | N2-[3,5- bis(methyloxy) phenyl]-N4-(3,4- dimethyl-1H- pyrazol-5-yl)-2,4- pyrimidinediamine hydrochloride | 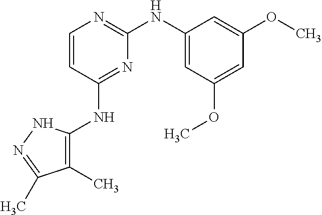 [SMILES: COc1cc(Nc2nccc(Nc3[nH]nc(C)c3C)n2)cc(OC)c1] | ¹H NMR (DMSO-d₆) δ 11.78-12.65 (m, 1H), 10.40-10.77 (m, 2H), 7.98 (d, J = 7.1 Hz, 1H), 6.59-6.86 (m, 2H),6.37- 6.49 (m, 1H), 6.31 (br. s., 1H), 3.57-3.89 (m, 6H), 2.17 (s, 3H), 1.78 (br. s., 3H) | 340 (M⁺) | G |
| 91 | N⁴-(4,5-dimethyl- 1H-pyrazol-3-yl)- N²-(1,1-dioxido- 2,3-dihydro-1,2- benzisothiazol-6- yl)-2,4- pyrimidine trifluoroacetate | 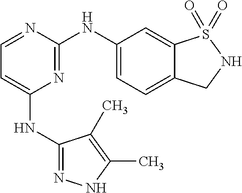 [SMILES: Cc1[nH]nc(Nc2ccnc(Nc3ccc4c(c3)S(=O)(=O)NC4)n2)c1C] | ¹H NMR (DMSO-d₆) δ 10.25 (br. s., 1H), 10.10 (br. s., 1H), 8.17 (br. s., 1H), 8.02 (d, J = 6.6 Hz, 1H), 7.77 (br. s., 2H), 7.46 (d, J = 8.5 Hz, 1H), 6.40 (d, J = 6.6 Hz, 1H), 4.34 (br. s., 2H), 2.18 (s, 3H), 1.83 (s, 3H) | 372 | H |
| 92 | N4-(3,4-dimethyl- 1H-pyrazol-5-yl)- N2-(2,2-dioxido- 1,3-dihydro-2- benzothien-5-yl)- 2,4- pyrimidinediamine hydrochloride | 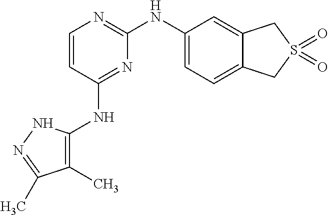 [SMILES: Cc1n[nH]c(Nc2ccnc(Nc3ccc4c(c3)CS(=O)(=O)C4)n2)c1C] | ¹H NMR (400 MHz, DMSO-d₆) δ ppm 1.82 (br. s., 3 H) 2.20 (s, 3 H) 4.39- 4.53 (m, 4 H) 6.46 (d, J = 7.07 Hz, 2 H) 6.79- 6.92 (m, 1 H) 7.28-7.41 (m, 1 H) 7.41-7.61 (m, 1 H) 7.65-7.75 (m, 1 H) 8.01 (d, J = 6.82 Hz, 1H) 10.47-10.72 (m, 2H) | 371 | G |
| 93 | N4-(3,4-dimethyl- 1H-pyrazol-5-yl)- N2-[3-(4- morpholinyl sulfonyl)phenyl]- 2,4- pyrimidinediamine hydrochloride | 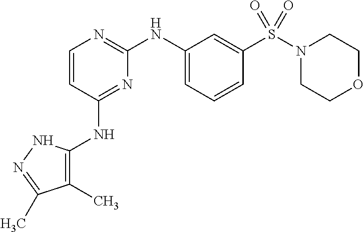 [SMILES: Cc1n[nH]c(Nc2ccnc(Nc3cccc(S(=O)(=O)N4CCOCC4)c3)n2)c1C] | ¹H NMR (DMSO-d₆) δ 10.61-10.79 (m, 1H), 10.29-10.52 (m, 1H), 8.09-8.21 (m, 1H), 8.05 (d, J = 6.8 Hz, 1H), 7.78- 7.92 (m, 1H), 7.51-7.65 (m, 1H), 7.37-7.46 (m, 1H), 6.50 (d, J = 6.8 Hz, 1H), 3.59-3.70 (m, 4H), 2.86 (br. s., 4H), 2.19 (s, 3H), 1.82 (br. s., 3H) | 429 (M⁺) | G |
| 94 | 1-{[3-({4-[(3,4- dimethyl-1H- pyrazol-5- yl)amino]-2- pyrimidinyl}amino) phenyl]sulfonyl}- 4-piperidinol hydrochloride | 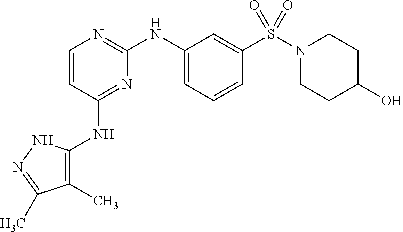 [SMILES: Cc1n[nH]c(Nc2ccnc(Nc3cccc(S(=O)(=O)N4CCC(O)CC4)c3)n2)c1C] | ¹H NMR (DMSO-d₆) δ 10.52-10.72 (m, 1H), 10.21-10.52 (m, 1H), 8.08-8.19 (m, 1H), 8.04 (d, J = 6.6 Hz, 1H), 7.78- 7.89 (m, 1H), 7.49-7.61 (m, 1H), 7.37-7.47 (m, 1H), 6.49 (d, J = 6.8 Hz, 1H), 3.48-3.59 (m, 1H), 3.06-3.16 (m, 2H), 2.69- 2.80 (m, 2H), 2.19 (s, 3H), 1.83 (br. s., 3H), 1.69- 1.79 (m, 2H), 1.38-1.50 (m, 2H) | 443 (M⁺) | G |
| 95 | N4-(3,4-dimethyl- 1H-pyrazol-5-yl)- N2-{3-[(1- methylethyl)thio] phenyl}-2,4- pyrimidinediamine | 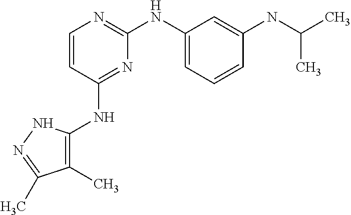 [SMILES: Cc1n[nH]c(Nc2ccnc(Nc3cccc(NC(C)C)c3)n2)c1C] | ¹H NMR (DMSO-d₆) δ 11.98-12.23 (m, 1H), 9.01-9.27 (m, 1H), 8.67- 9.01 (m, 1H), 7.98 (br. s., 1H), 7.76 (br. s., 2H), 7.62- 7.72 (m, 1H), 7.15 (br. s., 1H), 6.86 (br. s., 1H), 6.18- 6.35 (m, 1H), 3.38-3.48 (m, 1H), 2.15 (br. s., 3H), 1.83 (br. s., 3H), 1.19- 1.31 (m, 6H) | 354 (M⁺) | G |
| 96 | ethyl 2-{[3-({4- [(3,4-dimethyl- 1H-pyrazol-5- yl)amino]-2- pyrimidinyl}amino) phenyl]thio}-2- methylpropanoate | 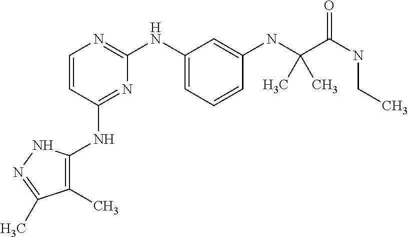 [SMILES: CCNC(=O)C(C)(C)Nc1cccc(Nc2nccc(Nc3[nH]nc(C)c3C)n2)c1] | ¹H NMR (DMSO-d₆) δ 12.30-12.60 (m, 1H), 10.23-10.52 (m, 1H), 8.00 (d, J = 6.8 Hz, 1H), 7.65-7.85 (m, 1H), 7.55- 7.65 (m, 1H), 7.27-7.39 (m, 1H), 7.08-7.20 (m, 1H), 6.45 (d, J = 6.8 Hz, 1H), 3.99-4.10 (m, 2H), 2.19 (s, 3H), 1.75-1.87 (m, 3H), 1.41 (s, 6H), 1.11 (s, 3H) | 426 (M⁺) | G |
| 97 | 2-{[3-({4-[(3,4- dimethyl-1H- pyrazol-5- yl)amino]-2- pyrimidinyl}amino) phenyl]thio}-2- methyl-1- propanol | 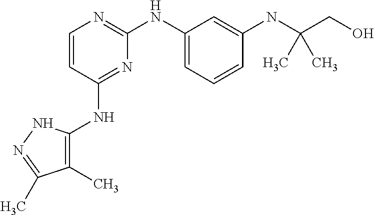 [SMILES: Cc1n[nH]c(Nc2ccnc(Nc3cccc(NC(C)(C)CO)c3)n2)c1C] | ¹H NMR (DMSO-d₆) δ 12.00-12.11 (m, 1H), 9.04-9.16 (m, 1H), 8.66- 8.80 (m, 1H), 7.93-8.00 (m, 1H), 7.87-7.93 (m, 1H), 7.85 (s, 2H), 7.08- 7.32 (m, 2H), 6.93-7.07 (m, 2H), 6.17-6.36 (m, 2H), 4.81-4.94 (m, 2H), 3.30 (d, J = 5.6 Hz, 2H), 2.16 (br. s., 3H), 1.81 (br. s., 3H), 1.14 (s, 6H) | 355 (M − H)⁺ | G |
| 98 | N4-(3,4-dimethyl- 1H-pyrazol-5-yl)- N2-[3-(1,3- oxazol-2- yl)phenyl]-2,4- pyrimidinediamine hydrochloride | 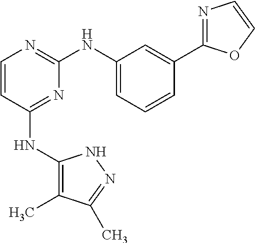 [SMILES: Cc1n[nH]c(Nc2ccnc(Nc3cccc(-c4ncco4)c3)n2)c1C] | ¹H NMR (DMSO-d₆) δ 10.91-11.09 (m, 1H), 10.55-10.88 (m, 1H), 8.24 (s, 1H), 8.06 (br. s., 2H), 7.68-7.81 (m, 1H), 7.44-7.57 (m, 1H), 7.41 (s, 1H), 6.48-6.57 (m, 1H), 2.11 (br. s., 3H), 1.79 (br. s., 3H) | 397 (M⁺) | G |
| 99 | N2-[3-(1,3- benzoxazol-2- yl)phenyl]-N4- (3,4-dimethyl-1H- pyrazol-5-yl)-2,4- pyrimidinediamine hydrochloride | 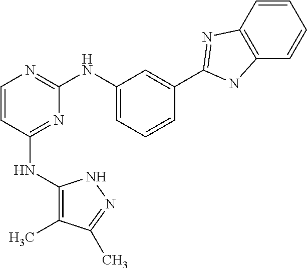 [SMILES: Cc1n[nH]c(Nc2ccnc(Nc3cccc(-c4nc5ccccc5[nH]4)c3)n2)c1C] | ¹H NMR (DMSO-d₆) δ 11.01-11.14 (m, 1H), 10.59-10.86 (m, 1H), 8.24-8.35 (m, 1H), 8.06- 8.15 (m, 1H), 7.93-8.03 (m, 2H), 7.77-7.88 (m, 3H), 7.51-7.64 (m, 1H), 7.40-7.51 (m, 3H), 6.50- 6.59 (m, 1H), 1.99-2.16 (m, 3H), 1.81 (br. s., 3H) | 384 (M⁺) | G |
| 100 | 4-(6,7-dimethyl- 2,3-dihydro-1H- imidazo[1,2- b]pyrazol-1-yl)-N- [3- (methylsulfonyl) phenyl]-2- pyrimidinediamine | 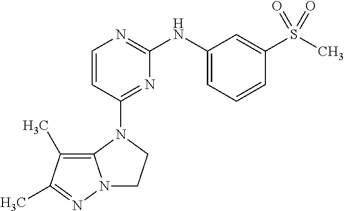 | ¹H NMR (METHANOL-d₄) δ 8.55-8.68 (m, 1H), 8.22 (d, J = 5.8 Hz, 1H), 7.78- 7.86 (m, 1H), 7.53-7.60 (m, 2H), 6.50 (d, J = 6.1 Hz, 1H), 4.62-4.71 (m, 2H), 4.22-4.30 (m, 2H), 3.12 (s, 3H), 2.19 (s, 3H), 2.14 (s, 3H) | 384 (M⁺) | A |
| 101 | N⁴-(3,4-dimethyl- 1H-pyrazol-5-yl)- N²-[4-methyl-3- (methylsulfonyl) phenyl]-2,4- pyrimidinediamine trifluoroacetate | 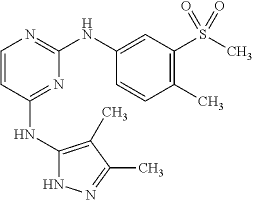 [SMILES: Cc1ccc(Nc2nccc(Nc3[nH]nc(C)c3C)n2)cc1S(C)(=O)=O] | ¹H NMR (DMSO-d₆) v 10.35 (br. s., 1H), 10.12- 10.29 (s, 1H), 7.99 (d, J = 6.8 Hz, 4H), 7.39 (br. s., 1H), 6.44 (d, J = 6.8 Hz, 1H), 2.61 (s, 3H), 2.18 (s, 3H), 2.08 (s, 3H), 1.81 (br. s. 3H) | 373 | H |
| 102 | N²-[3,5- bis(methylsulfonyl) phenyl]-N⁴-(3,4- dimethyl-1H- pyrazol-5-yl)-2,4- pyrimidinediamine trifluoroacetate | 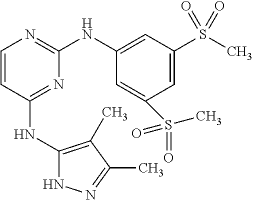 [SMILES: Cc1n[nH]c(Nc2ccnc(Nc3cc(S(C)(=O)=O)cc(S(C)(=O)=O)c3)n2)c1C] | ¹H NMR (DMSO-d₆) δ 10.31 (br. s., 1H), 9.70 (br. s., 1H), 8.47-8.64 (m, 2H), 8.06-8.16 (m, 1H), 7.85-8.01 (m, 2H), 6.41 (br. s., 1H), 3.27 (s, 6H), 2.12-2.21 (s, 3H), 1.80 (s, 3H) | 437 | H |
| 103 | N4-(1-cyclohexyl- 3,4-dimethyl-1H- pyrazol-5-yl)-N2- [4-fluoro-3- (methylsulfonyl) phenyl]-2,4- pyrimidinediamine | 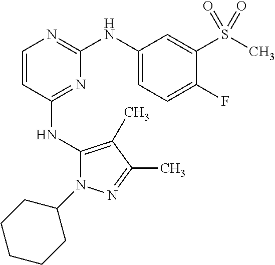 [SMILES: Cc1nn(C2CCCCC2)c(Nc2ccnc(Nc3ccc(F)c(S(C)(=O)=O)c3)n2)c1C] | ¹H NMR (DMSO-d₆) δ 9.47-9.63 (m, 1H), 8.79- 8.94 (m, 1H), 8.16-8.25 (m, 1H), 7.99-8.07 (m, 1H), 7.21-7.41 (m, 1H), 3.82-3.96 (m, 1H), 3.29 (s, 3H), 2.10 (s, 3H), 1.73 (br. s., 9H), 1.22 (none, 4H) | 458 (M⁺) | A |
| 104 | N²-[3,4- bis(methyloxy) phenyl]-N⁴-{3,4- dimethyl-1-[5- (methyloxy)-2- pyridinyl]-1H- pyrazol-5-yl}-2,4- pyrimidinediamine trifluoroacetate | 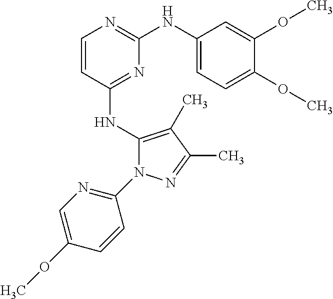 | ¹H NMR (DMSO-d₆) δ 10.47 (br. s., 1H), 10.08 (br. s., 1H), 8.05 (d, J = 2.8 Hz, 1H), 7.88 (br. s., 1H), 7.46-7.62 (m, 2H), 7.04 (br. s., 1H), 6.89 (br. s., 2H), 6.31 (br. s., 1H), 3.84 (s, 3H), 3.75 (s, 6H), 2.21 (s, 3H), 1.83 (s, 3H) | 448 | C |
| 105 | N⁴-{3,4-dimethyl- 1-[5-(methyloxy)- 2-pyridinyl]-1H- pyrazol-5-yl}-N²- [3- (methylsulfonyl) phenyl]-2,4- pyrimidinediamine trifluoroacetate | 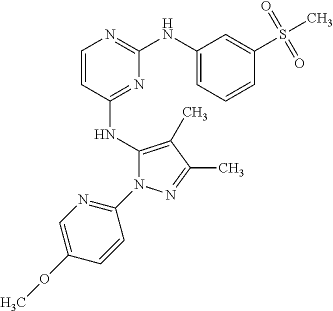 | ¹H NMR (DMSO-d₆) δ 9.96 (br. s., 1H), 9.74- 9.91 (m, 1H), 7.98-8.13 (m, 3H), 7.90 (br. s., 1H), 7.41-7.61 (m, 4H), 6.25 (br. s., 1H), 3.82 (s, 3H), 3.16 (s, 3H), 2.23 (s, 3H), 1.85 (s, 3H) | 466 | C |
| 106 | N²-[3,4- bis(methyloxy) phenyl]-N⁴-{3,4- dimethyl-1-[6- (methyloxy)-3- pyridinyl]-1H- pyrazol-5-yl}-2,4- pyrimidinediamine trifluoroaceate | 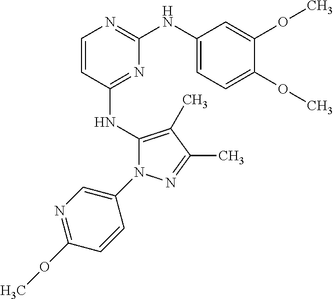 | ¹H NMR (DMSO-d₆) δ 10.17 (br. s., 1H), 8.20 (br. s., 1H), 7.81-7.96 (m, 1H), 7.73 (br. s., 1H), 7.10 (br. s., 1H), 6.91 (d, J = 8.8 Hz, 4H), 6.35 (br. s., 1H), 3.75 (s, 6H), 3.61 (br. s., 3H), 2.21 (s, 3H), 1.81 (s, 3H) | 448 | J |
| 107 | N⁴-{3,4-dimethyl- 1-[6-(methyloxy)- 3-pyridinyl]-1H- pyrazol-5-yl}-N²- [3- (methylsulfonyl) phenyl]-2,4- pyrimidinediamine trifluoroacetate | 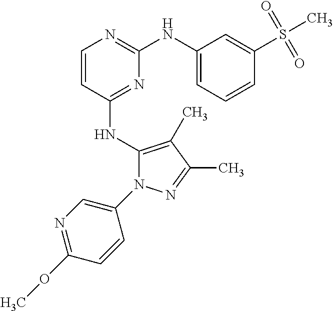 | ¹H NMR (DMSO-d₆) δ 9.85 (br. s., 1H), 9.51 (br. s., 1H), 8.20 (br. s., 1H), 8.08-8.17 (m, 1H), 8.03 (d, J = 6.1 Hz, 1H), 7.85- 7.99 (m, 1H), 7.76 (br. s., 1H), 7.48 (br. s., 2H), 6.87 (d, J = 8.8 Hz, 1H), 3.83 (s, 3H), 3.14 (br. s., 3H), 2.22 (s, 3H), 1.85 (s, 3H) | 466 | J |
| 108 | N⁴-(3,4-dimethyl- 1H-pyrazol-5-yl)- N²-[3-methyl-5- (methylsulfonyl) phenyl]-2,4- pyrimidinediamine hydrochloride | 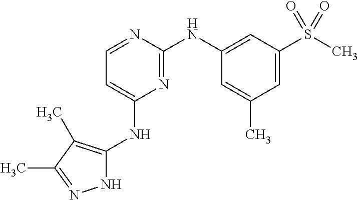 | ¹H NMR (DMSO-d₆) δ 10.86 (br. s., 1H), 10.62 (br. s., 1H), 8.06 (d, J = 7.1 Hz, 1H), 7.88 (br. s., 1H), 7.68-7.82 (m, 1H), 7.49 (br. s., 1H), 6.50 (d, J = 1.0 Hz, 1H), 3.18 (br. s., 3H), 2.27-2.44 (m, 3H), 2.20 (s, 3H), 1.82 (br. s., 3H) | 373 | K |
| 109 | N⁴-(3,4-dimethyl- 1H-pyrazol-5-yl)- N²-[3- (methylsulfonyl) phenyl]-2,4- pyrimidinediamine hydrochloride | 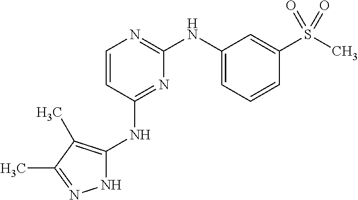 | ¹H NMR (DMSO-d₆) δ 11.03 (br. s., 1H), 10.67 (br. s., 1H), 8.09 (d, J = 7.1 Hz, 3H), 7.65 (br. s., 2H), 6.54 (d, J = 6.8 Hz, 1H), 3.20 (br. s., 3H), 2.21 (s, 3H), 1.84 (s, 3H) | 359 | K |
| 110 | 3-({4-[(3,4- DIMETHYL-1H- PYRAZOL-5- YL)AMINO]-2- PYRIMIDINYL} AMINO)-4- (METHYLOXY) BENZENE SULFONAMIDE | 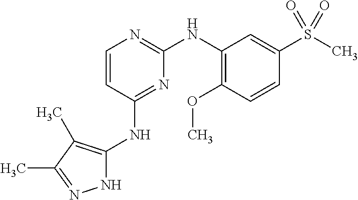 | ¹H NMR (400 MHz, DMSO-d₆) δ 10.65 (br. s., 1H), 10.14 (br. s., 1H), 8.40 (br. s., 2H), 8.10 (d, J = 6.32 Hz, 1H), 7.74 (br. s., 2H), 6.51 (d, J = 6.32 Hz, 1H), 3.72 (m, 2H), 2.84- 3.18 (m, 3H), 2.20 (s, 3H), 1.84 (br. s., 3H) | 390 | H |
| 111 | N4-(3,4-dimethyl- 1H-pyrazol-5-yl)- N2-{3- [(trifluoromethyl) sulfonyl] phenyl}- 2,4- pyrimidinediamine | 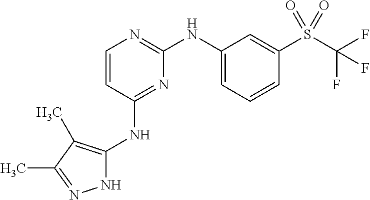 [SMILES: Cc1n[nH]c(Nc2ccnc(Nc3cccc(S(=O)(=O)C(F)(F)F)c3)n2)c1C] | ¹H NMR (400 MHz, DMSO-d₆) δ 10.82 (br. s., 1H), 10.27 (br. s., 1H), 8.39 (br. s., 2H), 8.04- 8.25 (m, 1H), 7.66-7.88 (m, 2H), 6.44-6.65 (m, 1H), 2.20 (s, 3H), 1.85 (br. s., 3H) | 413 | H |
| 112 | N4-(3,4- DIMETHYL-1H- PYRAZOL-5-YL)- N2-{3-METHYL- 5-[(1- METHYLETHYL) SULFONYL] PHENYL}-2,4- PYRIMIDINE DIAMINE | 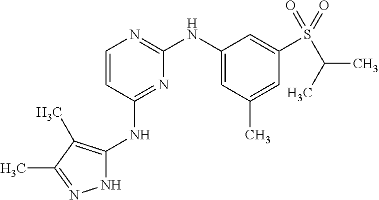 [SMILES: Cc1cc(Nc2nccc(Nc3[nH]nc(C)c3C)n2)cc(S(=O)(=O)C(C)C)c1] | ¹H NMR (400 MHz, DMSO-d₆) δ 9.65-10.40 (m, 1H), 7.90-8.13 (m, 2H), 7.61-7.86 (m, 1H), 7.22-7.40 (m, 1H), 6.26- 6.45 (m, 1H), 2.34 (br. s., 3H), 2.17 (s, 3H), 1.80 (br. s., 3H), 1.16 (d, 6H) | 401 | H |
| 113 | N⁴-(4,5-dimethyl- 1H-pyrazol-3-yl)- N²-[3- (methyloxy)-5- (methylsulfonyl) phenyl]-2,4- pyrimidinediamine hydrochloride | 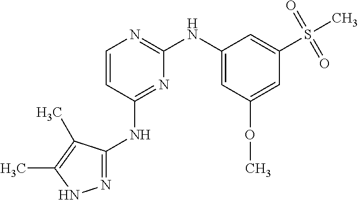 | ¹H NMR (DMSO-d₆) δ 10.79 (br. s., 1H), 10.48 (br. s., 1H), 8.07 (d, J = 6.8 Hz, 1H), 7.69 (br. s., 1H), 7.57 (br. s., 1H), 7.15 (br. s., 1H), 6.49 (d, J = 6.6 Hz, 1H), 3.74 (s, 3H), 2.19 (s, 3H), 1.82 (br. s., 3H), 1.05 (s, 3H) | 389 | H |
| 114 | 2-{[5-({4-[(4,5- dimethyl-1H- pyrazol-3- yl)amino]-2- pyrimidinyl}amino)- 2-methylphenyl] sulfonyl}-2- methyl-1- propanol hydrochloride | 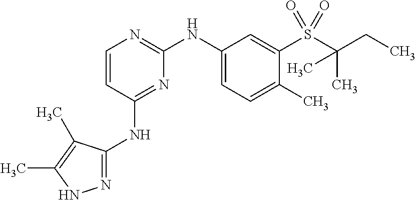 [SMILES: CCC(C)(C)S(=O)(=O)c1cc(Nc2nccc(Nc3n[nH]c(C)c3C)n2)ccc1C] | ¹H NMR (DMSO-d₆) δ 10.57 (br. s., 1H), 10.29- 10.48 (m, 1H), 8.05-8.19 (m, 1H), 8.00 (d, J = 6.8 Hz, 1H), 7.78 (br. s., 1H), 7.35 (br. s., 1H), 6.48 (d, J = 7.1 Hz, 1H), 3.54 (s, 4H), 2.60 (s, 3H), 2.19 (s, 3H), 1.81 (br. s., 3H), 1.22 (s, 7H) | 431 | H |
| 115 | 7-({4-[(4,5- dimethyl-1H- pyrazol-3- yl)amino]-2- pyrimidinyl}amino)- 2,3-dihydro-1- benzofuran-5- sulfonamide | 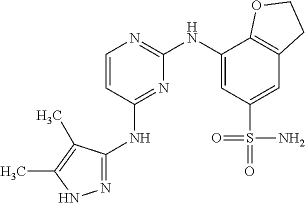 [SMILES: Cc1[nH]nc(Nc2ccnc(Nc3cc(S(N)(=O)=O)cc4c3OCC4)n2)c1C] | ¹H NMR (DMSO-d₆) δ 10.68 (br. s., 1H), 10.38 (s, 1H), 8.52 (br. s., 1H), 7.93-8.06 (m, 2H), 7.31- 7.41 (m, 1H), 7.18 (br. s., 1H), 6.33 (br. s., 1H), 4.69 (br. s., 2H), 2.07-2.21 (m, 5H), 1.85 (br. s., 3H) | 402 | H |
| 116 | N⁴-(4,5-dimethyl- 1H-pyrazol-3-yl)- N²-{4-fluoro-3- [(1-methylethyl) sulfonyl] phenyl}- 2,4- pyrimidinediamine | 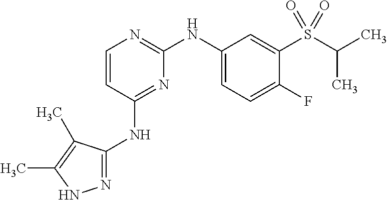 [SMILES: Cc1[nH]nc(Nc2ccnc(Nc3ccc(F)c(S(=O)(=O)C(C)C)c3)n2)c1C] | ¹H NMR (DMSO-d₆) δ 11.85 (s, 1H), 9.49 (s, 1H), 8.34 (br. s., 1H), 8.12 (dd, J = 5.9, 2.7 Hz, 1H), 8.01 (d, J = 5.6 Hz, 1H), 7.35 (br. s., 1H), 6.30 (br. s., 1H), 3.42-3.51 (m, 1H), 2.15 (s, 3H), 1.78 (s, 3H), 1.21 (d, J = 6.8 Hz, 6H) | 405 | H |
| 117 | N⁴-(4,5-dimethyl- 1H-pyrazol-3-yl)- N²-[5- (ethylsulfonyl)-2- (methyloxy) phenyl]-2,4- pyrimidinediamine | 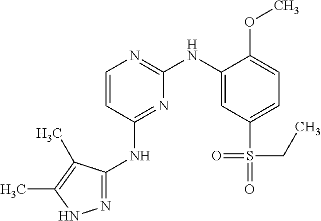 [SMILES: CCS(=O)(=O)c1ccc(OC)c(Nc2nccc(Nc3n[nH]c(C)c3C)n2)c1] | ¹H NMR (DMSO-d₆) δ 12.20 (br. s., 1H), 9.56 (br. s., 1H), 8.58 (br. s., 1H), 8.01 (d, J = 6.1 Hz, 1H), 7.57 (br. s., 1H), 7.29 (d, J = 8.3 Hz, 1H), 6.40 (d, J = 5.4 Hz, 1H), 3.97 (s, 3H), 3.17 (m, 2H), 2.14 (s, 3H), 1.80 (br. s., 3H), 1.11 (t, J = 7.2 Hz, 3H) | 403 | K |
| 118 | N²-1-benzothien- 4-yl-N⁴-(4,5- dimethyl-1H- pyrazol-3-yl)-2,4- pyrimidinediamine | 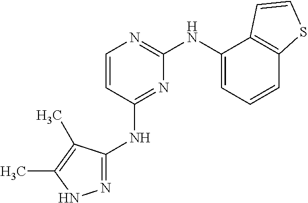 [SMILES: Cc1[nH]nc(Nc2ccnc(Nc3cccc4sccc34)n2)c1C] | ¹H NMR (DMF) δ 12.99 (br. s., 1H), 10.79-11.28 (m, 2H), 8.04-8.52 (m, 5H), 7.57-7.99 (m, 1H), 6.90 (d, J = 7.3 Hz, 1H), 2.60 (s, 3H), 2.24 (br. s., 3H) | 337 | K |
| 119 | 3-({4-[(4,5- dimethyl-1H- pyrazol-3- yl)amino]-2- pyrimidinyl}amino)- N,N-diethyl-2- (methyloxy) benzene sulfonamide | 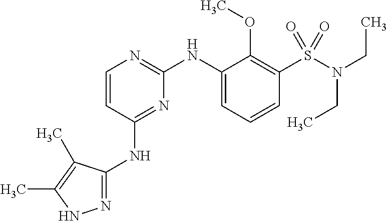 [SMILES: CCN(CC)S(=O)(=O)c1cccc(Nc2nccc(Nc3n[nH]c(C)c3C)n2)c1OC] | ¹H NMR (DMSO-d₆) δ 8.74 (br. s., 1H), 8.02 (d, J = 5.9 Hz, 1H), 7.40 (br. s., 1H), 7.18 (d, J = 8.5 Hz, 1H), 6.40 (d, J = 5.4 Hz, 1H), 3.95 (s, 3H), 3.13 (q, J = 6.8 Hz, 4H), 2.13 (s, 3H), 1.82 (br. s., 3H), 1.05 (t, 6H) | 446 | K |
| 120 | N²-[2,3-dimethyl- 5- (methylsulfonyl) phenyl]-N⁴-(4,5- dimethyl-1H- pyrazol-3-yl)-2,4- pyrimidinediamine | 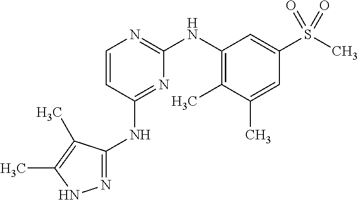 | ¹H NMR (DMSO-d₆) δ 12.05 (br. s., 1H), 7.75- 8.00 (m, 2H), 7.49 (br. s., 1H), 6.24 (br. s., 1H), 3.14 (s, 3H), 2.35 (s, 3H), 2.16 (br. s., 3H), 2.10 (s, 3H), 1.75 (br. s., 3H) | 387 | K |
| 121 | N4-(4,5-dimethyl- 1H-pyrazol-3-yl)- N4-methyl-N2-{3- [(1-methylethyl)- sulfonyl]phenyl}- 2,4- pyrimidinediamine | 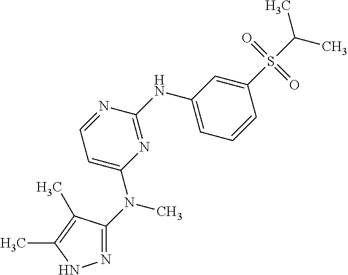 [SMILES: Cc1[nH]nc(N(C)c2ccnc(Nc3cccc(S(=O)(=O)C(C)C)c3)n2)c1C] | ¹H NMR (400 MHz, DMSO-d₆) δ 12.39 (s, 1H), 9.67 (s, 1H), 8.67 (br. s., 1H), 7.87-8.01 (m, 2H), 7.51 (t, J = 7.83 Hz, 1H), 7.34 (d, J = 8.08 Hz, 1H), 5.71 (br. s., 1H), 3.39 (s, 3H), 3.31-3.37 (m, 1H), 2.19 (s, 3H), 1.74 (s, 3H), 1.17 (d, J = 6.82 Hz, 6H) | 401 | G |
| 122 | N²-{3-[(1- methylethyl) sulfonyl]phenyl}- N⁴-(1,3,4- trimethyl-1H- pyrazol-5-yl)-2,4- pyrimidinediamine | 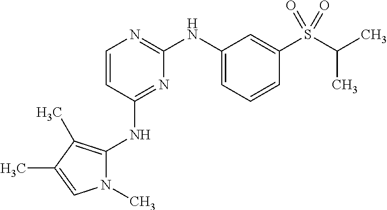 [SMILES: Cc1cn(C)c(Nc2ccnc(Nc3cccc(S(=O)(=O)C(C)C)c3)n2)c1C] | ¹H NMR (METHANOL-d₄) δ 8.13 (br. s., 1H), 8.05 (d, J = 5.8 Hz, 1H), 7.98 (d, J = 6.3 Hz, 1H), 7.32- 7.48 (m, 2H), 6.10 (d, J = 10.1 Hz, 1H), 3.61 (s, 3H), 3.25-3.31 (m, 1H), 2.20 (s, 3H), 1.87 (s, 3H), 1.26 (d, J = 6.8 Hz, 6H) | 401 | K |
| 123 | N4-(4,5-dimethyl- 1H-pyrazol-3-yl)- N2-[3- (methylsulfonyl)- 5-(1-pyrrolidinyl) phenyl]-2,4- pyrimidinediamine | 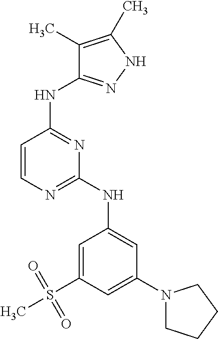 [SMILES: Cc1[nH]nc(Nc2ccnc(Nc3cc(N4CCCC4)cc(S(C)(=O)=O)c3)n2)c1C] | ¹H NMR (CHLOROFORM- d) δ 9.36 (br. s., 1H), 8.55 (br. s., 1H), 8.11 (d, J = 5.8 Hz, 1H), 7.71 (br. s., 1H), 7.40 (s, 1H), 6.64 (s, 1H), 6.13 (d, J = 5.8 Hz, 1H), 3.32-3.42 (m, 4H), 3.12 (s, 3H), 2.27 (s, 3H), 1.99- 2.11 (m, 4H), 1.91 (s, 3H) | 428 | K |
| 124 | N4-(4,5- DIMETHYL-1H- PYRAZOL-3-YL)- N3-METHYL-N2- [3-(METHYL- SULFONYL) PHENYL]-2,4- PYRIMIDINE- DIAMINE | 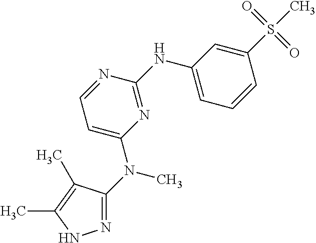 [SMILES: Cc1[nH]nc(N(C)c2ccnc(Nc3cccc(S(C)(=O)=O)c3)n2)c1C] | ¹H NMR (400 MHz, DMSO-d₆) δ 12.38 (s, 1H), 9.66 (s, 1H), 8.72 (br. s., 1H), 7.85-7.96 (m, 2H), 7.50 (t, J = 7.96 Hz, 1H), 7.42 (d, J = 7.58 Hz, 1H), 5.72 (br. s., 1H), 3.37 (s, 3H), 3.17 (s, 3H), 2.19 (s, 3H), 1.74 (s, 3H) | 373 | G |
| 125 | N²-{4-methyl-3- [(1-methylethyl) sulfonyl]phenyl}- N⁴-(1,3,4- trimethyl-1H- pyrazol-5-yl)-2,4- pyrimidinediamine trifluoroacetamide | 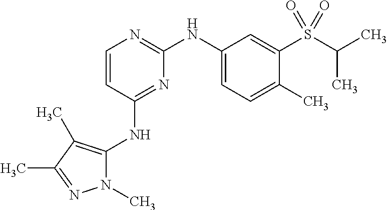 [SMILES: Cc1ccc(Nc2nccc(Nc3c(C)c(C)nn3C)n2)cc1S(=O)(=O)C(C)C] | ¹H NMR (DMSO-d₆) δ 10.17 (br. s., 1H), 9.84 (br. s., 1H), 8.03 (d, J = 6.3 Hz, 1H), 7.91 (br. s., 2H), 7.29 (br. s., 2H), 3.33 (m, 1H), 3.48 (s, 3H), 2.55 (s, 3H), 2.11 (s, 3H), 1.77 (s, 3H), 1.16 (d, J = 6.8 Hz, 6H) | 415 | K |
| 126 | 2-methyl-2-{[2- methyl-5-({4- [(1,3,4-trimethyl- 1H-pyrazol-5- yl)amino]-2- pyrimidinyl}amino) phenyl]sulfonyl}- 1-propanol trifluoroacetate | 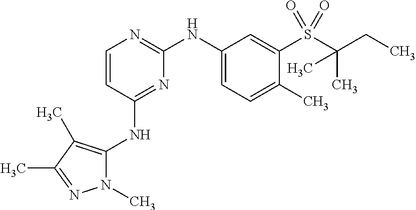 [SMILES: CCC(C)(C)S(=O)(=O)c1cc(Nc2nccc(Nc3c(C)c(C)nn3C)n2)ccc1C] | ¹H NMR (DMSO-d₆) δ 10.18 (br. s., 1H), 9.88 (br. s., 1H), 8.02 (d, J = 6.5 Hz, 1H), 7.85 (br. s., 2H), 7.25 (br. s., 1H), 4.21 (m, 2H), 3.52 (s, 3H), 2.57 (s, 3H), 2.11 (s, 3H), 1.77 (s, 3H), 1.21 (s, 6H) | 445 | K |
| 127 | N²-[3-methyl-5- (methylsulfonyl) phenyl]-N⁴- (1,3,4-trimethyl- 1H-pyrazol-5-yl)- 2,4- pyrimidinediamine trifluoroacetate | 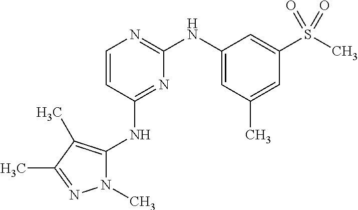 | ¹H NMR (DMSO-d₆) δ 10.31 (br. s., 1H), 9.98 (br. s., 1H), 8.08 (d, J = 6.5 Hz, 1H), 7.68-7.88 (m, 2H), 7.40 (br. s., 1H), 3.50 (s, 3H), 3.16 (s, 3H), 2.31 (br. s., 3H), 2.10 (s, 3H), 1.80 (s, 3H) | 387 | K |
| 128 | N4-(4,5-dimethyl- 1H-pyrazol-3-yl)- N2-[4-methyl-3- (methylsulfonyl)- 5-(1-pyrrolidinyl) phenyl]-2,4- pyrimidinediamine | 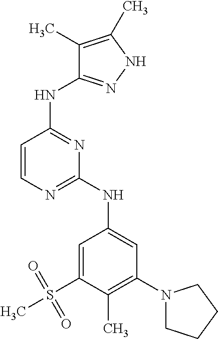 [SMILES: Cc1[nH]nc(Nc2ccnc(Nc3cc(N4CCCC4)c(C)c(S(C)(=O)=O)c3)n2)c1C] | ¹H NMR (METHANOL-d₄) δ 7.97 (br. s., 2H), 7.66 (br. s., 1H), 6.23 (br. s., 1H), 3.07-3.20 (m, 7H), 2.57 (s, 3H), 2.22 (s, 3H), 1.82-2.02 (m, 7H) | 442 | K |
| 129 | N2-[3- [(difluoromethyl) oxy]-5- (methylsulfonyl) phenyl]-N4-(4,5- dimethyl-1H- pyrazol-3-yl)-2,4- pyrimidinediamine | 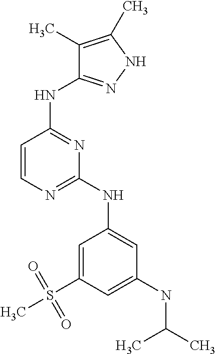 [SMILES: Cc1[nH]nc(Nc2ccnc(Nc3cc(NC(C)C)cc(S(C)(=O)=O)c3)n2)c1C] | ¹H NMR (DMSO-d₆) δ 12.04-12.42 (m, 1H), 9.66 (br. s., 1H), 8.93 (br. s., 1H), 8.00-8.16 (m, 3H), 7.11-7.55 (m, 2H), 6.27 (br. s., 1H), 3.22 (s, 3H), 2.16 (br. s., 3H), 1.80 (br. s., 3H) | 425 | K |
| 130 | 3-({4-[(4,5- dimethyl-1H- pyrazol-3- yl)amino]-2- pyrimidinyl}amino)- 5- (methylsulfonyl) phenol | 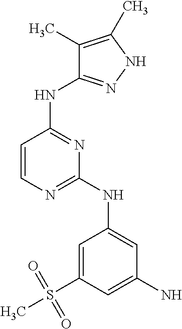 [SMILES: Cc1[nH]nc(Nc2ccnc(Nc3cc(N)cc(S(C)(=O)=O)c3)n2)c1C] | ¹H NMR (METHANOL-d₄) δ 8.00 (d, J = 4.5 Hz, 1H), 7.76 (s, 1H), 7.49-7.52 (m, 1H), 6.92 (t, J = 1.8 Hz, 1H), 6.26 (br. s., 1H), 3.09 (s, 3H), 2.24 (s, 3H), 1.93 (s, 3H) | | K |
| 131 | N²-[3-methyl-5- (methylsulfonyl) phenyl]-N⁴- (1,3,4-trimethyl- 1H-pyrazol-5-yl)- 2,4- pyrimidinediamine trifluoroacetate | 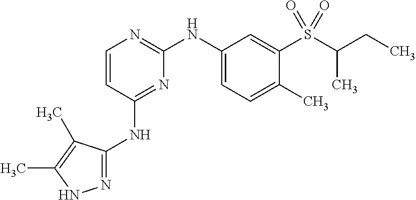 [SMILES: CCC(C)S(=O)(=O)c1cc(Nc2nccc(Nc3n[nH]c(C)c3C)n2)ccc1C] | ¹H NMR (DMSO-d₆) δ 12.07 (s, 1H), 9.39 (s, 1H), 8.74 (br. s., 1H), 8.21 (br. s., 1H), 8.09 (s, 1H), 7.86- 8.07 (m, 1H), 7.28 (br. s., 1H), 6.27 (br. s., 1H), 4.95 (m, 2H), 3.70 (br. s., 1H), 3.41-3.50 (m, 3H), 2.14 (br. s., 3H), 1.83 (br. s., 3H), 1.15-1.27 (m, 3H) | 417 | K |
| 132 | N⁴-(4,5-dimethyl- 1H-pyrazol-3-yl)- N²-[2-methyl-5- (methyloxy)-3- (methylsulfonyl) phenyl]-2,4- pyrimidinediamine trifluoroacetate | 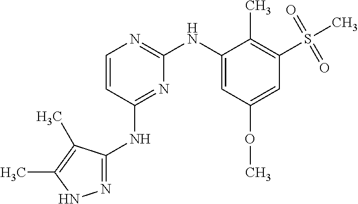 | ¹H NMR (METHANOL-d₄) δ 7.82 (d, J = 7.1 Hz, 1H), 7.62 (br. s., 1H), 7.33 (br. s., 1H), 6.48 (d, J = 7.3 Hz, 1H), 3.84 (d, J = 5.3 Hz, 3H), 3.17 (br. s., 3H), 2.53 (br. s., 3H), 2.22 (s, 3H), 1.82 (br. s., 3H) | 403 | K |
| 133 | N4-(4,5- DIMETHYL-1H- PYRAZOL-3-YL)- N4-METHYL-N2- [3-METHYL-5- (METHYL SULFONYL) PHENYL]-2,4- PYRIMIDINE- DIAMINE | 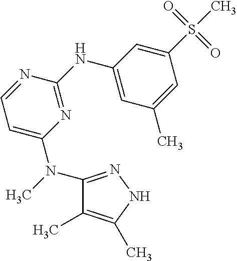 [SMILES: Cc1cc(Nc2nccc(N(C)c3n[nH]c(C)c3C)n2)cc(S(C)(=O)=O)c1] | ¹H NMR (400 MHz, DMSO-d₆) δ 12.38 (s, 1H), 9.59 (s, 1H), 8.50 (br. s., 1H), 7.91 (d, J = 5.81 Hz, 1H), 7.77 (s, 1H), 7.26 (s, 1H), 5.71 (br. s., 1H), 3.36 (s, 3H), 3.15 (s, 3H), 2.36 (s, 3H), 2.19 (s, 3H), 1.74 (s, 3H) | 387 | G |
| 134 | N2-[3- (CYCLOPENTYL SULFONYL) PHENYL]-N4- (4,5-DIMETHYL- 1H-PYRAZOL-3- YL)-N4- METHYL-2,4- PYRIMIDINE- DIAMINE | 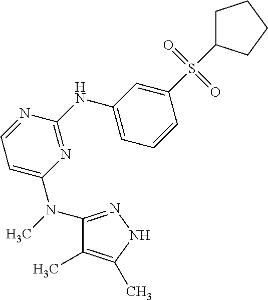 [SMILES: Cc1[nH]nc(N(C)c2ccnc(Nc3cccc(S(=O)(=O)C4CCCC4)c3)n2)c1C] | ¹H NMR (400 MHz, DMSO-d₆) δ 12.39 (s, 1H), 9.66 (s, 1H), 8.69 (br. s., 1H), 7.87-7.95 (m, 2H), 7.50 (t, J = 7.96 Hz, 1H), 7.37 (d, J = 8.08 Hz, 1H), 5.71 (br. s., 1H), 3.64- 3.76 (m, 1H), 3.35-3.39 (m, 3H), 2.19 (s, 3H), 1.78- 1.93 (m, 4H), 1.75 (s, 3H), 1.48-1.68 (m, 4H) | 427 | G |
| 135 | N⁴-[3,4-dimethyl- 1-(tetrahydro-2H- pyran-4-yl)-1H- pyrazol-5-yl]-N²- [3- (methylsulfonyl) phenyl]-2,4- pyrimidinediamine trifluoroacetate | 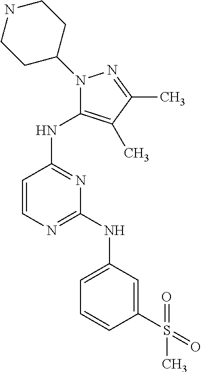 [SMILES: Cc1nn(C2CCNCC2)c(Nc2ccnc(Nc3cccc(S(C)(=O)=O)c3)n2)c1C] | ¹H NMR (DMSO-d₆) δ 10.09 ( br. s., 1H), 9.57 (br. s., 1H), 8.07 (d, J = 6.3 Hz, 3H), 7.57-7.69 (m, 1H), 7.51 (br. s., 1H), 5.75 (s, 1H), 3.87 (br. s., 1H), 3.34 (s, 4H), 3.16 (br. s., 3H), 2.13 (s, 3H), 1.90- 1.97 (m, 2H), 1.76 (s, 3H), 1.60-1.70 (m, 2H) | 443 | E |
| 136 | N⁴-{3,4-dimethyl- 1-[2- (methyloxy)ethyl]- 1H-pyrazol-5-yl}- N²-[3- (methylsulfonyl) phenyl]-2,4- pyrimidinediamine | 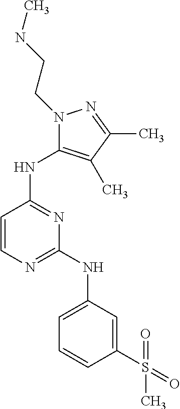 [SMILES: CNCCn1nc(C)c(C)c1Nc1ccnc(Nc2cccc(S(C)(=O)=O)c2)n1] | ¹H NMR (METHANOL-d₄) δ 8.01 (d, J = 7.1 Hz, 2H), 7.73-7.82 (m, 2H), 7.55- 7.62 (m, 1H), 6.51-6.55 (m, 1H), 4.09 (t, J = 5.1 Hz, 2H), 3.61 (s, 2H), 3.19 (s, 3H), 3.12 (br. s., 3H), 2.23 (s, 3H), 1.86 (s, 3H) | 417 | E |
| 137 | N~2~-[3- (CYCLOHEXYL SULFONYL) PHENYL]-N-~4~- (4,5-DIMETHYL- 1H-PYRAZOL-3- YL)-N~4~- METHYL-2,4- PYRIMIDINE- DIAMINE | 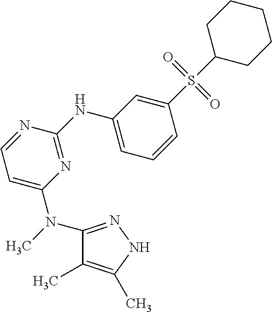 [SMILES: Cc1[nH]nc(N(C)c2ccnc(Nc3cccc(S(=O)(=O)C4CCCCC4)c3)n2)c1C] | ¹H NMR (400 MHz, DMSO-d₆) δ 12.39 (s, 1H), 9.66 (s, 1H), 8.64 (br. s., 1H), 7.91 (d, J = 6.32 Hz, 2H), 7.50 (t, J = 7.96 Hz, 1H), 7.32 (d, J = 8.08 Hz, 1H), 5.71 (br. s., 1H), 3.37 (s, 3H), 3.10 (br. m., 1H), 2.19 (s, 3H), 1.88- 1.96 (m, 2H), 1.70-1.79 (m, 5H), 1.58 (br. m., 1H), 1.16-1.34 (m, 4H), 1.08 (m, 1H) | 441 | G |
| 138 | N2-{3-[(1,1- dimethylethyl)sulfonyl] phenyl}-N4- (4,5-dimethyl-1H- pyrazol-3-yl)-N4- methyl-2,4- pyrimidinediamine | 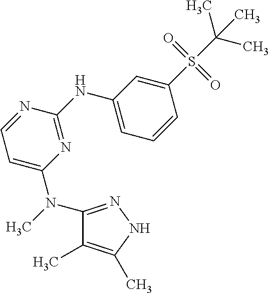 [SMILES: Cc1[nH]nc(N(C)c2ccnc(Nc3cccc(S(=O)(=O)C(C)(C)C)c3)n2)c1C] | ¹H NMR (400 MHz, DMSO-d₆) δ 12.38 (s, 1H), 9.55 (s, 1H), 8.63 (br. s., 1H), 7.94-8.01 (m, 1H), 7.90 (d, J = 5.81 Hz, 1H), 7.51 (t, J = 7.96 Hz, 1H), 7.31 (d, J = 7.83 Hz, 1H), 5.71 (br. s., 1H), 3.36 (s, 3H), 2.19 (s, 3H), 1.74 (s, 3H), 1.26 (s, 9H) | 415 | G |
| 139 | N²-{3-[(1,1- dimethylethyl) sulfonyl]-5- methylphenyl}- N⁴-(4,5-dimethyl- 1H-pyrazol-3yl)- N⁴-methyl-2,4- pyrimidinediamine | 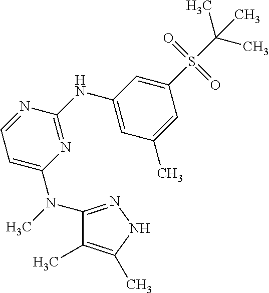 [SMILES: Cc1cc(Nc2nccc(N(C)c3n[nH]c(C)c3C)n2)cc(S(=O)(=O)C(C)(C)C)c1] | ¹H NMR (DMSO-d₆) δ 12.38 (s, 1H), 9.56 (s, 1H), 8.39 (br. s., 1H), 7.75- 7.98 (m, 2H), 7.14 (s, 1H), 5.70 (br. s., 1H), 3.36 (s, 3H), 2.37 (s, 3H), 2.19 (s, 3H), 1.74 (s, 3H), 1.26 (s, 9H) | 429 | K |
| 140 | N⁴-(4,5-dimethyl- 1H-pyrazol-3-yl)- N²-(4-methyl-3- {[(3S)-3-methyl- 4-morpholinyl] sulfonyl}phenyl)- 2,4- pyrimidinediamine | 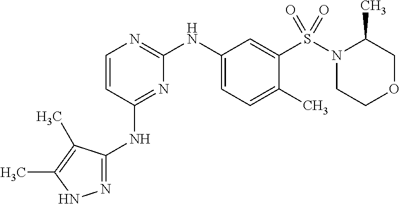 [SMILES: Cc1ccc(Nc2nccc(Nc3n[nH]c(C)c3C)n2)cc1S(=O)(=O)N1CCOC[C@@H]1C] | ¹H NMR (DMSO-d₆) δ 12.07 (br. s., 1H), 9.39 (br. s., 1H), 8.94 (br. s., 1H), 7.85-8.05 (m, 3H), 7.10 (s, 1H), 6.25 (br. s., 1H), 3.68-3.80 (m, 2H), 3.46- 3.55 (m, 1H), 3.41 (dd, J = 11.5, 2.7 Hz, 1H), 3.14- 3.23 (m, 2H), 2.31 (s, 3H), 2.14 (s, 3H), 1.82 (s, 3H), 1.06 (d, J = 6.8 Hz, 3H) | 458 | K |
| 141 | N⁴-(4,5-dimethyl- 1H-pyrazol-3-yl)- N²-(4-methyl-3- {[(3R)-3-methyl- 4-morpholinyl] sulfonyl}phenyl)- 2,4- pyrimidinediamine | 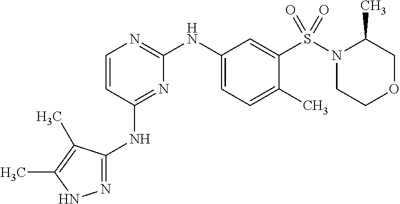 [SMILES: Cc1ccc(Nc2nccc(Nc3n[nH]c(C)c3C)n2)cc1S(=O)(=O)N1CCOC[C@@H]1C] | ¹H NMR (DMSO-d₆) δ 12.06 (br. s., 1H), 9.30 (br. s., 1H), 8.77 (br. s., 1H), 8.00 (br. s., 3H), 7.09 (br. s., 1H), 6.24 (br. s., 1H), 3.65-3.85 (m, 2H), 3.46- 3.54 (m, 1H), 3.39-3.45 (m, 1H), 3.21 (br. s., 2H), 2.31 (br. s., 3H), 2.14 (br. s., 3H), 1.81 (br. s., 3H), 1.06 (d, J = 6.8 Hz, 3H) | 458 | K |
| 142 | ethyl 3- (methylsulfonyl)- 5-({4-[(1,3,4- trimethyl-1H- pyrazol-5- yl)amino]-2- pyrimidinyl}amino) benzoate | 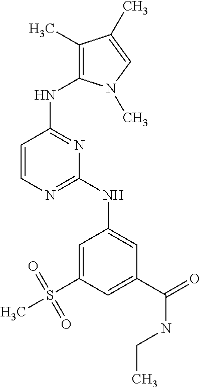 [SMILES: CCNC(=O)c1cc(Nc2nccc(Nc3c(C)c(C)cn3C)n2)cc(S(C)(=O)=O)c1] | ¹H NMR (DMSO-d₆) δ 9.76 (s, 1H), 8.99 (br. s., 1H), 8.67 (s, 1H), 8.59 (s, 1H), 8.11 (d, J = 5.6 Hz, 1H), 7.89 (s, 1H), 4.36 (q, J = 7.1 Hz, 2H), 3.53 (s, 3H), 3.21 (s, 3H), 2.08 (m, 3H), 1.78 (s, 3H), 1.35 (t, J = 7.2 Hz, 3H) | 445 | G |
| 143 | N²-[3,4-bis (methyloxy) phenyl]-N⁴-[3,4- dimethyl-1- (tetrahydro-2H- pyran-4- ylmethyl)-1H- pyrazol-5-yl]-2,4- pyrimidinediamine | 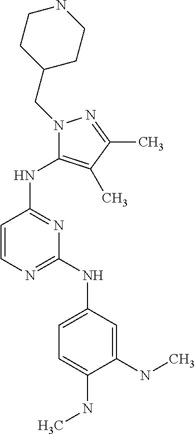 [SMILES: CNc1ccc(Nc2nccc(Nc3c(C)c(C)nn3CC3CCNCC3)n2)cc1NC] | ¹H NMR (CHLOROFORM- d) δ 7.96-8.05 (m, 1H), 7.18 (d, J = 2.3 Hz, 1H), 7.10-7.15 (m, 1H), 6.99- 7.07 (m, 1H), 6.82 (d, J = 8.8 Hz, 1H), 6.36-6.48 (m, 1H), 5.59-5.76 (m, 1H), 3.87-3.94 (m, 2H), 3.86 (s, 6H), 3.75-3.80 (m, 2H), 3.23-3.35 (m, 2H), 2.20 (s, 3H), 1.80- 1.84 (m, 3H), 1.40-1.50 (m, 2H), 1.28-1.32 (m, 3H) | 439 | E |
| 144 | N²-[3,4- bis(methyloxy) phenyl]-N⁴-[3,4- dimethyl-1- (tetrahydro-2H- pyran-4-yl)-1H- pyrazol-5-yl]-2,4- pyrimidinediamine | 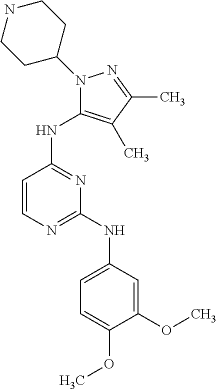 [SMILES: COc1ccc(Nc2nccc(Nc3c(C)c(C)nn3C3CCNCC3)n2)cc1OC] | ¹H NMR (DMSO-d₆) δ 8.90 (s, 1H), 8.61-8.79 (m, 1H), 7.97 (d, J = 5.6 Hz, 1H), 7.34- 7.47 (m, 1H), 7.04-7.26 (m, 1H), 6.66-6.83 (m, 1H), 4.15 (tt, J = 11.3, 4.2 Hz, 1H), 3.89 (dd, J = 11.2, 3.4 Hz, 2H), 3.65-3.70 (m, 3H), 3.59 (br. s., 2H), 3.33 (s, 3H), 2.11 (s, 3H), 1.88-2.04 (m, 2H), 1.74 (s, 3H), 1.69 (br. s., 2H) | 425 | E |
| 145 | N²-[2,5-dimethyl- 3- (methylsulfonyl) phenyl]-N⁴-(4,5- dimethyl-1H- pyrazol-3-yl)-2,4- pyrimidinediamine | 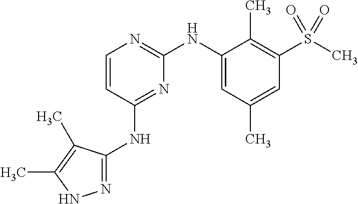 | ¹H NMR (METHANOL-d₄) δ 7.89 (br. s., 1H), 7.81 (d, J = 7.3 Hz, 1H), 7.56 (br. s., 1H), 6.46 (d, J = 7.1 Hz, 1H), 3.11-3.22 (m, 3H), 2.57 (br. s., 3H), 2.42 (br. s., 3H), 2.21 (br. s., 3H), 1.82 (br. s., 3H) | 387 | K |
| 146 | N⁴-[3,4-dimethyl- 1-(tetrahydro-2H- pyran-4-yl)-1H- pyrazol-5-yl]-N²- [3,4,5- tris(methyloxy) phenyl]-2,4- pyrimidinediamine trifluoroacetate | 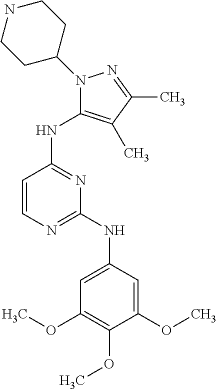 [SMILES: COc1cc(Nc2nccc(Nc3c(C)c(C)nn3C3CCNCC3)n2)cc(OC)c1OC] | ¹H NMR (DMSO-d₆) δ 10.28 (br. s., 2H), 7.96 (br. s., 1H), 6.63-6.99 (m, 3H), 6.39-6.56 (m, 1H), 4.14 (br. s., 1H), 3.88 (br. s., 2H), 3.53-3.71 (m, 9H), 3.26-3.43 (m, 2H), 2.10 (s, 3H), 1.87-2.02 (m, 2H), 1.73 (br. s., 3H), 1.64 (none, 2H) | 455 | E |
| 147 | N⁴-[3,4-dimethyl- 1-(tetrahydro-2H- pyran-4- ylmethyl)-1H- pyrazol-5-yl]-N²- [3,4,5- tris(methyloxy) phenyl]-2,4- pyrimidinediamine trifluoroacetate | 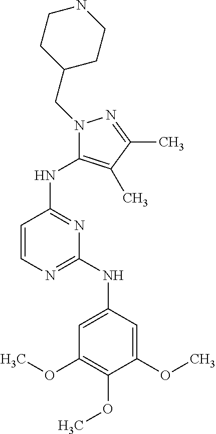 [SMILES: COc1cc(Nc2nccc(Nc3c(C)c(C)nn3CC3CCNCC3)n2)cc(OC)c1OC] | ¹H NMR (DMSO-d₆) δ 10.29 (br. s., 2H), 7.97 (br. s., lH), 6.60-6.96 (m, 3H), 6.39-6.56 (m, 1H), 3.68-3.82 (m, 7H), 3.63 (s, 6H), 3.14 (br. s., 2H), 2.08 (s, 3H), 1.96 (br. s., 1H), 1.74 (s, 3H), 1.34 (br. s., 2H), 1.04-1.20 (m, 2H) | 469 | E |
| 148 | N⁴-{3,4-dimethyl- 1-[2- (methyloxy)ethyl]- 1H-pyrazol-5-yl}- N²-[3,4,5- tris(methyloxy) phenyl]-2,4- pyrimidinediamine trifluoroacetate | 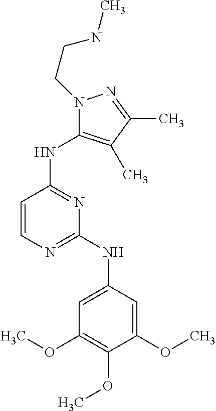 [SMILES: CNCCn1nc(C)c(C)c1Nc1ccnc(Nc2cc(OC)c(OC)c(OC)c2)n1] | ¹H NMR (DMSO-d₆) δ 9.95-10.45 (m, 2H), 7.85- 8.06 (m, 1H), 6.68-7.02 (m, 3H), 6.27-6.61 (m, 1H), 3.98 (br. s., 2H), 3.60- 3.70 (m, 9H), 3.55 (s, 2H), 3.08-3.16 (m, 3H), 2.09 (s, 3H), 1.74 (s, 3H) | 429 | E |
| 149 | (1S,2S)-2-{3,4- dimethyl-5-[(2- {[3,4,5- tris(methyloxy) phenyl]amino}-4- pyrimidinyl)amino]- 1H-pyrazol-1- yl}cyclohexanol trifluoroacetate | 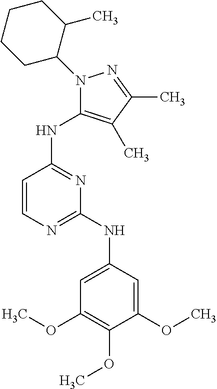 [SMILES: COc1cc(Nc2nccc(Nc3c(C)c(C)nn3C3CCCCC3C)n2)cc(OC)c1OC] | ¹H NMR (DMSO-d₆) δ 10.07-10.42 (m, 2H), 7.93 (br. s., 1H), 6.71 (br. s., 3H), 6.45-6.60 (m, 1H), 3.72-3.88 (m, 2H), 3.64 (br. s., 9H), 2.09 (br. s., 3H), 1.91 (s, 1H), 1.72 (br. s., 8H), 1.23 (br. s., 2H) | 469 | E |
| 150 | N⁴-(4,5-dimethyl- 1H-pyrazol-3-yl)- N²-[3-[(2- methyltetrahydro- 3- furanyl)sulfonyl]- 5-(trifluoromethyl) phenyl]-2,4- pyrimidinediamine | 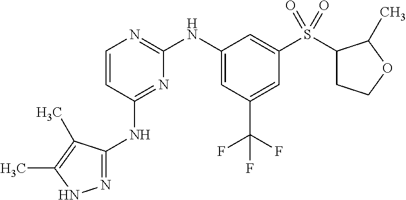 [SMILES: Cc1[nH]nc(Nc2ccnc(Nc3cc(C(F)(F)F)cc(S(=O)(=O)C4CCOC4C)c3)n2)c1C] | ¹H NMR (METHANOL-d₄) δ 8.52 (br. s., 1H), 8.41 (s, 1H), 8.05 (d, J = 5.8 Hz, 1H), 7.66 (s, 1H), 6.31 (d, J = 5.8 Hz, 1H), 4.20- 4.38 (m, 1H), 3.96-4.12 (m, 2H), 3.65 (q, J = 8.3 Hz, 1H), 2.24-2.35 (m, 1H), 2.23 (s, 3H), 2.11 (m, 1H), 1.90 (s, 3H), 1.59 (d, J = 6.6 Hz, 3H) | 497 | K |
| 151 | 3-({4-[(4,5- dimethyl-1H- pyrazol-3- yl)amino]-2- pyrimidinyl}amino) benzene sulfonamide hydrochloride | 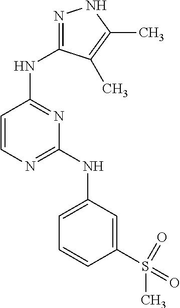 [SMILES: Cc1[nH]nc(Nc2ccnc(Nc3cccc(S(C)(=O)=O)c3)n2)c1C] | ¹H NMR (DMSO-d₆) δ 11.78-13.44 (m, 1H), 11.06 (br. s., 1H), 10.50- 10.81 (m, 1H), 8.10 (d, J = 6.8 Hz, 1H), 8.01 (d, J = 7.6 Hz, 2H), 7.32-7.63 (m, 4H), 6.54 (d, J = 6.3 Hz, 1H), 2.21 (s, 3H), 1.85 (br. s., 3H) | 360 | D |
| 152 | 5-({4-[(4,5- dimethyl-1H- pyrazol-3- yl)amino]-2- pyrimidinyl]amino)- 2- methylbenzene sulfonamide | 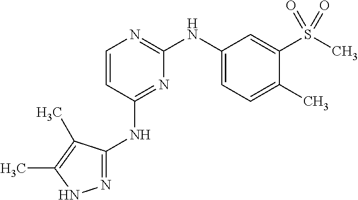 | ¹H NMR (DMSO-d₆) δ 12.08-12.39 (m, 1H), 9.62-9.89 (m, 1H), 8.11- 8.24 (m, 1H), 7.96-8.05 (m, 1H), 7.86-7.94 (m, 1H), 7.36 (s, 2H), 7.20- 7.27 (m, 1H), 6.29-6.39 (m, 1H), 3.26-3.44 (m, 3H), 2.16 (s, 3H), 1.83 (br. s., 3H) | 374 | G |
| 153 | 2-chloro-5-({4- [(4,5-dimethyl- 1H-pyrazol-3- yl)amino]-2- pyrimidinyl}amino) benzene sulfonamide hydrochloride | 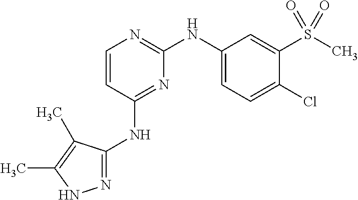 | ¹H NMR (DMSO-d₆) δ 10.98-11.09 (m, 1H), 10.58-10.72 (m, 1H), 8.06-8.18 (m, 3H), 7.62- 7.71 (m, 2H), 7.50-7.60 (m, 1H), 6.51-6.60 (m, 1H), 2.21 (s, 3H), 1.86 (s, 3H) | 394 | G |
| 154 | N²-[3,4- bis(methyloxy) phenyl]-N⁴-(3,4- dimethyl-5- isoxazolyl)-2,4- pyrimidinediamine trifluoroacetate | 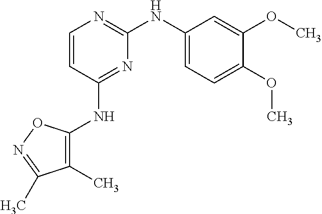 [SMILES: COc1ccc(Nc2nccc(Nc3onc(C)c3C)n2)cc1OC] | ¹H NMR (METHANOL-d₄) δ 7.85-7.94 (m, 1H), 6.92- 7.06 (m, 3H), 6.45 (d, J = 7.1 Hz, 1H), 3.87 (s, 3H), 3.79 (s, 3H), 2.23 (s, 3H), 1.81 (br. s., 3H) | 342 | B |
| 155 | N⁴-(3,4-dimethyl- 5-isoxazolyl)-N²- [3,4,5- tris(methyloxy) phenyl]-2,4- pyrimidinediamine trifluoroacetate | 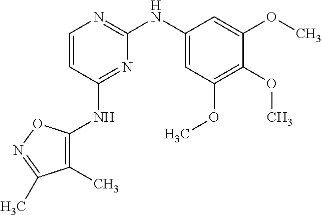 [SMILES: COc1cc(Nc2nccc(Nc3onc(C)c3C)n2)cc(OC)c1OC] | ¹H NMR (METHANOL-d₄) δ 7.90-8.00 (m, 1H), 6.78 (s, 2H), 6.44 (d, J = 7.1 Hz, 1H), 3.80 (s, 6H), 3.79 (s, 3H), 2.24 (s, 3H), 1.80 (s, 3H) | 372 | B |
| 156 | 3-({4-[(3,4- dimethyl-5- isoxazolyl)amino]- 2-pyrimidinyl} amino)-N,N- dimethylbenzene sulfonamide trifluoroacetate | 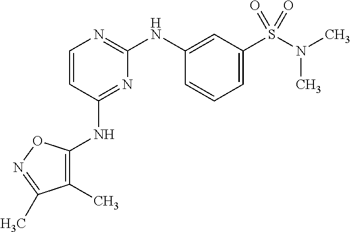 [SMILES: Cc1noc(Nc2ccnc(Nc3cccc(S(=O)(=O)N(C)C)c3)n2)c1C] | ¹H NMR (DMSO-d₆) δ 9.99 (br. s., 1H), 9.71 (br. s., 1H), 8.15 (d, J = 5.9 Hz, 2H), 8.01 (s, 1H), 7.46 (t, J = 8.1 Hz, 1H), 7.27 (d, J = 7.6 Hz, 1H), 6.30 (d, J = 5.9 Hz, 1H), 2.60 (s, 6H), 2.19 (s, 3H), 1.80 (s, 3H) | 389 | J |
| 157 | N⁴-(3,4-dimethyl- 5-isoxazolyl)-N²- [3-(1-pyrrolidinyl sulfonyl)phenyl]- 2,4- pyrimidinediamine trifluoroacetate | 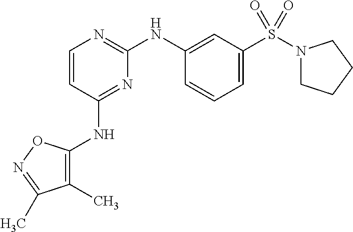 [SMILES: Cc1noc(Nc2ccnc(Nc3cccc(S(=O)(=O)N4CCCC4)c3)n2)c1C] | ¹H NMR (DMSO-d₆) δ 10.01 (br. s., 1H), 9.70 (br. s., 1H), 8.15 (d, J = 5.9 Hz, 2H), 8.04 (s, 1H), 7.44 (t, J = 7.9 Hz, 1H), 7.32 (d, J = 7.8 Hz, 1H), 6.30 (d, J = 5.9 Hz, 1H), 3.13 (t, J = 6.6 Hz, 4H), 2.19 (s, 3H), 1.80 (s, 3H), 1.65 (t, 4H) | 415 | J |
| 158 | N4-(3,4-dimethyl- 5-isoxazolyl)-N2- [2-methyl-5- (methylsulfonyl) phenyl]-2,4- pyrimidinediamine | 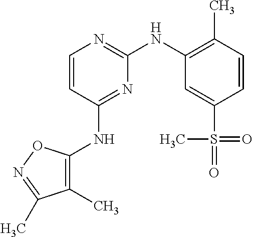 [SMILES: Cc1ccc(S(C)(=O)=O)cc1Nc1nccc(Nc2onc(C)c2C)n1] | ¹H NMR (METHANOL-d₄) δ 8.32 (d, J = 1.5 Hz, 1H), 8.08 (d, J = 5.8 Hz, 1H), 7.59 (dd, J = 8.1, 1.8 Hz, 1H), 7.47 (d, J = 7.8 Hz, 1H), 6.32 (d, J = 5.8 Hz, 1H), 3.10 (s, 3H), 2.39 (s, 3H), 2.22 (s, 3H), 1.81 (s, 3H) | 374 | D |
| 159 | 2-[3-({4-[(3,4- dimethyl-5- isoxazolyl)amino]- 2-pyrimidinyl}- amino)phenyl]-2- methylpropane- nitrile | 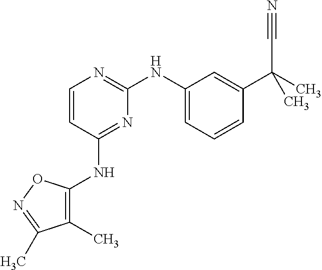 [SMILES: Cc1noc(Nc2ccnc(Nc3cccc(C(C)(C)C#N)c3)n2)c1C] | ¹H NMR (DMSO-d₆) δ 9.75 (br. s., 1H), 9.30 (s, 1H), 8.12 (d, J = 5.6 Hz, 1H), 7.70-7.87 (m, 2H), 7.24 (t, J = 8.1 Hz, 1H), 7.03 (d, J = 7.8 Hz, 1H), 6.22 (d, J = 5.6 Hz, 1H), 2.18 (s, 3H), 1.80 (s, 3H), 1.63 (s, 6H) | 349 | C |
| 160 | N4-(3,4- DIMETHYL-5- ISOXAZOLYL)- N4-METHYL-N2- [3-(METHYL SULFONYL) PHENYL]-2,4- PYRIMIDINE- DIAMINE | 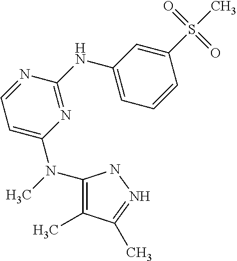 [SMILES: Cc1n[nH]c(N(C)c2ccnc(Nc3cccc(S(C)(=O)=O)c3)n2)c1C] | ¹H NMR (400 MHz, DMSO-d₆) δ 9.87 (s, 1H), 8.57 (s, 1H), 8.15 (d, J = 6.06 Hz, 1H), 7.90 (d, J = 8.59 Hz, 1H), 7.40-7.55 (m, 2H), 6.03 (d, J = 5.81 Hz, 1H), 3.41 (s, 3H), 3.18 (s, 3H), 2.24 (s, 3H), 1.79 (s, 3H); | 374 | G |
| 161 | N4-(3,4- DIMETHYL-5- ISOXAZOLYL)- N4-METHYL-N2- {3-[(1- METHYLETHYL) SULFONYL] PHENYL}-2,4- PYRIMIDINE- DIAMINE | 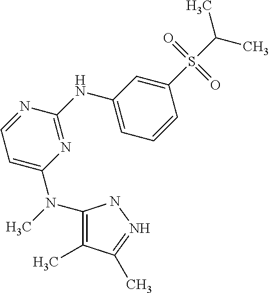 [SMILES: Cc1n[nH]c(N(C)c2ccnc(Nc3cccc(S(=O)(=O)C(C)C)c3)n2)c1C] | ¹H NMR (400 MHz, DMSO-d₆) δ 9.87 (s, 1H), 8.51 (s, 1H), 8.15 (d, J = 5.81 Hz, 1H), 7.94 (dd, J = 1.26, 8.08 Hz, 1H), 7.51 (t, J = 7.96 Hz, 1H), 7.37 (d, J = 8.34 Hz, 1H), 6.03 (d, J = 5.81 Hz, 1H), 3.41 (s, 3H), 3.27-3.38 (m, 1H), 2.24 (s, 3H), 1.79 (s, 3H), 1.17 (d, J = 6.82 Hz, 6H) | 402 | G |
| 162 | N4-(3,4- DIMETHYL-5- ISOXAZOLYL)- N2-{3-[(1- METHYLETHYL) SULFONYL] PHENYL}-2,4- PYRIMIDINE- DIAMINE | 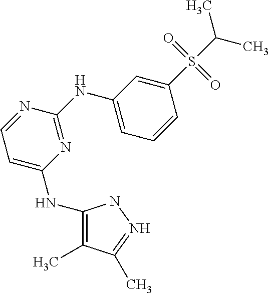 [SMILES: Cc1n[nH]c(Nc2ccnc(Nc3cccc(S(=O)(=O)C(C)C)c3)n2)c1C] | ¹H NMR (400 MHz, CHLOROFORM-d) δ 8.24 (d, J = 1.77 Hz, 1H), 8.21 (d, J = 5.81 Hz, 1H), 7.86 (dt, J = 1.89, 7.58 Hz, 1H), 7.47-7.58 (m, 2H), 7.45 (br. s., 1H), 6.97 (br. s., 1H), 6.43 (d, J = 5.81 Hz, 1H), 3.24 (quin, J = 6.82 Hz, 1H), 2.28 (s, 3H), 1.91 (s, 3H), 1.34 (d, J = 7.07 Hz, 6H) | 388 | G |
| 163 | N4-(3,4- DIMETHYL-5- ISOXAZOLYL)- N4-METHYL-N2- [3-METHYL-5- (METHYL SULFONYL) PHENYL]-2,4- PYRIMIDINE- DIAMINE | 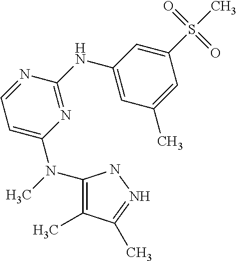 [SMILES: Cc1cc(Nc2nccc(N(C)c3[nH]nc(C)c3C)n2)cc(S(C)(=O)=O)c1] | ¹H NMR (400 MHz, DMSO-d₆) δ 9.79 (s, 1H), 8.34 (s, 1H), 8.15 (d, J = 5.81 Hz, 1H), 7.76 (s, 1H), 7.29 (s, 1H), 6.03 (d, J = 5.81 Hz, 1H), 3.40 (s, 3H), 3.15 (s, 3H), 2.36 (s, 3H), 2.23 (s, 3H), 1.79 (s, 3H) | 388 | G |
| 164 | N4-(3,4- DIMETHYL-5- ISOXAZOLYL)- N2-[3-METHYL- 5-(METHYL SULFONYL) PHENYL]-2,4- PYRIMIDINE- DIAMINE | 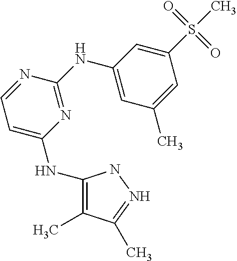 [SMILES: Cc1cc(Nc2nccc(Nc3[nH]nc(C)c3C)n2)cc(S(C)(=O)=O)c1] | ¹H NMR (400 MHz, DMSO-d₆) δ 9.60 (s, 1H), 8.17 (d, J = 5.56 Hz, 1H), 7.99 (m, 2H), 7.25 (s, 1H), 6.27 (d, J = 5.56 Hz, 1H), 3.13 (s, 3H), 2.34 (s, 3H), 2.19 (s, 3H), 1.81 (s, 3H) | 374 | G |
| 165 | N2-[3- (CYCLOPENTYL SULFONYL) PHENYL]-N4- (3,4-DIMETHYL- 5-ISOXAZOLYL)- N4-METHYL-2,4- PYRIMIDINE- DIAMINE | 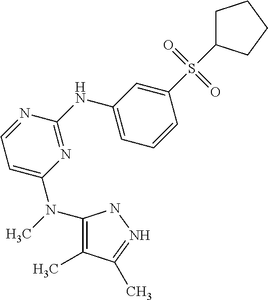 [SMILES: Cc1n[nH]c(N(C)c2ccnc(Nc3cccc(S(=O)(=O)C4CCCC4)c3)n2)c1C] | ¹H NMR (400 MHz, DMSO-d₆) δ 9.86 (s, 1H), 8.53 (s, 1H), 8.15 (d, J = 6.06 Hz, 1H), 7.92 (dd, J = 1.26, 8.34 Hz, 1H), 7.50 (t, J = 7.96 Hz, 1H), 7.40 (d, J = 8.34 Hz, 1H), 6.03 (d, J = 5.81 Hz, 1H), 3.65- 3.77 (m, 1H), 3.40 (s, 3H), 2.24 (s, 3H), 1.80-1.92 (m, 4H), 1.79 (s, 3H), 1.48- 1.67 (m, 4H) | 428 | G |
| 166 | N4-(3,4- DIMETHYL-5- ISOXAZOLYL)- N4-METHYL-N2- [3- (METHYLOXY)- 5-(METHYL- SULFONYL) PHENYL]-2,4- PYRIMIDINE- DIAMINE | 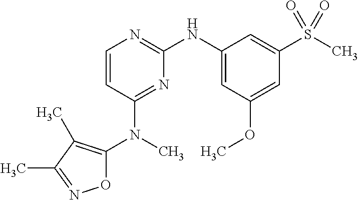 | ¹H NMR (500 MHz, DMSO-d₆) δ 9.71-10.03 (m, 1H), 8.15 (d, J =5.86 Hz, 1H), 8.06 (br. s., 1H), 7.56-7.71 (m, 1H), 6.94- 7.11 (m, 1H), 5.99-6.14 (m, 1H), 3.82 (s, 3H), 3.40 (s, 3H), 3.18 (s, 3H), 2.22 (s, 3H), 1.78 (s, 3H) | 404 | G |
| 167 | N4-(3,4- DIMETHYL-5- ISOXAZOLYL)- N2-[3- (METHYLOXY)- 5-(METHYL- SULFONYL) PHENYL]-2,4- PYRIMIDINE- DIAMINE | 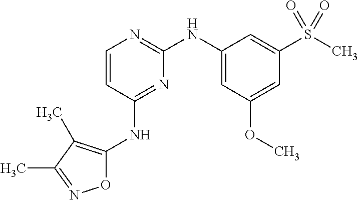 | ¹H NMR (400 MHz, DMSO-d₆) δ 10.12 (br. s., 1H), 9.82 (br. s., 1H), 8.17 (d, J = 6.02 Hz, 1H), 7.74 (br. s., 2H), 7.00 (s, 1H), 6.31 (d, J = 6.02 Hz, 1H), 3.79 (s, 3H), 3.18 (s, 3H), 2.19 (s, 3H), 1.79 (s, 3H) | 390 | H |
| 168 | N2-[3- (CYCLOPENTYL SULFONYL) PHENYL]-N4- (3,4-DIMETHYL- 5-ISOXAZOLYL)- 2,4- PYRIMIDINE- DIAMINE | 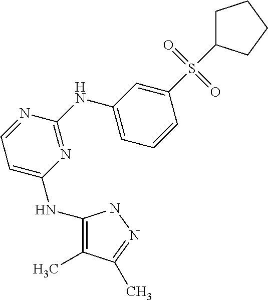 [SMILES: Cc1n[nH]c(Nc2ccnc(Nc3cccc(S(=O)(=O)C4CCCC4)c3)n2)c1C] | ¹H NMR (400 MHz, DMSO-d₆) δ 9.87 (s, 1H), 9.63 (s, 1H), 8.13-8.25 (m, 3H), 7.42-7.50 (m, 1H), 7.33-7.41 (m, 1H), 6.28 (d, J = 5.81 Hz, 1H), 3.60-3.73 (m, 1H), 2.19 (s, 3H), 1.72-1.92 (m, 7H), 1.46-1.67 (m, 4H) | 414 | G |
| 169 | N~2~-[3- (CYCLOHEXYL SULFONYL) PHENYL]-N~4~- (3,4-DIMETHYL- 5-ISOXAZOLYL)- N~4~-METHYL- 2,4- PYRIDIMINE- DIAMINE | 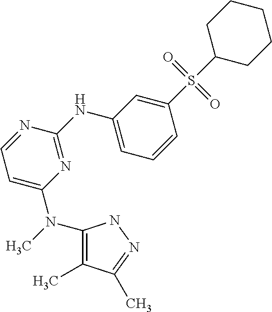 [SMILES: Cc1n[nH]c(N(C)c2ccnc(Nc3cccc(S(=O)(=O)C4CCCCC4)c3)n2)c1C] | ¹H NMR (400 MHz, DMSO-d₆) δ 9.85 (s, 1H), 8.48 (s, 1H), 8.15 (d, J = 5.81 Hz, 1H), 7.93 (d, J = 9.60 Hz, 1H), 7.50 (t, J = 8.08 Hz, 1H), 7.35 (d, J = 7.58 Hz, 1H), 6.02 (d, J = 5.81 Hz, 1H), 3.41 (s, 3H), 3.11 (br. m., 1H), 2.23 (s, 3H), 1.90 (m, 2H), 1.79 (s, 3H), 1.75 (m, 2H), 1.58 (br. m., 1H), 1.15-1.34 (m, 4H), 1.08 (m, 1) | 442 | G |
| 170 | N2-[3-[(1,1- dimethylethyl) sulfonyl]phenyl}- N4-(3,4-dimethyl- 5-isoxazolyl)-2,4- pyrimidinediamine | 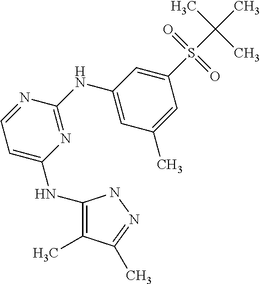 [SMILES: Cc1cc(Nc2nccc(Nc3[nH]nc(C)c3C)n2)cc(S(=O)(=O)C(C)(C)C)c1] | ¹H NMR (400 MHz, DMSO-d₆) δ 9.86 (s, 1H), 9.63 (s, 1H), 8.25 (dd, J = 1.26, 8.34 Hz, 1H), 8.10- 8.19 (m, 2H), 7.47 (t, J = 7.96 Hz, 1H), 7.31 (d, J = 8.34 Hz, 1H), 6.28 (d, J = 5.81 Hz, 1H), 2.20 (s, 3H), 1.82 (s, 3H), 1.23 (s, 9H) | 402 | G |
| 171 | N2-[3-[(1,1- dimethylethyl) sulfonyl]-5- (methylphenyl}- N4-(3,4-dimethyl- 5-isoxazolyl)-2,4- pyrimidinediamine | 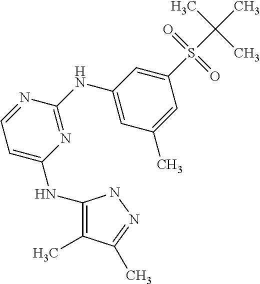 [SMILES: Cc1cc(Nc2nccc(Nc3[nH]nc(C)c3C)n2)cc(S(=O)(=O)C(C)(C)C)c1] | ¹H NMR (400 MHz, DMSO-d₆) δ 9.85 (s, 1H), 9.57 (s, 1H), 8.10-8.19 (m, 2H), 7.86 (s, 1H), 7.12 (s, 1H), 6.27 (d, J = 5.81 Hz, 1H), 2.34 (s, 3H), 2.19 (s, 3H), 1.81 (s, 3H), 1.23 (s, 9H) | 416 | G |
| 172 | N2-[3-[(1,1- dimethylethyl) sulfonyl]-phenyl}- N3-(3,4-dimethyl- 5-isoxazol)-N4- methyl-2,4- pyrimidinediamine | 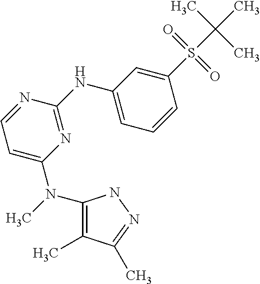 [SMILES: Cc1n[nH]c(N(C)c2ccnc(Nc3cccc(S(=O)(=O)C(C)(C)C)c3)n2)c1C] | ¹H NMR (400 MHz, DMSO-d₆) δ 9.85 (s, 1H), 8.46 (s, 1H), 8.14 (d, J = 5.81 Hz, 1H), 7.99 (dd, J = 1.26, 8.34 Hz, 1H), 7.51 (t, J = 7.96 Hz, 1H), 7.34 (d, J = 8.34 Hz, 1H), 6.03 (d, J = 5.81 Hz, 1H), 3.41 (s, 3H), 2.24 (s, 3H), 1.79 (s, 3H), 1.25 (s, 9H) | 416 | G |
| 173 | N2-[3-[(1,1- dimethylethyl) sulfonyl]-5- (methylphenyl}- N4-(3,4-dimethyl- 5-isoxazolyl)-N4- methyl-2,4- pyrimidinediamine | 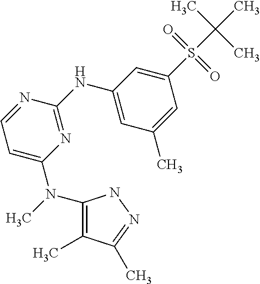 [SMILES: Cc1cc(Nc2nccc(N(C)c3[nH]nc(C)c3C)n2)cc(S(=O)(=O)C(C)(C)C)c1] | ¹H NMR (400 MHz, DMSO-d₆) δ 9.77 (s, 1H), 8.23 (s, 1H), 8.14 (d, J = 5.81 Hz, 1H), 7.85 (s, 1H), 7.16 (s, 1H), 6.03 (d, J = 5.81 Hz, 1H), 3.40 (s, 3H), 2.36 (s, 3H), 2.23 (s, 3H), 1.79 (s, 3H), 1.25 (s, 9H) | 430 | G |
| 174 | N2-[3-[(1,1- dimethylethyl) sulfonyl]-5- (trifluoromethyl) phenyl]-N4-(3,4- dimethyl-5- isoxazolyl)-2,4- pyrimidinediamine | 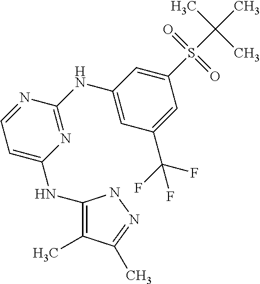 [SMILES: Cc1n[nH]c(Nc2ccnc(Nc3cc(C(F)(F)F)cc(S(=O)(=O)C(C)(C)C)c3)n2)c1C] | ¹H NMR (400 MHz, DMSO-d₆) δ 9.99 (s, 1H), 9.95 (s, 1H), 8.64 (s, 1H), 8.43 (s, 1H), 8.21 (d, J = 5.56 Hz, 1H), 7.47 (s, 1H), 6.34 (d, J = 5.56 Hz, 1H), 2.18 (s, 3H), 1.80 (s, 3H), 1.26 (s, 9H) | 470 | G |
| 175 | N4-(3,4- DIMETHYL-5- ISOXAZOLYL)- N2-[3-METHYL- 5- (TETRAHYDRO- 2H-PYRAN-4- YLSULFONYL) PHENYL]-2,4- PYRIMIDINE- DIAMINE | 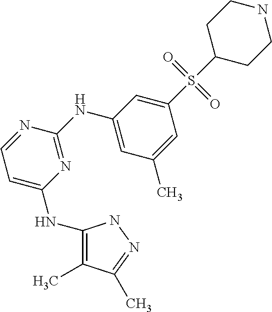 [SMILES: Cc1cc(Nc2nccc(Nc3[nH]nc(C)c3C)n2)cc(S(=O)(=O)C2CCNCC2)c1] | ¹H NMR (400 MHz, DMSO-d₆) δ 9.86 (s, 1H), 9.58 (s, 1H), 8.16 (d, J = 5.56 Hz, 1H), 8.09 (s, 1H), 7.89 (s, 1H), 7.16 (s, 1H), 6.28 (d, J = 5.56 Hz, 1H), 3.89 (dd, J = 3.66, 11.24 Hz, 2H), 3.23-3.35 (m, 3H), 2.34 (s, 3H), 2.19 (s, 3H), 1.81 (s, 3H), 1.72 (m, 2H), 1.46-1.60 (m, 2H) | 444 | G |
| 176 | N4-(3,4- DIMETHYL-5- ISOXAZOLYL)- N4-METHYL-N2- [3-METHYL-5- (TETRAHYDRO- 2H-PYRAN-4- YLSULFONYL) PHENYL]-2,4- PYRIMIDINE- DIAMINE | 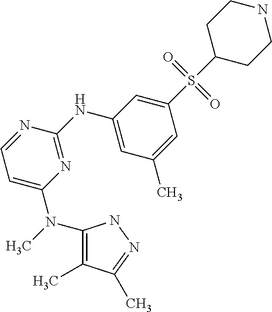 [SMILES: Cc1cc(Nc2nccc(N(C)c3[nH]nc(C)c3C)n2)cc(S(=O)(=O)C2CCNCC2)c1] | ¹H NMR (400 MHz, DMSO-d₆) δ 10.23 (br. s., 1H), 8.22 (br. s., 1H), 8.16 (d, J = 6.32 Hz, 1H), 7.77 (s, 1H), 7.29 (s, 1H), 6.12 (d, J = 6.32 Hz, 1H), 3.90 (dd, J = 3.66, 11.24 Hz, 2H), 3.45-3.53 (m, 1H), 3.42 (s, 3H), 3.23-3.33 (m, 2H), 2.38 (s, 3H), 2.23 (s, 3H), 1.80 (s, 3H), 1.70- 1.78 (m, 2H), 1.47-1.62 (m, 2H) | 458 | G |
| 177 | N4-(3,4- DIMETHYL-5- ISOXAZOLYL)- N2-[3- (METHYLOXY)- 5- (TETRAHYDRO- 2H-PYRAN-4- YLSULFONYL) PHENYL]-2,4- PYRIMIDINE- DIAMINE | 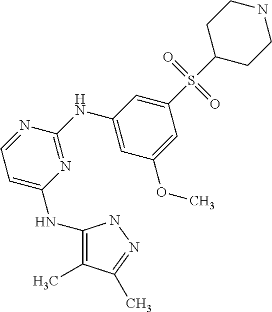 [SMILES: COc1cc(Nc2nccc(Nc3[nH]nc(C)c3C)n2)cc(S(=O)(=O)C2CCNCC2)c1] | ¹H NMR (400 MHz, DMSO-d₆) δ 9.86 (s, 1H), 9.62 (s, 1H), 8.17 (d, J = 5.56 Hz, 1H), 7.87 (t, J = 2.15 Hz, 1H), 7.72 (s, 1H), 6.84 (m, 1H), 6.28 (d, J = 5.81 Hz, 1H), 3.90 (m, 2H), 3.79 (s, 3H), 3.42- 3.52 (m, 1H), 3.28 (m, 2H), 2.19 (s, 3H), 1.80 (s, 3H), 1.72 (m, 2H), 1.48- 1.62 (m, 2H) | 460 | G |
| 178 | N4-(3,4-dimethyl- 5-isoxazolyl)-N4- methyl-N2-[3- (methyloxy)-5- (tetrahydro-2H- pyran-4- ylsulfonyl) phenyl]-2,4- pyrimidinediamine | 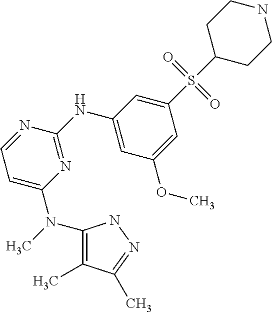 [SMILES: COc1cc(Nc2nccc(N(C)c3[nH]nc(C)c3C)n2)cc(S(=O)(=O)C2CCNCC2)c1] | ¹H NMR (400 MHz, DMSO-d₆) δ 9.82 (s, 1H), 8.15 (d, J = 5.81 Hz, 1H), 8.04 (s, 1H), 7.68 (t, J = 2.02 Hz, 1H), 6.88 (m, 1H), 6.04 (d, J = 5.81 Hz, 1H), 3.91 (m, 2H), 3.82 (s, 3H), 3.46-3.55 (m, 1H), 3.40 (s, 3H), 3.29 (m, 2H), 2.23 (s, 3H), 1.78 (s, 3H), 1.73 (m, 2H), 1.49-1.63 (m, 2H) | 474 | G |

### Pharmaceutical Compositions

### Example A

Tablets are prepared using conventional methods and are formulated as follows:

| Ingredient | Amount per tablet | |
|---|---|---|
| Compound of Example I | 5 | mg |
| Microcrystalline cellulose | 100 | mg |
| Lactose | 100 | mg |
| Sodium starch glycollate | 30 | mg |
| Magnesium stearate | 2 | mg |
| Total | 237 | mg |

### Example B

Capsules are prepared using conventional methods and are formulated as follows:

| Ingredient | Amount per tablet | |
|---|---|---|
| Compound of Example 3 | 15 | mg |
| Dried starch | 178 | mg |
| Magnesium stearate | 2 | mg |
| Total | 195 | mg |

### Biological In Vitro Assay:

A fluorescent polarization based binding assay was developed to quantitate interaction of novel test compounds at the ATP binding pocket of RIPK2, by competition with a fluorescently labeled ATP competitive ligand. Full length FLAG His tagged RIPK2 was purified from a Baculovirus expression system and was used at a final assay concentration of twice the KDapparent. A fluorescent labeled ligand (5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid, prepared as described below) was used at a final assay concentration of 5 nM. Both the enzyme and ligand were prepared in solutions in 50 mM HEPES pH7.5, 150 mM NaCl, 10 mM MgCl2, 1 mM DTT, and 1 mM CHAPS. Test compounds were prepared in 100% DMSO and 100 nL was dispensed to individual wells of a multiwell plate. Next, 5 ul RIPK2 was added to the test compounds at twice the final assay concentration, and incubated at room temperature for 10 minutes. Following the incubation, 5 ul of the fluorescent labeled ligand solution, was added to each reaction, at twice the final assay concentration, and incubated at room temperature for at least 10 minutes. Finally, samples were read on an instrument capable of measuring fluorescent polarization. Test compound inhibition was expressed as percent (%) inhibition of internal assay controls.

For concentration response experiments, normalized data were fit and pIC₅₀s determined using conventional techniques. For example, the following four parameter logistic equation may be used: y=A+((B−C))/(1+(10^{x})/(10^{C})^{D}), where: y is the % activity (% inhibition) at a specified compound concentration; A is the minimum % activity; B is the maximum % activity; C=log₁₀(IC₅₀); D=Hill slope; x=log₁₀ (compound concentration [M]); and pIC₅₀=(−C).

The pIC₅₀s are averaged to determine a mean value, for a minimum of 2 experiments. As determined using the above method, the compounds of Examples 1-178 exhibited a pIC₅₀ greater than or equal to 6.0. For instance, the compounds of Example 1 and Example 21 inhibited RIP2 kinase in the above method with a mean pIC₅₀ of 8.0 and 6.6 respectively.

### FLAG His Tagged RIPK2 Preparation:

Full-length human RIPK2 (receptor-interacting serine-threonine kinase 2) cDNA was purchased from Invitrogen (Carlsbad, Calif., USA, Clone ID:IOH6368, RIPK2-pENTR 221). Gateway® LR cloning was used to site-specifically recombine RIPK2 downstream to an N-terminal FLAG-6His contained within the destination vector pDEST8-FLAG-His6 according to the protocol described by Invitrogen. Transfection into Spodoptera frugiperda (Sf9) insect cells was performed using Cellfectin® (Invitrogen), according to the manufacturer's protocol.

Sf9 cells were grown in Excell 420 (SAFC Biosciences, Lenexa, Kans., US; Andover, Hampshire UK) growth media at 27° C., 80 rpm in shake flask until of a sufficient volume to inoculate a bioreactor. The cells were grown in a 50 litre working volume bioreactor (Applikon, Foster City, Calif., US; Schiedam, Netherlands) at 27° C., 30% dissolved oxygen and an agitation rate of 60-140 rpm until the required volume was achieved with a cell concentration of approximately 3.7×e6 cells/ml. The insect cells were infected with Baculovirus at a multiplicity of infection (MOI) of 12.7. The cultivation was continued for a 43 hour expression phase. The infected cells were removed from the growth media by centrifugation at 2500 g using a Viafuge (Carr) continuous centrifuge at a flow rate of 80 litres/hour. The cell pellet was immediately frozen and subsequently supplied for purification.

9.83×10¹⁰ Insect cells were re-suspended in 1.4 L lysis buffer (50 mM Tris (pH 8.0), 150 mM NaCl, 0.5 mM NaF, 0.1% Triton X-100, 1 mL/litre Protease Inhibitor Cocktail Set III (available from EMD Group; CalBiochem/Merck Biosciences, Gibbstown, N.J., US; Damstadt, Germany) and processed by dounce homogenization on ice. The suspension was then clarified by centrifugation at 47,900 g for 2 hours, at 4° C. The lysate was decanted from the insoluble pellet and loaded at a linear flow rate of 16 cm/h onto a 55 mL FLAG-M2 affinity column (2.6×10.4 cm) that had been pre-equilibrated with 10 column volumes buffer A (50 mM Tris (pH 8.0), 150 mM NaCl, 0.5 mM NaF, 1 mL/litre Protease Inhibitor Cocktail Set III). The column was then washed with 15 column volumes buffer A, and eluted with 6 column volumes buffer B (buffer A+150 μg/mL 3×FLAG peptide) at a linear flow rate of 57 cm/h. Fractions identified by SDS-PAGE as containing protein of interest were dialyzed to remove the 3× FLAG peptide from the preparation against 5 L of Buffer A (not containing the Protease Inhibitor Cocktail) overnight, using 10 kDa MWCO SnakeSkin Pleated Dialysis Tubing. The purification process yielded 11.3 mg of total protein, with the RIPK2 present at 40% purity by gel densitometry scanning, and identity confirmed by peptide mass fingerprinting. The main contaminating proteins in the preparation were identified as lower molecular weight degraded species of RIPK2.

### Fluorescent Ligand Preparation:

### 2-Methyl-5-(2-propen-1-yloxy)aniline

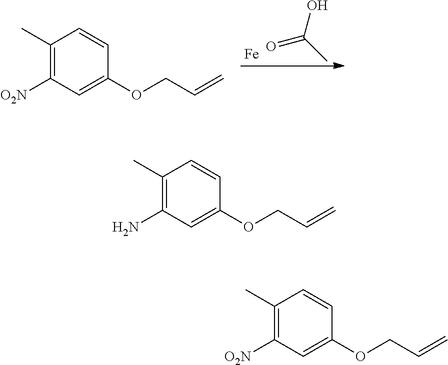

1-Methyl-2-nitro-4-(2-propen-1-yloxy)benzene (25.2 g, 130 mmol) was dissolved in ethanol (280 ml), water (28 ml), and acetic acid (5.6 ml, 98 mmol). Iron (29.1 g, 522 mmol) was added in six portions. The reaction was stirred for 72 hours, and then additional acetic acid (5.6 ml, 98 mmol) and 4 eq. of iron were added. The mixture was filtered through celite rinsing with EtOH and water and the filtrates were concentrated to remove EtOH. Diethylether (300 mL) was added along with 100 mL of 2 N HCl. The layers were separated and the ether layer was extracted with 2×100 mL of 2 N HCl. The acidic aqueous layer was slowly made pH 9 with NaOH pellets, and then dichloromethane (DCM, 300 mL) was added. The resulting emulsion was filtered using a Buchner funnel. The layers were separated and the aqueous layer extracted with DCM (2×100 mL). The combined extracts were dried over MgSO₄), filtered, and concentrated to a dark red oil (15.2 g). The crude material was purified via flash chromatography using a 120 g silica cartridge eluting with 5-15% EtOAc/hexanes for 30 min then 15-30% EtOAc/hexanes for 10 min. to give the titled compound as a red oil. MS (m/z) ¹H NMR (400 MHz, CHLOROFORM-d) δ ppm 2.23 (s, 3H) 4.51 (dt, J=5.29, 1.51 Hz, 2H) 5.29 (dd, J=10.45, 1.38 Hz, 1H) 5.38-5.46 (m, 1H) 5.99-6.12 (m, 1H) 6.01-6.10 (m, 1H) 6.46 (dd, J=8.31, 2.52 Hz, 1H) 6.56 (d, J=2.52 Hz, 1H) 7.01 (d, J=8.56 Hz, 1H); 164 (M+H+).

### 2-Chloro-N-[2-methyl-5-(2-propen-1-yloxy)phenyl]-4-pyrimidinamine

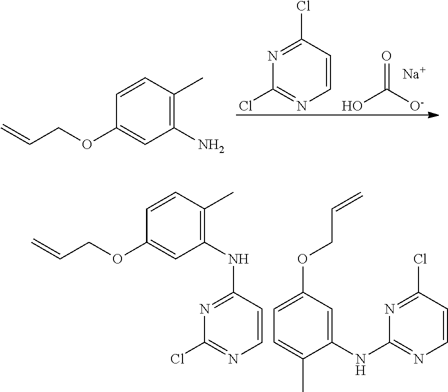

2-Methyl-5-(2-propen-1-yloxy)aniline (11.8 g, 72.3 mmol) was dissolved in tert-butanol (103 ml) and 2,4-dichloropyrimidine (10.77 g, 72.3 mmol) was added followed by sodium bicarbonate (18.22 g, 217 mmol). The reaction was heated at 80° C. for 17 hrs then additional 1,4-dichloropyrimidine (5.38 g, 36.6 mmol) was added and the reaction was stirred for 6 days. Additional 2,4-dichloropyrimidine (2.69 g, 17.8 mmol) was added and the reaction stirred for 2 days. The reaction was cooled to room temp diluting with EtOAc (200 mL) and water (200 mL). The layers were separated and the aqueous layer extracted with EtOAc (2×100 mL). The combined extracts were washed with brine (100 mL), dried over Na₂SO₄, filtered, and concentrated. The crude material was purified via flash chromatography using a 330 g silica cartridge eluting with 1-20% EtOAc/hexanes for 30 min then 20% EtOAc/hexanes for 50 min to give the titled compound (15.1 g). ¹H NMR (400 MHz, CHLOROFORM-d) δ ppm 2.20 (s, 3H) 4.54 (d, J=5.29 Hz, 2H) 5.32 (dd, J=10.45, 1.38 Hz, 1H) 5.42 (dd, J=17.37, 1.51 Hz, 1H) 5.99-6.12 (m, 1H) 6.35 (d, J=5.79 Hz, 1H) 6.83 (dd, J=8.44, 2.64 Hz, 1H) 6.89 (d, J=2.52 Hz, 6H) 7.14 (br. s., 6H) 7.21 (d, J=8.56 Hz, 7H) 8.10 (d, J=5.79 Hz, 6H); MS (m/z) 276 (M+H+).

### 3-[(4-{([2-Methyl-5-(2-propen-1-yloxy)phenyl]amino}-2-pyrimidinyl)amino]benzoic acid

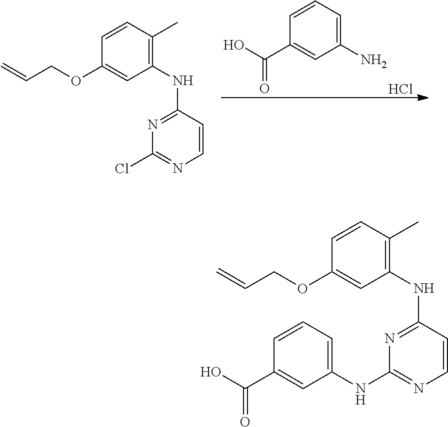 [SMILES: C=CCOc1ccc(C)c(Nc2ccnc(Cl)n2)c1 ; Nc1cccc(C(=O)O)c1 ; Cl ; C=CCOc1ccc(C)c(Nc2ccnc(Nc3cccc(C(=O)O)c3)n2)c1]

2-Chloro-N-[2-methyl-5-(2-propen-1-yloxy)phenyl]-4-pyrimidinamine (8 g, 29.0 mmol), 3-aminobenzoic acid (3.98 g, 29.0 mmol), and HCl (14.51 ml, 29.0 mmol) were dissolved in acetone (58.0 ml) and water (58.0 ml). The reaction was heated to 60° C. for 48 hrs. The reaction was cooled to room temperature passing air over it and a solid crashed out. Water (150 mL) was added and the solid was filtered washing with 3×50 mL water. The solid was dried in the vacuum funnel overnight affording the desired compound (10.9 g). ¹H NMR (400 MHz, METHANOL-d₄) □ ppm 2.21 (s, 3H) 4.47 (d, J=5.04 Hz, 2H) 5.24 (dd, J=10.58, 1.51 Hz, 1H) 5.37 (dd, J=17.25, 1.64 Hz, 1H) 5.97-6.09 (m, 4H) 6.29-6.39 (m, 1H) 6.89 (dd, J=8.44, 2.64 Hz, 4H) 6.96 (d, J=2.77 Hz, 1H) 7.04 (none, 0H) 7.23 (d, J=8.56 Hz, 1H) 7.34-7.41 (m, 1H) 7.75-7.79 (m, 1H) 7.81 (s, 1H) 7.85 (d, J=7.30 Hz, 3H) 7.98-8.09 (m, 3H); MS (m/z) 377 (M+H+).

### 1,1-Dimethylethyl {2-[({3-[(4-{[2-methyl-5-(2-propen-1-yloxy)phenyl]amino}-2-pyrimidinyl)amino]phenyl}carbonyl)amino]ethyl}carbamate

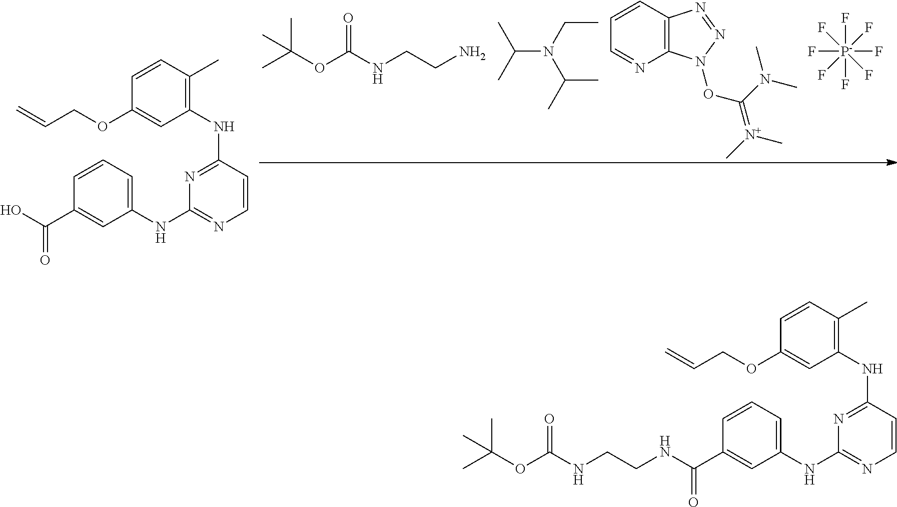

A solution of 3-[(4-{[2-methyl-5-(2-propen-1-yloxy)phenyl]amino}-2-pyrimidinyl)amino]benzoic acid (6.83 g, 18.15 mmol) and DIEA (9.51 ml, 54.4 mmol) in N,N-Dimethylformamide (DMF) (51.8 ml). was treated with N-(2-aminoethyl) carbamic acid tert-butyl ester (3.20 g, 19.96 mmol) and HATU (8.28 g, 21.77 mmol). EtOAc/Et₂O (400 mL, 1:1) was added and the layers separated. The organic layer was washed with water (3×300 mL) and brine (100 mL), dried over Na₂SO₄, filtered, and concentrated to give the titled compound (8.3 g). ¹H NMR (400 MHz, DMSO-d₆) 5 ppm 1.38 (s, 9H) 2.15 (s, 3H) 3.09 (q, J=6.19 Hz, 2H) 3.27 (q, J=6.19 Hz, 2H) 4.51 (d, J=5.27 Hz, 2H) 5.24 (dd, J=10.54, 1.51 Hz, 1H) 5.37 (dd, J=17.32, 1.76 Hz, 1H) 6.02 (m, J=17.29, 10.51, 5.18, 5.18 Hz, 1H) 6.13 (d, J=5.77 Hz, 1H) 6.73 (dd, J=8.41, 2.63 Hz, 1H) 6.90 (t, J=5.65 Hz, 1H) 7.09 (d, J=2.51 Hz, 1H) 7.15 (d, J=8.28 Hz, 1H) 7.17-7.22 (m, 1H) 7.28 (d, J=7.78 Hz, 1H) 7.94-7.99 (m, 2H) 7.99-8.05 (m, 2H) 8.26 (t, J=5.65 Hz, 1H) 8.66 (s, 1H) 9.17 (s, 1H); MS (m/z) 519 (M+H+).

### 1,1-Dimethylethyl [2-({[3-({4-[(5-hydroxy-2-methylphenyl)amino]-2-pyrimidinyl}amino)phenyl]carbonyl}amino)ethyl]carbamate

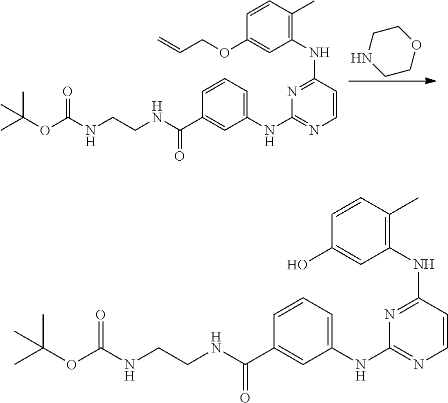 [SMILES: C=CCOc1ccc(C)c(Nc2ccnc(Nc3cccc(C(=O)NCCNC(=O)OC(C)(C)C)c3)n2)c1 ; C1COCCN1 ; Cc1ccc(O)cc1Nc1ccnc(Nc2cccc(C(=O)NCCNC(=O)OC(C)(C)C)c2)n1]

1,1-Dimethylethyl {2-[({3-[(4-{[2-methyl-5-(2-propen-1-yloxy)phenyl]amino}-2-pyrimidinyl)amino]phenyl}carbonyl)amino]ethyl}carbamate (5.5 g, 10.61 mmol) and morpholine (1.016 ml, 11.67 mmol) were dissolved in N,N-dimethylformamide (DMF) (42.4 ml) The atmosphere was exchanged for nitrogen and then it was treated with Tetrakis (1.226 g, 1.061 mmol). The reaction was heated to 80° C. for 3 hrs. The reaction was diluted with EtOAc (250 mL) and washed with water (3×200 mL) then brine (100 mL). The organic layer was dried over Na2SO4, filtered, and concentrated to about 50 mL and let stand overnight. A solid formed and to the suspension was added 50 mL ether. The solid was filtered washing with ether to give the desired product as an orange solid (4.75 g). ¹H NMR (400 MHz, METHANOL-d₄) δ ppm 1.42 (s, 9H) 2.17 (s, 3H) 3.29 (t, J=6.04 Hz, 2H) 3.46 (t, J=6.17 Hz, 2H) 6.04 (d, J=6.04 Hz, 1H) 6.65 (dd, J=8.31, 2.52 Hz, 1H) 6.87 (d, J=2.52 Hz, 1H) 7.09 (d, J=8.31 Hz, 1H) 7.27-7.33 (m, 1H) 7.35-7.41 (m, 1H) 7.53-7.61 (m, 1H) 7.62-7.70 (m, 2H) 7.75 (d, J=8.06 Hz, 1H) 7.91 (d, J=6.04 Hz, 1H) 8.11 (s, 1H); MS (m/z) 479 (M+H+).

### N-(2-Aminoethyl)-3-({4-[(5-hydroxy-2-methylphenyl)amino]-2-pyrimidinyl}amino)benzamide

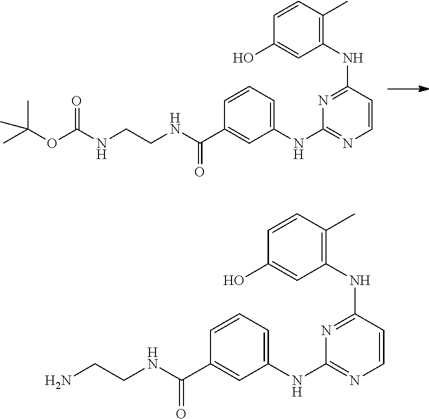 [SMILES: Cc1ccc(O)cc1Nc1ccnc(Nc2cccc(C(=O)NCCNC(=O)OC(C)(C)C)c2)n1 ; Cc1ccc(O)cc1Nc1ccnc(Nc2cccc(C(=O)NCCN)c2)n1]

1,1-Dimethylethyl [2-({[3-({4-[(5-hydroxy-2-methylphenyl)amino]-2-pyrimidinyl}amino)phenyl]carbonyl}amino)ethyl]carbamate (4.75 g, 8.93 mmol) (contaminated with tetrakis or related entities) was dissolved in dichloromethane (DCM) (28.6 ml) and trifluoroacetic acid (TFA) (7.15 ml). The reaction concentrated to give the desired product as the TFA salt containing the same impurities going into the reaction (6.5 g) MS (m/z) 379 (M+H+).

### 5-({[2-({[3-({4-[(5-Hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid

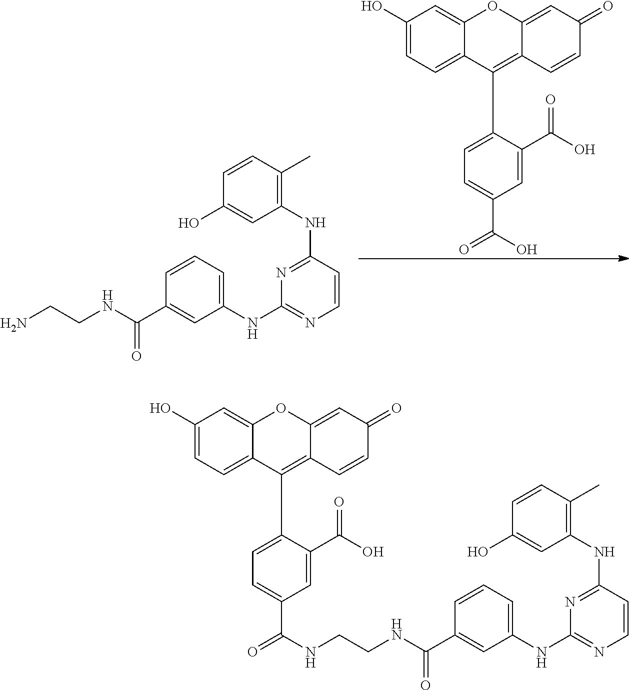 [SMILES: Cc1ccc(O)cc1Nc1ccnc(Nc2cccc(C(=O)NCCN)c2)n1 ; O=C(O)c1ccc(-c2c3ccc(=O)cc-3oc3cc(O)ccc23)c(C(=O)O)c1 ; Cc1ccc(O)cc1Nc1ccnc(Nc2cccc(C(=O)NCCNC(=O)c3ccc(-c4c5ccc(=O)cc-5oc5cc(O)ccc45)c(C(=O)O)c3)c2)n1]

To a suspension of N-(2-aminoethyl)-3-({4-[(5-hydroxy-2-methylphenyl)amino]-2-pyrimidinyl}amino)benzamide (1 g, 1.319 mmol) in N,N-dimethylformamide (DMF) (13.19 ml) was added 5-FAM (5-carboxyfluorescein single isomer) (0.397 g, 1.055 mmol), triethylamine (0.919 ml, 6.60 mmol), EDC (0.506 g, 2.64 mmol), and HOBT (0.202 g, 1.319 mmol). The reaction was stirred overnight then the pH was adjusted to 3 with 2 N HCl. The solution was extracted with EtOAc (100 mL) and the organic layer washed with water (1×50 mL), dried over Na₂SO₄, filtered, and concentrated to give the titled compound. MS (m/z) 737 (M+H+).

### Biological In Vivo Assay

The efficacy of the RIP2 inhibitors of this invention may also be evaluated in vivo in rodents. Intraperitoneal (i.p.) or intravenous (i.v.) administration of L18-MDP in mice has been shown to induce an inflammatory response through activation of the NOD2 signaling pathway (Rosenweig, H. L., et al. 2008. Journal of Leukocyte Biology 84:529-536). The level of the inflammatory response in the L18-MDP treated mice/rats is monitored using conventional techniques by measuring increases in cytokine levels (IL8, TNFα, IL6 and IL-1β) in serum and/or peritoneal lavage fluid and by measuring neutrophil influx into the peritoneal space (when L18-MDP is dosed i.p.). Inhibition of the L18-MDP induced inflammatory response in treated rodents may be shown by orally pre-dosing with selected compounds of this invention, then measuring and comparing cytokine levels (IL8, TNFα, IL6 and IL-1β) in serum and/or peritoneal lavage fluid and neutrophil influx into the peritoneal space (when L18-MDP is dosed i.p.) using conventional techniques.

1-30. (canceled)

31. A compound according to Formula (I): 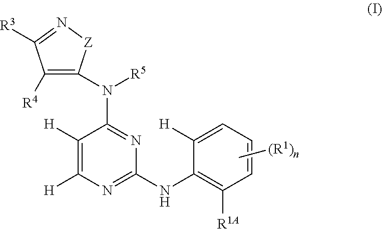
wherein:
R^{1A} is H, methyl or methoxy;
n is 1, 2 or 3;
each R¹ is independently selected from halogen, hydroxy, (C₁-C₆)alkyl, cyano, cyano(C₁-C₆)alkyl-, halo(C₁-C₆)alkyl, (C₁-C₄ alkyl)(C₁-C₄ alkyl)amino-halo(C₂-C₆)alkyl, OR^{x}, —SR^{x}, —SO₂R^{x}, —NR^{z}SO₂R^{x}, —COOR^{x}, —CONR^{y}R^{z}, —SO₂NR^{y}R^{z}, —SO₂-heterocycloalkyl, heterocycloalkyl, oxazolyl or benzoxazolyl,
and wherein any of said heterocycloalkyl (that is, the heterocycloalkyl group and the heterocycloalkyl moiety of the —SO₂heterocycloalkyl and —NH-heterocycloalkyl groups) is a 4-7 membered non-aromatic ring containing one heteroatom selected from N, O and S, or containing one nitrogen atom and one additional heteroatom selected from N, O and S; which heterocycloalkyl is optionally substituted by 1-5 substituents independently selected from hydroxy, halogen, (C₁-C₆)alkyl, halo(C₁-C₄)alkyl, —CO(C₁-C₆)alkyl, —SO₂(C₁-C₆)alkyl, amino(C₁-C₄)alkyl-, (C₁-C₄ alkyl)amino(C₁-C₄)alkyl-, (C₁-C₄ alkyl)(C₁-C₄ alkyl)amino(C₁-C₄)alkyl- and oxo,
R^{x} is selected from (C₁-C₆)alkyl, halo(C₁-C₆)alkyl, (C₃-C₇)cycloalkyl, hydroxy(C₂-C₆)alkyl-, (C₁-C₆)alkoxyCO(C₁-C₆)alkyl-, amino(C₂-C₆)alkyl-, ((C₁-C₄)alkyl)amino(C₂-C₆)alkyl-, and ((C₁-C₄)alkyl)((C₁-C₄)alkyl)amino(C₂-C₆)alkyl-,
R^{y} is selected from H, (C₁-C₆)alkyl, (C₃-C₇)cycloalkyl, hydroxy(C₂-C₆)alkyl-, amino(C₂-C₆)alkyl-, ((C₁-C₄)alkyl)amino(C₂-C₆)alkyl-, and ((C₁-C₄)alkyl)((C₁-C₄)alkyl)amino(C₂-C₆)alkyl-, and
R^{z} is H or (C₁-C₆)alkyl;
or one of R^{1A}, taken together with an adjacent R¹ group and the carbon atoms connecting the R^{1A} and R¹ groups form a 5-6 membered, aromatic or non-aromatic heterocyclic ring containing 1 or 2 heteroatom ring moieties independently selected from —NH—, —O—, —S— and —SO₂—, or two adjacent R¹ groups taken together with the carbon atoms connecting the two groups form a 5-6 membered, aromatic or non-aromatic heterocyclic ring containing 1 or 2 heteroatom ring moieties independently selected from —NH—, —O—, —S— and —SO₂—,
Z is O or NR²;
R² is H, (C₁-C₄)alkyl, (C₃-C₆)cycloalkyl, hydroxy(C₂-C₄)alkyl-, (C₁-C₆)alkoxy(C₂-C₄)alkyl-, amino(C₂-C₄)alkyl-, (C₁-C₆)alkylamino(C₂-C₄)alkyl-, ((C₁-C₆)alkyl)((C₁-C₆)alkyl)amino(C₂-C₄)alkyl-, —CO₂H, —CO₂(C₁-C₆)alkyl, —CONH₂, —CONH(C₁-C₆)alkyl, —CON((C₁-C₆)alkyl)((C₁-C₆)alkyl), —(C₁-C₄)alkylCO₂H, —(C₁-C₄)alkylCO₂(C₁-C₆)alkyl, —(C₁-C₄)alkylCONH₂, —(C₁-C₄)alkylCONH(C₁-C₆)alkyl, —(C₁-C₄)alkylCON((C₁-C₆)alkyl)((C₁-C₆)alkyl), phenyl, 5-6 membered heterocycloalkyl, 5-6 membered heterocycloalkyl-(C₁-C₄)alkyl-, or 5-6 membered heteroaryl, where said (C₃-C₆)cycloalkyl, phenyl, 5-6 membered heterocycloalkyl, 5-6 membered heterocycloalkyl-(C₁-C₄)alkyl-, or 5-6 membered heteroaryl is optionally substituted by 1-3 substituents independently selected from halogen, hydroxy, (C₁-C₆)alkyl, (C₁-C₄)haloalkyl, (C₁-C₆)alkoxy, (C₁-C₄)haloalkoxy, —CO₂H, —CO₂(C₁-C₄)alkyl and —CO₂(C₁-C₄)alkylphenyl;
R³ is selected from H, methyl, trifluoromethyl and phenyl;
R⁴ is selected from H and methyl; or
R³ and R⁴ taken together with the atoms through which they are attached form, a 5-6 membered non-aromatic carbocyclic ring; and
R⁵ is H or (C₁-C₄)alkyl; or
R⁴ and R⁵ taken together with the atoms through which they are attached form a 5-6 membered, unsubstituted non-aromatic heterocyclic ring;
wherein at least one of R², R³, and R⁴ is not H;
provided that the compound is not:
N²-(3-{[2-(diethylamino)ethyl]oxy}phenyl)-N⁴-(1,3-dimethyl-1H-pyrazol-5-yl)-2,4-pyrimidinediamine;
N²-[3-{[2-(diethylamino)ethyl]oxy}-4-(methyloxy)phenyl]-N-(1-ethyl-1H-pyrazol-5-yl)-2,4-pyrimidinediamine; or
N²-[3-{[2-(diethylamino)ethyl]oxy}-4-(methyloxy)phenyl]-N⁴-(3-methyl-1-phenyl-1H-pyrazol-5-yl)-2,4-pyrimidinediamine;
or a salt thereof.

32. A compound according to Formula (I-B): 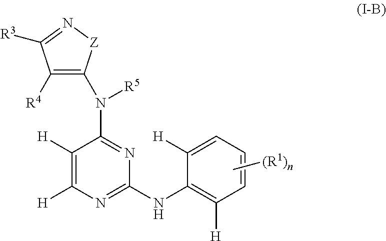
wherein:
n is 1, 2 or 3;
R¹ is halogen, (C₁-C₆)haloalkoxy, —OR^{x}—SO₂R^{x}, —SO₂NR^{y}R^{z} or heterocycloalkyl,
wherein said heterocycloalkyl is a 5-6 membered non-aromatic ring containing one heteroatom selected from N, O and S, or containing one nitrogen atom and optionally containing 1 additional heteroatom selected from N, O and S; which is optionally substituted by 1-5 substituents independently selected from (C₁-C₆)alkyl, (C₁-C₄)haloalkyl, —CO(C₁-C₆)alkyl, aminoC₁-C₄ alkyl-, (C₁-C₄ alkyl)aminoC₁-C₄ alkyl-, (C₁-C₄ alkyl)(C₁-C₄ alkyl)aminoC₁-C₄ alkyl-, and oxo; and
wherein R^{x} and R^{y} are selected from H, (C₁-C₆ alkyl), (C₃-C₇)cycloalkyl, amino(C₂-C₆ alkyl)-, (C₁-C₄ alkyl)amino(C₂-C₆ alkyl)-, and (C₁-C₄ alkyl)(C₁-C₄ alkyl)amino(C₂-C₆ alkyl)-, and
R^{z} is H or (C₁-C₆)alkyl, or
R^{y} and R^{z} taken together with the nitrogen atom to which they are attached form a 4-7 membered non-aromatic heterocyclic ring optionally containing 1 additional heteroatom selected from N, O and S; which is optionally substituted by 1-5 substituents independently selected from (C₁-C₆)alkyl, (C₁-C₄)haloalkyl, —CO(C₁-C₆)alkyl, amino(C₁-C₄ alkyl)-, (C₁-C₄ alkyl)amino(C₁-C₄ alkyl)-, (C₁-C₄ alkyl)(C₁-C₄ alkyl)amino(C₁-C₄ alkyl)-, and oxo;
Z is O or NR²;
R² is H, (C₁-C₄)alkyl, (C₃-C₆)cycloalkyl, hydroxy(C₂-C₄)alkyl-, (C₁-C₆)alkoxy(C₂-C₄)alkyl-, amino(C₂-C₄)alkyl-, (C₁-C₆)alkylamino(C₂-C₄)alkyl-, ((C₁-C₆)alkyl)((C₁-C₆)alkyl)amino(C₂-C₄)alkyl, —CO₂H, —CO₂(C₁-C₆)alkyl, —CONH₂, —CONH(C₁-C₆)alkyl, —CON((C₁-C₆)alkyl)((C₁-C₆)alkyl), —(C₁-C₄)alkylCO₂H, —(C₁-C₄)alkylCO₂(C₁-C₆)alkyl, —(C₁-C₄)alkylCONH₂, —(C₁-C₄)alkylCONH(C₁-C₆)alkyl, —(C₁-C₄)alkylCON((C₁-C₆)alkyl)((C₁-C₆)alkyl), phenyl or 5-6 membered heteroaryl, where said phenyl or 5-6 membered heteroaryl is optionally substituted by 1-3 substituents independently selected from halogen, (C₁-C₆)alkyl, (C₁-C₄)haloalkyl, (C₁-C₆)alkoxy, (C₁-C₄)haloalkoxy, —CO₂H and —CO₂(C₁-C₄)alkyl;
R³ is selected from H, methyl, trifluoromethyl and phenyl;
R⁴ is selected from H and methyl; or
R³ and R⁴ taken together with the atoms through which they are attached form, a 5-6 membered non-aromatic carbocyclic ring; and
R⁵ is H or (C₁-C₄)alkyl;
wherein at least one of R², R³, and R⁴ is not H; or preferably, at least two of R², R³, and R⁴ are not H;
provided that the compound is not:
N²-(3-{[2-(diethylamino)ethyl]oxy}phenyl)-N⁴-(1,3-dimethyl-1H-pyrazol-5-yl)-2,4-pyrimidinediamine;
N²-[3-{[2-(diethylamino)ethyl]oxy}-4-(methyloxy)phenyl]-N-(1-ethyl-1H-pyrazol-5-yl)-2,4-pyrimidinediamine; or
N²-[3-{[2-(diethylamino)ethyl]oxy}-4-(methyloxy)phenyl]-N⁴-(3-methyl-1-phenyl-1H-pyrazol-5-yl)-2,4-pyrimidinediamine;
or a salt thereof.

33. The compound or salt according to claim 31, wherein each R¹ is independently selected from halogen, (C₁-C₄)alkoxy, —SO₂(C₁-C₄)alkyl, —SO₂NR^{y}R^{z}, and an optionally substituted 6-membered non-aromatic heterocyclic ring,
wherein R^{y} is H, (C₁-C₂ alkyl), or (C₁-C₂ alkyl)(C₁-C₂ alkyl)amino(C₂-C₃ alkyl)-, and R^{z} is H or (C₁-C₂ alkyl), or R^{y} and R^{z}, taken together are —CH₂CH₂CH₂CH₂—,
and wherein the 6-membered non-aromatic heterocyclic ring contains one heteroatom selected from N, O and S, or contains one nitrogen atom and one additional heteroatom selected from N, O and S, and is optionally substituted by 1-4 substituents independently selected from (C₁-C₄)alkyl and when the 6-membered non-aromatic heterocyclic rings contains a nitrogen atom, the nitrogen atom is optionally substituted by (C₁-C₄)alkyl, —CO(C₁-C₆)alkyl, amino(C₂-C₄ alkyl)-, (C₁-C₄ alkyl)amino(C₂-C₄ alkyl)-, or (C₁-C₄ alkyl)(C₁-C₄ alkyl)amino(C₂-C₄ alkyl)-.

34. The compound or salt according to claim 31, wherein R¹ is —SO₂R^{x}, —SO₂NR^{y}R^{z}, —SO₂-heterocycloalkyl or heterocycloalkyl, wherein
R^{x} is (C₁-C₄)alkyl, trifluoromethyl, hydroxy(C₂-C₄)alkyl-, cyclopentyl, cyclohexyl;
R^{y} is H, (C₁-C₂)alkyl, hydroxy(C₂-C₃)alkyl-, (C₁-C₂ alkyl)(C₁-C₂ alkyl)amino(C₂-C₃ alkyl)-, cyclopentyl, or piperidinyl, where the piperidinyl is optionally substituted by 1 or 2 substituents independently selected from hydroxy and (C₁-C₂)alkyl;
R^{z} is H or (C₁-C₂ alkyl),
any of said heterocycloalkyl is an optionally substituted 5-6 membered non-aromatic heterocyclic ring, wherein the 5 or 6-membered non-aromatic heterocyclic ring contains one heteroatom selected from N and O, or contains one nitrogen atom and one additional heteroatom selected from N and O, and is optionally substituted by 1-3 independently selected (C₁-C₂)alkyl substituents,
and, when n is 2 or 3, each other R¹ is independently selected from halogen, (C₁-C₂)alkyl, halo(C₁-C₂)alkyl, hydroxy, (C₁-C₂) alkoxy, halo(C₁-C₂)alkoxy, and —SO₂(C₁-C₄)alkyl.

35. The compound or salt according to claim 31, wherein each R¹ is independently selected from hydroxy, cyano, chloro, fluoro, —OCH₃, —OCH₂CH₃, —OCHF₂, —CH₃, —CF₃, —CH(CF₃)N(CH₃)₂, —CN, —C(CN)(CH₃)₂, —CONH₂, —CO₂CH₂CH₃, —S—CH(CH₃)₂, —S—C(CH₃)₂CH₂OH, —S—C(CH₃)₂CO₂CH₂CH₃, —SO₂CH₃, —SO₂CF₃, —SO₂CH₂CH₃, —SO₂CH(CH₃)₂, —SO₂C(CH₃)₃, —SO₂CH₂CH₂OH, —SO₂C(CH₃)₂CH₂OH, —SO₂CH(CH₃)CH₂OH, —SO₂-cyclopentyl, —SO₂-cyclohexyl, —SO₂NH₂, —SO₂N(CH₃)₂, —NHSO₂CH₃, —SO₂N(CH₂CH₃)₂, —SO₂NH-cyclopentyl, —SO₂NHCH₂CH₂OH, —SO₂N(CH₃)(CH₂CH₂N(CH₃)₂), —SO₂-pyrrolidin-1-yl, —SO₂-morpholin-4-yl, —SO₂-(3R)-3-methyl-morpholin-4-yl, —SO₂-(3S)-3-methyl-morpholin-4-yl, —SO₂-tetrahydropyran-4-yl, —SO₂-(2-methyl-tetrahydrofuran-3-yl), —SO₂-(4-hydroxy-piperidin-1-yl), -pyrrolidin-1-yl, 4-methyl-piperazin-1-yl, oxazol-2-yl and benzoxazol-2-yl.

36. The compound or salt according to claim 31, wherein R¹ is —SO₂CH₃, and R^{1A} taken together with an adjacent R¹ group form a —OCH₂CH₂— moiety.

37. The compound or salt according to claim 31, wherein R^{1A} taken together with an adjacent R¹ group form a —CH═CH₂S— moiety or two adjacent R¹ groups form a —SO₂NHCH₂— or —CH₂SO₂CH₂— moiety.

38. The compound or salt according to claim 31, wherein Z is O.

39. The compound or salt according to claim 31, wherein Z is NR²; where R² is H, (C₁-C₄)alkyl, hydroxy(C₂-C₄)alkyl-, (C₁-C₆)alkoxy(C₂-C₄)alkyl-, amino(C₂-C₄)alkyl-, (C₁-C₆)alkylamino(C₂-C₄)alkyl-, ((C₁-C₆)alkyl)((C₁-C₆)alkyl)amino(C₂-C₄)alkyl, 5-6 membered cycloalkyl, phenyl, 5-6 membered heterocycloalkyl, 5-6 membered heterocycloalkylmethyl-, or 5-6 membered heteroaryl, where said 5-6 membered cycloalkyl, phenyl, 5-6 membered heterocycloalkyl, 5-6 membered heterocycloalkylmethyl-, or 5-6 membered heteroaryl is optionally substituted by 1-3 substituents independently selected from halogen, hydroxy, (C₁-C₄)alkyl, (C₁-C₄)haloalkyl, (C₁-C₄)alkoxy, (C₁-C₄)haloalkoxy, —CO₂H, —CO₂(C₁-C₄)alkyl and —CO₂(C₁-C₄)alkylphenyl.

40. The compound or salt according to claim 31, wherein Z is NR²; where R² is H, (C₁-C₄)alkyl, hydroxy(C₂-C₄)alkyl-, (C₁-C₄)alkoxy(C₂-C₄)alkyl-, or an optionally substituted cyclohexyl, phenyl, tetrahydropyranyl, tetrahydropyranylmethyl-, piperidinyl, or pyridyl, where the optionally substituted cyclohexyl, phenyl, tetrahydropyranyl, piperidinyl, or pyridyl is optionally substituted by 1-2 substituents independently selected from halogen, hydroxy, (C₁-C₄)alkyl, (C₁-C₄)alkoxy, and carboxy.

41. The compound or salt according to claim 31, wherein Z is NR²; where H, methyl, 2-hydroxyethyl-, 2-methoxyethyl-, cyclohexyl, 2-hydroxy-cyclohexyl, 1-benzyloxycarbonyl-piperidin-4-yl, phenyl, 3-chloro-phenyl, 4-chloro-phenyl, 3-chloro-4-methyl-phenyl, 3-carboxy-phenyl, 2-methyl-phenyl, 3-methyl-phenyl, 4-methyl-phenyl, 3,4-dimethyl-phenyl, 3-methoxy-phenyl, 4-methoxy-phenyl, pyrid-2-yl, pyrid-3-yl, pyrid-4-yl, 5-chloro-pyrid-2-yl, 6-methyl-pyrid-2-yl, 6-methyl-pyrid-3-yl, 6-methoxy-pyrid-3-yl, 5-methyl-pyrid-2-yl, 5-methoxy-pyrid-2-yl, tetrahydropyran-4-yl or tetrahydropyran-4-yl-methyl-.

42. The compound or salt according to claim 31, wherein R³ is H or R³ is methyl.

43. The compound or salt according to claim 31, wherein R³ is trifluoromethyl or R³ is phenyl.

44. The compound or salt according to claim 31, wherein R⁴ is H or R⁴ is methyl.

45. The compound or salt according to claim 31, wherein R³ and R⁴ taken together with the atoms through which they are attached form a 5 or 6 membered unsubstituted non-aromatic carbocyclic ring.

46. The compound or salt according to claim 31, wherein R⁵ is H or R⁵ is methyl.

47. The compound or salt according to claim 31, wherein R⁴ and R⁵ taken together with the atoms through which they are attached form a 5 or 6 membered unsubstituted non-aromatic heterocyclic ring.

48. The compound or salt according to claim 31, wherein at least two of R², R³, and R⁴ are not H.

49. A compound which is:
N⁴-(5-fluoro-1H-indazol-3-yl)-N⁴-methyl-N²-[3,4,5-tris(methyloxy)phenyl]-2,4-pyrimidinediamine,
N²-[3,4-bis(methyloxy)phenyl]-N⁴-1H-pyrazolo[3,4-b]pyridin-3-yl-2,4-pyrimidinediamine,
3-({4-[(5-fluoro-1H-indazol-3-yl)amino]-2-pyrimidinyl}amino)-N,N-dimethyl-benzenesulfonamide,
N⁴-(5-fluoro-1H-indazol-3-yl)-N²-[3-(methoxy)-5-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3-methyl-5-(methylsulfonyl)phenyl]pyrimidine-2,4-diamine,
N⁴-(5-fluoro-1H-pyrazolo[3,4-b]pyridin-3-yl)-N⁴-methyl-N²-[3-(methyloxy)-5-(methylsulfonyl)phenyl]-2,4-pyrimidinediamine,
2-(ethyloxy)-5-({4-[(5-fluoro-1H-indazol-3-yl)amino]-2-pyrimidinyl}amino)-N,N-dimethylbenzenesulfonamide,
N²-(1,1-dioxido-1-benzothien-4-yl)-N⁴-(5-fluoro-1H-indazol-3-yl)-2,4-pyrimidinediamine,
3-((4-((5-fluoro-6-methyl-1H-indazol-3-yl)(methyl)amino)pyrimidin-2-yl)amino)-5-methylbenzenesulfonamide,
N²-[2,3-dimethyl-5-(methylsulfonyl)phenyl]-N⁴-(5-fluoro-1H-indazol-3-yl)-2,4-pyrimidinediamine,
2-{[3-({4-[(5-fluoro-1H-indazol-3-yl)amino]-2-pyrimidinyl}amino)phenyl]sulfinyl}ethanol,
N²-[3,4-bis(methyloxy)phenyl]-N⁴-[4-(methyloxy)-1H-indazol-3-yl]-2,4-pyrimidinediamine,
N²-[3,4-bis(methyloxy)phenyl]-N⁴-(6-methyl-1H-indazol-3-yl)-2,4-pyrimidinediamine,
N²-[3,4-bis(methyloxy)phenyl]-N⁴-{6-[(phenylmethyl)oxy]-1H-indazol-3-yl}-2,4-pyrimidinediamine,
N²-[3,4-bis(methyloxy)phenyl]-N⁴-1H-indazol-3-yl-2,4-pyrimidinediamine,
N⁴-[5-(methyloxy)-1H-indazol-3-yl]-N²-[3,4,5-tris(methyloxy)phenyl]-2,4-pyrimidinediamine,
N²-[3,4-bis(methyloxy)phenyl]-N⁴-(6-chloro-1H-indazol-3-yl)-2,4-pyrimidinediamine,
N²-[3,4-bis(methyloxy)phenyl]-N⁴-(5-chloro-1H-indazol-3-yl)-2,4-pyrimidinediamine,
N²-[3,4-bis(methyloxy)phenyl]-N⁴-[5-(methyloxy)-1H-indazol-3-yl]-2,4-pyrimidinediamine,
N²-[3,4-bis(methyloxy)phenyl]-N⁴-(7-fluoro-1H-indazol-3-yl)-2,4-pyrimidinediamine,
N⁴-1H-indazol-3-yl-N²-[4-(4-methyl-1-piperazinyl)phenyl]-2,4-pyrimidinediamine,
N²-[3,4-bis(methyloxy)phenyl]-N⁴-(4-fluoro-1H-indazol-3-yl)-2,4-pyrimidinediamine,
N²-[3,4-bis(methyloxy)phenyl]-N⁴-[6-(methyloxy)-1H-indazol-3-yl]-2,4-pyrimidinediamine,
N²-[3,4-bis(methyloxy)phenyl]-N⁴-(4,5-dichloro-1H-indazol-3-yl)-2,4-pyrimidinediamine,
N²-[3,4-bis(methyloxy)phenyl]-N⁴-(6-chloro-1-methyl-1H-indazol-3-yl)-2,4-pyrimidinediamine,
3-[(4-{[5-(methyloxy)-1H-indazol-3-yl]amino}-2-pyrimidinyl)amino]benzenesulfonamide,
N²-[3,4-bis(methyloxy)phenyl]-N⁴-(5-fluoro-1H-indazol-3-yl)-2,4-pyrimidinediamine,
N²-[3,4-bis(methyloxy)phenyl]-N⁴-(5,7-difluoro-1H-indazol-3-yl)-2,4-pyrimidinediamine,
N²-[3,4-bis(methyloxy)phenyl]-N⁴-(6,7-difluoro-1H-indazol-3-yl)-2,4-pyrimidinediamine,
N⁴-(5-fluoro-1H-indazol-3-yl)-N²-[3-(1-pyrrolidinylsulfonyl)phenyl]-2,4-pyrimidinediamine,
N²-[4-fluoro-3-(methylsulfonyl)phenyl]-N⁴-[5-(methyloxy)-1H-indazol-3-yl]-2,4-pyrimidinediamine,
N⁴-(5-fluoro-1H-indazol-3-yl)-N²-[4-fluoro-3-(methylsulfonyl)phenyl]-2,4-pyrimidinediamine,
N⁴-(5-fluoro-1H-indazol-3-yl)-N⁴-methyl-N²-[3,4,5-tris(methyloxy)phenyl]-2,4-pyrimidinediamine,
N⁴-(5-fluoro-1H-indazol-3-yl)-N⁴-methyl-N²-[3-(methylsulfonyl)phenyl]-2,4-pyrimidinediamine,
N²-[3,4-bis(methyloxy)phenyl]-N⁴-[1-methyl-5-(methyloxy)-1H-indazol-3-yl]-2,4-pyrimidinediamine,
N⁴-[5-(methyloxy)-1H-indazol-3-yl]-N²-[3,4,5-tris(methyloxy)phenyl]-2,4-pyrimidinediamine,
N²-[4-fluoro-3-(methyloxy)phenyl]-N⁴-[5-(methyloxy)-1H-indazol-3-yl]-2,4-pyrimidinediamine,
N-[2-(dimethylamino)ethyl]-N-methyl-3-[(4-{[5-(methyloxy)-1H-indazol-3-yl]amino}-2-pyrimidinyl)amino]benzenesulfonamide,
N,N-dimethyl-3-[(4-{[5-(methyloxy)-1H-indazol-3-yl]amino}-2-pyrimidinyl)amino]benzenesulfonamide,
N⁴-[5-(methyloxy)-1H-indazol-3-yl]-N²-[3-(1-pyrrolidinylsulfonyl)phenyl]-2,4-pyrimidinediamine,
N²-{3-[1-(dimethylamino)-2,2,2-trifluoroethyl]phenyl}-N⁴-(5-fluoro-1H-indazol-3-yl)-2,4-pyrimidinediamine,
2-[3-({4-[(5-fluoro-1H-indazol-3-yl)amino]-2-pyrimidinyl}amino)phenyl]-2-methylpropanenitrile,
N⁴-(5-fluoro-1H-indazol-3-yl)-N²-[3-(methylsulfonyl)phenyl]-2,4-pyrimidinediamine,
N²-(2,3-dihydro-1,4-benzodioxin-6-yl)-N⁴-(5-fluoro-1H-indazol-3-yl)-2,4-pyrimidinediamine,
N⁴-(5-fluoro-1H-indazol-3-yl)-N²-[4-methyl-3-(methylsulfonyl)phenyl]-2,4-pyrimidinediamine,
2-{[3-({4-[(5-fluoro-1H-indazol-3-yl)amino]-2-pyrimidinyl}amino)phenyl]sulfonyl}-2-methyl-1-propanol,
4-({4-[(5-fluoro-1H-indazol-3-yl)amino]-2-pyrimidinyl}amino)-2,6-bis(methyloxy)phenol,
N⁴-(5-fluoro-1H-indazol-3-yl)-N²-1H-indol-6-yl-2,4-pyrimidinediamine,
N⁴-(5-fluoro-1H-indazol-3-yl)-N²-1H-indol-4-yl-2,4-pyrimidinediamine,
N⁴-(5-fluoro-1H-indazol-3-yl)-N²-[1-(methylsulfonyl)-1H-indol-6-yl]-2,4-pyrimidinediamine,
N⁴-(5-fluoro-1H-indazol-3-yl)-N²-[2-methyl-3-(methylsulfonyl)phenyl]-2,4-pyrimidinediamine,
N⁴-(5-fluoro-1H-indazol-3-yl)-N²-[1-(methylsulfonyl)-1H-indol-5-yl]-2,4-pyrimidinediamine,
N⁴-(7-chloro-1H-indazol-3-yl)-N²-[4-methyl-3-(methylsulfonyl)phenyl]-2,4-pyrimidinediamine,
N²-[4-methyl-3-(methylsulfonyl)phenyl]-N⁴-[7-(trifluoromethyl)-1H-indazol-3-yl]-2,4-pyrimidinediamine,
N⁴-(5-fluoro-1H-indazol-3-yl)-N²-[3-methyl-5-(methylsulfonyl)phenyl]-2,4-pyrimidinediamine,
N⁴-(5-fluoro-1H-indazol-3-yl)-N²-[4-(methylsulfonyl)phenyl]-2,4-pyrimidinediamine,
N⁴-(5-fluoro-1H-indazol-3-yl)-N²-[3-(4-morpholinylsulfonyl)phenyl]-2,4-pyrimidinediamine,
3-({4-[(5-fluoro-1H-indazol-3-yl)amino]-2-pyrimidinyl}amino)-N-(methyloxy)benzenesulfonamide,
3-({4-[(5-fluoro-1H-indazol-3-yl)amino]-2-pyrimidinyl}amino)-N-methyl-N-(methyloxy)benzenesulfonamide,
5-({4-[(5-fluoro-1H-indazol-3-yl)amino]-2-pyrimidinyl}amino)-N,2-dimethyl-N-(methyloxy)benzenesulfonamide,
N²-[3-(ethyloxy)-5-(methylsulfonyl)phenyl]-N⁴-(5-fluoro-1H-indazol-3-yl)-2,4-pyrimidinediamine,
N⁴-(5-fluoro-1H-indazol-3-yl)-N²-{3-[(1-methylethyl)sulfonyl]phenyl}-2,4-pyrimidinediamine,
N²-(3,5-dimethylphenyl)-N⁴-(5-fluoro-1H-indazol-3-yl)-2,4-pyrimidinediamine,
N⁴-(5-fluoro-1H-indazol-3-yl)-N²-{4-methyl-3-[(1-methylethyl)sulfonyl]phenyl}-2,4-pyrimidinediamine,
N⁴-(5-fluoro-1H-indazol-3-yl)-N²-[4-methyl-3-(methylsulfonyl)phenyl]-2,4-pyrimidinediamine,
2-(ethyloxy)-5-({4-[(5-fluoro-1H-indazol-3-yl)amino]-2-pyrimidinyl}amino)-N,N-dimethylbenzenesulfonamide,
N⁴-(5-fluoro-1H-indazol-3-yl)-N²-[3-(methyloxy)-5-(methylsulfonyl)phenyl]-2,4-pyrimidinediamine,
N²-[3-(ethylsulfonyl)-5-(methyloxy)phenyl]-N⁴-(5-fluoro-1H-indazol-3-yl)-2,4-pyrimidinediamine,
N²-{4-(ethyloxy)-3-[(1-methylethyl)sulfonyl]phenyl}-N⁴-(5-fluoro-1H-indazol-3-yl)-2,4-pyrimidinediamine,
N⁴-(5-fluoro-1H-indazol-3-yl)-N⁴-methyl-N²-[4-methyl-3-(methylsulfonyl)phenyl]-2,4-pyrimidinediamine,
N⁴-(5-fluoro-1H-indazol-3-yl)-N⁴-methyl-N²-[3-methyl-5-(methylsulfonyl)phenyl]-2,4-pyrimidinediamine,
N⁴-ethyl-N⁴-(5-fluoro-1H-indazol-3-yl)-N²-[3-methyl-5-(methylsulfonyl)phenyl]-2,4-pyrimidinediamine,
N⁴-(5-fluoro-1H-indazol-3-yl)-N²-[3-(methylsulfonyl)-5-(4-morpholinyl)phenyl]-2,4-pyrimidinediamine,
N⁴-(5-fluoro-1H-indazol-3-yl)-N²-(1-methyl-1H-imidazol-2-yl)-2,4-pyrimidinediamine,
N⁴-(5-fluoro-1H-indazol-3-yl)-N²-[3-(methyloxy)-5-(methylsulfonyl)phenyl]-2,4-pyrimidinediamine,
N⁴-(5-fluoro-1H-indazol-3-yl)-N²-[3-fluoro-5-(methylsulfonyl)phenyl]-2,4-pyrimidinediamine,
2-{[5-({4-[(5-fluoro-1H-indazol-3-yl)amino]-2-pyrimidinyl}amino)-2-methylphenyl]sulfonyl}-2-methyl-1-propanol,
N⁴-(5-fluoro-1H-indazol-3-yl)-N²-{4-fluoro-3-[(1-methylethyl)sulfonyl]phenyl}-2,4-pyrimidinediamine,
N⁴-(5-fluoro-1H-indazol-3-yl)-N⁴-methyl-N²-[3-(methyloxy)-5-(methylsulfonyl)phenyl]-2,4-pyrimidinediamine,
N⁴-(5-fluoro-1H-indazol-3-yl)-N²-[3-fluoro-4-methyl-5-(methylsulfonyl)phenyl]-2,4-pyrimidinediamine,
N-[3-({4-[(5-fluoro-1H-indazol-3-yl)amino]-2-pyrimidinyl}amino)phenyl]-N-methylmethanesulfonamide,
N²-[3-(dimethylamino)-5-(methylsulfonyl)phenyl]-N⁴-(5-fluoro-1H-indazol-3-yl)-2,4-pyrimidinediamine,
3-({4-[(5-fluoro-1H-indazol-3-yl)amino]-2-pyrimidinyl}amino)benzamide,
N⁴-(5-fluoro-1H-indazol-3-yl)-N²-[2-fluoro-4-(methylsulfonyl)phenyl]-2,4-pyrimidinediamine,
N⁴-(5-fluoro-1H-indazol-3-yl)-N²-[3-(methylsulfonyl)-5-(1-pyrrolidinyl)phenyl]-2,4-pyrimidinediamine,
N²-[3-(ethyloxy)-5-(ethylsulfonyl)phenyl]-N⁴-(5-fluoro-1H-indazol-3-yl)-2,4-pyrimidinediamine,
N⁴-(5-fluoro-1H-indazol-3-yl)-N²-[4-(methylsulfonyl)-2,3-dihydro-1-benzofuran-6-yl]-2,4-pyrimidinediamine,
N⁴-(5-fluoro-1H-indazol-3-yl)-N²-[4-fluoro-3-(methylsulfonyl)phenyl]-N⁴-methyl-2,4-pyrimidinediamine,
N⁴-(5-fluoro-1H-indazol-3-yl)-N⁴-methyl-N²-[2-methyl-3-(methylsulfonyl)phenyl]-2,4-pyrimidinediamine,
N²-[3,4-dimethyl-5-(methylsulfonyl)phenyl]-N⁴-(5-fluoro-1H-indazol-3-yl)-N⁴-methyl-2,4-pyrimidinediamine,
N⁴-(5-fluoro-1H-indazol-3-yl)-N²-[2-(methyloxy)-5-(methylsulfonyl)phenyl]-2,4-pyrimidinediamine,
3-({4-[(5-fluoro-1H-indazol-3-yl)amino]-2-pyrimidinyl}amino)-4-(methyloxy)benzenesulfonamide,
N⁴-(5-fluoro-1H-indazol-3-yl)-N²-{3-[(trifluoromethyl)sulfonyl]phenyl}-2,4-pyrimidinediamine,
N²-1-benzothien-4-yl-N⁴-(5-fluoro-1H-indazol-3-yl)-2,4-pyrimidinediamine,
N²-[2,4-dimethyl-5-(methylsulfonyl)phenyl]-N⁴-(5-fluoro-1H-indazol-3-yl)-2,4-pyrimidinediamine,
7-({4-[(5-fluoro-1H-indazol-3-yl)amino]-2-pyrimidinyl}amino)-2,3-dihydro-1-benzofuran-5-sulfonamide,
N⁴-(5-fluoro-1H-indazol-3-yl)-N²-[2-fluoro-3-(methylsulfonyl)phenyl]-2,4-pyrimidinediamine,
N²-[4-methyl-3-(methylsulfonyl)phenyl]-N⁴-[6-(trifluoromethyl)-1H-indazol-3-yl]-2,4-pyrimidinediamine,
N⁴-(6,7-difluoro-1H-indazol-3-yl)-N²-[4-methyl-3-(methylsulfonyl)phenyl]-2,4-pyrimidinediamine,
N⁴-(6-methyl-1H-indazol-3-yl)-N²-[4-methyl-3-(methylsulfonyl)phenyl]-2,4-pyrimidinediamine,
N⁴-(5-chloro-1H-indazol-3-yl)-N²-[4-methyl-3-(methylsulfonyl)phenyl]-2,4-pyrimidinediamine,
N⁴-(4-chloro-1H-indazol-3-yl)-N²-[4-methyl-3-(methylsulfonyl)phenyl]-2,4-pyrimidinediamine,
N⁴-(7-fluoro-1H-indazol-3-yl)-N²-[4-methyl-3-(methylsulfonyl)phenyl]-2,4-pyrimidinediamine,
N⁴-(5,7-difluoro-1H-indazol-3-yl)-N²-[4-methyl-3-(methylsulfonyl)phenyl]-2,4-pyrimidinediamine,
N⁴-(5,6-difluoro-1H-indazol-3-yl)-N²-[4-methyl-3-(methylsulfonyl)phenyl]-2,4-pyrimidinediamine,
N⁴-(5-fluoro-1H-indazol-3-yl)-N²-[2-methyl-5-(methylsulfonyl)phenyl]-2,4-pyrimidinediamine,
N⁴-(5-fluoro-1H-indazol-3-yl)-N²-[3,4,5-tris(methyloxy)phenyl]-2,4-pyrimidinediamine,
N²-[4-methyl-3-(methylsulfonyl)phenyl]-N⁴-[5-(methyloxy)-1H-indazol-3-yl]-2,4-pyrimidinediamine,
N⁴-[3-({4-[(5-fluoro-1H-indazol-3-yl)amino]-2-pyrimidinyl}amino)phenyl]-N,N-dimethylsulfamide,
3-({4-[(5-fluoro-1H-indazol-3-yl)amino]-2-pyrimidinyl}amino)benzenesulfonamide,
5-({4-[(5-fluoro-1H-indazol-3-yl)amino]-2-pyrimidinyl}amino)-2-methylbenzenesulfonamide,
N⁴-(6,7-difluoro-1H-indazol-3-yl)-N⁴-methyl-N²-[4-methyl-3-(methylsulfonyl)phenyl]-2,4-pyrimidinediamine,
N⁴-(6,7-difluoro-1H-indazol-3-yl)-N⁴-methyl-N²-[3-methyl-5-(methylsulfonyl)phenyl]-2,4-pyrimidinediamine,
N⁴-(6,7-difluoro-1H-indazol-3-yl)-N⁴-methyl-N²-[3,4,5-tris(methyloxy)phenyl]-2,4-pyrimidinediamine,
N⁴-(7-fluoro-1H-indazol-3-yl)-N⁴-methyl-N²-[4-methyl-3-(methylsulfonyl)phenyl]-2,4-pyrimidinediamine,
N⁴-(7-fluoro-1H-indazol-3-yl)-N⁴-methyl-N²-[3-methyl-5-(methylsulfonyl)phenyl]-2,4-pyrimidinediamine,
N⁴-(7-fluoro-1H-indazol-3-yl)-N⁴-methyl-N²-[3-(methyloxy)-5-(methylsulfonyl)phenyl]-2,4-pyrimidinediamine,
N⁴-(7-fluoro-1H-indazol-3-yl)-N⁴-methyl-N²-[3,4,5-tris(methyloxy)phenyl]-2,4-pyrimidinediamine,
N⁴-(5,6-difluoro-1H-indazol-3-yl)-N⁴-methyl-N²-[3,4,5-tris(methyloxy)phenyl]-2,4-pyrimidinediamine,
N⁴-(5,6-difluoro-1H-indazol-3-yl)-N⁴-methyl-N²-[3-(methyloxy)-5-(methylsulfonyl)phenyl]-2,4-pyrimidinediamine,
N⁴-(5,6-difluoro-1H-indazol-3-yl)-N⁴-methyl-N²-[4-methyl-3-(methylsulfonyl)phenyl]-2,4-pyrimidinediamine,
N⁴-(5,6-difluoro-1H-indazol-3-yl)-N⁴-methyl-N²-[3-methyl-5-(methylsulfonyl)phenyl]-2,4-pyrimidinediamine,
N⁴-(6,7-difluoro-1H-indazol-3-yl)-N⁴-methyl-N²-[3-(methyloxy)-5-(methylsulfonyl)phenyl]-2,4-pyrimidinediamine,
N⁴-(1H-indazol-3-yl)-N²-(3-methyl-5-(methylsulfonyl)phenyl)pyrimidine-2,4-diamine,
N⁴-(1H-indazol-3-yl)-N²-(4-methyl-3-(methylsulfonyl)phenyl)pyrimidine-2,4-diamine,
N⁴-(7-fluoro-1H-indazol-3-yl)-N²-(3-methyl-5-(methylsulfonyl)phenyl)pyrimidine-2,4-diamine,
N⁴-(4-fluoro-1H-indazol-3-yl)-N⁴-methyl-N²-(4-methyl-3-(methylsulfonyl)phenyl)pyrimidine-2,4-diamine,
N⁴-(6,7-difluoro-1H-indazol-3-yl)-N²-(3-methyl-5-(methylsulfonyl)phenyl)pyrimidine-2,4-diamine,
N⁴-(4-fluoro-1H-indazol-3-yl)-N⁴-methyl-N²-(3-methyl-5-(methylsulfonyl)phenyl)pyrimidine-2,4-diamine,
N⁴-(5-fluoro-6-methyl-1H-indazol-3-yl)-N⁴-methyl-N²-[3-methyl-5-(methylsulfonyl)phenyl]-2,4-pyrimidinediamine,
N⁴-(7-fluoro-6-methyl-1H-indazol-3-yl)-N⁴-methyl-N²-[3-methyl-5-(methylsulfonyl)phenyl]-2,4-pyrimidinediamine,
N⁴-(5,6-difluoro-1H-indazol-3-yl)-N²-[3-methyl-5-(methylsulfonyl)phenyl]-2,4-pyrimidinediamine,
N⁴-(5,6-difluoro-1H-indazol-3-yl)-N²-[3-(methyloxy)-5-(methylsulfonyl)phenyl]-2,4-pyrimidinediamine,
N⁴-(5,6-difluoro-1H-indazol-3-yl)-N²-[3,4,5-tris(methyloxy)phenyl]-2,4-pyrimidinediamine,
N⁴-(6-fluoro-5-methyl-1H-indazol-3-yl)-N⁴-methyl-N²-[3-methyl-5-(methylsulfonyl)phenyl]-2,4-pyrimidinediamine,
2-chloro-5-({4-[(5-fluoro-1H-indazol-3-yl)amino]-2-pyrimidinyl}amino)benzenesulfonamide,
N⁴-(5-fluoro-6-methyl-1H-indazol-3-yl)-N⁴-methyl-N²-[3-methyl-5-(methylsulfonyl)phenyl]-2,4-pyrimidinediamine,
3-({4-[(7-fluoro-1H-indazol-3-yl)(methyl)amino]-2-pyrimidinyl}amino)-5-methylbenzenesulfonamide,
N⁴-(7-fluoro-1H-indazol-3-yl)-N⁴-methyl-N²-(3-(methylsulfonyl)-5-(pyrrolidin-1-yl)phenyl)pyrimidine-2,4-diamine,
3-({4-[(6,7-difluoro-1H-indazol-3-yl)(methyl)amino]-2-pyrimidinyl}amino)-5-methylbenzenesulfonamide,
N⁴-(7-fluoro-1H-indazol-3-yl)-N⁴-methyl-N²-(3-methyl-5-(pyrrolidin-1-yl)phenyl)pyrimidine-2,4-diamine,
N⁴-(7-fluoro-1H-indazol-3-yl)-N⁴-methyl-N²-(3-methyl-5-morpholinophenyl)pyrimidine-2,4-diamine,
N⁴-(7-fluoro-6-methyl-1H-indazol-3-yl)-N⁴-methyl-N²-[3-(methyloxy)-5-(methylsulfonyl)phenyl]-2,4-pyrimidinediamine,
N⁴-(5-fluoro-6-methyl-1H-indazol-3-yl)-N⁴-methyl-N²-[3-(methyloxy)-5-(methylsulfonyl)phenyl]-2,4-pyrimidinediamine,
N⁴-(6,7-difluoro-1H-indazol-3-yl)-N²-(1,1-dioxido-1-benzothien-6-yl)-N⁴-methyl-2,4-pyrimidinediamine,
N⁴-methyl-N⁴-(5-methyl-1H-indazol-3-yl)-N²-[3-(methyloxy)-5-(methylsulfonyl)phenyl]-2,4-pyrimidinediamine,
N⁴-methyl-N⁴-(5-methyl-1H-indazol-3-yl)-N²-[3-methyl-5-(methylsulfonyl)phenyl]-2,4-pyrimidinediamine,
3-({4-[(7-fluoro-6-methyl-1H-indazol-3-yl)(methyl)amino]-2-pyrimidinyl}amino)-5-methylbenzenesulfonamide,
N⁴-(7-fluoro-1H-indazol-3-yl)-N²-(3-methoxy-5-(methylsulfonyl)phenyl)pyrimidine-2,4-diamine,
5-((4-((7-fluoro-1H-indazol-3-yl)(methyl)amino)pyrimidin-2-yl)amino)-2-methoxybenzenesulfonamide,
N⁴-(6-methyl-1H-indazol-3-yl)-N²-[3-methyl-5-(methylsulfonyl)phenyl]-2,4-pyrimidinediamine,
N⁴-(6,7-difluoro-1H-indazol-3-yl)-N²-[3-(methyloxy)-5-(methylsulfonyl)phenyl]-2,4-pyrimidinediamine,
3-((4-((5,6-difluoro-1H-indazol-3-yl)(methyl)amino)pyrimidin-2-yl)amino)-5-methylbenzenesulfonamide,
N⁴-(5,6-difluoro-1H-indazol-3-yl)-N²-(4-methoxy-3-(methylsulfonyl)phenyl)-N⁴-methylpyrimidine-2,4-diamine,
N⁴-(7-fluoro-1H-indazol-3-yl)-N²-(4-methoxy-3-(methylsulfonyl)phenyl)-N⁴-methylpyrimidine-2,4-diamine,
N⁴-(7-fluoro-1H-indazol-3-yl)-N⁴-methyl-N²-(3-methyl-5-((tetrahydro-2H-pyran-4-yl)sulfonyl)phenyl)pyrimidine-2,4-diamine,
3-({4-[(6,7-difluoro-1H-indazol-3-yl)(methyl)amino]-2-pyrimidinyl}amino)-5-(methyloxy)benzenesulfonamide,
3-((4-((5,6-difluoro-1H-indazol-3-yl)(methyl)amino)pyrimidin-2-yl)amino)-5-methoxybenzenesulfonamide,
3-({4-[(5-fluoro-6-methyl-1H-indazol-3-yl)(methyl)amino]-2-pyrimidinyl}amino)-5-(methyloxy)benzenesulfonamide,
3-({4-[(7-fluoro-6-methyl-1H-indazol-3-yl)(methyl)amino]-2-pyrimidinyl}amino)-5-(methyloxy)benzenesulfonamide,
3-({4-[(6,7-difluoro-1H-indazol-3-yl)amino]-2-pyrimidinyl}amino)-5-methylbenzenesulfonamide,
3-((4-((7-fluoro-1H-indazol-3-yl)amino)pyrimidin-2-yl)amino)-5-methoxybenzenesulfonamide,
3-((4-((7-fluoro-1H-indazol-3-yl)(methyl)amino)pyrimidin-2-yl)amino)-5-methoxybenzenesulfonamide,
N²-[4-(ethyloxy)-3-(methylsulfonyl)phenyl]-N⁴-(7-fluoro-1H-indazol-3-yl)-N⁴-methyl-2,4-pyrimidinediamine,
3-methoxy-5-(4-(methyl(7-(trifluoromethyl)-1H-indazol-3-yl)amino)pyrimidin-2-yl)amino)benzenesulfonamide,
3-methyl-5-[(4-{methyl[6-(trifluoromethyl)-1H-indazol-3-yl]amino}-2-pyrimidinyl)amino]benzenesulfonamide,
3-(methyloxy)-5-[(4-{methyl[6-(trifluoromethyl)-1H-indazol-3-yl]amino}-2-pyrimidinyl)amino]benzenesulfonamide,
3-methyl-5-(4-(methyl(7-(trifluoromethyl)-1H-indazol-3-yl)amino)pyrimidin-2-yl)amino)benzenesulfonamide,
N,N-dimethyl-3-{[4-(1H-pyrazolo[3,4-b]pyridin-3-ylamino)-2-pyrimidinyl]amino}benzenesulfonamide,
N⁴-(5-methyl-1H-pyrazolo[3,4-b]pyridin-3-yl)-N²-[3-(methylsulfonyl)phenyl]-2,4-pyrimidinediamine,
N²-[4-methyl-3-(methylsulfonyl)phenyl]-N⁴-(5-methyl-1H-pyrazolo[3,4-b]pyridin-3-yl)-2,4-pyrimidinediamine,
N²-[4-fluoro-3-(methylsulfonyl)phenyl]-N⁴-(5-methyl-1H-pyrazolo[3,4-b]pyridin-3-yl)-2,4-pyrimidinediamine,
N⁴-(5-fluoro-1H-pyrazolo[3,4-b]pyridin-3-yl)-N²-[3-(methylsulfonyl)phenyl]-2,4-pyrimidinediamine,
N²-[4-fluoro-3-(methylsulfonyl)phenyl]-N⁴-(5-fluoro-1H-pyrazolo[3,4-b]pyridin-3-yl)-2,4-pyrimidinediamine,
N⁴-(5-fluoro-1H-pyrazolo[3,4-b]pyridin-3-yl)-N²-[4-methyl-3-(methylsulfonyl)phenyl]-2,4-pyrimidinediamine,
N⁴-(5-fluoro-1H-pyrazolo[3,4-b]pyridin-3-yl)-N²-{4-methyl-3-[(1-methylethyl)sulfonyl]phenyl}-2,4-pyrimidinediamine,
N⁴-(5-fluoro-1H-pyrazolo[3,4-b]pyridin-3-yl)-N⁴-methyl-N²-[3-(methylsulfonyl)phenyl]-2,4-pyrimidinediamine,
N⁴-(5-fluoro-1H-pyrazolo[3,4-b]pyridin-3-yl)-N²-{4-methyl-3-[(1-methylethyl)sulfonyl]phenyl}-2,4-pyrimidinediamine,
N⁴-(5-fluoro-1H-pyrazolo[3,4-b]pyridin-3-yl)-N²-[3-(methyloxy)-5-(methylsulfonyl)phenyl]-2,4-pyrimidinediamine,
N⁴-(5-fluoro-1H-pyrazolo[3,4-b]pyridin-3-yl)-N⁴-methyl-N²-[3-methyl-5-(methylsulfonyl)phenyl]-2,4-pyrimidinediamine,
N⁴-(5-fluoro-1H-pyrazolo[3,4-b]pyridin-3-yl)-N⁴-methyl-N²-[4-methyl-3-(methylsulfonyl)phenyl]-2,4-pyrimidinediamine,
N⁴-(5-fluoro-1H-pyrazolo[3,4-b]pyridin-3-yl)-N²-[3-(methylsulfonyl)-5-(4-morpholinyl)phenyl]-2,4-pyrimidinediamine,
N⁴-(5-fluoro-1H-pyrazolo[3,4-b]pyridin-3-yl)-N²-[4-methyl-3-(methylsulfonyl)phenyl]-2,4-pyrimidinediamine,
N²-[3,4-dimethyl-5-(methylsulfonyl)phenyl]-N⁴-(5-fluoro-1H-pyrazolo[3,4-b]pyridin-3-yl)-2,4-pyrimidinediamine,
N⁴-(5-fluoro-1H-pyrazolo[3,4-b]pyridin-3-yl)-N⁴-methyl-N²-[4-methyl-3-(methylsulfonyl)-5-(1-pyrrolidinyl)phenyl]-2,4-pyrimidinediamine,
N⁴-(6-methyl-1H-pyrazolo[3,4-b]pyridin-3-yl)-N²-[3-(methylsulfonyl)phenyl]-2,4-pyrimidinediamine,
N²-[3-[(difluoromethyl)oxy]-5-(methylsulfonyl)phenyl]-N⁴-(5-fluoro-1H-pyrazolo[3,4-b]pyridin-3-yl)-N⁴-methyl-2,4-pyrimidinediamine,
N²-[3-(3,3-difluoro-1-pyrrolidinyl)-5-(methylsulfonyl)phenyl]-N⁴-(5-fluoro-1H-pyrazolo[3,4-b]pyridin-3-yl)-N⁴-methyl-2,4-pyrimidinediamine,
N⁴-(5-fluoro-1H-pyrazolo[3,4-b]pyridin-3-yl)-N⁴-methyl-N²-[4-(methylsulfonyl)-2,3-dihydro-1-benzofuran-6-yl]-2,4-pyrimidinediamine,
N⁴-(5-fluoro-1H-pyrazolo[3,4-b]pyridin-3-yl)-N²-[3,4,5-tris(methyloxy)phenyl]-2,4-pyrimidinediamine,
N⁴-(5-fluoro-1H-pyrazolo[3,4-b]pyridin-3-yl)-N⁴-methyl-N²-[3,4,5-tris(methyloxy)phenyl]-2,4-pyrimidinediamine,
N²-[4-methyl-3-(methylsulfonyl)phenyl]-N⁴-(6-methyl-1H-pyrazolo[3,4-b]pyridin-3-yl)-2,4-pyrimidinediamine,
N²-{3-[(1,1-dimethylethyl)sulfonyl]-5-methylphenyl}-N⁴-(5-fluoro-1H-pyrazolo[3,4-b]pyridin-3-yl)-2,4-pyrimidinediamine,
N²-[3-[(1,1-dimethylethyl)sulfonyl]-5-(trifluoromethyl)phenyl]-N⁴-(5-fluoro-1H-pyrazolo[3,4-b]pyridin-3-yl)-2,4-pyrimidinediamine,
N⁴-(5-fluoro-6-methyl-1H-pyrazolo[3,4-b]pyridin-3-yl)-N⁴-methyl-N²-[3-(methyloxy)-5-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3-methyl-5-(methylsulfonyl)phenyl]pyrimidine-2,4-diamine,
N⁴-(5-fluoro-6-methyl-1H-pyrazolo[3,4-b]pyridin-3-yl)-N²-[3-(methyloxy)-5-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3-(methylsulfonyl)-5-(1-pyrrolidinyl)phenyl]-2,4-pyrimidinediamine,
N²-[3-[(difluoromethyl)oxy]-5-(methylsulfonyl)phenyl]-N⁴-(5-fluoro-6-methyl-1H-pyrazolo[3,4-b]pyridin-3-yl)-N⁴-methyl-2,4-pyrimidinediamine,
N⁴-(5-fluoro-6-methyl-1H-pyrazolo[3,4-b]pyridin-3-yl)-N⁴-methyl-N²-[4-methyl-3-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3,4,5-tris(methyloxy)phenyl]-2,4-pyrimidinediamine,
N²-{3-[(1,1-dimethylethyl)sulfonyl]-5-methylphenyl}-N⁴-(5-fluoro-6-methyl-1H-pyrazolo[3,4-b]pyridin-3-yl)-N⁴-methyl-2,4-pyrimidinediamine,
N⁴-(5-fluoro-1,6-dimethyl-1H-pyrazolo[3,4-b]pyridin-3-yl)-N⁴-methyl-N²-[3-methyl-5-(methylsulfonyl)phenyl]-2,4-pyrimidinediamine,
3-({4-[(5-fluoro-6-methyl-1H-pyrazolo[3,4-b]pyridin-3-yl)(methyl)amino]-2-pyrimidinyl}amino)benzenesulfonamide,
3-({4-[(5-fluoro-1H-pyrazolo[3,4-b]pyridin-3-yl)amino]-2-pyrimidinyl}amino)benzenesulfonamide,
N⁴-(5-fluoro-6-methyl-1H-pyrazolo[3,4-b]pyridin-3-yl)-N²-[3-fluoro-5-(methylsulfonyl)phenyl]-N⁴-methyl-2,4-pyrimidinediamine,
3-({4-[(5-fluoro-1H-pyrazolo[3,4-b]pyridin-3-yl)(methyl)amino]-2-pyrimidinyl}amino)benzenesulfonamide,
2-chloro-5-({4-[(5-fluoro-1H-pyrazolo[3,4-b]pyridin-3-yl)amino]-2-pyrimidinyl}amino)benzenesulfonamide,
2-chloro-5-({4-[(5-fluoro-1H-pyrazolo[3,4-b]pyridin-3-yl)(methyl)amino]-2-pyrimidinyl}amino)benzenesulfonamide,
5-({4-[(5-fluoro-1H-pyrazolo[3,4-b]pyridin-3-yl)(methyl)amino]-2-pyrimidinyl}amino)-2-methylbenzenesulfonamide,
N⁴-(5-fluoro-7-oxido-1H-pyrazolo[3,4-b]pyridin-3-yl)-N⁴-methyl-N²-[3-methyl-5-(methylsulfonyl)phenyl]-2,4-pyrimidinediamine,
5-fluoro-6-methyl-3-(methyl(2-((3-methyl-5-(methylsulfonyl)phenyl)amino)pyrimidin-4-yl)amino)-1H-pyrazolo[3,4-b]pyridine 7-oxide, or
N⁴-(5-fluoro-6-methyl-1H-pyrazolo[3,4-b]pyridin-3-yl)-N⁴-methyl-N²-(3-methyl-5-((tetrahydro-2H-pyran-4-yl)sulfonyl)phenyl)pyrimidine-2,4-diamine,
or a pharmaceutically acceptable salt thereof.

50. A pharmaceutical composition comprising the compound according to claim 31, or a pharmaceutically acceptable salt thereof, and a pharmaceutically acceptable excipient.
